(19) United States
(12) Patent Application Publication
(10) Pub. No.: US 2023/0136613 A1
(43) Pub. Date: May 4, 2023

(54) COMPOSITIONS AND METHODS FOR TREATING OR AMELIORATING INFECTIONS

(71) Applicant: San Diego State University (SDSU) Foundation, dba San Diego State University Research Foundation, San Diego, CA (US)

(72) Inventors: Faramarz VALAFAR, San Diego, CA (US); Samuel James MODLIN, San Diego, CA (US); Sarah M. RADECKE, San Diego, CA (US); Emma WESTIN, San Diego, CA (US); Deepika GUNASEKARAN, Dublin, CA (US)

(21) Appl. No.: 17/914,662

(22) PCT Filed: Mar. 26, 2021
(86) PCT No.: PCT/US2021/024528

§ 371 Date: Sep. 26, 2022

Related U.S. Application Data
(60) Provisional application No. 63/000,014, filed on Mar. 26, 2020.

(51) Int. Cl. A61K 31/713; A61K 31/7125

(52) U.S. Cl. CPC A61K 31/713; A61K 31/7125

(57) ABSTRACT

In alternative embodiments, provided are products of manufacture and kits, and methods, that comprise or comprise use of inhibitory molecules for treating or ameliorating, or providing clinical decision support for the treatment of, a bacterial infection, for example, a Mycobacterium tuberculosis (MTB) infection, or an infection by any member species of the M. tuberculosis (MTBC) complex. In alternative embodiments, the inhibitory molecules inhibit the expression of one or more of the 38 PE/PPE MTB genes as identified herein, or one or more of the 366 identical MTB genes and proteins common to a global set of MTB isolates as identified herein, or the inhibitory molecules inhibit the expression of a transcript or a polypeptide gene product thereof, or inhibit the changing (and hence evading the immune system) of one or more of the 52 special immune-evading MTB PE/PPE genes as identified herein

### RELATED APPLICATIONS

This Patent Convention Treaty (PCT) International Application claims benefit of priority of U.S. Provisional Application Ser. No. (USSN) 63/000,014 filed Mar. 26, 2020. The aforementioned application is expressly incorporated herein by reference in its entirety and for all purposes.

### STATEMENT AS TO FEDERALLY SPONSORED RESEARCH

This invention was made with government support under grant No. R01AI105185-06 awarded by NIH. The government has certain rights in the invention.

### TECHNICAL FIELD

This invention generally relates to infectious diseases and microbial genomics. In alternative embodiments, provided are products of manufacture and kits, and methods, that comprise or comprise use of inhibitory molecules for treating or ameliorating, or providing clinical decision support for the treatment of, a bacterial infection, for example, a Mycobacterium tuberculosis (MTB) infection, or an infection by any member species of the M. tuberculosis (MTBC) complex. In alternative embodiments, the inhibitory molecules inhibit the expression of one or more of the 38 PE/PPE MTB genes as identified herein, or one or more of the 366 identical MTB genes and proteins common to a global set of MTB isolates as identified herein, or the inhibitory molecules inhibit the expression of a transcript or a polypeptide gene product thereof, or inhibit the changing (and hence evading the immune system) of one or more of the 52 special immune-evading MTB PE/PPE genes as identified herein.

### BACKGROUND

Mycobacterium tuberculosis has recently surpassed HIV as the deadliest pathogen in the world¹. The success of M. tuberculosis lies in its exceptional ability to evade host immunity and resist antibiotic treatment. However, M. tuberculosis is assumed to have low genetic diversity between strains, often reported to be caused by the lack of horizontal acquisition of genetic material³ and low recombination rates⁴. Gene gain is an important source of diversity, providing a way for organisms to adapt to shifting environmental conditions⁵. Gene gain mechanisms include acquisition of external genetic content, such as horizontal gene transfer (HGT), and duplication⁵. These gain events are freed from selective constraints and can accumulate mutations that produce several fates: maintenance of exact copies, partitioned function, new function, or loss of function⁶. Because M. tuberculosis strains lack HGT³, gene loss is a proposed mechanism of diversity in M. tuberculosis⁷. However, gene duplications have been observed in several M. tuberculosis strains that have influenced various phenotypes^{8,9}, suggesting gene loss is not a dominating source of genetic diversity.

Multigene families are created by repeated duplication events¹⁰ and are susceptible to recombination due to high sequence homology among members. Recombination hotspots¹¹ and gene conversion¹² have been described in two protein families unique to Mycobacteria, PE and PPE¹³. These proteins (characterized by Pro-Glu (PE) at codons 7 and 8 or Pro-Pro-Glu (PPE) at codons 7, 8, and 9¹³) are considered antigens in M. tuberculosis¹⁴ and represent popular vaccine candidates¹⁵. However, the mechanisms driving antigenic variation in the PE/PPE proteins is poorly characterized. Proteins in a PE subfamily, PE_PGRS (polymorphic GC-rich sequences), contain a hypervariable region (PGRS) expected to interact with immune cells, particularly T cells¹⁶. Yet, the PGRS domain is not involved in evasion of T cell recognition due to a significant lack of T cell epitopes in this hypervariable domain¹⁷. Conversely, antigens with tandem repeats have been proposed to elicit a T-cell-independent response¹⁸, which stimulate a B cell response independent of T cell activation¹⁹. The PGRS domain has a tandemly repetitive amino acid motif²⁰, indicating a possible presence of B cell epitopes. Furthermore, recombination may be altering these epitopes to promote evasion, similar to several other pathogens²¹. Previous studies have developed creative methods to elucidate these mechanisms of diversity in M. tuberculosis^{9,11} but have had several limitations and lack resolution. This is largely attributed to the wide-scale use of short-read sequencing, which cannot confidently capture repetitive²² or GC-rich²³ regions (well-known genomic signatures of M. tuberculosis¹³).

Additionally, traditional processing of short-read sequencing relies on a reference-based alignment²⁴, which assumes a similar genome structure as the reference²⁵. Long-read sequencing allows for de novo assembly of a bacterial genome into a single, contiguous sequence, which has revealed genomic changes in a limited number of M. tuberculosis genomes²⁶⁻²⁹. However, these studies lack the sample size to describe the diversity of M. tuberculosis in these regions.

### SUMMARY

In alternative embodiments, provided are methods for treating, preventing or ameliorating infection by a member of the Mycobacterium tuberculosis complex (MTBC), comprising administering to an individual in need thereof at least one inhibitory molecule or composition of:
a. the expression or activity of one or more of the 38 Pro-Glu (PE)/Pro-Pro-Glu (PPE) TB or MTBC genes as identified in TABLE A, or a transcript or a polypeptide encoded by a gene as identified in TABLE A,
b. the expression or activity of one or more of the 366 identical MTBC genes as identified in Table C (see FIG. 33), or a transcript or a polypeptide encoded by a gene as identified in TABLE C; or
c. the antigenic variation of one or more of 52 immune-evading MTBC PE/PPE genes as identified in TABLE B (see FIG. 32), or a transcript or a polypeptide encoded by a gene as identified in TABLE B.

In alternative embodiments, of methods as provided herein:
the inhibitory molecule or composition is formulated as a complementary or a sole therapeutic for treating, preventing or ameliorating an MTBC infection;

the inhibitory molecule or composition acts as a NAC (Non-Antibiotic Chemotherapeutic) molecule that inhibits the MTBC PE/PPE genes listed in 1(c) from changing and evading the immune system, wherein optionally the inhibitory molecule acts as a NAC (Non-Antibiotic Chemotherapeutic) molecule that inhibits targets comprising: the antigenic variation of one or more of 52 immune-evading MTBC PE/PPE genes as identified in TABLE B (see FIG. 32), or a transcript or a polypeptide encoded by the gene, from changing and evading the immune system;
the inhibitory molecule:
(a) is or comprises an inhibitory small molecule, an inhibitory nucleic acid (optionally the inhibitory nucleic acid comprises a miRNA or an antisense molecule), an inhibitory polypeptide or peptide, and optionally the inhibitory polypeptide comprises or is an antibody or antigen binding protein capable of specifically binding to a polypeptide encoded by any of the genes listed in 1(a), (b), or (c); or
(b) is contained in or is expressed by a phage, and optionally the inhibitory peptide or polypeptide is expressed on the surface of the phage;
the inhibitory nucleic acid is or comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme;
the inhibitory molecule or composition is formulated as a pharmaceutical composition or formulation, or is formulated for administration in vivo; or is formulated for enteral or parenteral administration, or for oral, intravenous (IV), intramuscular (IM), or intrathecal (IT) administration, wherein optionally the pharmaceutical compound or formulation is administered orally, parenterally, by inhalation spray, nasally, topically, intrathecally, intrathecally, intracerebrally, epidurally, intracranially or rectally;

the inhibitory molecule or composition, or the formulation or pharmaceutical composition, is contained in or on, or expressed on, or is carried in: a nanoparticle, a particle, a micelle or a liposome or lipoplex, a polymersome, a polyplex, a phage or a dendrimer;
the inhibitory molecule or composition, or the formulation or pharmaceutical composition, is formulated as, or contained in or expressed on: a tablet, a pill, a capsule, a gel, a geltab, a liquid, a powder, an emulsion, a lotion, an aerosol, a spray, a lozenge, an aqueous or a sterile or an injectable solution, or an implant; and/or

the inhibitory nucleic acid is contained in a nucleic acid construct or a chimeric or a recombinant nucleic acid, or an expression cassette, vector, plasmid, phagemid or artificial chromosome, optionally stably integrated into a TB cell's chromosome, or optionally stably episomally expressed in a TB cell;

In alternative embodiments, provided are kits for or treating, preventing or ameliorating an MTBC infection, comprising an inhibitory molecule or composition used in any of the preceding claims, and optionally further comprising instructions for practicing a method as provided herein.

In alternative embodiments, provided are uses of an inhibitory molecule or composition as provided herein for: treating, preventing or ameliorating an MTBC infection, or, for the manufacture of a medicament for treating, preventing or ameliorating a TB infection.

In alternative embodiments, provided are inhibitory molecules or composition as provided herein for use in treating, preventing or ameliorating an MTBC infection.

In alternative embodiments, provided are methods for selecting environmentally-derived or a chimeric genetically engineered phage for formulating a phage or a cocktail of phages that can act as a therapeutic for treating, preventing or ameliorating an MTBC infection,

wherein the environmentally-derived phage or chimeric genetically engineered phage recognizes at least one peptide or a polypeptide encoded by:
(a) one or more of the 38 Pro-Glu (PE)/Pro-Pro-Glu (PPE) TB or M. tuberculosis genes as identified in TABLE A, or a transcript or a polypeptide encoded by a gene as identified in TABLE A,
(b) one or more of the 366 identical MTBC genes as identified in Table C (see FIG. 33), or a transcript or a polypeptide encoded by a gene as identified in TABLE C; or
(c) one or more of 52 special immune-evading MTBC PE/PPE genes as identified in TABLE B (see FIG. 32), or a transcript or a polypeptide encoded by a gene as identified in TABLE B,

wherein optionally the at least one peptide or a polypeptide recognized by the environmentally-derived phage or chimeric genetically engineered phage is expressed on the cell surface of an MTBC, or the at least one peptide or a polypeptide recognized by the environmentally-derived or chimeric genetically engineered phage is not expressed on the cell surface of an MTBC bacterium,

and optionally the at least one peptide or a polypeptide is responsible for synthesis and/or localization of surface molecules and targeted by the phage, optionally a mycobacteriophage,

wherein the method comprises: (i) selecting a set of environmentally-derived or chimeric genetically engineered phages that can attack a desired gene target(s), and/or using the gene targets as a guide to engineer new phages that can target the desired genes; (ii) combinatorial selecting subsets of the selected phages for therapy; (iii) delivering the phage subsets to an MTBC-infected tissue for elimination or amelioration of TB infection, or delivering the subsets to healthy tissues for prevention of an MTBC infection; and, (iv) selecting the effective or most effective subsets for therapeutic application.

In alternative embodiments, the environmentally-derived phage or chimeric genetically engineered phage is a lytic phage or a non-lytic phage, optionally a mycobacteriophage, or the phage therapeutic comprises a plurality of phages, comprising environmentally-derived phage or phages, and/or chimeric genetically engineered phage or phages, that are synergistically effective for treating, prevention or ameliorating TB or MTBC infection.

In alternative embodiments, the phage encodes or comprises a protein toxic to or inhibitory to: (a) one or more of the 38 Pro-Glu (PE)/Pro-Pro-Glu (PPE) TB genes as identified in TABLE A, or a transcript or a polypeptide encoded by a gene as identified in TABLE A; (b) one or more of the 366 identical TB genes as identified in Table C (see FIG. 33), or a transcript or a polypeptide encoded by a gene as identified in TABLE C; or, (c) one or more of 52 special immune-evading TB PE/PPE genes as identified in TABLE B (see FIG. 32), or a transcript or a polypeptide encoded by a gene as identified in TABLE B.

In alternative embodiments, of methods as provided herein:
the phage is a chimeric phage or chimeric genetically engineered phage, and optionally the chimeric phage or chimeric genetically engineered phage is engineered through phage refactoring, wherein optionally the chimeric or genetically engineered phage is a lytic chimeric phage comprising a DNA conferring lytic properties and/or a DNA conferring phage-target recognition, and optionally the DNA conferring lytic capabilities is derived from a lytic mycobacteriophage and the DNA responsible for phage-target recognition is derived from a lysogenic phage; and/or
the phage is formulated into a combination therapy of a phage therapeutic and an existing chemotherapeutic, wherein optionally the mechanism of action of the existing chemotherapeutic and the mechanism of action of the phage act antagonistically with respect to one another.

In alternative embodiments, provided are methods for analyzing sequencing data that can be assembled, de novo, into single contig genomes from DNA isolated from samples of a subspecies, species, collection of species, or genus of interest to identify and prioritize genomic elements as targets for various research and industrial applications where genomic elements' suitability as targets for the application of interest are defined by their essentiality for survival of the organisms,

wherein the method comprises providing or having provided (for example from publicly available databases) DNA sequencing data, such as that obtained from long-read single molecule sequencing of DNA isolated from a collection of samples of interest isolated for a subspecies, species, collection of species, or genus of interest, and for each sample among the set, the method further comprises:

(i) assembling the DNA sequence or sequences that comprise the genome from the sequencing data without reference to a previously known genome structure,

(ii) refining the assembled genome by correcting probable errors through mapping of sequencing reads onto the assembled DNA sequence and correcting the consensus where a majority of mapped reads contradict the consensus sequence; and iteratively repeating this process until convergence (no more consensus corrections) or oscillation between consensus states, or true heterogeneity is otherwise supported. In regions of heterogeneity, alternative consensus sequences are offered representing differences between subpopulations.

(iii) inferring coordinates defining genomic elements within the assembled DNA sequence through a hierarchical annotation prioritizing transfer of coordinates from the annotated genome of a related, well-characterized strain, and secondarily integrating annotations assigned through ab initio and/or orthology-based approaches, and

(iv) determining the core and accessory sets of genomic elements of the set of genomes under examination.

In alternative embodiments, of methods as provided herein:
the methods further comprise use of Artificial Intelligence (AI), Machine Learning (ML), or statistical pattern recognition to analyze the accessory genome of the genomes under examination and to determine a basis for the accessory genome of any species with an open genome to identify patterns that precede and constitute creation of a novel genomic element (optionally a gene);
the methods identify mechanisms of new genomic element creation, and thereby identifying which genomic elements are actively creating new elements; these identified genomic elements will create new genomic elements in at least two senses: first, in the sense that their genetic material are used to create the new genomic elements or features through a wide range of processes (for example mutation, duplication, recombination, gene conversion, fusing, splitting, inversion, or some combination thereof); and, second, in the sense that the ultimate functional products these genomic elements encode (in the case of proteins, for example) or features they comprise (for example, in the case of G4-quadruplexes and other genomic elements with consequences for DNA structure, conformation, or configuration), catalyze, are required for, increase the frequency of events required for, or are otherwise involved in mechanisms of creating new genomic elements, and genomic elements may fulfill both these senses, such as IS6110 movement in M. tuberculosis, wherein the IS6110 element comprises both the DNA sequence being duplicated or transposed and encodes the enzyme catalyzing the requisite biochemical steps for its transposition, and/or
the methods comprise or further comprise predicting upcoming genomic elements and their categorization according to their potential effect on clinical outcome, and/or their clinical utility, wherein potential effects are predicted through one or more of:
d. biological function, as inferred by orthology, homology, or experimentation
e. membership of the predicted element to a mutually exclusive set that includes a member or members with known clinical utility or effect on clinical outcome. This would be taken to imply that the predicted element possesses the same attributes.
f. use metabolic or regulatory modeling to predict the clinical consequence.

In alternative embodiments of methods as provided herein:
the methods are be used as prognostic tools and development of preventative therapeutics or measures. These methods will predict new accessory genes and help in development of preventative therapeutics to target any new genomic element that might attempt to evade the immune system, and/or to target and inhibit the underlying mechanism(s) driving creation of new elements;
the genomic elements are analyzed across samples to discern sets of genomic elements defined according to their pattern of variability among the samples comprising:
g. invariably present genomic elements with little to no sequence variability;
h. invariably present elements with extensive variability in sequence across the entire element or a segment of the element; and

elements that are functionally identical, but different in sequence, and collectively invariably present (one is always present), but invariably, or nearly invariably, mutually exclusive within any single genome;
the methods comprise use of a series of sequencing data processing steps that take sequencing data from a collection of samples for a species of interest and returns or identifies a prioritized list of genomic elements as targets for an application of interest, and tailored according to the species and application of interest, by the class of genomic element and type of variability suitable or desired for the application, for example, expression and invariability for antigenic constituents in vaccine formulations; or conservation and essentiality for drug targets;
the prioritized list of genomic elements comprises priority targets for: (a) intervening in the viability or behavior in a prokaryotic species of interest; or (b) rendering a prokaryotic species of interest non-infective either through: (i) killing the organisms of the species of interest; (ii) rendering the organisms of the species of interest hypersusceptible to common mechanisms of host immunity; or (iii) inoculating the host to the species of interest through exposure to peptides encoded by the identified targets and the molecules they synthesize;
the prokaryotic species of interest is a bacterial pathogen, and optionally the bacterial pathogen is of the genus Mycobacterium, and optionally a species with the genus Mycobacterium is a member species of the Mycobacterium tuberculosis complex;
the host of the bacterial pathogen is a human being;
the bacterial pathogen is formulated in or into an inoculum, and optionally the inoculum is or comprises an acellular vaccine or pharmaceutical formulation, and optionally the inoculum is or comprises a live attenuated vaccine or pharmaceutical formulation; and/or
the identified prioritized list of genomic elements comprises a minimal set for engineering viable strains for an industrial application,

and optionally the industrial application is to efficiently or specifically yield a chemical species, and optionally the chemical species comprises or is an antimicrobial compound,

and optionally the industrial application is to efficiently or specifically degrade a chemical species, and optionally the chemical species is or comprises a pollutant, and optionally the pollutant is or comprises a petroleum-derived hydrocarbon.

In alternative embodiments, provide are vaccines or pharmaceutical formulation for providing immunity against a Mycobacterium tuberculosis complex (MTBC) infection, comprising an immunologically protective dose of an inoculum and a vehicle,

wherein optionally the vehicle is for subdural, intravenous, intranasal, aerosol, or intramuscular delivery, administration to an immunocompetent individual,

wherein the inoculum or dosage unit comprises at least one of:

(a) an acellular vaccine or pharmaceutical formulation comprising a peptide or a polypeptide encoded by at least one of:

(i) the 38 PE/PPE MTBC genes as identified in TABLE A; or

(ii) the 366 identical MTBC genes as identified in TABLE C (see FIG. 33); or

(b) a live attenuated vaccine comprising attenuated Mycobacterium tuberculosis (MTB) engineered through functional deletion in the M. tuberculosis (MTB) one or more of:
(i) the 38 PE/PPE MTBC genes as identified in TABLE A; or
(ii) the conserved region of the 52 special immune-evading MTBC PE/PPE genes as identified in TABLE B (see FIG. 32).

In alternative embodiments, provided are methods for engineering phage therapeutics comprising use of any one of the 456 genes in claim 1(a), (b), or (c) for engineering phage therapeutics, the method comprising:

(a) confirming essentiality of the genes among the 38 listed in Claim 1a and the 366 listed in Claim 1b that by deleting the targeted gene using CRISPR or equivalent and culturing the resulting cell, and selecting for the next steps those genes whose deletion causes cell death; and

(b) engineering phages that can target (specifically bind to) at least one of the genes identified in (a), or claim 1(c), using homologous recombineering, bacteriophage recombineering of electroporated DNA (BRED), CRISPR-cas-based phage engineering, rebooting phages using assembled phage genomic DNA, or any equivalents thereof.

In alternative embodiments, provided are methods for sensitive heterogeneity analysis for detection of small but clinically relevant subpopulations, comprising:
a. Assembling sequencing reads into a contiguous sequence de novo, or optionally selecting a reference sequence against which to map reads,
b. Mapping sequencing reads back onto the de novo assembled or selected reference sequence and tabulating the composition of minor variants discordant with the sequence whereon reads were mapped for comparison,
c. Determining which minor variants are likely due to error (for example human, procedure, instrument) and which are likely due to true biological subpopulations by using dynamic posteriori error profiles, wherein dynamic error profiles are defined according to the type of error, error profile of the sequencing instrument, and error profiles of DNA library, and wherein the minimum criteria for calling heterogeneous biological subpopulations is adjusted using hypothesis-based maximum likelihood based on the specifications of sequencing instrument, library preparation, and the sequencing context.

In alternative embodiments, provided are methods of identifying heterogeneity based on oscillatory behavior during the iterative consensus sequence refinement described in the de novo assembly polishing step as provided herein.

In alternative embodiments, provided are methods of identifying heterogeneous genomic elements through annotating heterogeneous segments of a genome and genomic elements that are affected by the heterogeneous segments through methods as provided herein.

In alternative embodiments, provided are methods of determining the clinical relevance of the subpopulations harboring the heterogeneous genomic element variations, as provided herein.

In alternative embodiments, provided are methods for prognosing emergence of new phenotype or phenotype-conferring sequence variations in pathogens for Clinical Decision Support (CDS) comprising the steps of:
a. If a knowledgebase does not yet exist:
(i) Sequencing of unamplified DNA isolated from pathogenic prokaryotic organisms of a species of interest directly from samples of tissue or biological fluid, collected serially from the infected host over time,
(ii) Assembling the sequencing data from each sample, de novo, into one or more complete or partial consensus genomes,
(iii) Calling minority variants within each serially collected sample with respect to the consensus genome(s) assembled from the sample, wherein variants include single or multiple-base polymorphisms, insertions, deletions, inversions, relocations, or translocations, and wherein the position(s) with minority variants are considered as “heterogeneous” when the existence of the minority variant(s) is supported by a number and/or proportion of reads greater than the discordance expected in a homogenous sample,
(iv) Calling variant DNA bases with respect to the consensus genome(s) of prior samples from the patient, wherein variants include single or multiple-base polymorphisms, insertions, deletions, inversions, relocations, or translocations,
(v) Constructing of a catalog of correlations between heterogeneous and consensus variants' with the emergence of new phenotype-conferring sequence variations or new phenotype in subsequent samples,
b. Once a knowledgebase exists:
(i) Sequencing of unamplified DNA isolated from pathogenic prokaryotic organisms of a species of interest directly from a sample or samples of tissue or biological fluid,
(ii) Assembling the sequencing data from each sample, de novo, into one or more complete or partial consensus genomes,
(iii) Calling minority variants within the sample or samples with respect to the consensus genome(s) assembled from the sample, wherein variants include single or multiple-base polymorphisms, insertions, deletions, inversions, relocations, or translocations, and wherein the position(s) with minority variants are considered as “heterogeneous” when the existence of the minority variant(s) is supported by a number and/or proportion of reads greater than the discordance expected in a homogenous sample,
(iv) Calling variant DNA bases with respect to the consensus genome(s) of prior samples from the patient, or from related samples, wherein variants include single or multiple-base polymorphisms, insertions, deletions, inversions, relocations, or translocations,
(v) Prospectively computing probabilities of emergence of new phenotype-conferring sequence variations or new phenotypes in subsequent samples according to the aforementioned catalog,
(vi) Classifying infections as likely to change phenotype according to the calculated probabilities of future emergent phenotypes and/or new phenotype-conferring sequence variations to classify, over multiple time frames.

In alternative embodiments of methods as provided herein:
the heterogeneous variants are called using the methods for sensitive heterogeneity analysis as described or provided herein;
the probabilities of emergence are computed using the data generated by methods in step v (prospectively computing probabilities of emergence of new phenotype-conferring sequence variations or new phenotypes in subsequent samples according to the aforementioned catalog), and further optionally incorporating information derived from the methods as provide herein, or as features;
computing probabilities of future emergent phenotypes and/or new phenotype-conferring sequence variations are accomplished using statistical inference techniques;
computing probabilities of future emergent phenotype and/or new phenotype-conferring sequence variations are accomplished using machine learning techniques, and optionally the phenotype is drug resistance, or persistence, or resistance to phage therapeutics;
the sequencing technology is single molecule real time (SMRT) sequencing;
the organism from which DNA is extracted and sequenced is a member of the Mycobacterium tuberculosis complex (MTBC) infecting a human being;
the organism from which DNA is extracted and sequenced is a member of the Mycobacterium tuberculosis complex (MTBC) infecting a human being;
the sample is sputum. resected lung tissue, or fluid collected from bronchoalveolar lavage, or the sample is sputum. resected lung tissue, or fluid collected from bronchoalveolar lavage;
the drugs for which emergence of phenotypic resistance or sequence variations known to cause phenotypic resistance are of interest are one or more of: Isoniazid, Rifampicin, Amikacin, Kanamycin, Capreomycin, fluoroquinolone therapeutics, Pyrazinamide, Bedaquiline, Delaminid, Lizezolid, Clofazimine, Pretomanid, Cycloserine, GSK 3036656, Macozinone, Nitazoxanide, Rifapentine, Auranofin, Delpazolid, Sutezolid, SQ109, Telacebec, TBI-223, SPR270, BTZ-043, TBA-7371, OPC-167832, TBI-166, Sanfetrinem, S-004992, GSK-286, Spectinamide 1810, and any combinations thereof; and/or
the drugs for which emergence of phenotypic resistance or sequence variations known to cause phenotypic resistance are of interest are one or more of: Isoniazid, Rifampicin, Amikacin, Kanamycin, Capreomycin, fluoroquinolone therapeutics, Pyrazinamide, Bedaquiline, Delaminid, Lizezolid, Clofazimine, Pretomanid, Cycloserine, GSK 3036656, Macozinone, Nitazoxanide, Rifapentine, Auranofin, Delpazolid, Sutezolid, SQ109, Telacebec, TBI-223, SPR270, BTZ-043, TBA-7371, OPC-167832, TBI-166, Sanfetrinem, S-004992, GSK-286, Spectinamide 1810, and any combinations thereof.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1 graphically illustrates gene ontology (GO) enrichment within the accessory and core genomes, genes that have been duplicated, and genes that are not in the reference strain, H37Rv, in 97 M. tuberculosis clinical isolates' genomes. Narrow bars represent shared GO terms between Types.

FIG. 2A-C graphically illustrate Pangenome analysis of de novo assembled genomes from 97 M. tuberculosis clinical strains and two M. tuberculosis H37 reference strains: H37Rv (NC000962.3) and H37Ra (CP016972.1). a) Trend of CDS count as new genomes are added to the pangenome analysis. b) The closest gene relative in M. tuberculosis H37Rv based on an all-against-all pairwise nucleotide alignment for all genes identified to not exist in H37Rv from a pangenome of 97 M. tuberculosis genomes and categorized into several groups based on alignment statistics. Relatives represented by more than five genes are present in the plot. c) Frequency of PE/PPE and non-PE/PPE genes not present in H37Rv distributed within each category from part b.

FIG. 3A-B graphically illustrate Evolutionary analysis of the duplicated Rv1319c gene (encodes an adenylyl cyclase) in 99 M. tuberculosis genomes. a) SNP maximum likelihood phylogenetic tree with the clades colored by the duplication status (has a duplicate (pink), has a remnant (two Types) (blue/green), or does not have a remnant or duplicate (gray)). The outer rim colors represent Lineage determined by MIRU-VNTR/spoligotype. Outgroups for the tree were M. bovis and M. canettii. b) A schematic of the synteny within the four types of gene duplication/remnant. rv1319c′ represents the copied version of the original rv1319c. Rows 2 and 3 represent different CDSs created by different frameshifts. The last row represents the lineage 1 and lineage 4 isolates with no duplicate or remnant CDS(s) (matches the gene order in H37Rv).

FIG. 4 graphically illustrates Gene loss (through pseudogenization) analysis in 97 M. tuberculosis clinical isolates' genomes, H37Rv, and H37Ra. Nucleotide identity of the alignment between accessory genes and putative pseudogenes and corresponding standard deviation (s.d.) of codon adaptation index (CAI) from the mean CAI of the core genome of the putative pseudogene. Horizontal black line represents the standard deviation threshold to classify a putative pseudogene.

FIG. 5A-B graphically illustrate Logo plots for amino acid sequence motifs for a) PE b) and PPE generated from 84 PE and 61 PPE amino acid sequences from M. tuberculosis H37Rv (Genbank accession AL123456.3).

FIG. 6 graphically illustrates a Mechanism of gene conversion schematic. DSB: double-stranded break.

FIG. 7A-D graphically illustrate cell epitope scores across the alignment of two recombinant PE_PGRS genes in two M. tuberculosis lineage 4 isolates, putative acceptor (PE_PGRS54), and putative donor (PE_PGRS57) a) SEA06535 and b) 2-0028. c) Recombined region from part (a) where gene conversion was detected using RDP4; d) Recombined region from part (b) where gene conversion was detected using RDP4. Blue boxes represent score alignment between recombinant and acceptor; red boxes represent region that was replaced by the donor gene.

FIG. 19 illustrates a workflow schematic of the general methods for assembling genomes, annotating genomic elements of a subspecies, species, set of species, or genus of interest, and identifying heterogeneous segments of a genome by oscillation during polishing; workflow components are follows: Hexagons: Decision points; Red boxes: End points of failure; white boxes: actions; yellow boxes: intermediate checkpoints; cylinders: processes with subworkflows, green boxes: final checkpoint of workflow.

FIG. 20 illustrates Table S1, describing Assembly statistics and status for 178 Mycobacterium tuberculosis genomes sequenced on Pacific Bioscience's RSII platform; overall quality score was calculated by averaging sequence quality, tRNA, rRNA, and Pfam-A scores; these scores were calculated based on the criteria described in Land et al. 2014.

FIG. 21 illustrates Table S2, which shows the statistics of the genome size, protein sequence (CDS) count, PE gene count, and PPE gene count of 97 Mycobacterium tuberculosis clinical genomes and reference genomes H37Rv (NC_000962.3) and H37Ra (CP016972.1) assembled de novo and annotated by a custom annotation pipeline, Annotate TUBerculosis (AnnoTUB).

FIG. 22 illustrates Table S3, which shows a pangenome (repertoire of all genes in a population) and categorization of core (genes shared across 99% of genomes in a population) or accessory genome (genes not shared by all genomes in a population) from 97 Mycobacterium tuberculosis clinical genomes, H37Rv, and H37Ra; essential genes were determined based on the H37Rv in vitro study by DeJesus et al. 2017 (mBio).

FIG. 23 illustrates Table S4, which shows proteins that are 100% identical at the amino acid level and 100% query and subject coverage within a lineage or across all 97 reference-quality M. tuberculosis clinical genomes.

FIG. 24 illustrates Table S5, which shows the frequency and enrichment of essential genes in genes determined to be 100% identical (amino acid identity and query and subject coverage) across 97 M. tuberculosis clinical genomes (see Table S4 for genes and functions); and, functional enrichment of transcription factors, PE/PPE genes, hypothetical proteins, and toxins/antitoxin within non-essential, identical genes; transcription factors were compared to those determined in Rustad et. al 2014 Genome Biology; PE/PPE genes were determined by the amino acid sequence motif described in the current study. Hypothetical proteins were determined based on the functional category described in Mycobrowser (mycobrowser.epfl.ch); toxin/antitoxins were described in Tandon et. al 2018 J. Bact. Ribosomal proteins were not analyzed for enrichment.

FIG. 25 illustrates Table S6, which shows protein-coding genes from 99 Mycobacterium tuberculosis clinical genomes that were duplicated in at least one of the 99 genomes.

FIG. 26 illustrates Table S7, which shows sensitivity and specificity of the originally described motifs to classify PE and PPE protines and the motif created within the current study using MEME on all PE and PPE proteins in M. tuberculosis H37Rv.

FIG. 27 illustrates Table S8, which shows PE/PPE genes from M. tuberculosis H37Rv (AL123456.3) that were excluded from generation of a sequence motif to classify each family (PE or PPE).

FIG. 28 illustrates Table S9, which shows Genes in a pangenome of 99 M. tuberculosis genomes that were identified as PE or PPE with a more accurate amino acid sequence motif created from the PE and PPE genes from M. tuberculosis H37Rv; Sublineage motifs were described previously (Cole et al. 1998) and were queried within all PE/PPE proteins

FIG. 29 illustrates Table S10, which shows Gene conversion events and breakpoints detected by RDP4 in all PE or PPE subfamilies in the pangenome of 99 M. tuberculosis genomes; five of seven methods must have identified the event with a p-value of less than 0.01 to be included here; PE and PPE genes were identified using a more specific and sensitive amino acid sequence motif. Subfamilies were classified using previously described motifs (Cole 1998). See Materials and Methods for details on methods chosen and parameters.

FIG. 30 illustrates Table S11, which shows the number of guanine triplet or quartet quadruplexes (G4s) detected in PE_PGRS genes in M. bovis BCG Pasteur (Genbank accession AM408590.1) using G4Hunter.

FIG. 31 illustrates Table S12, which shows enrichment of B cell epitopes within all subfamilies of the PE and PPE protein families in 99 M. tuberculosis genomes. iBCE-EL was used to determine enrichment.

FIG. 32 illustrates Table B, which shows 52 PE/PPE genes for which we have found signatures of gene conversion, a special type of genomic change, including the nucleic acid sequences SEQ ID NOs:1-29, as discussed, below.

FIG. 33 illustrates Table C, which shows protein-encoding genes with identical amino acid sequence in all examined strains, including SEQ ID Nos:30-39.

FIG. 34 illustrates a table showing the in vitro gene essentiality and clinical relevance (when known) of eleven invariable genes expressed in both active and inactive human macrophage across the four main lineages of M. tuberculosis.

FIG. 35A-D shows the chromosomal and functional distribution of genes encoding identical proteins: FIG. 35A-C shown are noteworthy clusters of genes: conserved 60 kb cluster of non-essential genes especially dense in toxin-antitoxin proteins (FIG. 35A), a 15 kb cluster of essential 505 and 30S ribosomal proteins all on the leading strand (FIG. 35B), and a 50 kb cluster of 12 conserved genes, many of which are hypothetical or unknown proteins, all of which are non-essential in vitro (FIG. 35C); note that arrows representing genes are centered of ORF start position and their length do not correspond to ORF length or overlap with other ORFs; (FIG. 35D) distribution of ORFs encoding invariably conserved proteins (“icpORFs”, red and blue arrows) across the H37Rv genome; local density of total icpORFs (ORFs on either strand, black solid line) and global density (dashed, colored lines) are depicted; the disparity between (+)-strand and (−)-strand ORFs (dashed lines) increases near the center of each chromosomal arm and switches directionality at the terminus (approximately 2.21 Mb), illustrating the penetrance of lagging strand mutability bias over evolutionary time scales.

The patent or application file contains at least one drawing executed in color. Copies of this patent or patent application publication with color drawing(s) will be provided by the Office upon request and payment of the necessary fee.

Like reference symbols in the various drawings indicate like elements.

### DETAILED DESCRIPTION

In alternative embodiments, provided are products of manufacture and kits, and methods, that comprise or comprise use of inhibitory molecules for treating or ameliorating a Mycobacterium tuberculosis (TB) infection. In alternative embodiments, the inhibitory molecules inhibit the expression of one or more of the 38 PE/PPE TB genes as identified herein (see Table A), or one or more of the 366 identical TB genes and proteins (see Table C) common to a global set of TB isolates as identified herein, or the inhibitory molecules inhibit the expression of a transcript or a polypeptide gene product thereof or inhibit the changing (and hence evading the immune system) of one or more of the 52 special immune-evading TB PE/PPE genes as identified herein (see Table B).

As described in Example, 1 we produced 97 M. tuberculosis genomes, the most reference-quality genomes and methylomes of M. tuberculosis published to-date. A hybrid annotation was applied and all protein-coding sequences were compiled (pangenome analysis) to quantify gene gain (gene duplication) and classify gene loss (pseudogenes). This analysis further revealed hundreds of genes not present in the primary reference strain, H37Rv, which indicates significant sequence divergence in the species. Our data also confirm significantly more genome contraction (pseudogenization) than amplification (duplication). Investigation into PE/PPE genes identified by a more accurate sequence motif confirmed T cell epitopes primarily reside in the PE domain of PE_PGRS proteins, possibly serving as decoy antigens. Finally, we observed strong evidence of gene conversion within PE_PGRS proteins that significantly altered B cell epitopes.

### In Silico Methods to Identify a Comprehensive Set of High-Potential Therapeutic Targets

Provided herein is a comprehensive method of DNA sequencing analysis for identifying genomic elements, for example, genes or other genomic elements defined by genomic coordinates, as targets for medical and industrial applications.

In alternative embodiments, term “genomic elements” encompasses genomic elements as described in Kanduri et al, Bioinformatics, Vol 35, Issue 9, 1 May 2019, Pages 1615-1624, or genomic features as described in https://docs.patricbrc.org/user_guides/data/data_types/genomic_features.html.

We have developed a method to identify genomic elements as high-confidence targets for various industrial applications in a given species. In alternative embodiments, methods as provided herein can be used for drug and vaccine development and for accelerating development of industrial applications; however, methods are provided herein are not limited in scope to applications for drug and vaccine targets.

In alternative embodiments, methods are provided herein are applied to the analysis of genomic sequences using extant, nascent, and yet to be developed technologies for ascertaining the sequence of nucleotides comprising the genome of an organism/sample of interest. Methods as provided herein can be applied to other techniques of ascertaining the DNA sequence and producing complete genomes, and assembling genomes de novo, for example, methods as provided herein can be applied to DNA sequencing that utilizes single-molecule sequencing technologies, for example, as defined by in the Encyclopedia of Biophysics, which states “Single-molecule sequencing refers to techniques that can read the base sequence directly from individual strands of DNA or RNA present in a sample of interest”.

In alternative embodiments, methods are provided herein are used with single-molecule sequencing technologies that can comprise multiple techniques, such as:

Sequencing-by-Synthesis:
using optical labels (extant), for example, as described by Ameur et al, Vol 37, Issue 1, January 2019, pages 72-85,
Using arrays of nanoscale field effect transistors (nascent), for example, as described in U.S. patent application no. US 2019/0106742 A1, and/or
using energy transfer detection (nascent), for example, as described in U.S. patent application no. US 2019/0062829 A1; and/or

Nanopore-Sequencing Technologies:
Biological nanopores (extant), for example, as described in, for example, as described by Ameur et al, Vol 37, Issue 1, January 2019, pages 72-85:
Solid-State Nanopores, through either Optical or Electrical Measurement (Nascent):
   Plasmonic nanopores, for example, as described by Garoli et al, Nano Lett. 2019, 19, 11, 7553-7562, and/or
   other solid-state nanopore fabrications, for example, as described by Goto et al, J Hum Genet 65, 69-77 (2020).

Like many industrial applications, drug and vaccine development is hindered by lack of appropriate targets mostly because current techniques involve extensive and time consuming laboratory experimentation implementing a broad blind search approach to identify target candidates. In alternative embodiments, methods as provided herein quickly provide such appropriate targets, for example, methods as provided herein can determine which genomic elements meet the requirements of an application.

For example, in alternative embodiments, methods as provided herein address the problem of having an efficient way to identify drug and vaccine targets, which can be an exceedingly slow and expensive process, partly due to lack of targets that meet the vaccine or drug requirements, for example conservation and accessibility. Conservation, almost always, is a required attribute for a target since its absence in any variety of the species will make the application not broadly applicable. The next most frequently important attribute is the variability of a target. Many applications require that the target be highly stable across the varieties of the species. An example of such applications is the development of antibiotics where the target needs to be present, essential, and as stable as possible. Other applications require that the target be partly hypervariable in a specific way.

Provided herein are in silico methods to quickly identify a comprehensive set of high-potential targets for a variety of industrial applications including vaccine and drug development that are conserved, their variability profile determined, and their essentiality deduced from their conservation and variability without lengthy laboratory experiments (which significantly increases the time and cost of a project). In alternative embodiments, these in silico methods comprise:
1. Collection of a sufficiently large and diverse set of samples to represent the diversity of a species.
2. Use of long-read sequencing techniques (for example, single-molecule sequencing) capable of producing reference-quality de novo assembled genomes.
3. Assemble the genomes of the collected samples de novo, perform extensive quality control (QC) (involving mapping the sequencing reads onto the assembled genome in order to identify misassemblies, heterogeneities, and errors), polish, and circularize the genomes (for circular genomes) resulting in reference-quality assemblies.
4. Annotate the resulting genomes in multiple steps:
   (a) Annotation transfer from a reference genome for stable regions of the genome that maintain synteny (relative co-localization of genetic loci within segments of the reference chromosome and the chromosome to which annotations are being mapped),
   (b) Followed by an ab initio annotation approach or approaches for annotation of non-syntenic regions. These ab initio approaches may comprise extant and yet to be invented methodologies, including but not limited to:
      i. Annotation by sequence similarity orthology to conserved, known sequences constituting genomic elements in other species.
      ii. Genomic element boundary classification by machine learning and neural networks.
      iii. Genomic element boundary classification by sequence composition through algorithms such as dynamic programming, linear discriminant analysis, hidden markov models and neural networks, among others such as, for example, Wang Z, Chen Y, Li Y. A brief review of computational gene prediction methods. Genomics Proteomics Bioinformatics. 2004; 2(4):216-221. doi:10.1016/s1672-0229(04)02028-5.

The general methodology of steps 1 to 4 is depicted as a workflow schematic in FIG. 18.
5. Use the annotation from step 4 to determine the core genome of the species.
   a. We define the core genome differently than traditionally, but rather as defined by step 7, below. We adopt this new definition because the current traditional definitions and methodologies are limited and do not fully represent the essential functions for a species. As such, determining the core genome comprises two steps:
      i. Use sequence homology to identify conserved genomic elements
      ii. Add to this a consensus sequence for each of the alternative sets identified in Step 7 below.
   b. Determine whether the core genome is closed or open. If open, the diversity of the selected strains is not sufficient. Go back to step 1 and increase diversity.
7. Assemble a complete accessory genome:
   c. Using the annotation from step 4, and the assembled genomes, identifying the set of genomic elements that do not appear in all genomes. This set comprises the draft accessory genome.
      i. Determine whether the draft accessory genome is closed or open. To determine this, the number of accessory genes (or any type of genomic element being analyzed) is plotted against the number of genomes examined. If this relationship (number of accessory genes over number of genomes) approximates a function with a horizontal asymptote, the draft accessory genome is considered closed. Otherwise, the draft accessory genome is considered open. For species with a closed draft accessory genome, this set will be the complete accessory genome (or simply accessory genome). For species with open draft accessory genomes, we define this as the “accessory genome basis”.
   d. For open genomes, we use machine learning (ML) to analyze the resulting accessory genome basis to discover patterns of novel genomic element (for example gene) creation. The ML model is trained using observations of new genomic elements and then is used to predict emergence of future novel elements.
   e. Through sequence similarity and application of ML we then identify genomic elements (mechanisms) involved in creation of novel genomic elements. This enables identification of mechanisms of new genomic element creation and the ability to predict new accessory genes and other genomic elements.
   f. For open genomes, we define the accessory genome to be all genes in accessory genome basis and all those predicted by the ML.
7. Determine alternative (mutually exclusive) genomic elements (genomic element mutual exclusivity) and their impact on the core genome: We define alternative genomic elements to be the set of elements that are distinct from one another in sequence, perform the same function, and do not appear together in the same genome (exclusivity of genomic elements). Such elements form an “alternative gene set”.
8. In each genome, there may be several and distinct alternative sets, representing distinct essential functions, but only one member of each set may be present in a given genome. Steps for determining the alternative sets are as follows:
   g. For each element in the accessory genome, identify the set of other accessory elements that never appear together with the element under consideration in any genome of diverse collection of genomes. These sets form the basis for the alternative genomic sets.
   h. Use sequence similarity and ML to identify the defining sequence features for each set. This will produce a set of sequence motifs that define each set.
   i. Add each element predicted by the ML engine to the appropriate alternative set. This is done using the sequence motifs identified in the previous step.
9. Our concept of mutual exclusivity expands the definition of genomic conservation. If at least one element from an alternative set is present in every genome of a species, that set represents a conserved functional element that can be embodied in sequence by any of the gene sequences in the alternative gene set. Although different in sequence, if the presence of at least one member of an alternative gene set is essential, then the function is conserved. Hence, for each alternative gene set, we use the union of all unique sequences comprising that alternative gene a set as an addition to the core genome. As such, we propose that the “core genome” of, at least, pathogenic species is larger than previous thought and here, we offer a method of detecting the complete core genome. In this context, it is entirely possible that a core genome to be open since some essential alternative sets may remain open. In such a scenario, ML will be used to predict the expanse of each alternative set.
10. Identify two sets of genes: one set containing the most stable genes in the core genome (invariable genes) and one containing the most variable genes or in alternative sets (see above) of the accessory genome (maximally changing essential genes):
   (a) Search the core genome for genes that are identical or minimally variable across all genomes. These comprise the set of invariable genes,
   (b) Search for genes in the core genome that maximally vary, and genes in alternative sets (identified in step 7) that maximally vary or are frequently deleted (or pseudogenized) across the genomes. These comprise the set of maximally changing essential genes. Some of these genes will be involved in, or products of gene conversion. For instance, the acceptor and the recombinant in a gene conversion event will be alternatives to each other in most cases. On the other hand, donors in gene conversion events will not necessarily be an alternative to acceptor or the donor, even though they participate in creating maximally changing genes. As such, donors may not be included in the analysis outlined in 7. However, in this step, we identify and include donors of gene conversion events in the list of maximally changing genes since they play a role in creating such genes.

Steps 5-8 are written above as applied to genes as the class of genomic element under analysis, but the method is designed to be applied to any class or combination of classes of genomic elements. Expanded to multiple classes, instead of the accessory genome basis as defined in the steps above, we would have the basis of the accessory set of genomic elements and defined analogously according to the steps above applied to all genomic elements.

In alternative embodiments, for some applications, not all the steps outlined above are required, and some can be skipped. For example, for the case of efficient chemical conversion by bacteria, engineering species with minimal genomes is important. In such an application, genomic elements identified in steps 4 and 6 would be sufficient for engineering a minimal genome species.

Provided herein is a rapid, and more accurate method for prediction of essentiality. Essentiality is typically determined by experiments and survival assays measuring whether the cells of interest continue replicating when a genomic element is deleted or disrupted while cultured in an artificial environment. This artificial environment fails to recapitulate the constraints levied in the true environment and produces essentiality classifications that are misleading when extrapolated to the organism's native environment. In alternative embodiment, provided herein is a rapid and accurate method for prediction of essentiality that relies on an element's hyperconservation, or the combination of its variability profile and its gene exclusivity profile (step 7 above) across samples of the species. In alternative embodiment, this method has numerous applications that spans multiple sectors, for example:
1. Using the concept of gene exclusivity for a more accurate definition of genomic element conservation and functional essentiality.
2. Application of machine learning (ML) to predict the accessory genomes and alternative sets of species with an open genome.
3. The entire process (sequence of methods) for vaccine and drug target identification.
4. The two-step annotation process for annotating de novo assemblies for all purposes (target identification and other applications).
5. The method of prioritizing drug and vaccine targets by identifying invariant genomic elements (for example genes, ncRNA, promoters, riboswitches, copy number)
6. The method of identifying conserved but hypervariable genomic elements (including those involved in gene conversion) with varying epitopes (antigenic variation).
7. The method of identifying sets of essential genetic elements in the native environment of an organism of interest (that/those in which it has evolved) through their conservation and invariability across diverse de novo assembled strains.

These exemplary methods are outlined in detailed and validated in Example 1. The results of its application to 97 different strains of M. tuberculosis is also described Example 1, where some useful drug and vaccine targets have been identified.

### Infectious Disease Applications of Exemplary Methods as Provided Herein:

Pathogens are successful in causing infections mostly due to their ability to manipulate and adapt their environment. These abilities allow pathogens to evade host immunity and survive chemotherapy. Pathogens' ability to dynamically adapt to immune and drug pressure is primarily attributed to their ability to change their genomes in response to the pressures that they encounter. For most pathogens, the complete map and nature of these changes are poorly understood due to limitations of popular sequencing technologies (short-read Whole Genome Sequencing, or srWGS). SrWGS imposes limitations on assembling the pathogen's genome, preventing a comprehensive genetic analysis. Because of these limitations, srWGS reads are mapped onto the genomic structure of a well-studied reference genome to identify changes with respect to the reference. The srWGS approach offers several shortcuts but also has a critical drawback. It assumes that the pathogen's genomic structure remains constant across members of the species. In fact, this assumption is invalid, and pathogens change their genomic structure in order to evade the immune system and chemotherapy pressures.

To properly study such genomic changes (for example for comprehensive understanding of mechanisms of antibiotic resistance), the genome must be assembled de novo. De novo assembly of srWGS presents several challenges, both in initial assembly and in downstream analyses:
1. Genome Annotation (identification of which genes are present in the genome and their exact coordinates)
2. Structural variation mapping (how the genome's structure is different from others in the same specie—identification of inversions, relocations, deletions, conversions events)
3. Detection of variant versus invariant segments of the genome for drug and vaccine target identification.

While some of the methods described have been used for infectious diseases, our method of annotation and the larger method of identifying novel targets for vaccines and drug targets is novel and non-obvious.

Beyond infectious diseases, the two-step, hybrid annotation approach of methods as provided herein, which include a prediction of the accessory genome, identification of alternative sets, and the method as a whole, have utility for identifying subsets of essential genes in the native environment from which samples are derived. In alternative embodiments, this addresses a fundamental problem in determining gene essentiality. In alternative embodiments, methods as provided herein are rapid, more accurate, and not rely on direct killing assays in an artificial environment optimized for growth.

In alternative embodiments of methods as provided herein, there are numerous applications in clinical, industrial, and synthetic microbiology for which identifying the set of essential genes in an organism's natural environment would accelerate or newly enable innovation. For example, in the application of engineering bacteria that breakdown petroleum, applying exemplary methods as provided herein to multiple strains would identify the set of highly conserved genes that would serve as a scaffold to construct a minimal genome of essential genes as a starting point for engineering strains of bacteria that efficiently breakdown petroleum into less toxic metabolites, accelerating oil spill recovery efforts, and reducing harm to the environment. In alternative embodiments, exemplary methods as provided herein also can be used for engineering strains of bacteria for other types of chemical conversion, including biofuel production, nanoparticle synthesis, and metal contaminant and waste remediation. The methods as provided herein are emerging possibilities that hold large promise for combatting challenging environmental issues of the 21^{st} century. Engineering efficient strains for these applications using natively essential genes as a starting point as provided herein has not been previously described. The prevalence and utility of exemplary methods as provided herein can be used for improvements to engineering tools to enable more precise genetic manipulation to engineer solutions to issues of excess waste, clean energy generation, and numerous other applications of bacteria-driven chemical conversion.

We have developed a method to more easily identify high-potential genomic targets for a number of industrial applications including vaccine and drug development, biofuel generation, toxic spill cleanup, and many other environmental challenges of the 21^{st} century. This will significantly reduce drug development cost and accelerate the development timeline. Methods as provided herein can also advance genomic analysis in general for research. Methods as provided herein can be used for the assembling of genomes exactly as they are without assumption, annotating them for further analysis, and downstream analysis for target identification.

We have demonstrated the application of exemplary methods as provided herein to tuberculosis, the deadliest human infection killing 1.5 million people a year. As a result, exemplary methods as provided herein can be used with novel non-antibiotic therapeutics and new vaccine development.

Exemplary methods as provided herein are also broadly applicable to identify sets of genes essential for survival of a specie in its native environment. Identification of such genes has numerous potential applications in synthetic biology, improving engineering of bacteria to clean up our landfills and for efficiently synthesizing complex materials or materials expensive to synthesize through current methods.

Provided herein are Non-Antibiotic Chemotherapeutics (NACs) that neither sterilize (for example, as bactericidal antibiotics) nor stop bacterial growth (for example, as bacteriostatic antibiotics). Rather, these NAC drugs disable the immune-evasion capabilities of the bacteria making them easier for the immune system to identify and eradicate.

In alternative embodiments, provided are effective targets for NACs which will help in engineering NAC compounds. Our findings identify a unique set of genes with evolutionary dynamics previously unknown in M. tuberculosis. These evolutionary dynamics offer opportunity for development of NACs.

In alternative embodiments, provided are effective targets for phage therapy of mycobacterial infections. After its initial introduction phage therapy has stalled mostly due to lack of effective targets for the phages to attack. Provided herein are phage targets that can be used to screen against targets of known phages and for directed engineering of novel phages.

By their nature, NACs are less susceptible to emergence of resistance and will offer a more permanent solution to TB control than with classical antibiotics alone. Phage resistance has been observed, but we believe that this has more to do with the targets selected for phages. For the first time, we have identified targets that are identical across all 97 strains of M. tuberculosis suggesting that change in these targets are detrimental to the bacteria. As such, these targets cannot change to resist phage treatment. Provided are the targets identified from finished M. tuberculosis genomes, which offer promising avenues to developing novel alternative therapeutic classes with lessened probability of developing resistance.

In alternative embodiments, provided are targets in M. tuberculosis for NAC and phage therapeutics:
1. 38 PE/PPE (Pro-Glu (PE) and Pro-Pro-Glu (PPE)) genes (see Table A) that exist in all 97 strains from the five major lineages of M. tuberculosis, the primary causative agent of TB, as phage and NAC targets. PE and PPE genes are two large families of Mycobacterium tuberculosis genes that comprise approximately 7% of the genome size, and the two families are based on the presence of conserved Pro-Glu (PE) and Pro-Pro-Glu (PPE) motifs at the N termini of the proteins) (see Example 1). PE and PPE family of genes are specific to Mycobacteria and are known to interact with the host immune system to evade and subvert human immunity. Over one hundred PE/PPE genes exist in each bacterium's genome. Because of the repetitive segments embedded in these genes, they are difficult to sequence and study. Furthermore, these genes undergo frequent changes ranging from small variations to truncation, deletion, and even recombination, which generates new PE/PPE genes. This dynamism creates substantial diversity among these genes across strains and their lineages.

Through our novel methods (described in detail in Example 1), we have fully captured these gene families for the first time in clinical isolates. We have applied this method to a diverse set of clinical M. tuberculosis isolates collected from 97 patients from six countries in Asia, Africa, and Europe. This set represents the five major lineages of M. tuberculosis. We have captured the full set of PE/PPE genes in each genome. Through the analysis described in Example 1, we have identified a subset of 38 PE/PPE genes (see Table A) that are conserved in all 97 clinical isolates. This, for the first time, identifies a stable set of candidate vaccine targets.
2. 366 specific genes that exist in all strains and are identical at the protein level as targets for phage therapy. For the first time, we have identified a set of proteins that remain identical in all lineages of M. tuberculosis. The discovery that these gene products remain unchanged in all strains, points to the fact that not only they are essential, but also highly sensitive to change. They need to remain exactly as they are for the bacterial cell to survive. These genes are ideal targets for phage therapy. Resistance to phage therapeutics that recognize these targets is exceedingly unlikely to happen.
4. 52 specific PE/PPE genes for which we have found signatures of gene conversion, a special type of genomic change. We have identified 52 such genes. Of these, 24 are acceptors and/or donors in the conversion process, and 28 are the products of this process. These genes harbor B cell epitopes that are continuously changing (new epitopes emerge, or order of epitopes change). This causes the immune system not to recognize these proteins. This is a common mechanism of immune evasion. PE/PPE genes are known to manipulate the host immune system. As such, these genes are ideal targets for NACs and possibly phages. Drugs that prevent epitope change or completely disable their expression will prevent immune evasion and significantly increase the possibility of immune system detection and eradication.

Accordingly, in alternative embodiments, provided are: identification of 456 TB genes useful for developing NACs; the use of these 456 genes for selecting environmentally-derived phages for formulating cocktails of phage therapeutics; the use of these 456 genes for engineering phage therapeutics; the use of these 456 genes above developing drug/phage combination therapies; and, the use of these 456 genes above for engineering phage therapeutics.

In alternative embodiments, methods as provided herein overcome several barriers for the development of NACs and for the use of the specified genes for phage therapeutics:
1. Difficulty in studying PE/PPE genes. PE and PPE genes are difficult to sequence and study. This difficulty arises from inherent difficulty in sequencing them with srWGS technologies and from their diversity and dynamism. Due to these factors, there has not been a study that describes the true diversity of these gene families. Exemplary methods as provided herein address this issue.
2. Lack of de novo assembled clinical genomes. Because of the prevalence of short read sequencing, most sequenced genomes are assembled using a published reference genome. This approach has precluded characterization of PE/PPE gene diversity: It assumes that the M. tuberculosis genomic structure remains constant, and therefore only changes to PE/PPE genes in the reference genome can be catalogued. Exemplary methods as provided herein provide for de novo assembly, and annotation of these genomes. Through these exemplary methods we have produced 97 high-fidelity, reference-quality, finished genomes (see Example 1) that present the first comprehensive account of the true genomic content across these diverse isolates' genomes. This knowledge has allowed us to study PE/PPE diversity across isolates and identify the 38 PE/PPE genes (see Table A) are conserved in all 97 isolates.
3. No previous comparison of proteins across de novo assembles. Due to absence of any pipeline as outlined in Example 1, including the idea of checking identity at protein level, a comprehensive set of identical proteins/genes has not been generated previously.
4. Phenomenon of converting PE/PPE genes was previously unknown. Gene conversion is a phenomenon known in other species to act as mechanism for immune evasion, but had not been demonstrated in M. tuberculosis in these genes. We have discovered that gene conversion occurs in genes that harbor B cell epitopes that affect host immune recognition of the infecting cells. A s such, it is not anticipated from existing work that such a mechanism exists in M. tuberculosis.

In alternative embodiment, NACs as identified using methods as provided herein can be treated as co-drugs along with existing antibiotics. More effective NACs can function as single drug therapeutics in immunocompetent patients. An additional advantage of NACs lies in the more challenging path that pathogens have to take to evolve resistance to NACs. As such, NAC resistance is not expected to emerge, or to emerge far less frequently than traditional antibiotics.

In alternative embodiments, provided herein are genomic targets for drug and vaccine development, where the identified targets can be classified into three classes, segregated by the type of protein and their genomic stability:
1. Identical proteins. Protein-encoding genes with identical amino acid sequence in all examined strains. Provided herein is the identification of 366 such proteins (see Table C).
2. Conserved PE/PPE genes. PE/PPE genes that are present in all genomes examined, with limited variability across the genomes. Provided herein is the identification of 38 such genes (see Table A).
3. Recombining PE/PPE genes. PE/PPE genes for which we have found signatures of gene conversion, a special type of genomic change. Provided herein is the identification of 52 such genes (see Table B); and, of these, 24 are acceptors and/or donors in the conversion process, and 28 are the products of this process.

In alternative embodiments, identified genomic targets, which can be used for live attenuated vaccine development, acellular vaccine development, and antibiotic development, have at least one or more of the following five attributes:
Stability. The amino acid sequence encoded by the target gene must remain identical, or especially conserved for its protein class to meet this criterion.
Immunogenicity. The protein encoded by the target gene must evoke an immune response in humans to meet this criterion.
Expression (in vivo). The target gene must be expressed into a protein to meet this criterion.
In vivo Essentiality. The target gene must be essential for M. tuberculosis survival, in vivo to meet this criterion.
In vitro Essentiality. The target gene must be essential for M. tuberculosis survival, in vitro to meet this criterion.

### Non-Antibiotic Chemotherapeutics (NACs) Development

In alternative embodiments, methods as provided herein comprise the following steps for developing NACs:
1. Identify NAC targets. Identify targets with maximum divergence across diverse strains that affect B or T cell epitopes. We have identified 52 genes involved in gene conversion that affects B cell epitopes.
2. Identify a strategy to prevent these genes from converting. One strategy would be to knock out the donor or acceptor (or both) to see whether the resulting mutant survives (if it does not, the gene becomes a target for antibiotic development see companion disclosure for novel antibiotic development, or for phage therapy, as described below), and is more easily eradicated by the immune system in an animal model.
3. Identify a compound candidate as a NAC. Identify a chemical compound that targets the genes identified in step b to either prevent or significantly reduce their expression. An alternative novel approach comprises engineering a compound that binds to the typical conversion sites and prevents the gene form converting.
4. Those compounds that satisfy both conditions in step c will be the choices for a clinical trial in humans.
   Exemplary Methods for Phage (bacteriophage) therapeutic development.

Common steps:
a. Identify stable targets: A first step comprises identifying targets that are stable in all strains and sensitive to change in amino acid sequence. We have identified 366 genes from among a total of over (at least) 4,000 (or more) which are identical at the protein level in all 97 strains. The fact that they are identical indicates that cells with a mutation in these genes either died or failed to proliferate. As such, these offer the ideal phage targets with minimal risk of phage resistance. In addition to the proteins synthesized by these gene target, any cell-surface molecules that are synthesized or localized to the cell-surface by target proteins will also be considered for downstream steps.
b. Curate mycobacteriophage candidate library. Several thousand mycobacteriophages have been isolated and deposited in collections housed in industrial, academic, and military organizations. In principle, these phages could be used to attack specific targets in M. tuberculosis. Phage hunts are ongoing and routinely identify new mycobacteriophages, which will continue to build this library. This library can also be supplemented with targeted phage hunts.

### Natural Phage Cocktail Therapeutic.

In alternative embodiments, provided are environmentally-derived phages to treat TB:
(a) Identify phage-target matches. For a natural phage cocktail, stable M. tuberculosis molecular targets from Step 2a will be screened against the library of mycobacteriophages in Step 2b. Suitability of matches will be proceed differently depending on whether they are lytic or nonlytic phages:
i. Lytic mycobacteriophages will continue development for natural phage cocktail therapeutics.
ii. Nonlytic mycobacteriophages will be sent to the engineered phage cocktail therapeutics pipeline.
(b) Formulate phage cocktails. Mycobacteriophages identified in step (a) will be formulated into synergistically effective phage cocktails, alternatively comprising multiple phages with distinct targets. These cocktails can then be screened for in vitro efficacy against M. tuberculosis.
(c) Evaluate phage therapeutic efficacy in animal models. Test efficacy and safety profiles of the phage cocktail formulations from step b in animal models of TB.

### Engineered Phage Cocktail Therapeutics.

The first instance of engineered phages curing a human infection was recently reported, and was engineered against a mycobacterial lung infection, providing precedent that justifies this approach as a viable development strategy for TB phage therapeutics:
(a) Identify surface-expressed phage targets. The next step is to identify which of the 366 genes expressed on the cell surface of M. tuberculosis or are responsible for synthesis and/or localization of surface molecules and targeted by mycobacteriophages as identified using methods as provided herein. All such genes are considered targets for phage therapeutics.
(b) Engineer effective phages against M. tuberculosis. The mycobacteriophages identified in step 2cii can be engineered through one of three strategies:
   (i) Engineer nonlytic phages into lytic phages. This exemplary strategy simply requires changing the nonlytic phage into a lytic phage. This was done for the first and only engineered phage that has cured infectious disease in human, a mycobacteriophage.
   (ii) Engineer nonlytic phages encoding toxic proteins. Nonlytic phages are typically ineffective for phage therapeutics because, unlike lytic phages, they do not quickly act as bactericides. However, bactericidal nonlytic phages can engineered by editing phage genomes to encode encoding proteins toxic to their bacterial host. This has been demonstrated for other bacteria. The problem with such systems in the past is that resistance developed to these phages. Stable targets as identified and provided herein circumvent this issue by providing targets unlikely to evolve phage resistance. Engineering nonlytic phages encoding proteins that affect these targets have significant, unexplored therapeutic potential. This option can target additional genes from the 366 stable targets that are not expressed on the cell surface.
   (iii) Engineer chimeric phages that are lytic and recognize stable targets. Phage genomes can be spliced and have their elements recombined through a method phage refactoring. Phage refactoring a chimeric phage comprising the DNA responsible for lytic capabilities from lytic mycobacteriophage that does not match the stable targets identified herein, and the DNA responsible for phage-target recognition from lysogenic matches as identified herein. The chimeric phages can be lytic and can recognize M. tuberculosis by the stable targets.
(c) Formulate phage cocktails. Phages engineered as described above are formulated into synergistically effective phage cocktails These cocktails are screened for in vitro efficacy against M. tuberculosis.
(d) Evaluate phage therapeutic efficacy in animal models. Efficacy and safety profiles of phage cocktails developed as described above are tested in animal models of TB.

### Synergistic Phage/Antibiotic Therapeutics.

In alternative embodiments, provided are phage/antibiotic combination therapies; these work best when mechanisms of resistance to phage and drug are mutually antagonistic:

Identify synergistic drug/phage pairs. In alternative embodiments, two method as provided herein for identifying effective phage/drug combinations for further development comprise:
High-throughput screening. In this exemplary approach, many combinations of candidate phage cocktails as identified above and/or those engineered as discussed above are combined with existing TB therapeutics or promising novel therapeutics, and killing assays are run against M. tuberculosis. The most effective combinations are advanced to subsequent steps in development.
Targeted engineering. In this exemplary approach, the 366 genes are screened against genes known to mediate acquired or intrinsic resistance in M. tuberculosis. For any matches, the most promising candidates are selected for phages to be engineered to target. The benefit of this approach is that many adaptations producing resistance to phages would decrease resistance to the drug therapy. For example, a promising potential target is efflux pumps, which are among the set of 366 stable targets identified herein; efflux pumps are key in surviving all TB drugs, and are common recognition targets of phages.
Evaluate drug/phage combination therapy in animal models. Efficacy and safety profiles of drug/phage combinations as identified herein are tested in animal models of TB.
Clinical trials for phage therapies for human TB. The final step for any of the three products is to advance therapeutics to clinical trial.

Stable M. tuberculosis genes are identified herein that are used for developing phage therapeutics, for example, as described herein, with significant therapeutic potential. M. tuberculosis is a target for developing phage therapeutics, for several reasons:
M. tuberculosis is a stable pathogen. M. tuberculosis is limited in its diversity compared to other problematic pathogens like Acinetobacter baumanii and Pseudomonas aeruginosa, simplifying the complexity of phage cocktails required to effectively sterilize infection. This is simplified further by our distillation of stable targets across the species.
Emerging TB drug-resistance. Although phage therapy might be prohibitively expensive for use against TB in developing nations, the rise of extensively drug-resistant and totally drug-resistant TB foreshadows an upcoming need for alternatives to chemotherapeutics to treat such cases in developed nations, where the price point of phage therapeutics is not prohibitive.
Lack of sterilizing therapy. TB chemotherapeutics do not sterilize M. tuberculosis, leaving a persistent infection that often recurs. Phages hold promise for sterilizing therapy, due to their specificity, ability to self-replicate, and to co-evolve with their bacterial target.
Huge market potential. With millions newly infected each year, the potential market for an effective TB phage therapeutic is among the largest of any infectious disease.
Alternative therapies are unappealing. Unlike many infections that can be cleared by antibiotics in a matter of weeks, the shortest of TB antibiotic regimens last months, and those for drug-resistant infection stretch over a year. This makes clinical trials for TB phage therapeutics more viable than for other infectious diseases. Past clinical trials have been cancelled because the control, antibiotic receiving group was healing in a matter of weeks. This would not be an issue for phage therapy against TB.

In studying nearly 100 clinical isolates of M. tuberculosis from around the world, the bacteria usually responsible for human TB infections have been analyzed with long-read sequencing technology and, unexpectedly, we have uncovered a large trove of genes that are identical across all strains, making these promising targets for developing new therapeutics. In addition, we also identified 52 genes that are very different across strains in a manner that evades the immune system but have conserved portions that do not change.

Provided herein are exemplary methods for using these identified genes as targets for developing novel therapeutic approaches to TB:

One exemplary method as provided herein provides a non-antibiotic chemotherapeutics (NACs) approach that does not kill or stop their growth like typical antibiotics do. Rather, NACs prevent the bacterial cells to evade the human immune system through their frequent B Cell epitope changes. In this way, the immune system will easily identify the infecting cells and destroy them.

A second exemplary method as provided herein provides for the phage therapy of a mycobacterial infection by providing new phage targets. In alternative embodiments, provided herein are targets for phage therapy and/or for engineering new phages.

In alternative embodiments, this new set of genomic targets as provided herein has enabled a three-pronged pipeline for phage therapeutics: First, where naturally occurring phages are turned on the TB-causing bacteria; second, where phages are engineered in a lab to kill the bacteria; and, a third where phages are combined in special ways with traditional anti-TB drugs that makes it far more challenging for the bacteria to develop resistance phage or bacteria. These new exemplary approaches use stable targets in the TB-causing bacteria to offer new solutions that are desperately needed against TB.

In alternative embodiments, this new set of genomic targets can be used for:

Live attenuated vaccine development: In alternative embodiments, provided are a class of vaccines or pharmaceutical formulations formulated using strains of M. tuberculosis engineered to be avirulent to build immunity against virulent M. tuberculosis strains. In alternative embodiments, provided are methods for developing M. tuberculosis live attenuated vaccine, and optionally steps in M. tuberculosis live attenuated vaccine development comprise:
1. Determine stability: The first step is to identify targets that are stable in all strains. We have identified 38 stable genes (see Table A) (the 38 core PE/PPE genes) as high-quality targets among over 4,000+ genes that exist in a typical M. tuberculosis genome. Additional candidates can be chosen from the set of 366 identical genes, see Example 1.
2. Engineer mutant strains to determine essentiality and immunogenicity. The next step is to knockout the targets identified in step a (individually or in combinations) and: test the resulting mutant for survivability and infectiousness, and test whether knocking out any of the targets identified in step a will allow bacteria to survive but be noninfectious (avirulent, or “attenuated”) and evoke an immune response. Genes selected in this step must not be required for evoking an immune response (i.e. the mutant strain must still evoke an immune response), and must not be essential in vitro.
3. Develop attenuated live vaccine or pharmaceutical formulation for TB. Formulate the avirulent strain from step c into a viable vaccine.
4. Evaluate vaccine efficacy in animal models. Before moving to clinical trials, the vaccine developed in step d is tested to demonstrate efficacy and non-infectiousness in animal models.
   Acellular vaccine or pharmaceutical formulation development. In alternative embodiments, provided is a class of vaccine comprising specific M. tuberculosis cell surface protein components, rather than whole avirulent live cells. In alternative embodiments, the vaccine or pharmaceutical formulation trains the immune system to rapidly recognize M. tuberculosis by the presence of these proteins expressed on their surface and generate a strong immune response against these proteins.

In alternative embodiments, provides are methods for M. tuberculosis acellular vaccine development, which can comprise the following steps:
a. Identify stable portions of immune evasion genes. The first step is to identify targets or sets of targets that are present or partially present in all strains yet exhibit variability indicative of immune evasion mechanisms. This variable portion is important because it suggests a strategy to evade the immune system. Through identification of gene conversion (a form of homologous recombination that can create antigenic diversity) loci, we have identified 52 genes that have a stable region (important for immune system identification) and frequently variable regions that contain B cell epitopes (important for immune evasion). These epitopes are the sites that the immune system uses to recognize foreign proteins. We have discovered that the conversion events repeatedly change either the order or the nature of the epitopes, a clear sign of immune evasion (see methods disclosure in Example 1). The 52 targets (see Table B) frequently have acted as donors, acceptors, or are the recombinants in gene conversion events that affected B cell epitopes. Members of the 38 core PE/PPE genes (see Table A) that have hypervariable regions within them also can be added to the list of 52 (see Table B) as targets.
b. Determine immunogenicity of stable regions. The targets identified in step a need to be analyzed for their stable regions. We have identified regions in each target that is stable across our diverse set of isolates. Next, the immunogenicity of these stable regions needs to be determined in vitro. This is to determine the utility of the region for invoking an immune response. If the response is weak, a conjugated vaccine or pharmaceutical formulation design will be preferential to boost recognition by the immune system. Immunogenicity will then need to be tested against the target as expressed by M. tuberculosis, where the immunogenicity of the targeted protein fragment might be influenced primarily by accessibility.
c. Engineer acellular vaccine or pharmaceutical formulation for TB. Engineer a vaccine cocktail that is well-tolerated, evokes a robust immune response, and includes the in vivo accessible fragments identified in step b along with any necessary conjugates, adjuvants, and stabilizers to comprise a well-tolerated, efficacious formulation.
d. Evaluate efficacy in animal models. Before moving to clinical trials, the vaccine or pharmaceutical formulation developed in step c is tested and demonstrate efficacy in animal models.
   Antibiotic development. In alternative embodiments, provide are methods for novel antibiotic drug development against M. tuberculosis, which can comprise the following steps:
1. Identify stable targets. Identify targets that are stable in all strains and are sensitive to change in amino acid sequence. We have identified 366 genes from among a total of over 4,000+ are identical at the protein level in all 97 strains. The fact that they are identical indicates that those strains that had a mutation in these proteins were eradicated by the human immune system. Novel antibiotics that target these genes are highly likely to be bactericidal or, at a minimum, bacteriostatic.
2. Determine essentiality of targets. The next step is to perform laboratory experiments that induce mutations/knockout the genes one at a time and observe the consequence in the bacterial cell:
   i. If the cell survives but becomes avirulent, the gene will be added to the list of candidates for engineering an attenuated vaccine or pharmaceutical formulation (steps a and b).
   ii. If the bacterial cell dies, or growth significantly slows, the gene becomes a viable target for a new bactericidal or bacteriostatic antibiotic drug.
3. Develop novel therapeutics. Identify or engineer compounds that can disable or hinder either the expression of the targets identified as described above, or their essential function, with strong efficacy in vivo.

Tuberculosis (TB) is now the most successful human infectious disease. It has killed over a billion people in the last 200 years. Its virulence and survival in the human host is largely a mystery. Despite decades of research, a TB vaccine has proven to be elusive. The PE/PPE family of genes are thought to interact with the human immune system in order to evade it.

For the first time, we have identified 38 PE/PPE genes (see Table A) (the TB family PE/PPE family of genes are thought to interact with the human immune system in order to evade it), 52 special immune-evading PE/PPE genes (see Table B, and 366 identical proteins common to a global set of isolates (see Table C) (97). The manipulation or knocking out of these genes will result is strains that are avirulent.

These avirulent strains hold promise for developing a vaccine, which has been challenging for TB. The 366 identical genes provide ideal targets for development of novel antibiotics. The fact that these genes remain identical across so many strains from across the world is an indication that bacteria are sensitive to changes in these genes and hence they provide the perfect target for antibiotic attack.

### Prognostics for Emergence of New Phenotype

In alternative embodiments, methods as provided herein comprise the following steps for prognosing emergence of new phenotypes, such as resistance to chemotherapeutics.
1. Method of detection of heterogeneous segments of a genome using hypothesis-based maximum likelihood.
2. Method of using dynamic posteriori error profiles to determine which minor variants are likely to be due to error (for example human, procedure, instrument) and which are likely due to biological subpopulations.
3. Method of defining the dynamic error profiles based on the type of error, instrument error profile, and library error profiles
4. Method of using hypothesis-based maximum likelihood to adjust minimum requirements for base calling based on the specifications of sequencing instrument, library preparation, and the sequencing context for sensitive heterogeneity analysis.
5. Method of identifying heterogeneity based on the oscillatory behavior of the polishing step in de novo assembly.
6. Method of using annotation to identify heterogeneous genomic element variation (GEV) from the heterogeneous loci or segments identified in steps 1 and 2.
7. Method of determining clinical relevance of the heterogeneous elements as described in claim 24.
8. Method of using steps 1-4 for detection of clinically consequential subpopulations early and provide disease prognosis for the current or proposed treatment regimens during the early stages of emergence of new clinically relevant phenotypes (for example resistance, persistence).
9. Method of spatiotemporal analysis of genomic and epigenetic variant emergence in serial samples using machine learning to identify spatiotemporal patterns of emergence of clinically relevant phenotypes. In this method, the connection between phenotypic emergence and appearance of variants both across space (the genomic coordinate system) and time. In other words, it is not simply the set of variants present at any one time that is considered for predicting future emergence of phenotypes, but also which variants have appeared already, and the order in which variants have appeared. This method is novel, and it extends the principle of epistasis to the temporal dimension. This is the first application of epistasis temporally. We refer to this principle as temporal epistasis. The utility of this principle lies in capturing specific evolutionary trajectories or families of evolutionary trajectories that often lead to emergence of a clinically important phenotype. When permitted by the technology used for sequencing, base modifications are also included in this spatiotemporal analysis, enabling analysis of their temporal epistatic effects with one another and with genetic variants to be discovered. Mounting evidence has suggested that DNA base modifications, such as DNA methylation, can precede genetic evolutionary steps toward a phenotype, and follow the steps as a compensatory mechanism to offset ancillary changes incurred by the newly evolved genotype that diminish fitness of the organism. Machine Learning techniques, specifically, recurrent Artificial Neural Networks (rANN) are used due to their ability to incorporate the temporal as well as spatial relationships in data:
   a. rANNs will be trained with time-series genomic and epigenetic data collected from patient samples.
   b. Input to the network will be the posteriori probabilities of genetic and epigenetic variants discovered at each timepoint detected through the hypothesis-based maximum likelihood method of step 1, as well as patient metadata. The posteriori probabilities will represent the dynamic variation and the evolution of subpopulation dynamics.
   c. After training of the system with the data of each timepoint, the network will be allowed to oscillate until it reaches a steady state. The output will be phenotypic data from the next time point.
   d. After achieving a steady state, the network will be trained with the next time point.
   e. This process will repeat for each patient.
      i. At this stage, if the network is performing accurately for the patient, it will be used to provide patient-specific prognostics.
      ii. If the network does not perform well, the process will repeat for additional patients, until network prediction accuracy reaches acceptable levels. At such a point, the model will be used as a general-purpose prognostic tool for all patients.
   f. Upon successful training of a model, it will be investigated for determination of significant variations in the network's performance. This will be done through network weight perturbation experiments.

This will determine which specific genetic or epigenetic variants are important at each time point for emergence of the new phenotype, drawing a spatiotemporal pattern of emergence for each new phenotype.
11. Method of using step 6 to provide disease prognosis for ongoing or proposed treatment regimens before the emergence of new clinically relevant phenotypes (for example resistance, persistence). Specifically, for prognostics, a molecular diagnostic tool will be used to detect the variants involved in each spatiotemporal pattern. The probability of emergence of the new phenotype will then be estimated based on the significance of the detected variants in the spatiotemporal patterns. This probability is used to provide clinical decision support, outputting a recommendation for continuing the current regimen, making sampling more frequent, or changing. This method can also be applied for heterogeneous variants, both qualitatively (detection of heterogeneity at any level) and quantitatively (estimation of the relative subpopulation sizes from the fraction of sequenced reads supporting the heterogeneous variant(s)).

### Clinical Decision Support for Antibiotic Regimens

In alternative embodiments, methods as provided herein, depicted in FIG. 19 and adopted from the steps for prognosing new phenotype emergence as described above for the phenotype of drug resistance, comprise the steps for prognosing emergence of resistance to antitubercular chemotherapeutics as a form of clinical decision support for recommending drug regimens against tuberculosis. Variations from the specific example implementation depicted in FIG. 19 include but are not limited to:
a. Non-drug therapeutic treatments, optionally phage therapeutic regimens, and optionally host-directed therapeutics.
b. The substitution or addition of epigenomic (for example DNA methylation) variants in place or in addition to genomic variants in spatiotemporal patterns, optionally as described above in step 6.
c. Inclusion of additional types of genomic variations, including single or multiple-base polymorphisms, insertions, deletions, inversions, relocations, or translocations.
d. Determination of heterogeneous variants by the methods described above, in step 1 and optionally in step 2. And optionally determination heterogeneous genomic element variations by the methods of step 3 above.

FIG. 19 conceptually illustrates the procedure for building a prognostic knowledgebase of harbinger mutations whose spatiotemporal patterns are predictive of subsequent emergence of a clinically relevant phenotype. In the figure, the procedure is implemented for prediction of drug resistance in Mycobacterium tuberculosis, and its use as a prognostic device and clinical decision support tool. (a) serial sampling and sequencing of infectious matter (for example sputa, bronchoalveolar lavage, biopsied or resected lung tissue) derived from patients during drug treatment. Parallel phenotyping of samples or by proxy measures of treatment efficacy or infection resistance. Prospective and/or retrospective serial samples can be used in building the knowledgebase. (b) Creation of knowledgebase mapping heterogeneous variants to future emergence of resistance or resistance conferring variants, comprising two steps: (i, top) For each sample, calling heterogeneity according to prevalence of minority variants and error profile of based on the type of error, instrument error profile, and library error profiles. Dashed lines for each variant type depict the threshold for proportion above which a variant would be considered truly heterogeneous. This threshold can be set according to the expected distribution of non-majority calls in homogenous samples based on estimates of the aforementioned error profile. (ii. bottom) Identifying harbinger mutations (feature selection). Harbinger mutations can be selected on the basis of i) high pairwise mutual information between heterogeneous or consensus variant in sample n and emergence of resistant phenotype or emergence of known resistance conferring mutation at time n+t, where t>0; ii) other measure of association between variant and subsequent phenotypic emergence; iii) methods of determining variable importance in machine learning models trained on variants as input and subsequent phenotypic resistance as output, such as optionally training rANNs as described below and performing network weight perturbation experiments (c) mock-up comparison of clinical outcome for a theoretical patient in which resistance emerges to the first drug regimen they are treated with regimens informed with (right) and without (left) prognostic clinical decision support (CDS).

In the example on the left, the patient continues on the original regimen until resistance emerges and treatment fails, as current phenotypic measures using only known resistance-conferring variants (stars, highlighted by red rectangle) did not identify resistance until it had already manifested phenotypically. In the scenario on the right, the level of heterogeneity (darkness of ovals) of harbinger mutations cued the prognostic CDS tool to recommend changing the treatment regimen after sample 4, 2 samples prior to emergence of resistance in the alternative scenario, leading to treatment success. The CDS output comes in the form of a 0-1 computed probability of emergent resistance (Prob), and one of three suggestions based on that probability: “continue treatment”, “Monitor closely”, which means Prob has been increasing and samples should be taken more frequently if possible, and “change regimen”, indicating high probability of impending resistance emergence if the current regimen continues. Prob can be computed using a recursive artificial neural network (rANN) trained to recognize spatiotemporal (variants across the genome and time) patterns predictive of future emergence of phenotypic resistance.

### Products of Manufacture and Kits

Provided are products of manufacture and kits for practicing methods as provided herein, and optionally further comprising instructions for practicing methods as provided herein.

Table A: Pro-Glu (PE)/Pro-Pro-Glu (PPE) TB or MTBC genes

The names of the following Pro-Glu (PE)/Pro-Pro-Glu (PPE) TB or MTBC genes (of Table A) can are their common names and are described for example, in known databases such as GenBank:

PPE18; PE33; PE_PGR; S1; PPE44; PPE11; PPE53; PE14; PPE51; PE25; PPE10; PE17; PE13; PE_PGRS59; PE15, PPE20; PPE22; PE12; PE5; PPE3; PE_PGRS18; PPE1; PPE45; PPE12; PPE19; PE4; PPE4; PE_PGRS44; PPE41; PE23; PE35; PPE68; PE16; lipX; PPE31; PE20; PPE32; PPE14; PPE15.
Table B: conserved region of the 52 special immune-evading MTBC PE/PPE genes

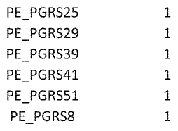

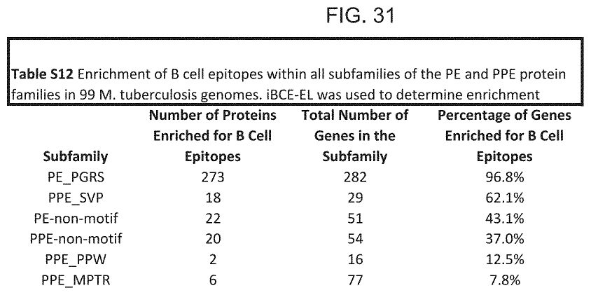

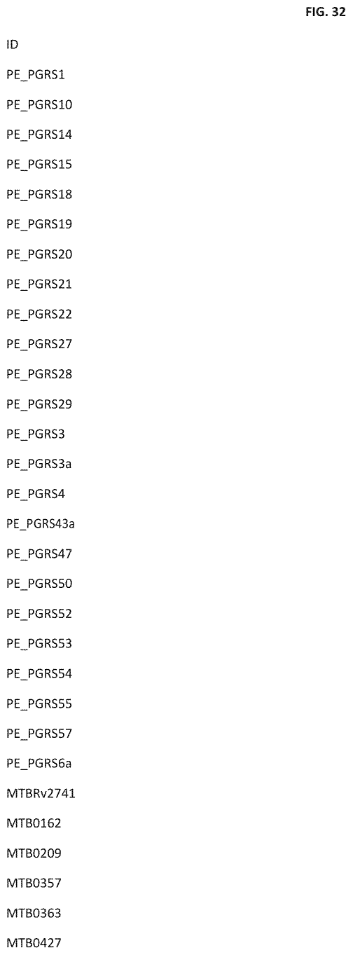

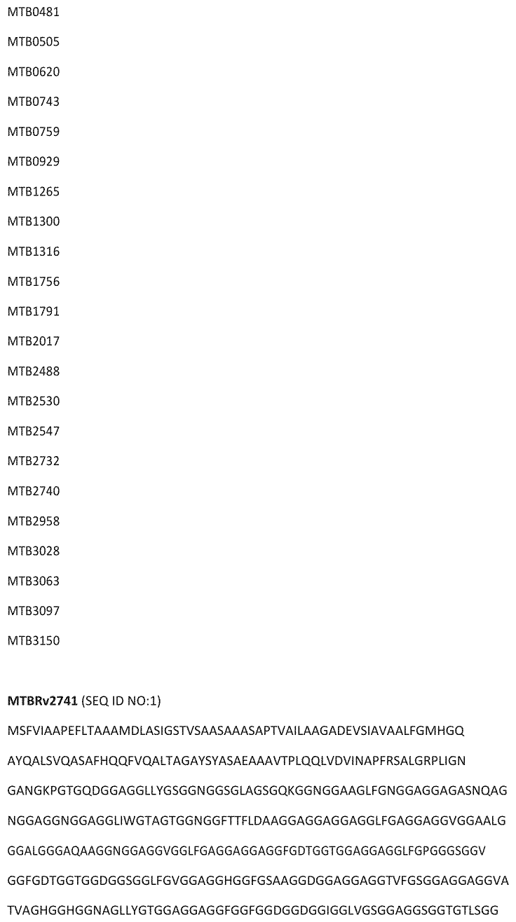

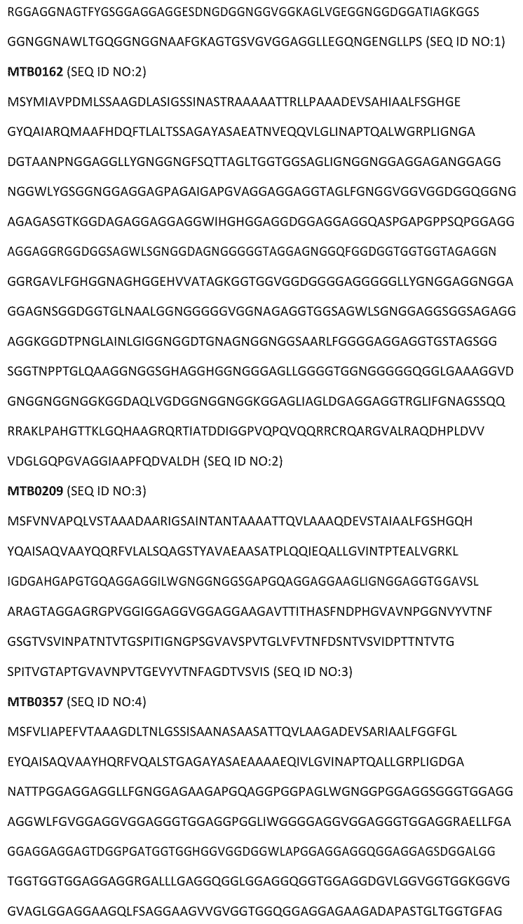

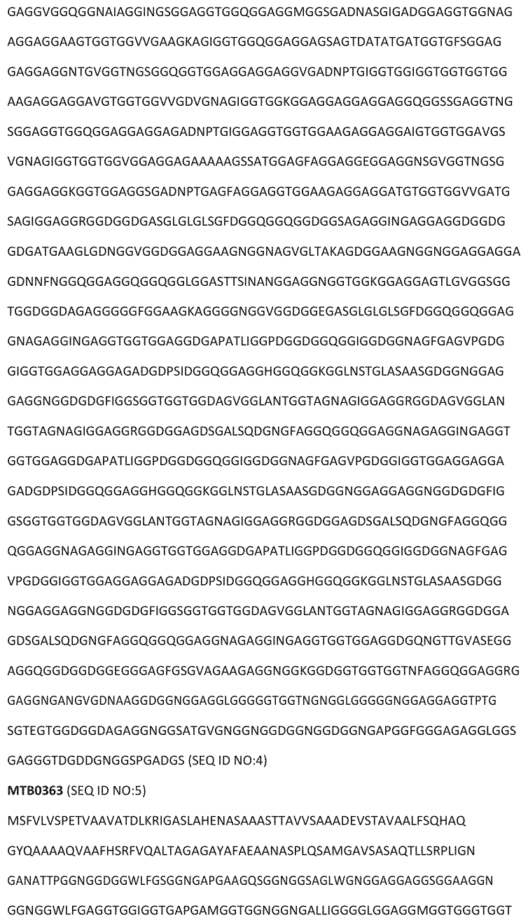

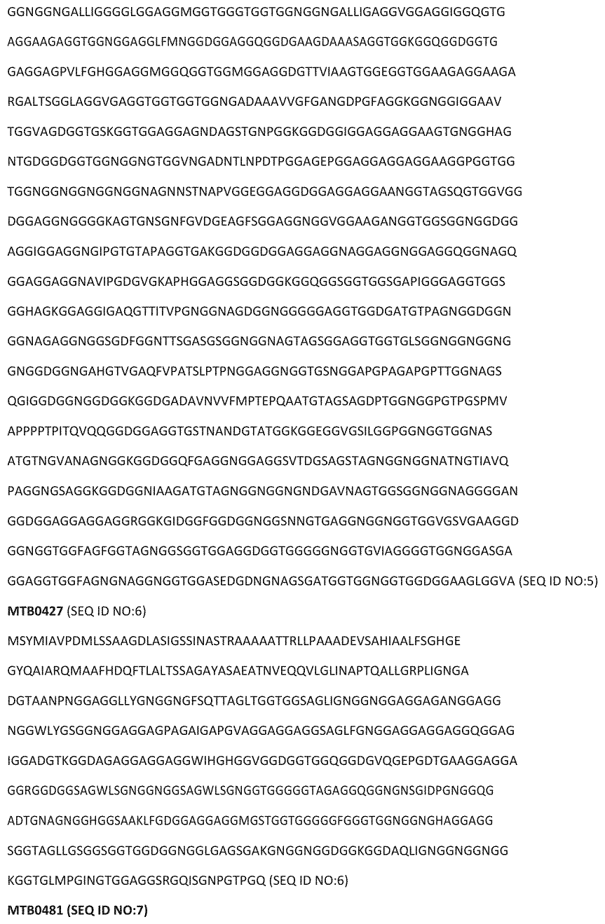

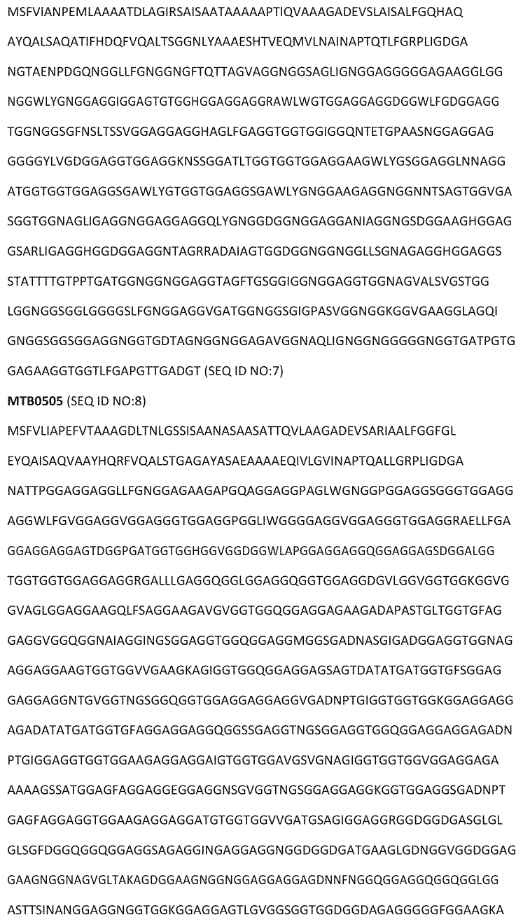

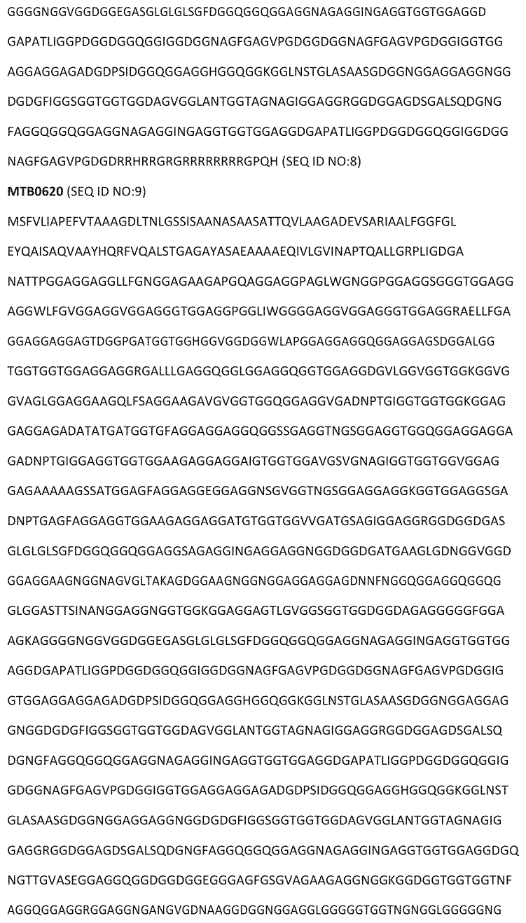

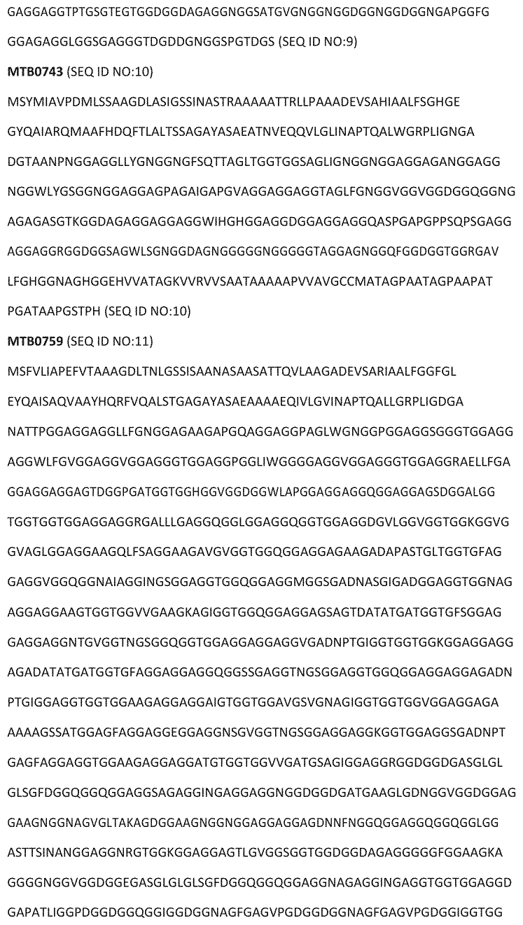

The names of the conserved region of the 52 special immune-evading MTBC PE/PPE genes of Table B, as illustrated in FIG. 32, are their common names and are described for example, in known databases such as GenBank.

Table C: MTBC genes

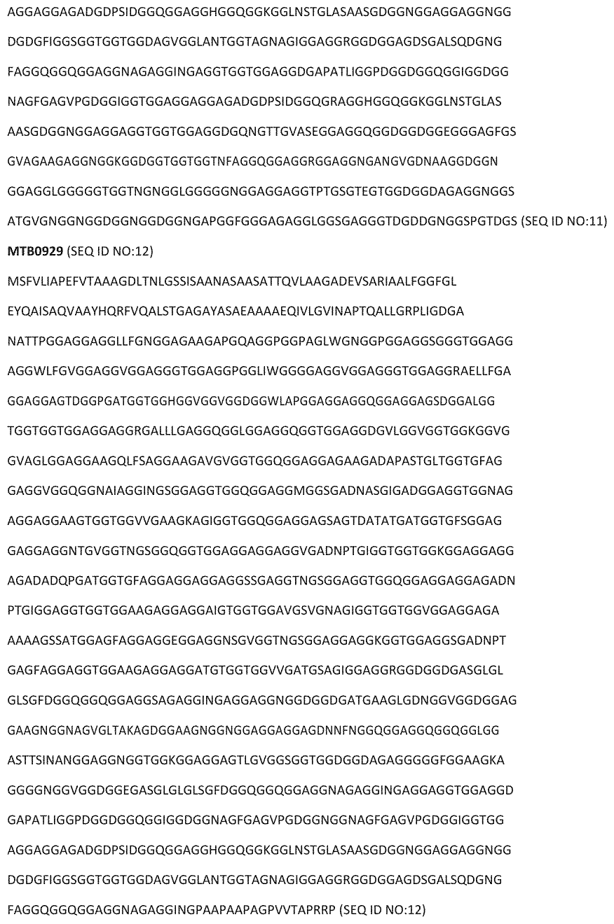

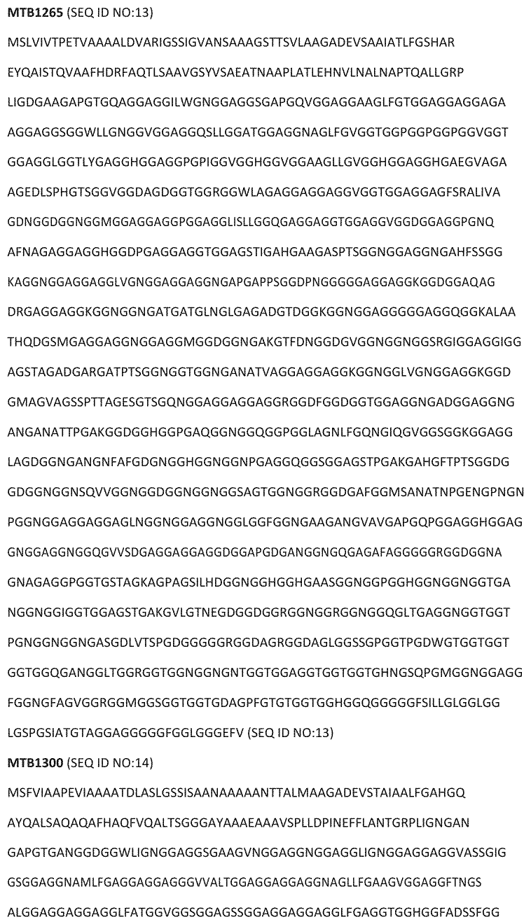

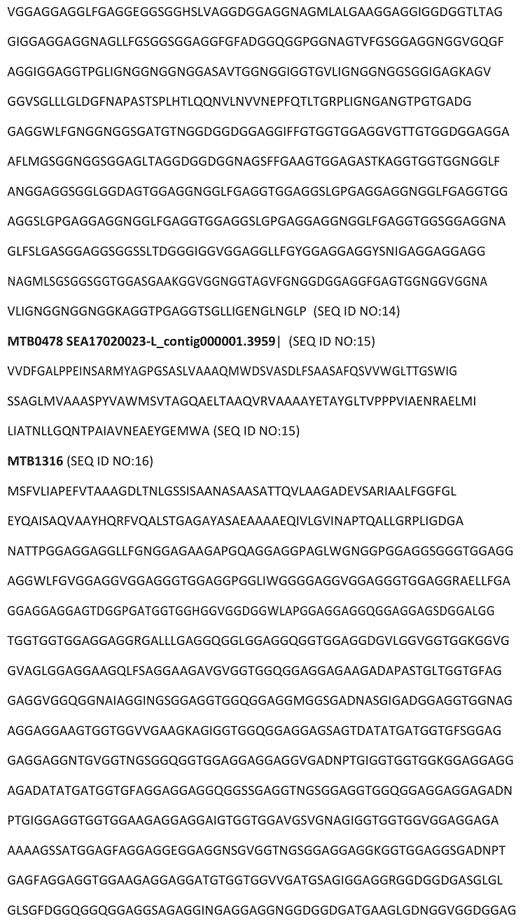

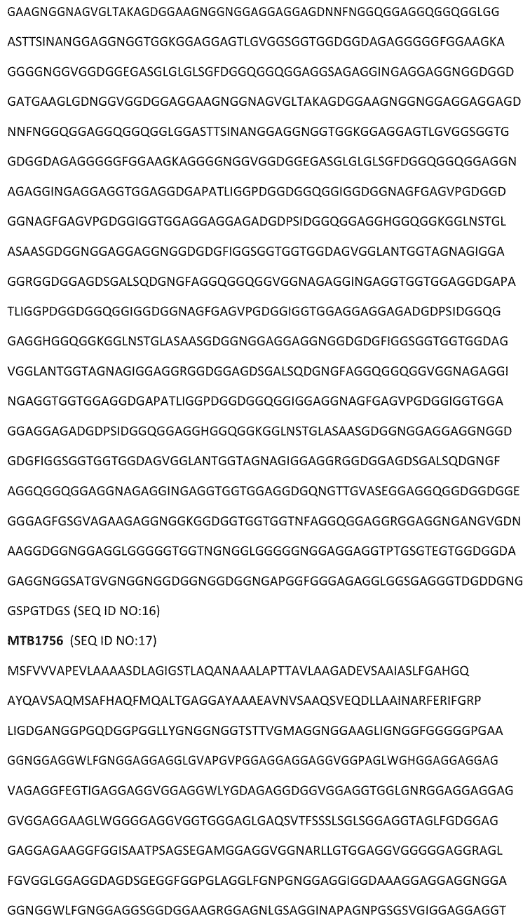

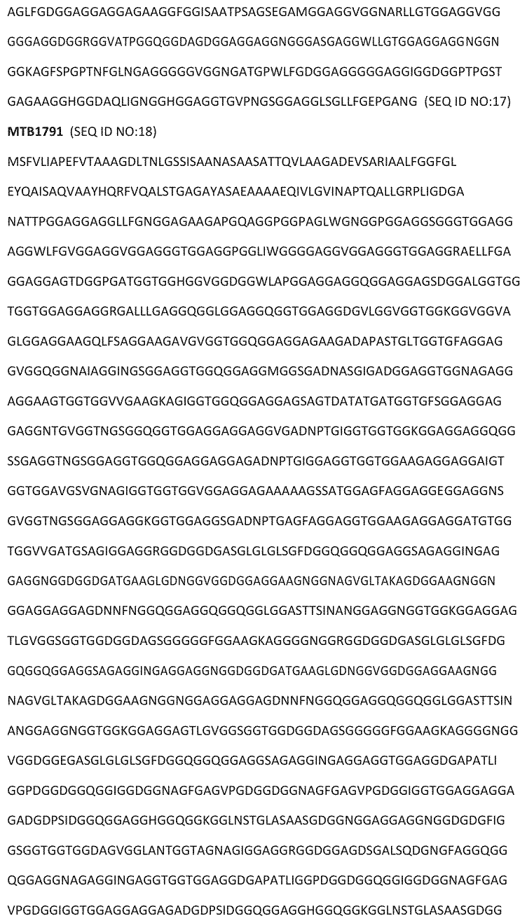

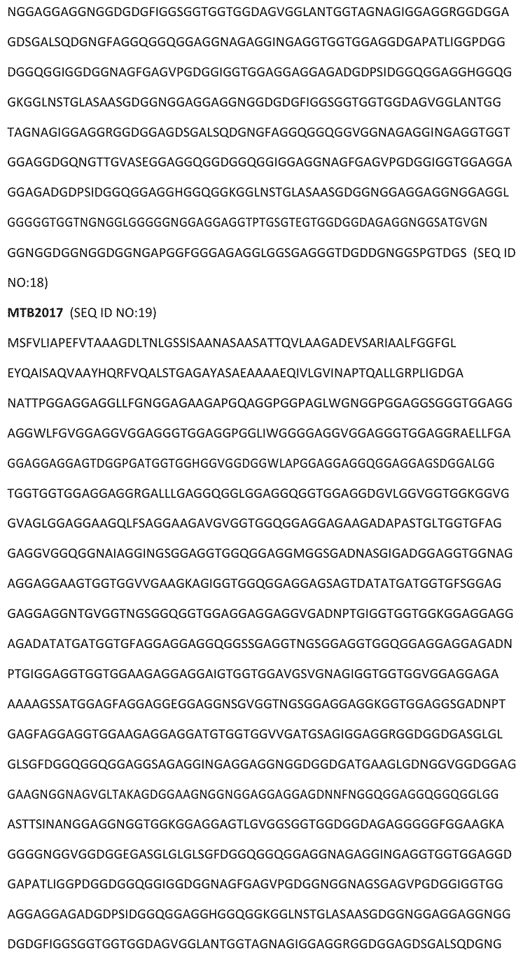

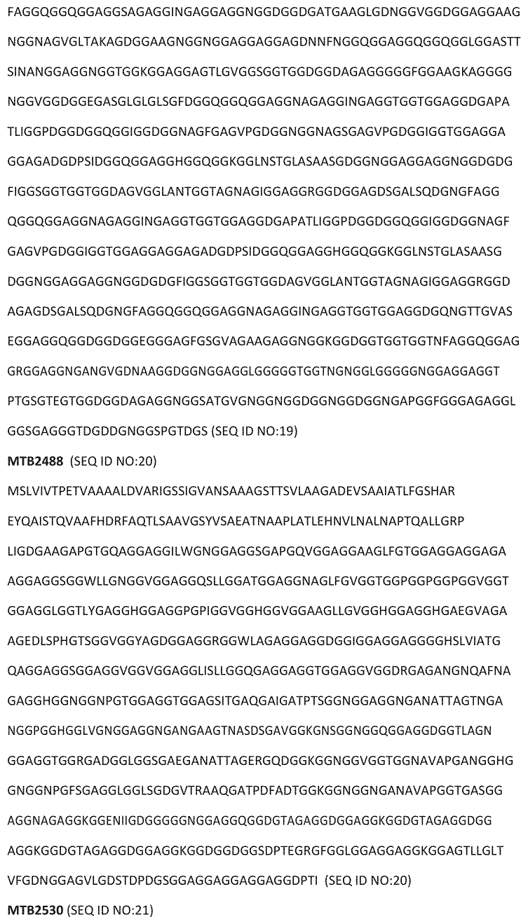

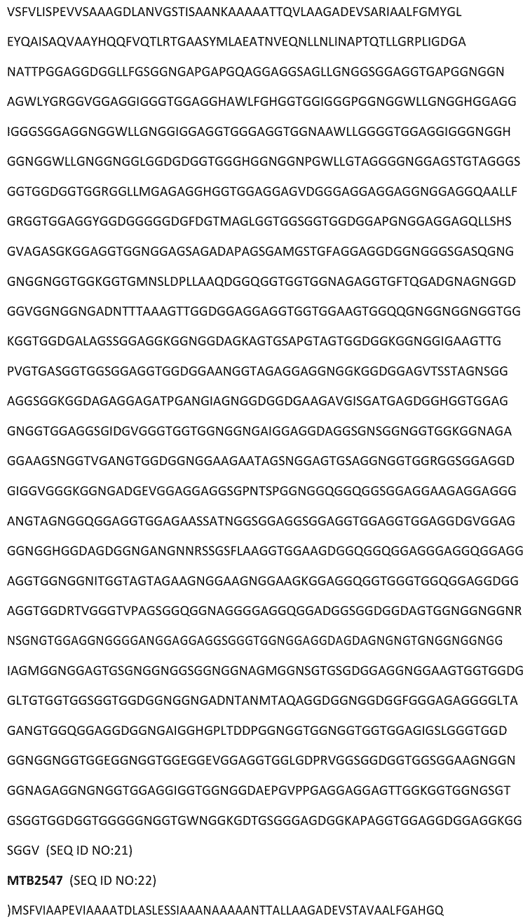

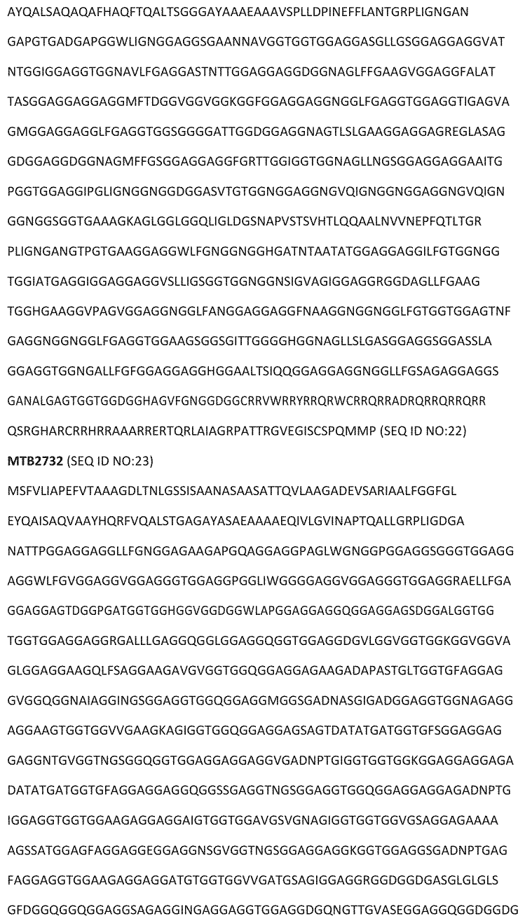

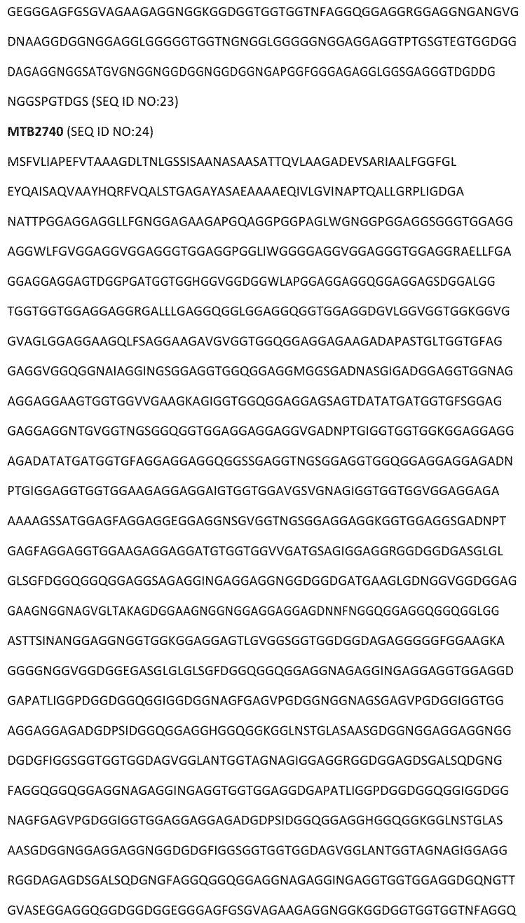

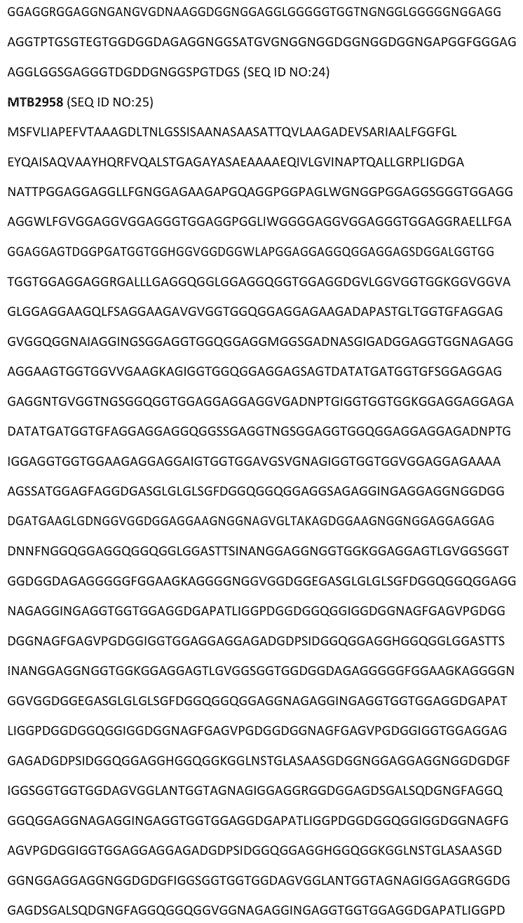

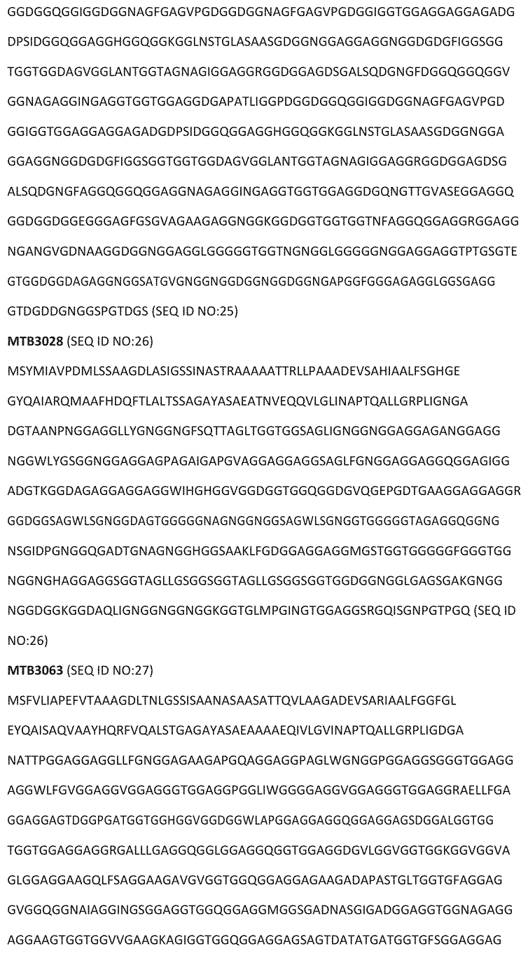

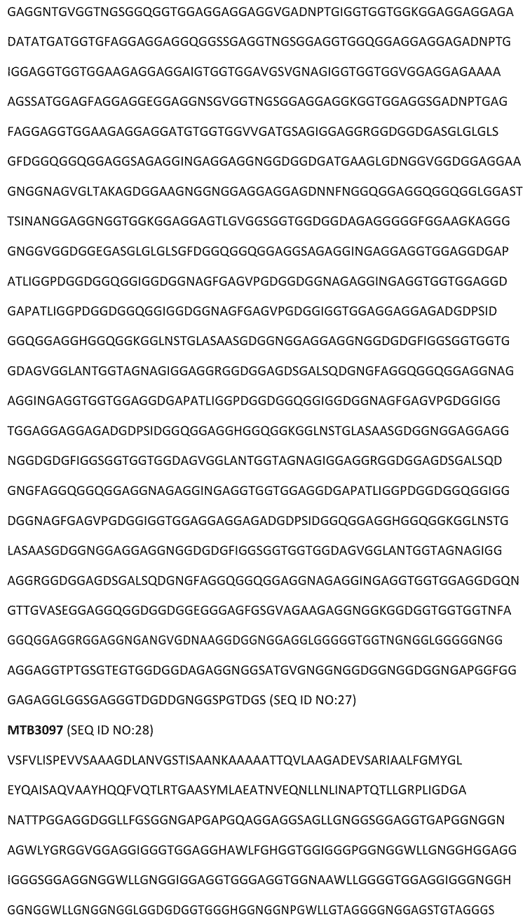

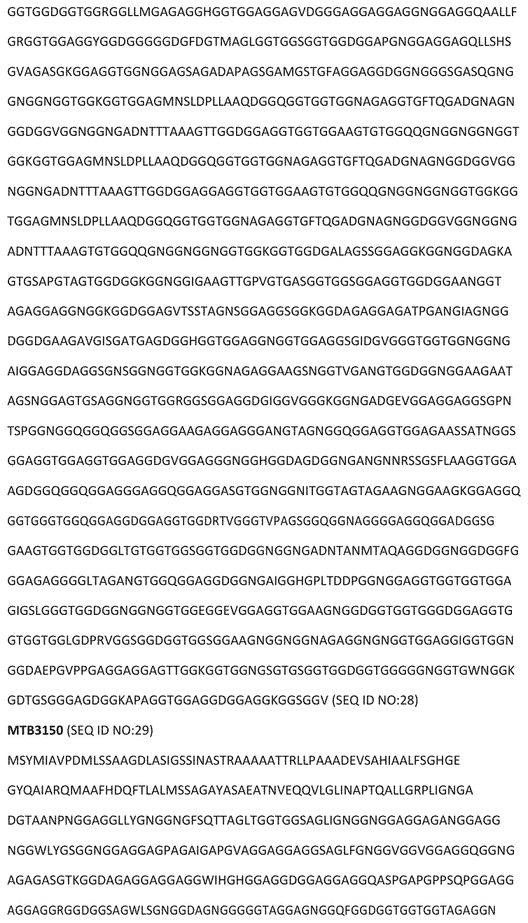

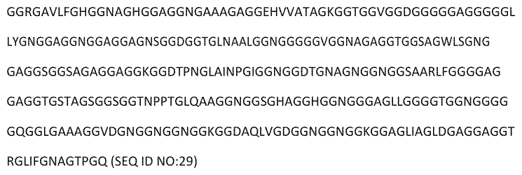

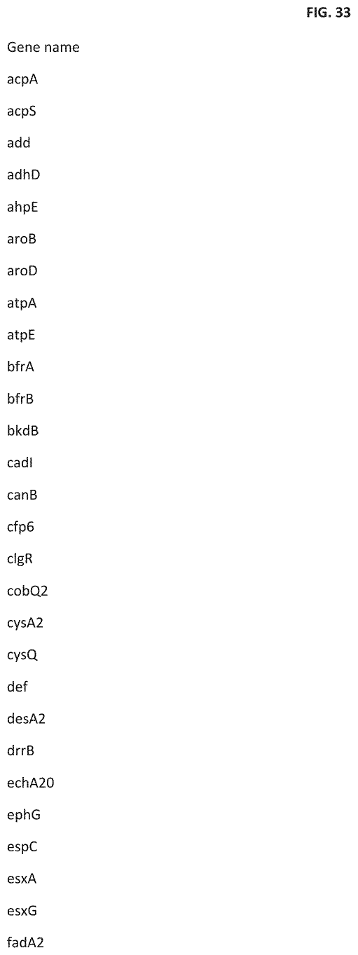

The names of the MTBC genes of Table C, as illustrated in FIG. 33, are their common names and are described for example, in known databases such as GenBank.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Reference-Quality Genomes Reveal Gene Gain, Loss, and Conversion Drive Genetic Diversity in Clinical Mycobacterium tuberculosis Strains

This example describes how to make and use alternative embodiments, as provided herein.

Mycobacterium tuberculosis is described to have low genetic diversity corroborated by consistent use of short-read sequencing. To elucidate the true genetic diversity, we present 97 reference-quality, circularized genomes sequenced on Pacific Biosciences from independent clinical M. tuberculosis isolates. A novel hybrid annotation approach was applied to all genomes, preserving reference annotations in regions of high homology but filling in gaps with an ab initio method. The resulting pangenome (6160 genes including 2061 not in H37Rv) revealed an accessory genome enriched for host interaction functions. The core genome (2556 genes) was enriched for housekeeping functions and contained 366 identical protein-coding genes in all genomes. Intriguingly, several toxin-antitoxin proteins were absolutely identical across all genomes, indicating high selective pressure to maintain these typically hypervariable genes. Sixty unique gene duplications were discovered, representing an important mechanism of gene gain in the species. However, gene loss through pseudogenization was significantly more prevalent than gene duplication, indicating more genome contraction than amplification. A per-genome calculation revealed an average of 1.5% of genes not in H37Rv, encompassing several PE/PPE genes. Remarkably, gene conversion was found to alter B cell epitopes in the PGRS domain of PE_PGRS proteins, denoting a potential mechanism of immune evasion in M tuberculosis. Although M. tuberculosis is considered a clonal bacterium, the differences we observed indicate a rapidly adapting genomic landscape that contributes to its success as a deadly pathogen.

### Results

Strict Quality Control Protocols Produce the Most Reference-Quality Genomes of M. tuberculosis To-Date

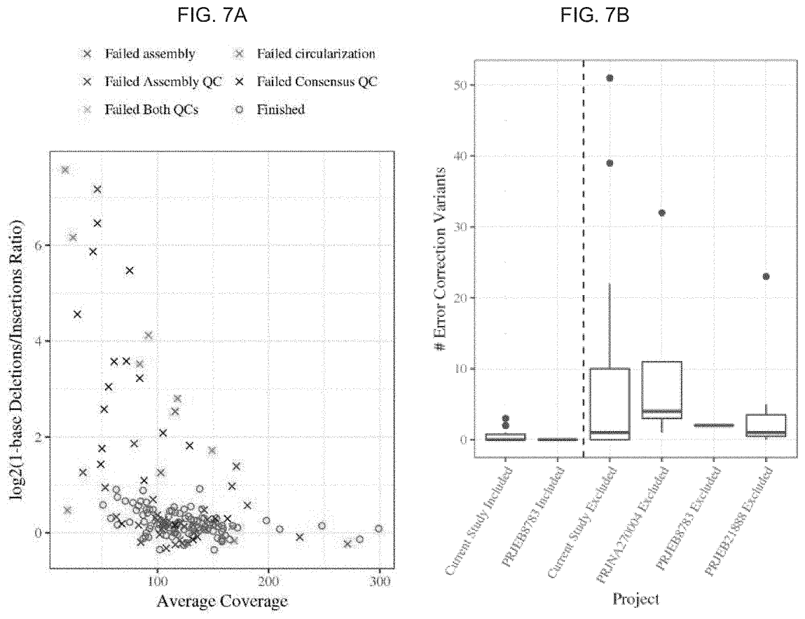

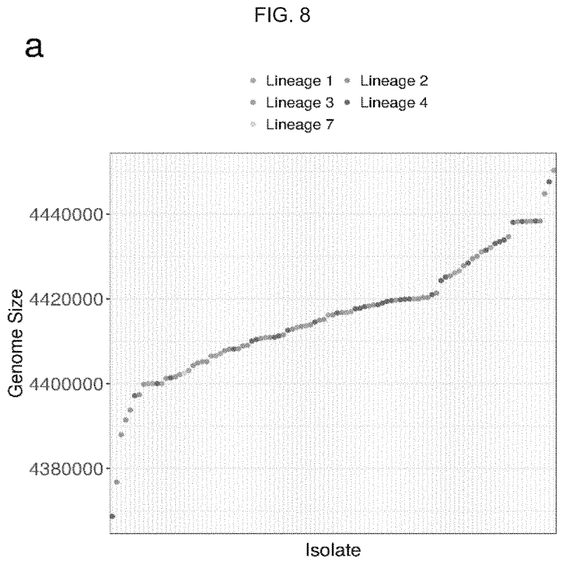

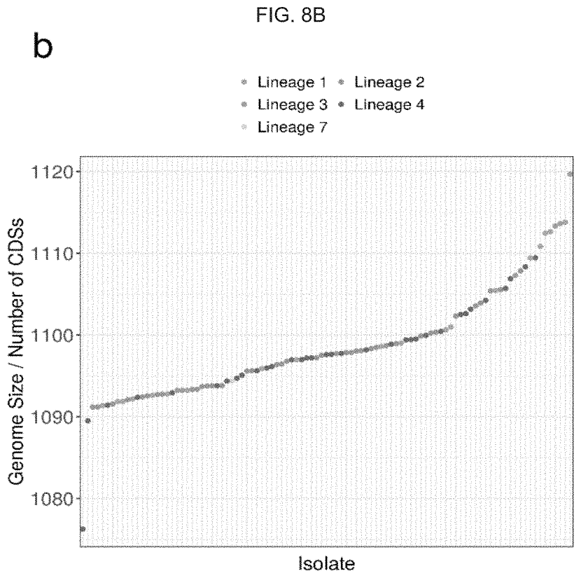

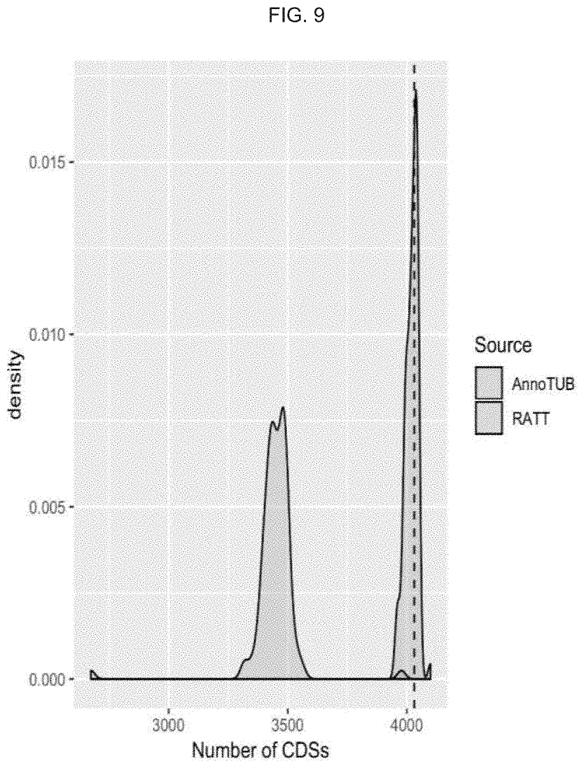

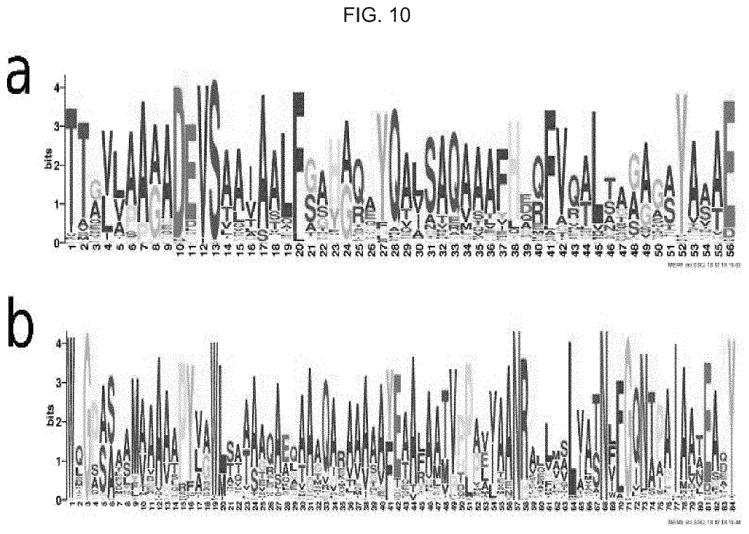

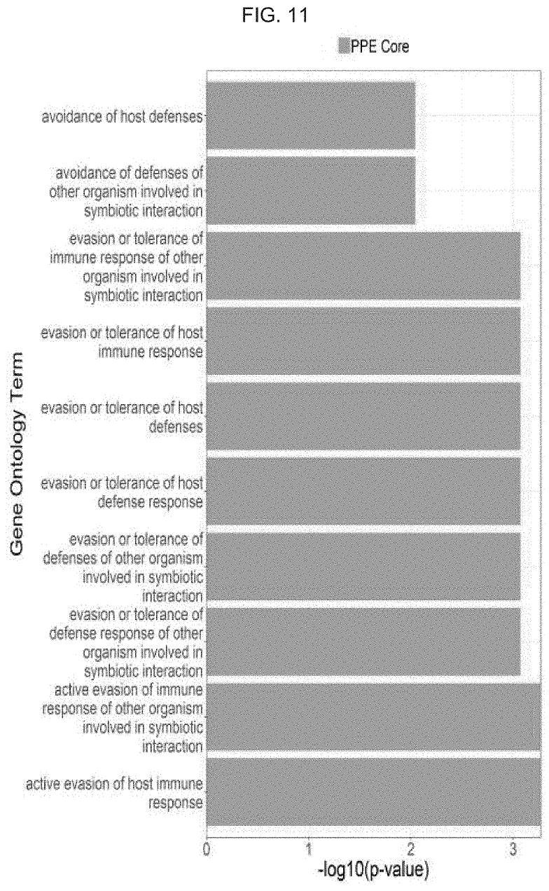

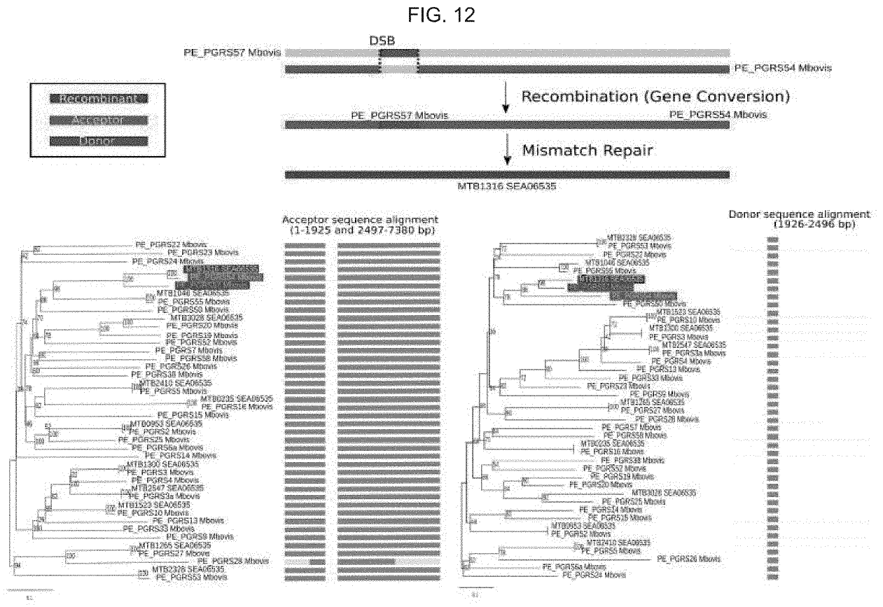

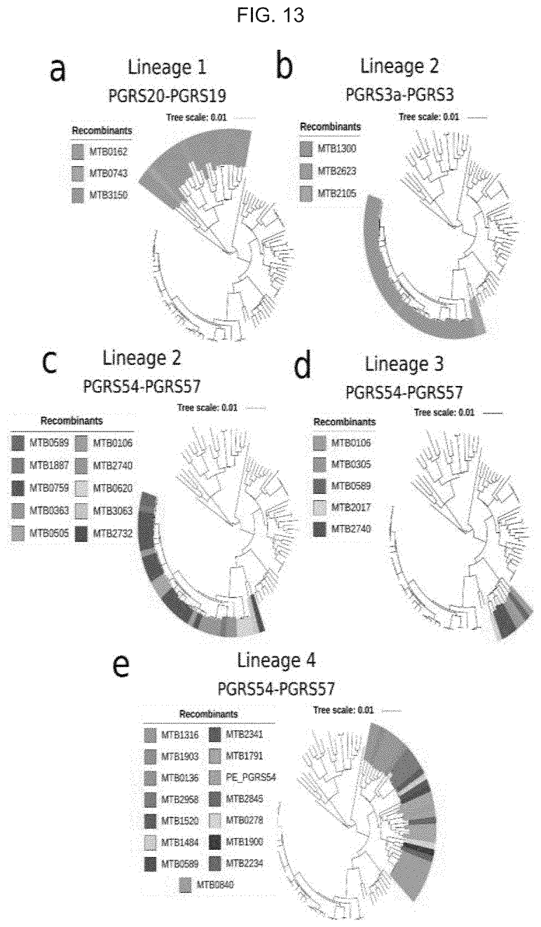

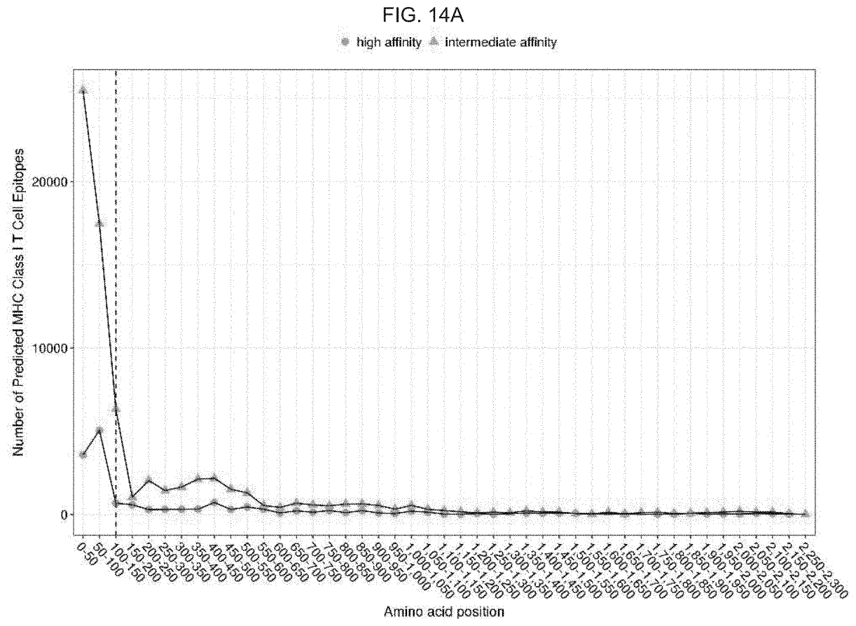

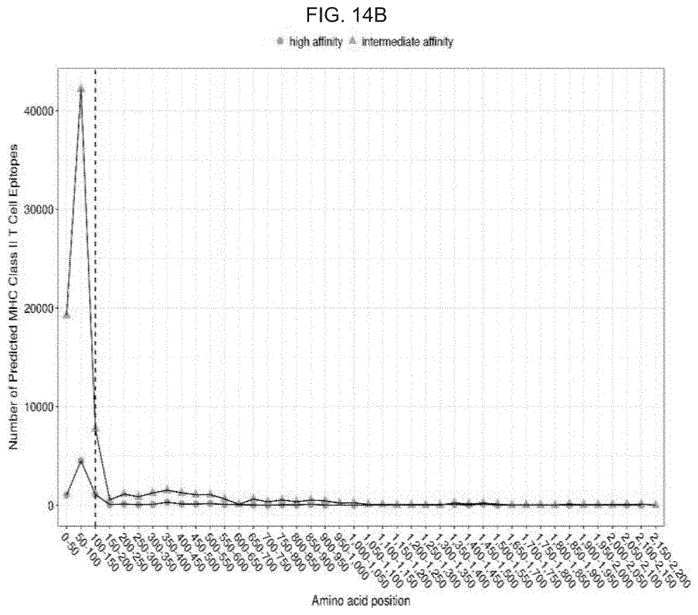

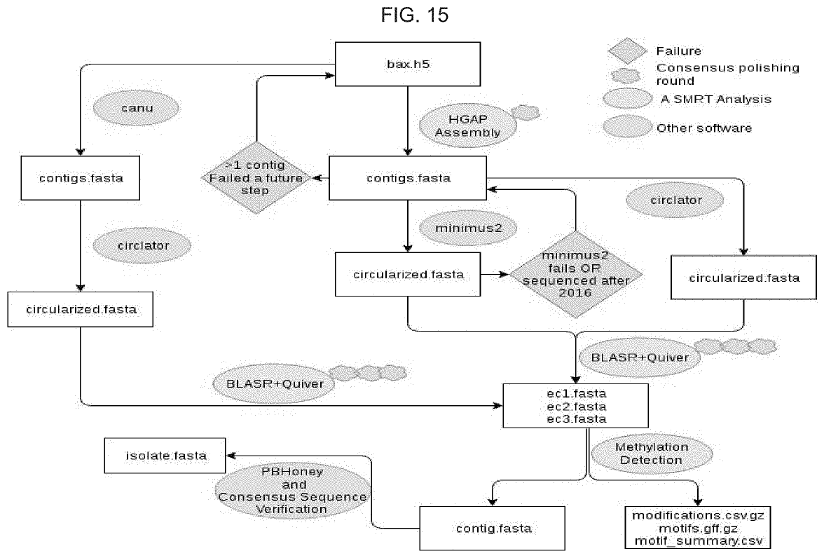

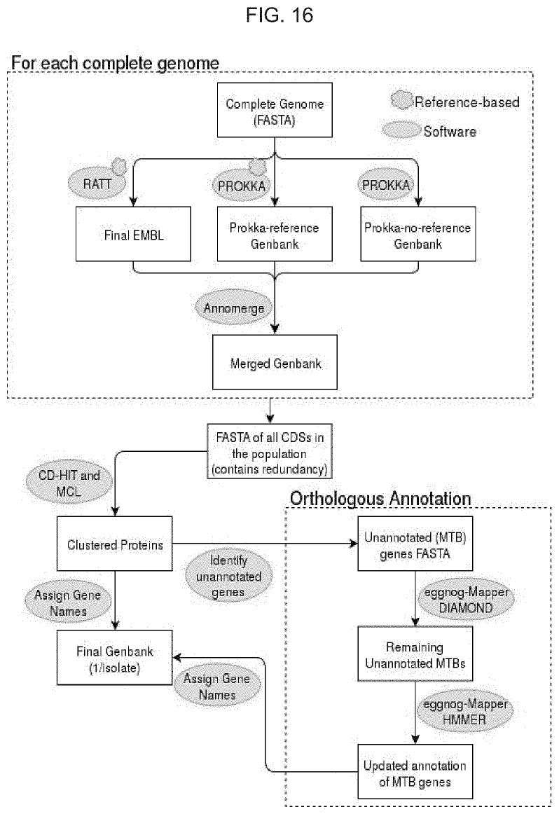

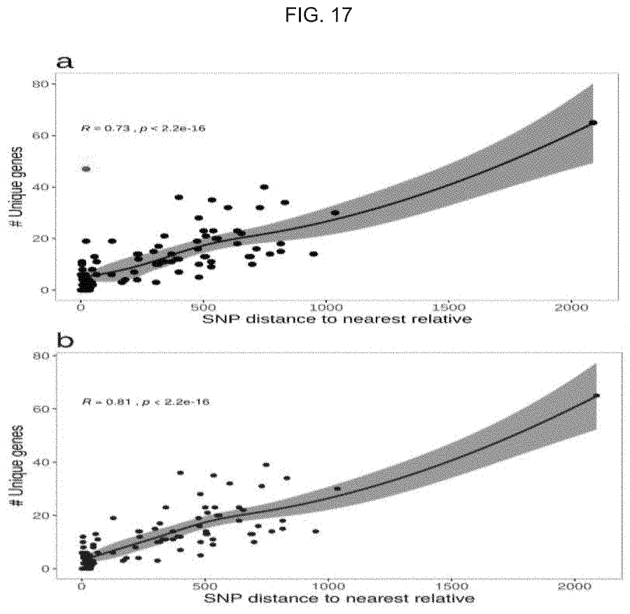

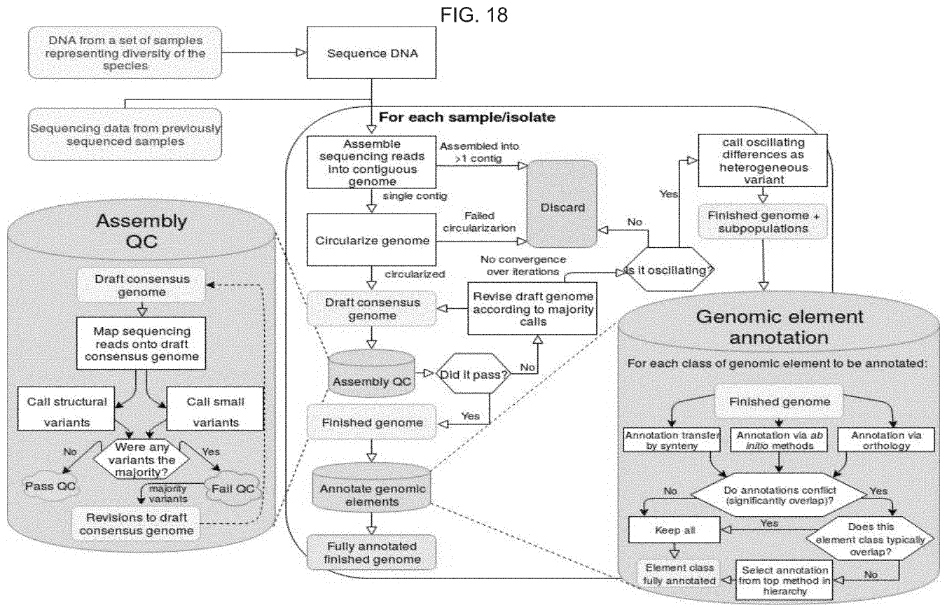

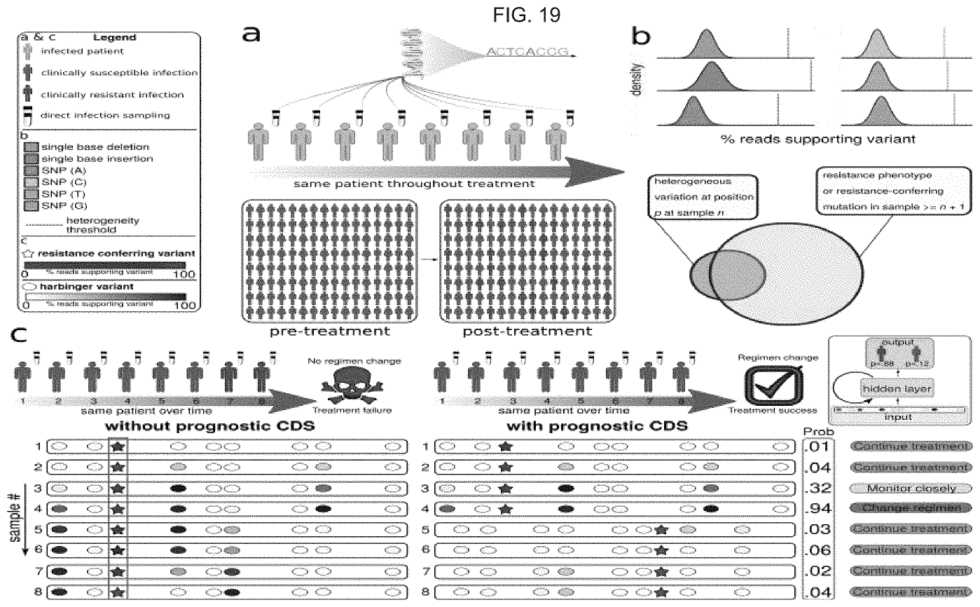

We compiled all available M. tuberculosis genomes (single patient isolates) that were sequenced on PacBio on the Sequence Read Archive database³⁰ (n=24) and sequenced an additional 154 genomes on PacBio RS II™. All genomes were assembled de novo and the quality of the consensus sequence was evaluated using strict exclusion criteria associated with each of the four assembly steps (described in “Genome Assembly with Quality Control” section). These criteria were used to exclude genomes with uncertainty at any point of their assembly (typically caused by single-base insertions or deletions). For statistics of excluded and included genomes, please refer to FIG. 7A-B.

FIG. 7A-B: Quality of 178 M. tuberculosis clinical isolates de novo assemblies a) The logarithmic-base 2 single base (1-base) deletion-vs-insertion ratio (as compared to H37Rv) as a function of average coverage in the assemblies with their corresponding de novo assembly status (QC: quality control; Failed assembly: could not assemble into at least one 4.4 Mbp contig; Failed circularization: could not confirm a circular genome; Failed Assembly QC: identified a structural break in the assembly with PBHoney that had high coverage support; Failed Consensus QC: could not resolve the consensus sequence in several regions of the genome; Failed Both QCs: failed assembly and consensus QC; Finished: assembled into a ˜4.4 Mbp contig, circularized, and passed all thresholds of QC). b) The number of variants identified in the third round of consensus error correction separated by study. Included: the group of de novo assembled genomes included in the pangenome study; Excluded: the group of de novo assembled genomes excluded from the pangenome study based on the failed assembly status from A.

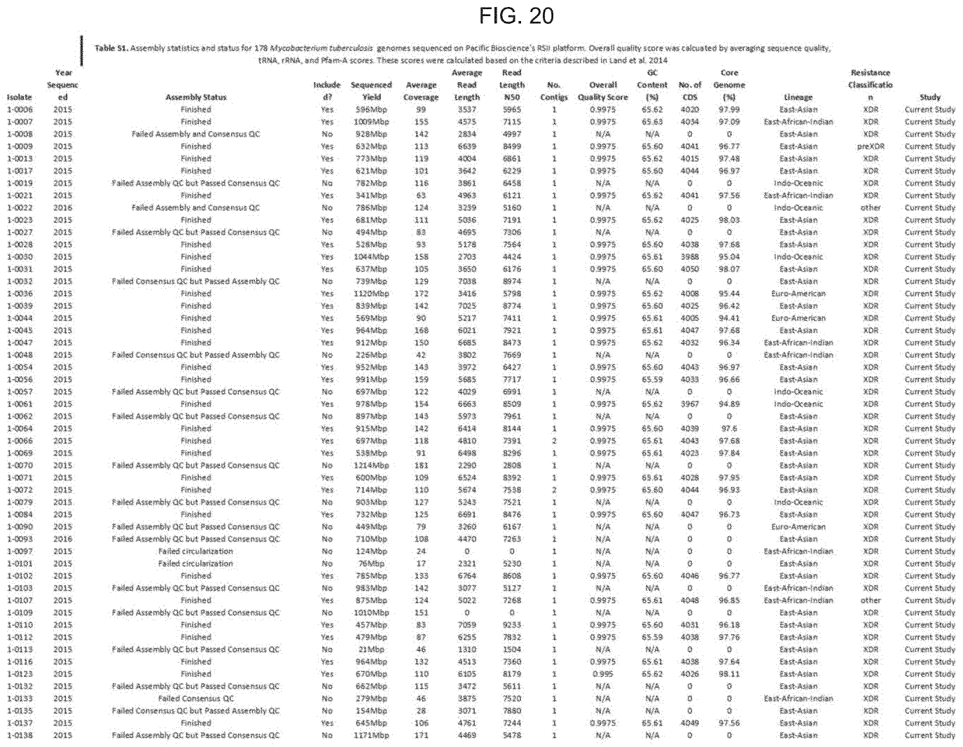

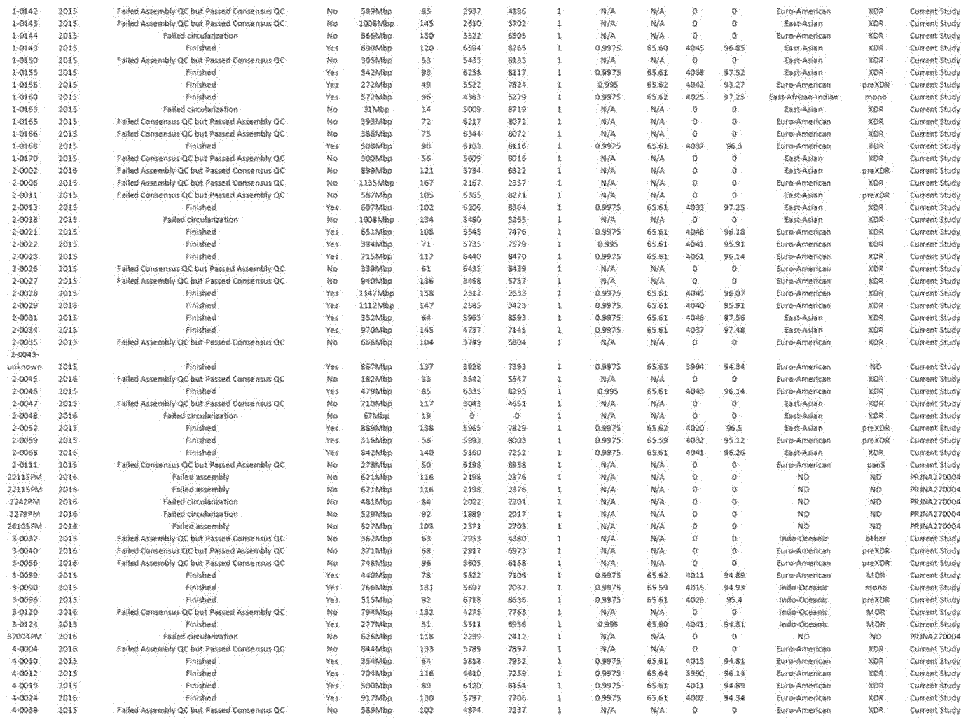

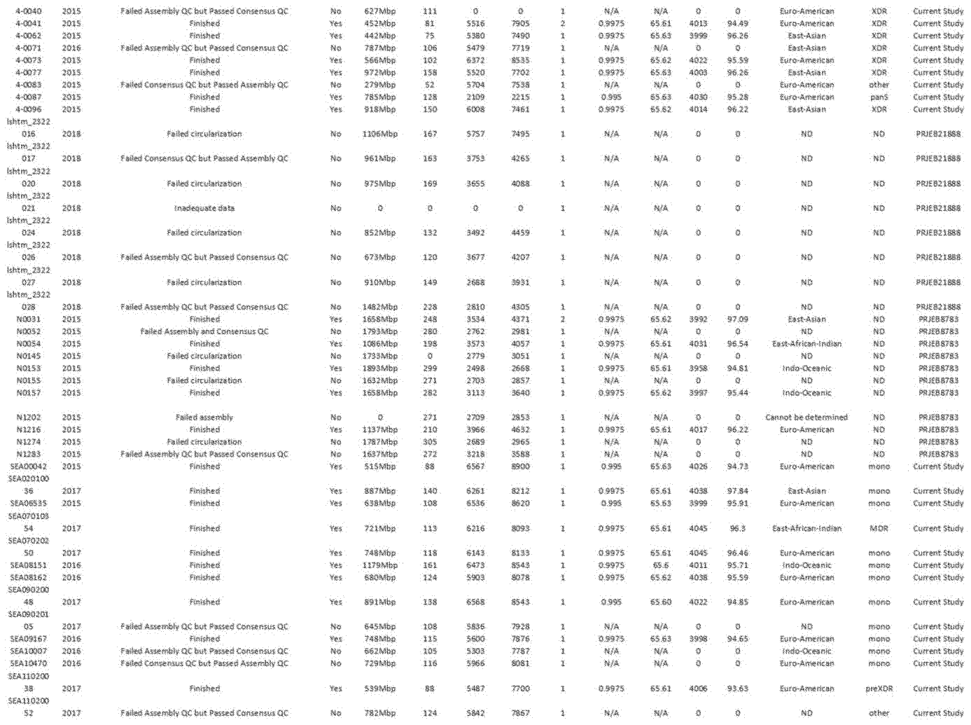

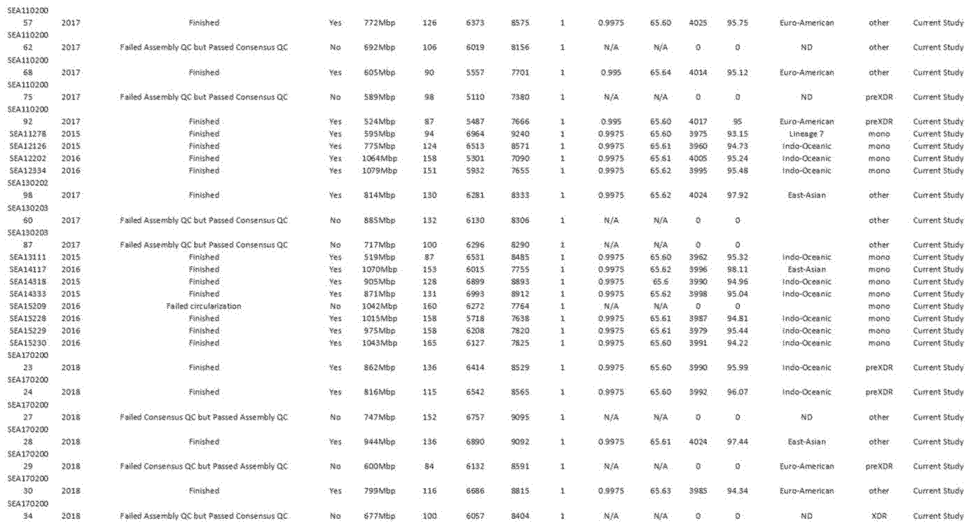

Applying these exclusion criteria identified 97 circularized genomes with a polished consensus sequence and no detectable mis-assemblies (Table S1, or FIG. 20). A further quantification of assembly quality was performed using previously described criteria³¹ using sequence quality, presence of full-length rRNAs (5S, 16S, and 23S), at least one tRNA for each amino acid, and presence of 102 essential domains from Pfam-A. Each category can have a maximal score of 1.0. All scores were 1.0 except Pfam-A essentiality scores, which was less than 1.0 in every genome (Table S1, or FIG. 20). A query revealed one Pfam domain absent from all genomes (Pfam accession PF07973), a threonyl and alanyl tRNA synthease. This absence could be attributed to not existing in M. tuberculosis or an error in annotation. Although this Pfam domain was absent in all genomes, the overall assembly quality scores were at least 0.995 across all 97 genomes, further supporting their use as reference genomes.

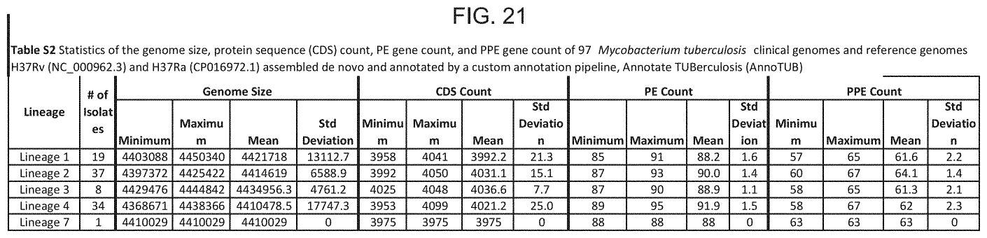

Genome sizes varied (minimum 4368671 bp, maximum 4450340 bp)) with over 80 kb difference between the largest and smallest genomes (FIG. 8A, Table S2, or FIG. 21).

FIG. 8A-B: Genome statistics for 97 M. tuberculosis clinical isolates' finished genomes, M. tuberculosis H37Rv (NC_000962.3), and M. tuberculosis H37Ra (CP016972.1). a) Genome sizes per isolate colored by lineage b) Proportion of genome size to number of protein coding sequences (CDSs) per isolate colored by lineage.

A phylogenetic tree was also produced to ensure sequencing errors did not affect the expected evolution of the included M. tuberculosis genomes. Similar to previous studies (Kato-Maeda et al. 2013; Stucki et al. 2012), a SNP tree was generated by, first, identifying all SNPs compared to reference genome, M. tuberculosis H37Rv; then a phylogenetic tree was created with a maximum likelihood method. MIRU-VNTR and spoligotyping data was referenced for accuracy.

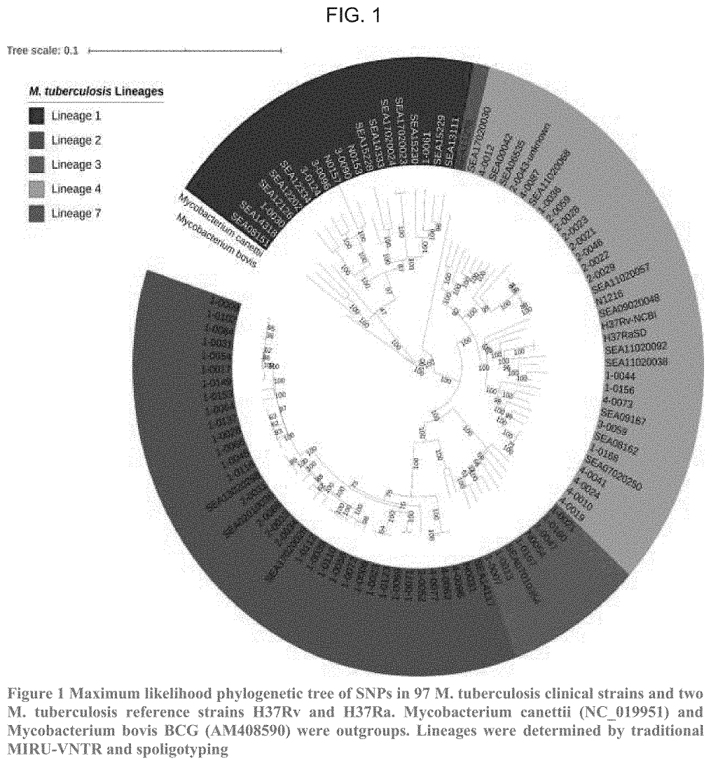

FIG. 1 demonstrates the expected phylogeny of M. tuberculosis strains with lineage 1 being the most ancestral followed by lineage 7, lineage 2, lineage 3, and lineage 4 (Gagneux and Small 2007; Comas et al. 2015).

FIG. 1 illustrates: Maximum likelihood phylogenetic tree of SNPs in 97 M. tuberculosis clinical strains and two M. tuberculosis reference strains H37Rv and H37Ra. Mycobacterium canettii (NC_019951) and Mycobacterium bovis BCG (AM408590) were outgroups. Lineages were determined by traditional MIRU-VNTR and spoligotyping

These reference-quality genomes were then queried for new genetic content through a hybrid annotation approach.

### Hybrid Annotation Approach Greatly Reduces Mis-Annotations and False Positive Annotations

We sought to preserve annotations from a well-studied reference strain (H37Rv³², implemented by RATT³³) and annotate new content ab initio (implemented by Prokka³⁴). Relying on a well-characterized reference annotation lowers the possibility of mis-annotations and false positive annotations, common in ab initio methods³⁵. The annotation transfer resulted in an average of 85% concordance with the H37Rv proteome across 97 genomes (Figure S3). However, relying solely on this type of annotation removes the possibility of identifying new genes with respect to the reference. An ab initio approach was used to annotate new genes not present in the reference, along with two custom filtering steps to avoid mis-annotations and false positive annotations. The first step minimized mis-annotation of gene coordinates (characterized by an incorrect protein-coding sequence (CDS) start position) by recalibrating the gene coordinates of CDSs likely to have an incorrect start coordinate (see Supplemental Information). The second step removed potential false positive annotations in genomic regions that are unannotated by RATT by excluding genes with unknown function (see Supplemental Information for details). These steps, a CDS sequence clustering step, and orthologous functional annotation with eggNOG comprise a custom annotation pipeline for M. tuberculosis genomes, Annotate TUBerculosis (AnnoTUB).

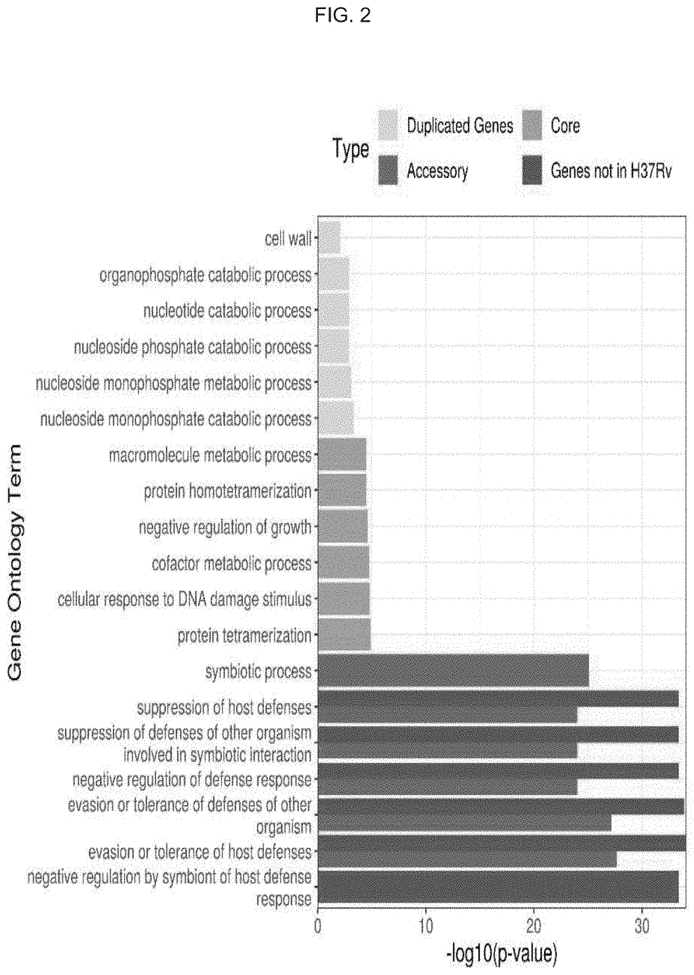

FIG. 2 illustrates gene ontology (GO) enrichment within the accessory and core genomes, genes that have been duplicated, and genes that are not in the reference strain, H37Rv, in 97 M. tuberculosis clinical isolates' genomes. Narrow bars represent shared GO terms between Types.

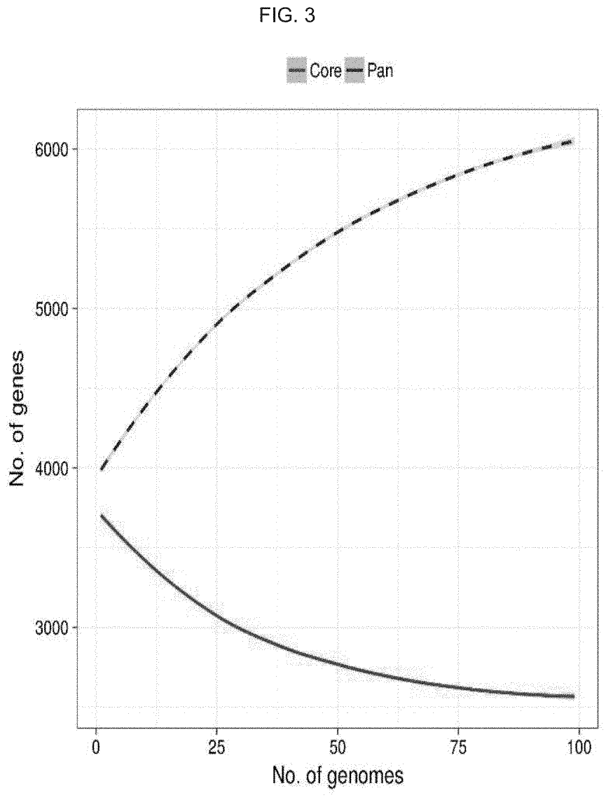

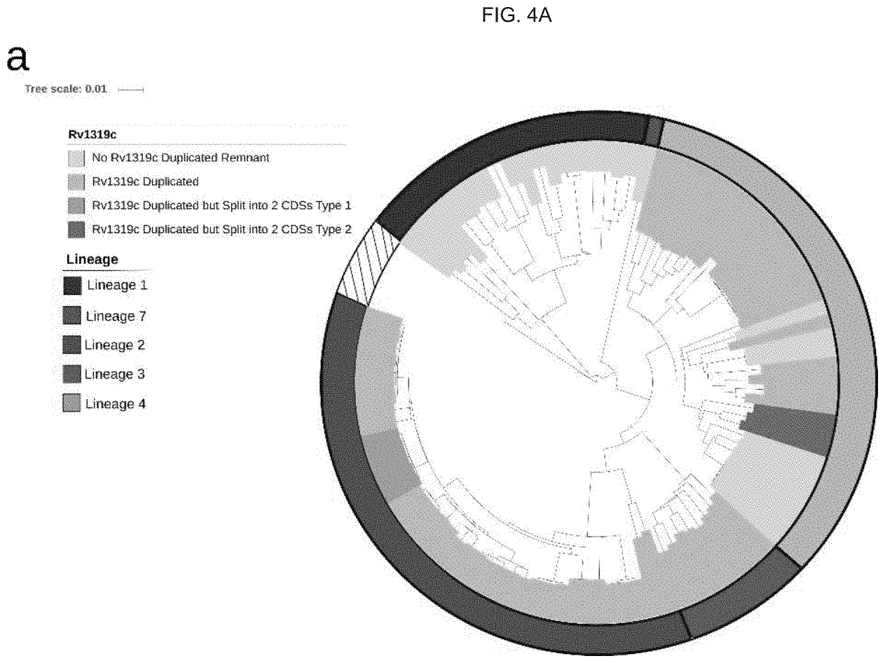

FIG. 3 illustrates the trend of CDS count as new genomes are added to a pangenome analysis of de novo assembled genomes from 97 M. tuberculosis clinical strains and two M. tuberculosis H37 reference strains: H37Rv (NC000962.3) and H37Ra (CP016972.1).

AnnoTUB identified an average of 4020 CDSs (range 3958-4051) in 97 genomes as well as the avirulent M. tuberculosis reference strain, H37Ra (CP016972.1), and re-annotation of H37Rv (NC000962.3) (Table 51, or FIG. 20). Interestingly, the proportion of genome size to CDS count associated with lineage. Specifically, lineage 1 isolates had the highest ratio of genome size to CDS count (FIG. 8B) caused by the low average CDS count (Table S2, or FIG. 21). Next, to identify gene duplications and pseudogenes and isolate potential recombination events that might explain these varying CDS counts, we compiled all orthologous proteins in a pangenome analysis.

M. tuberculosis Pangenome Reveals Hidden Genetic Diversity and Hyperconservation

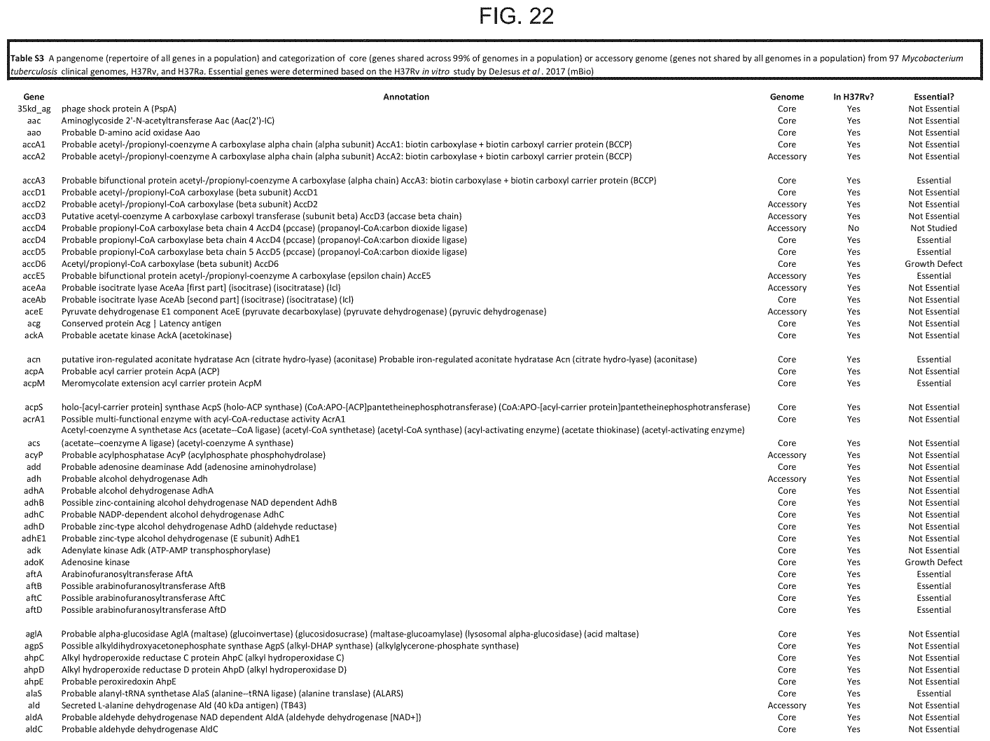

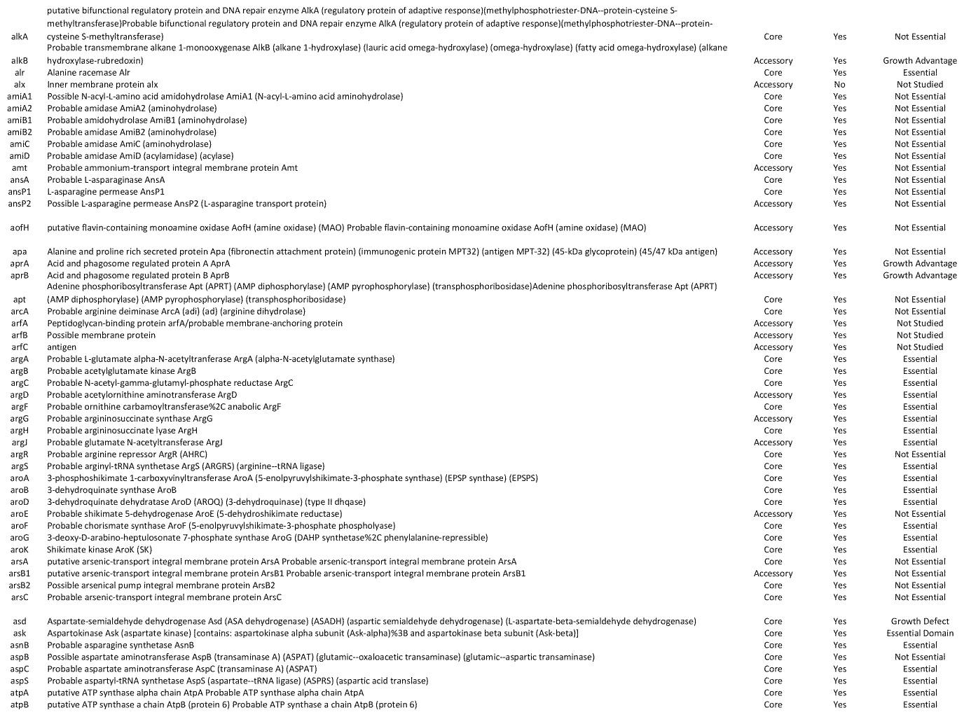

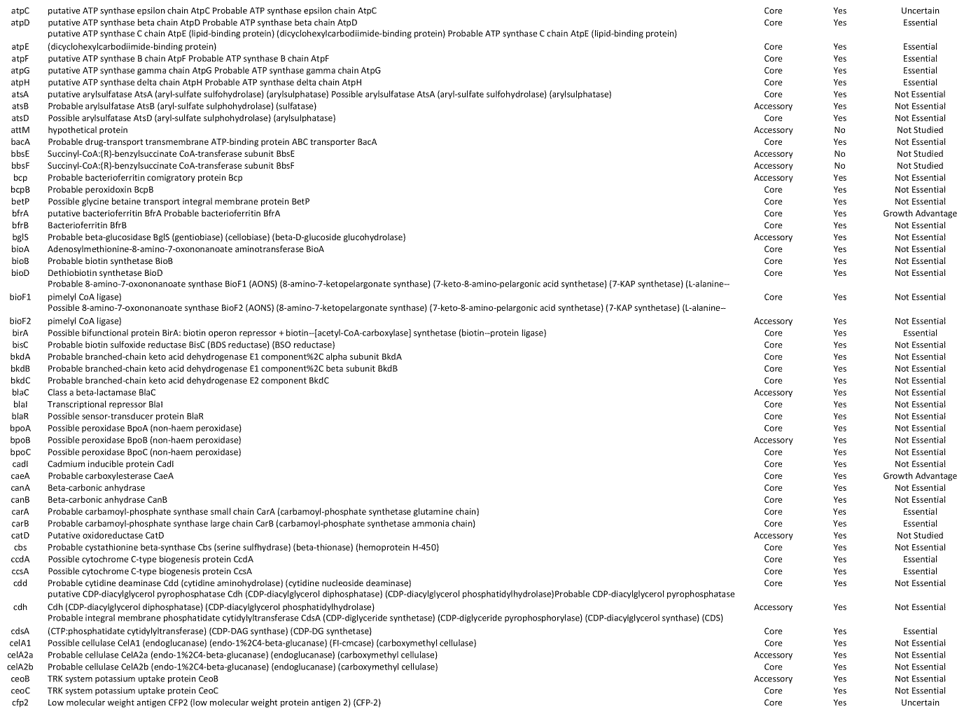

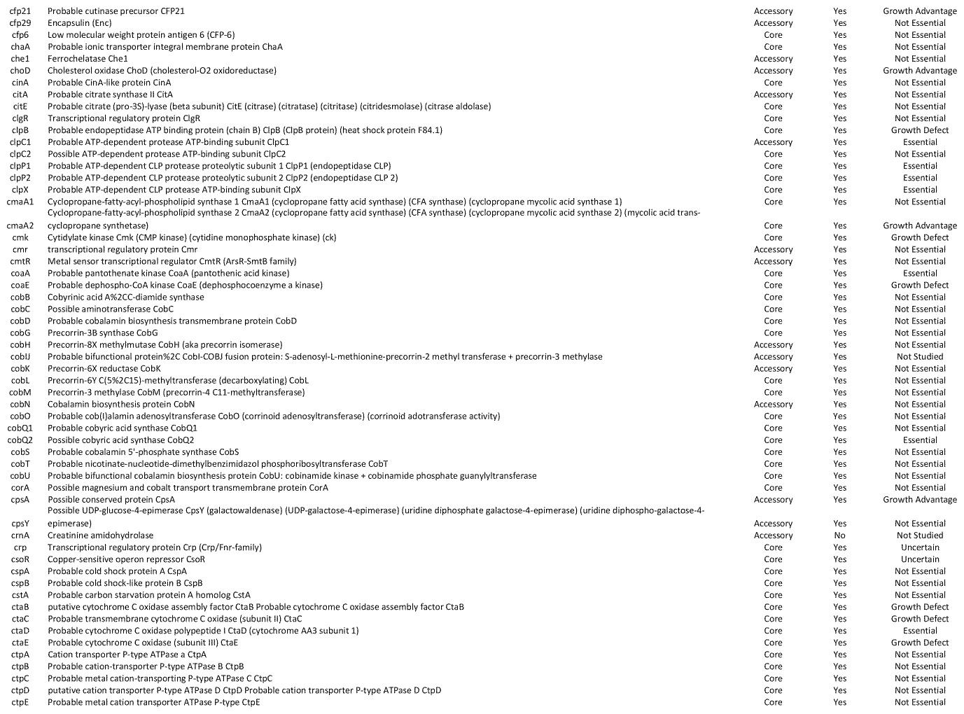

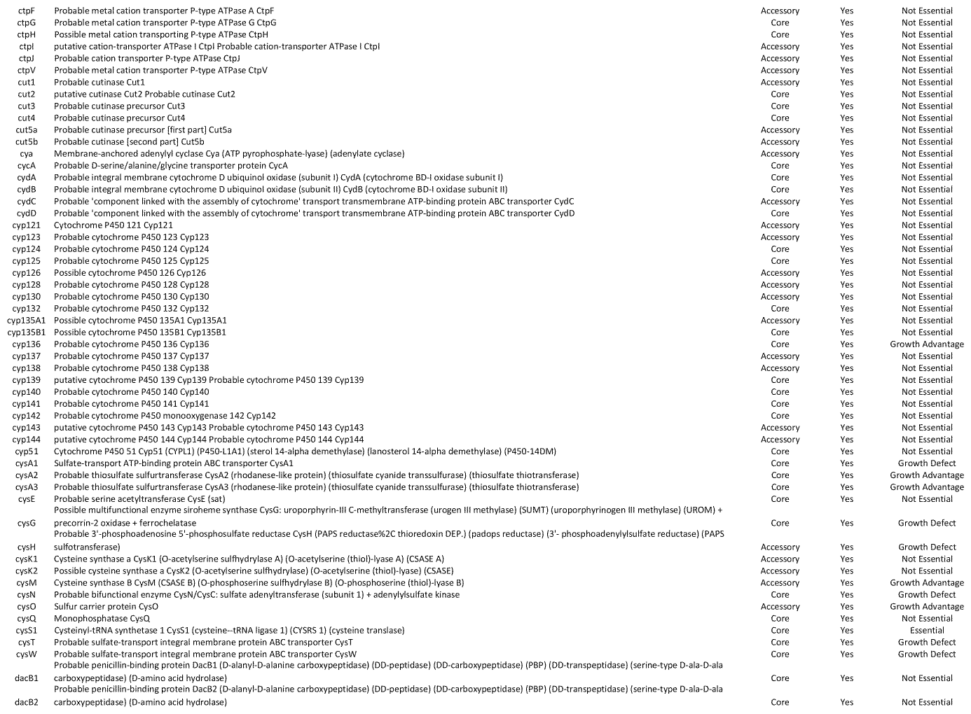

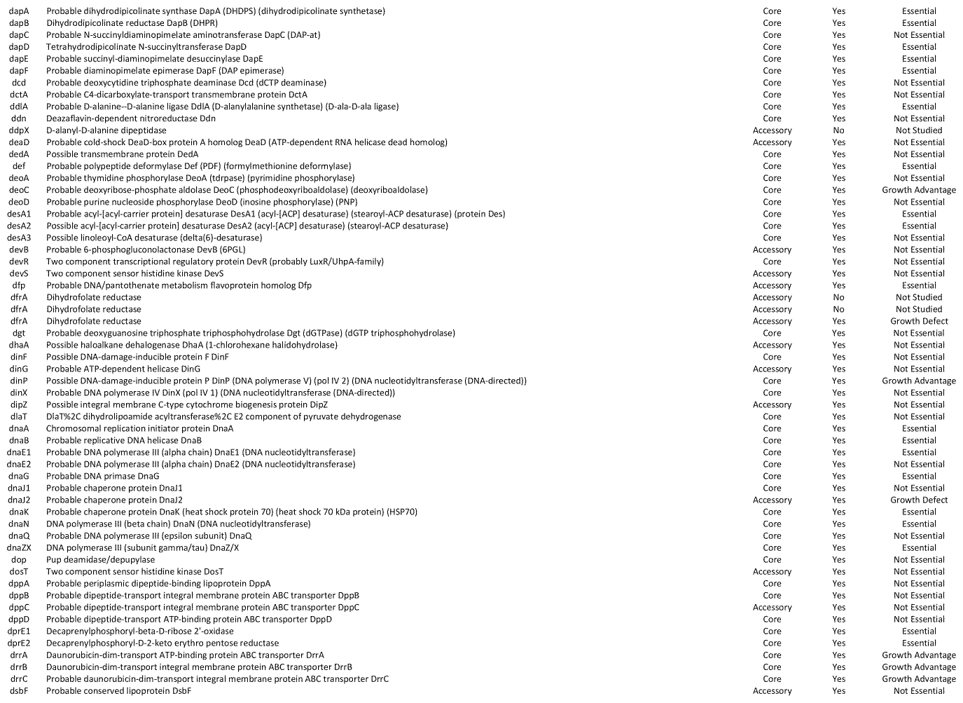

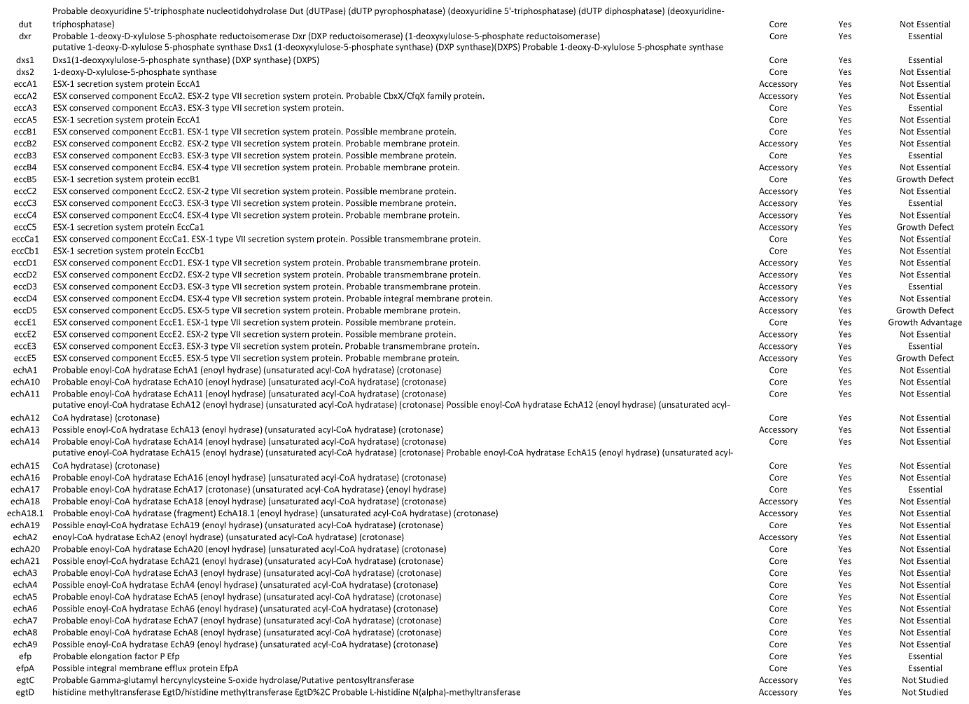

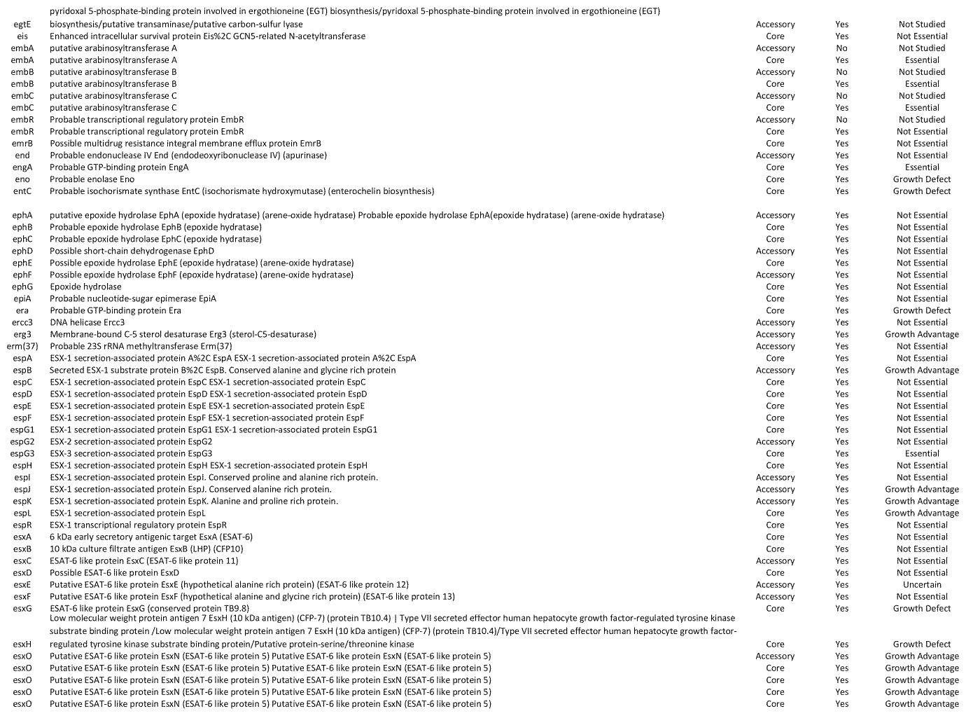

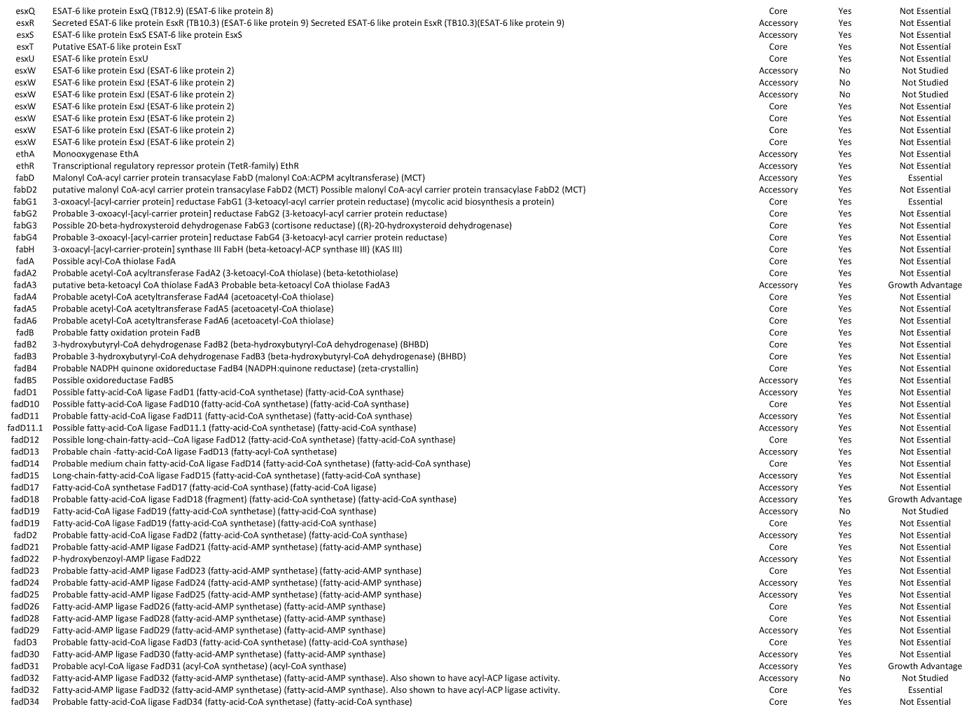

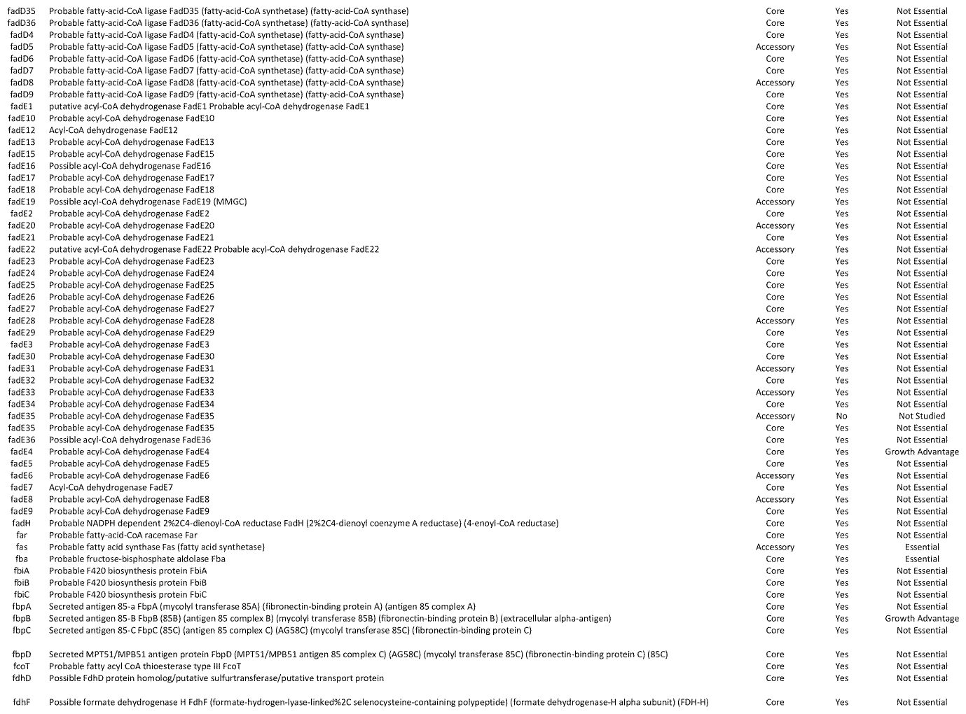

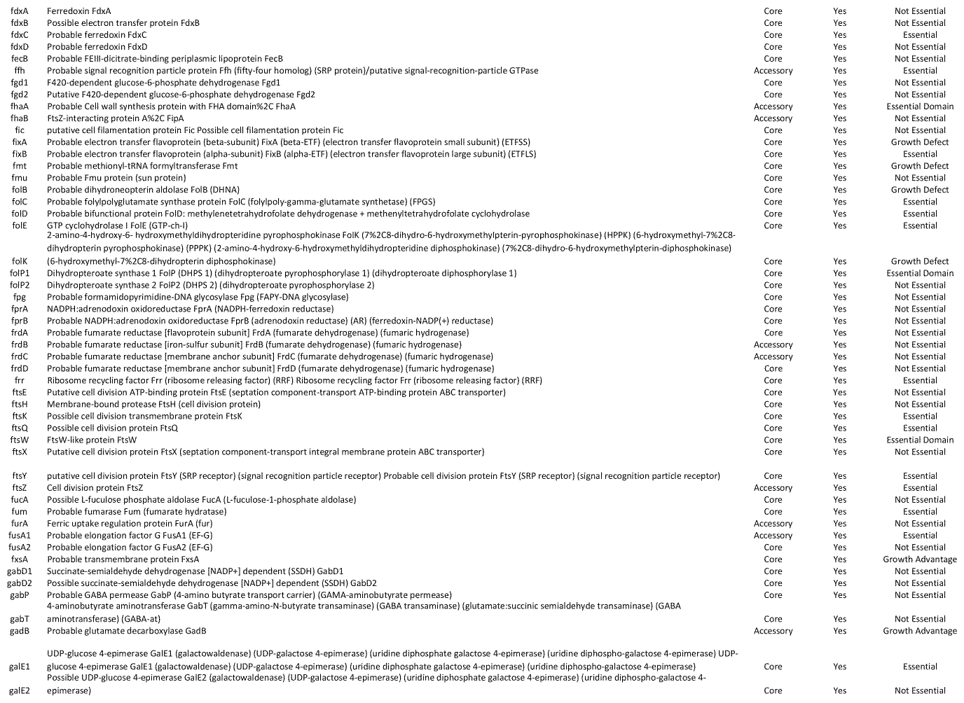

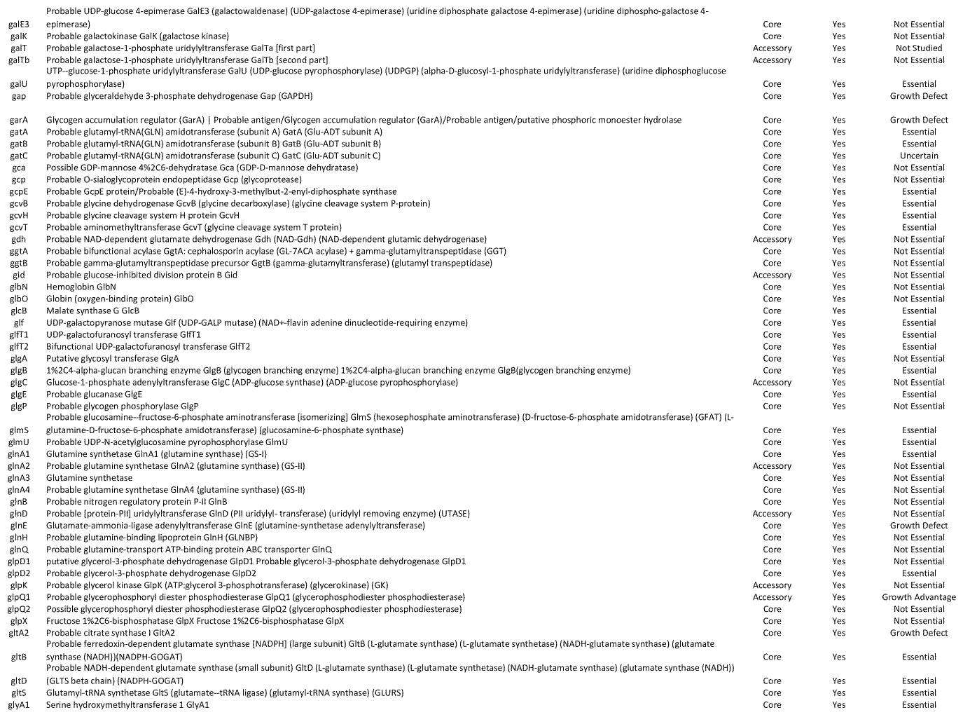

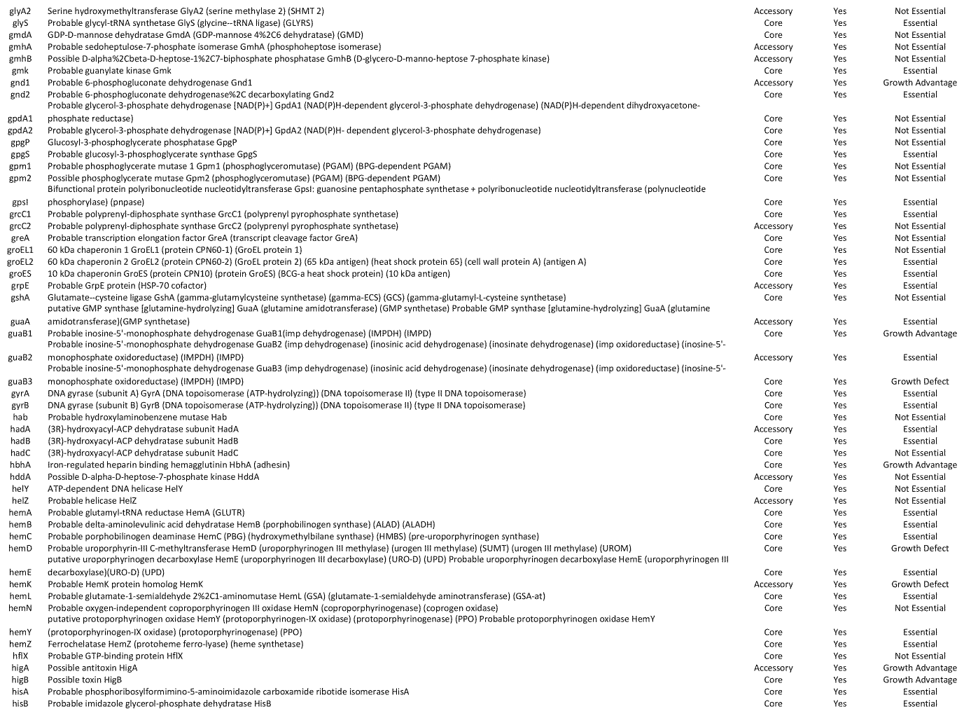

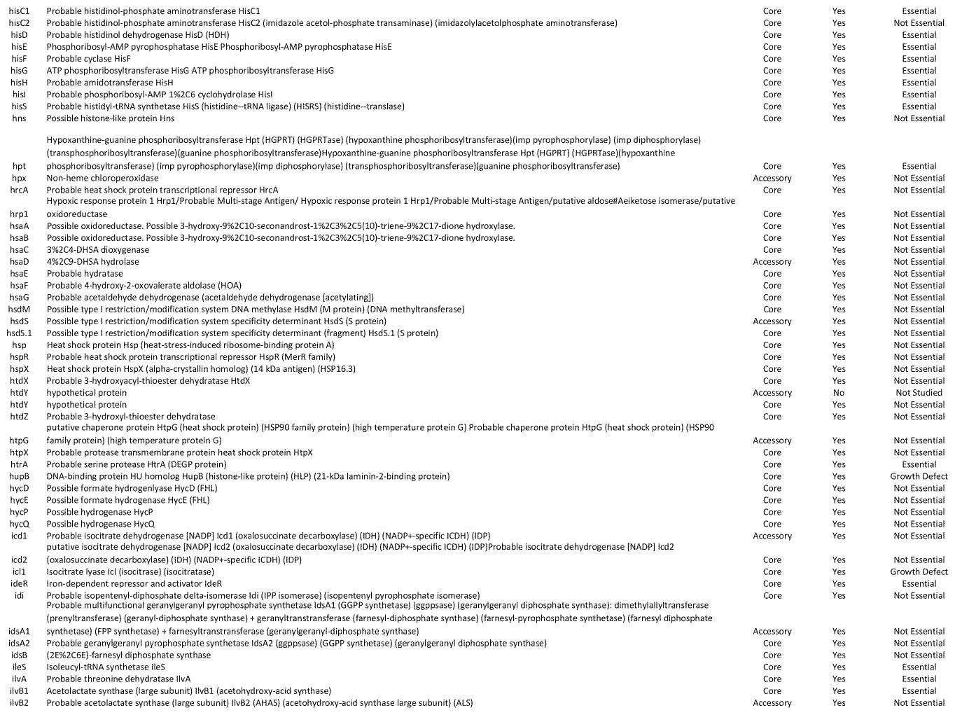

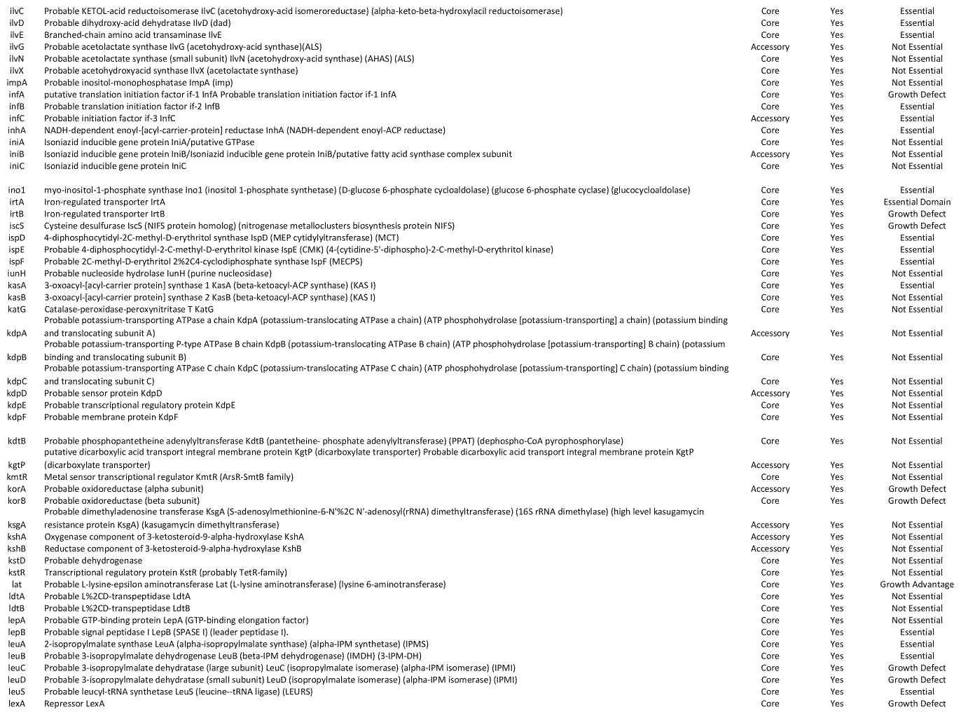

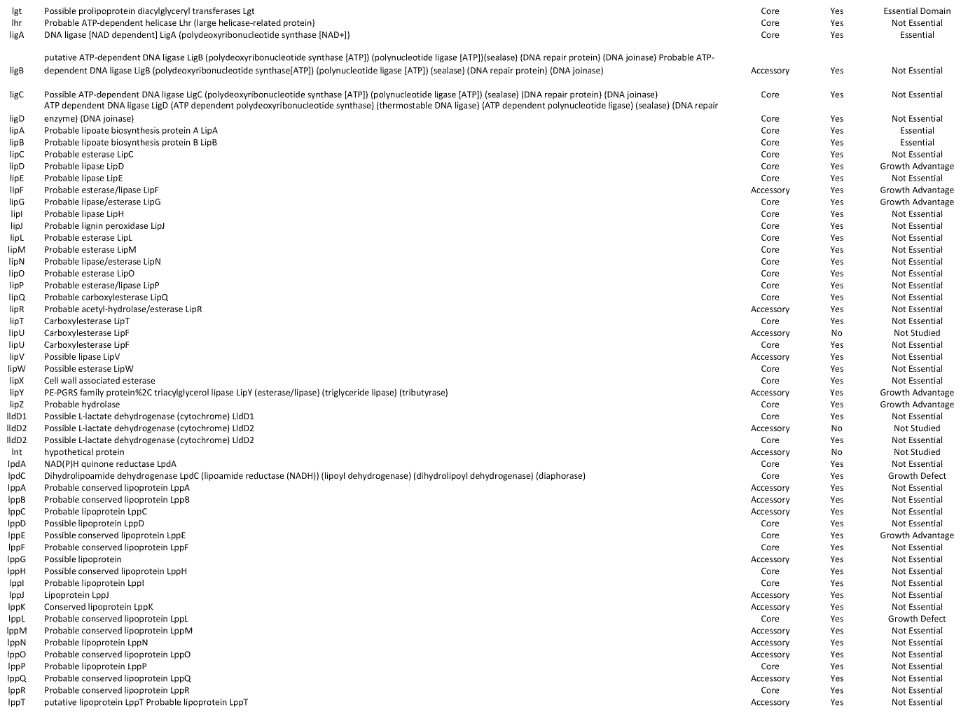

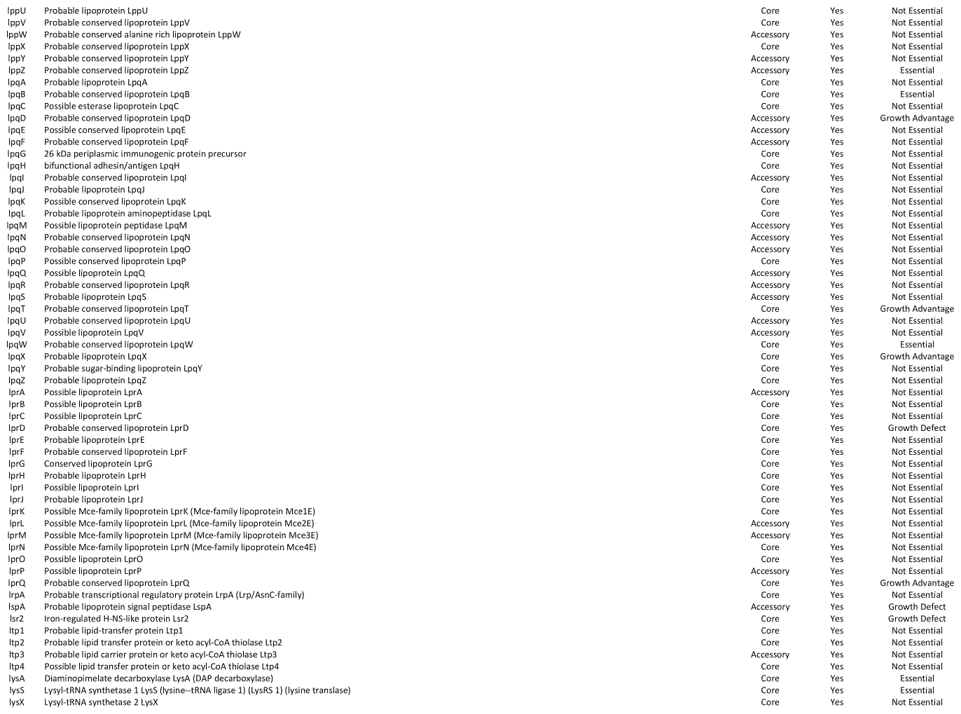

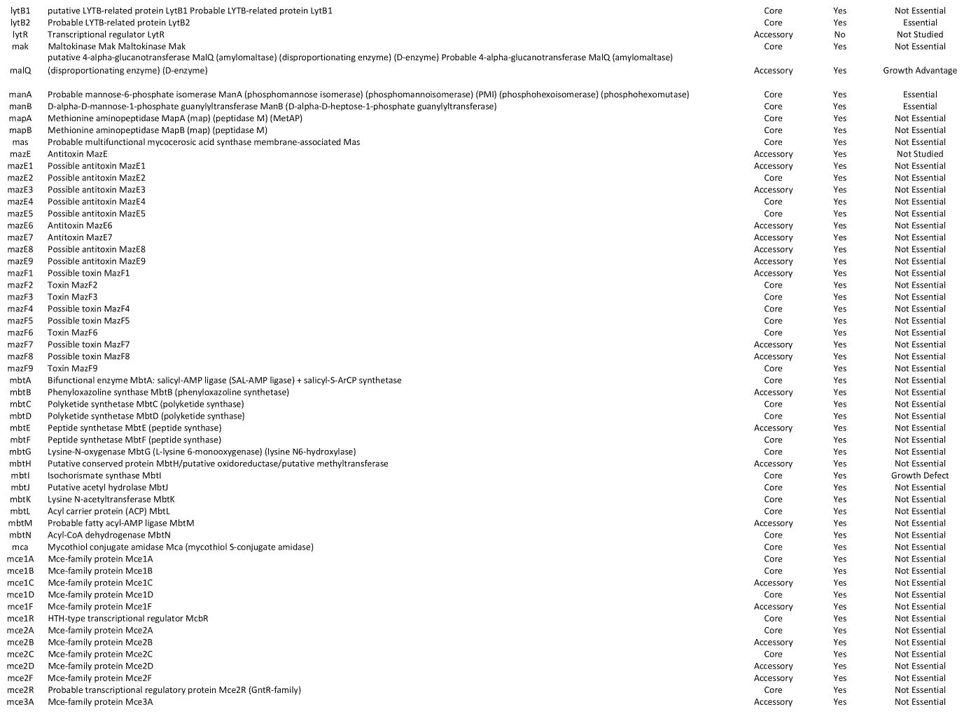

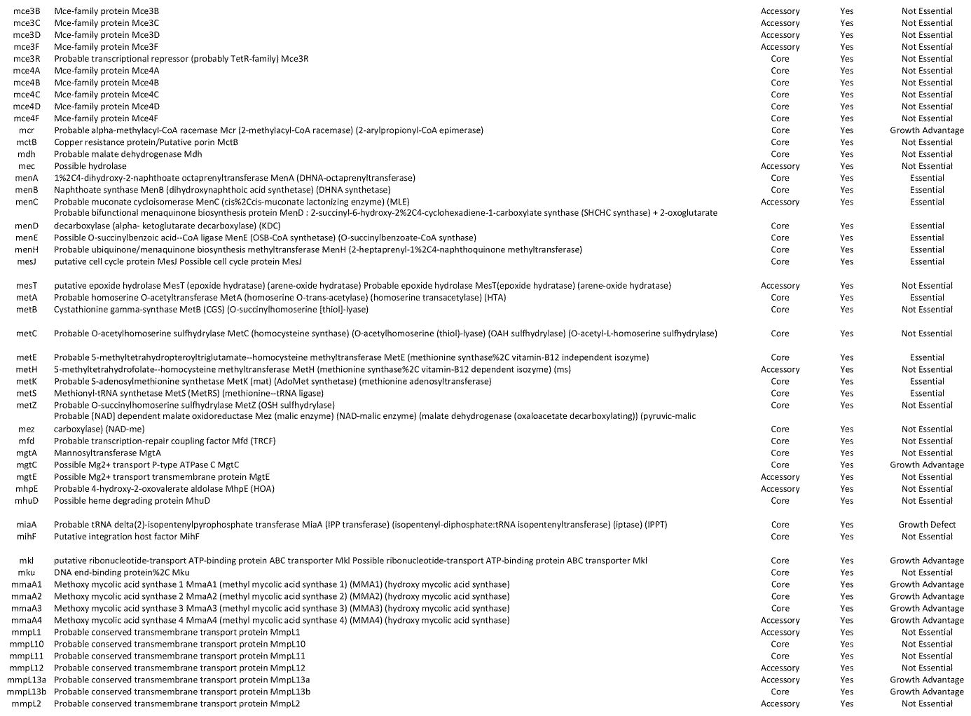

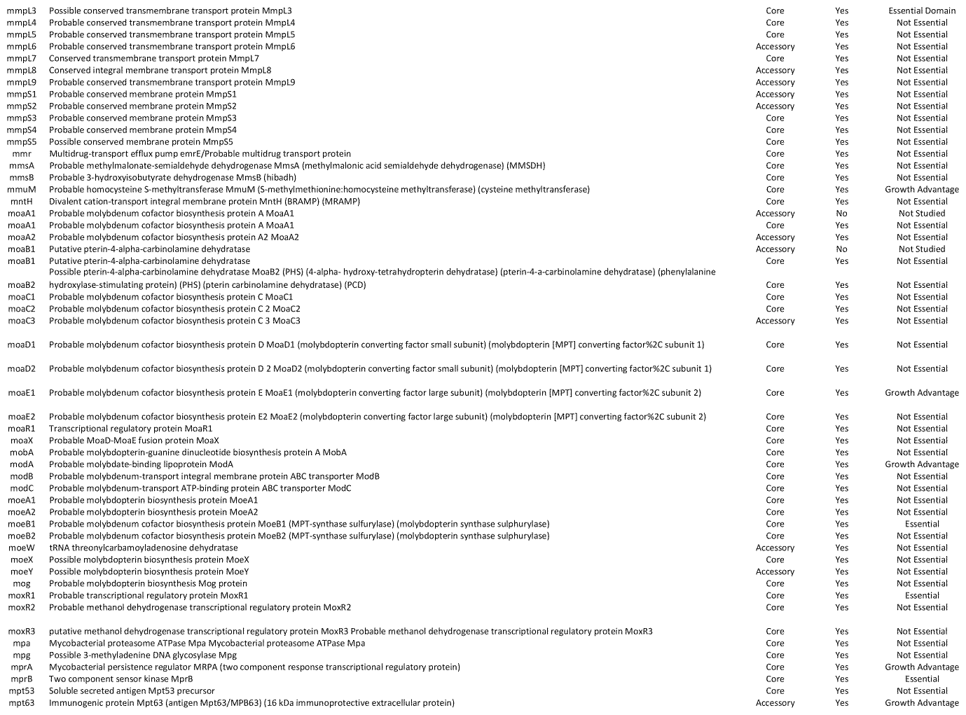

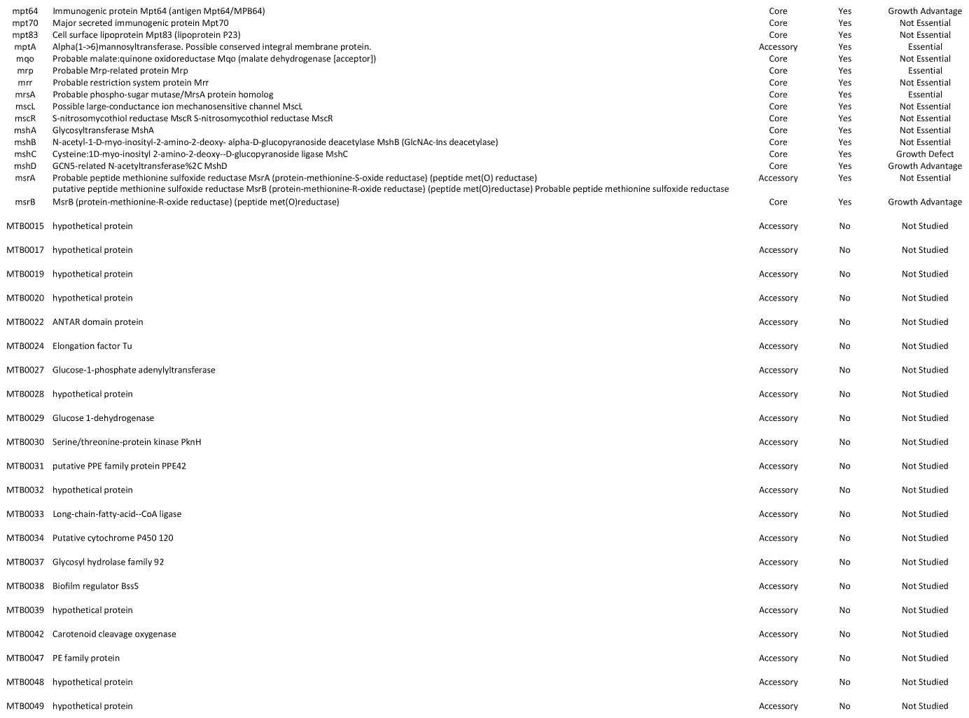

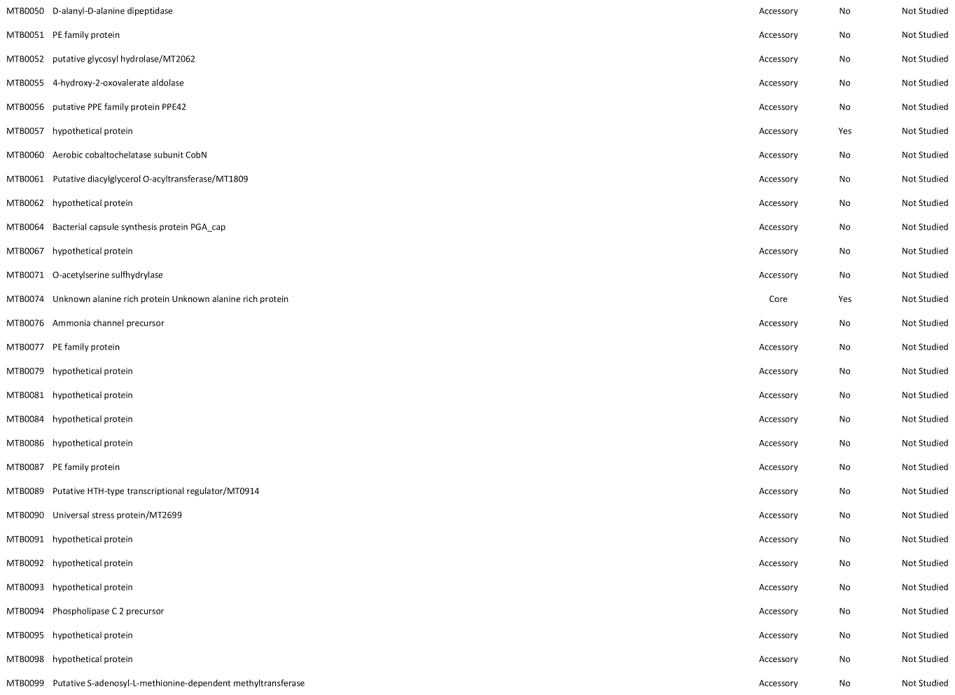

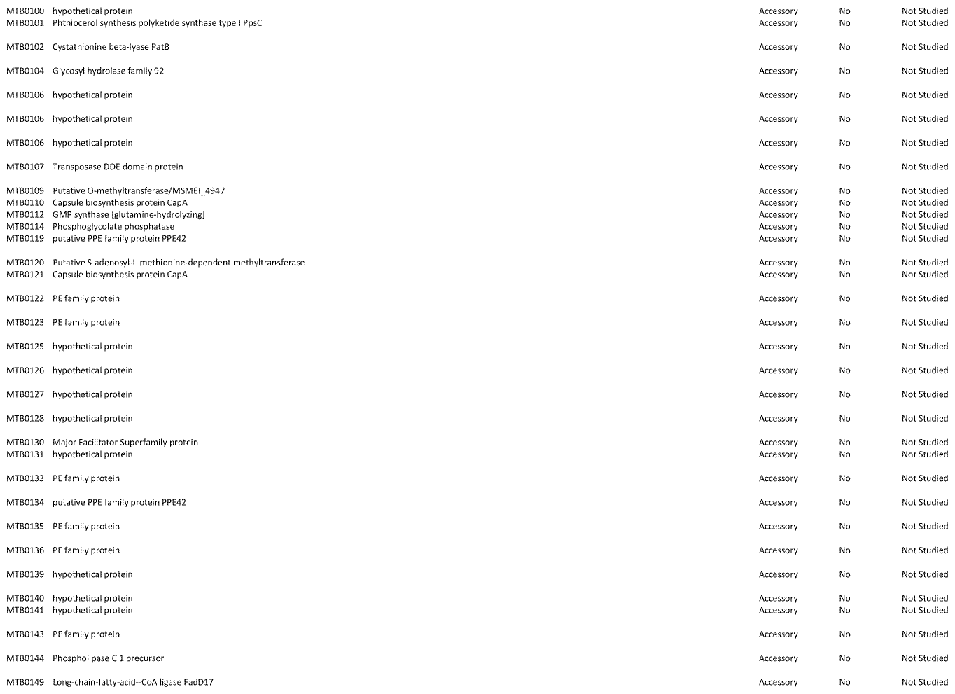

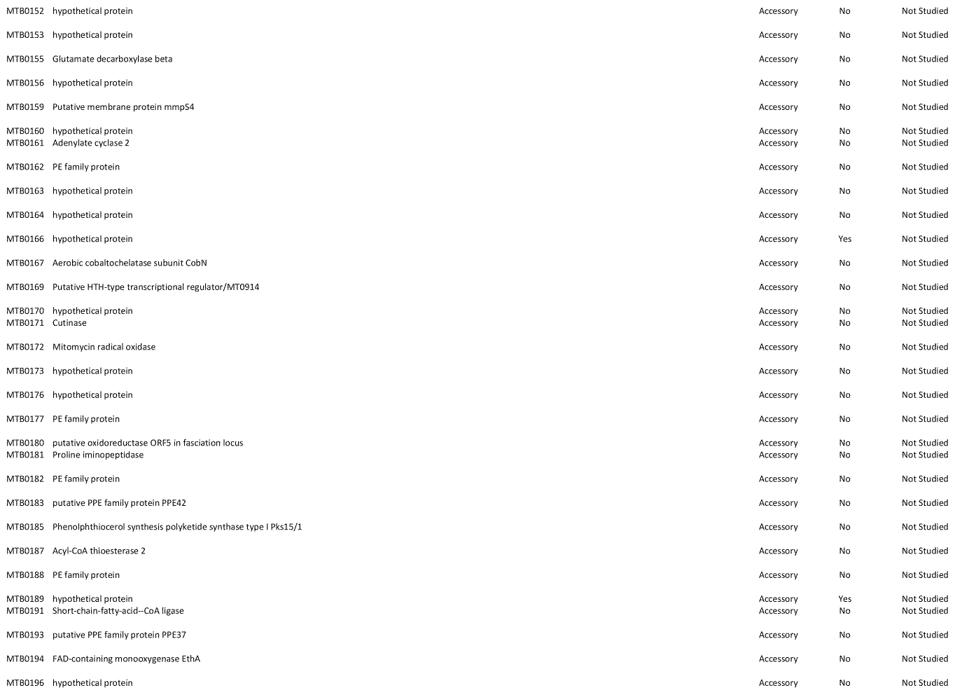

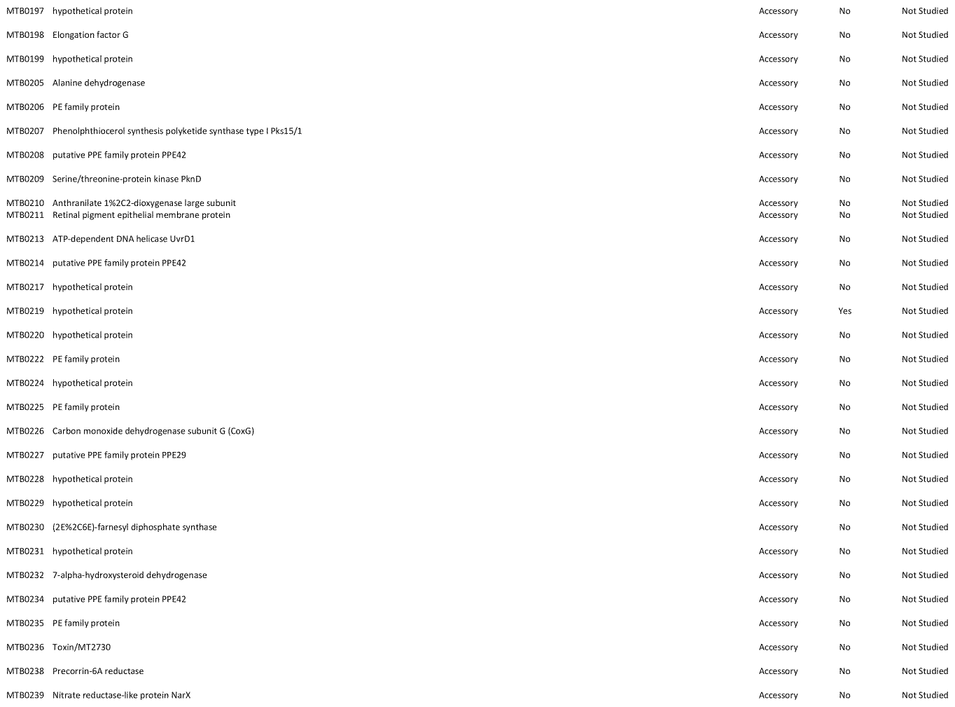

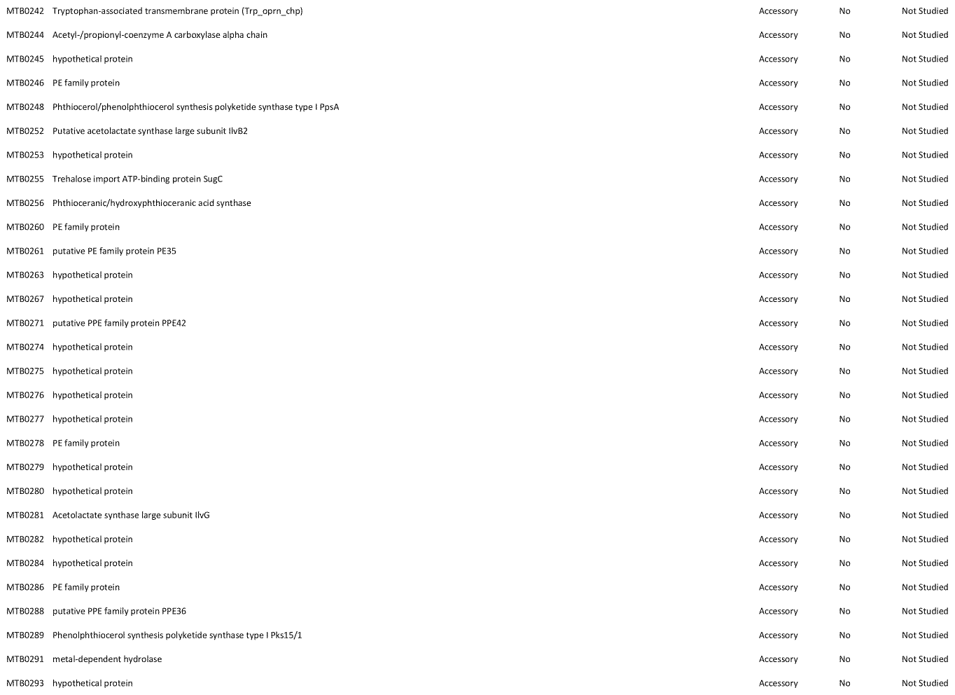

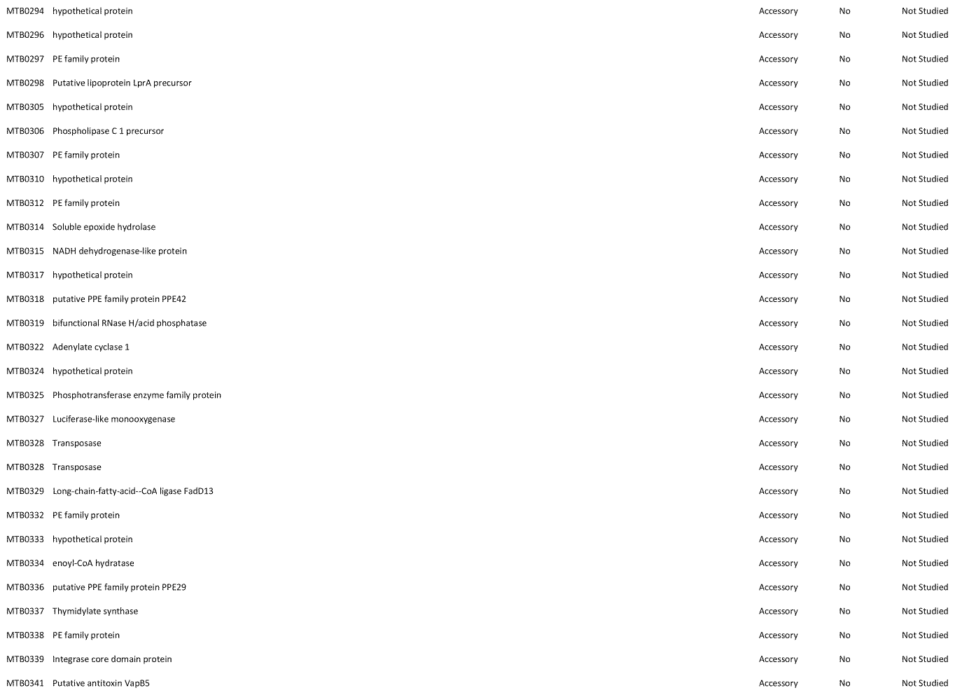

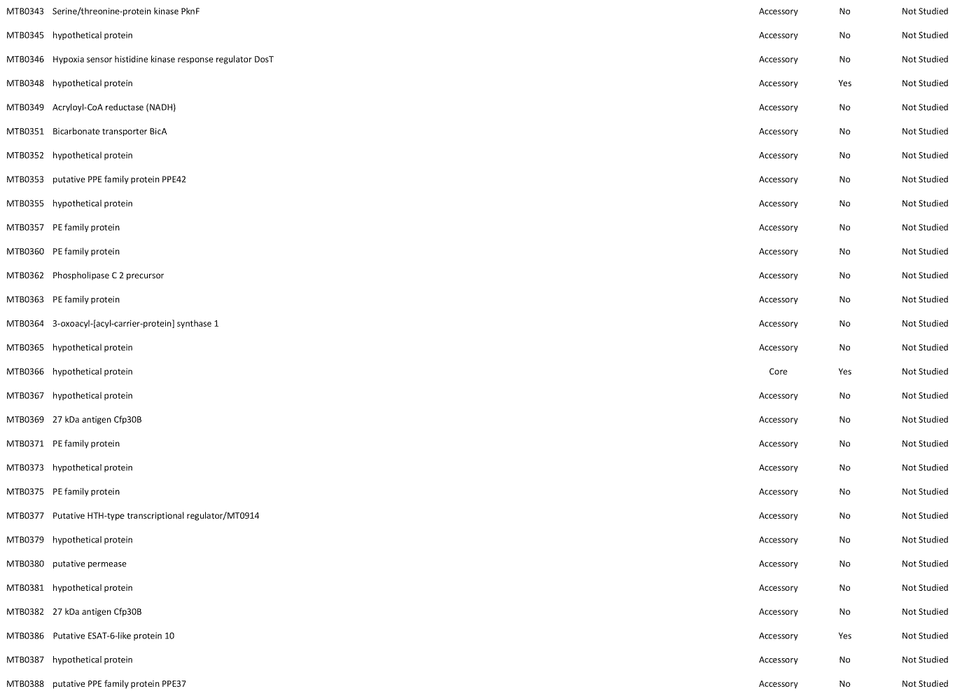

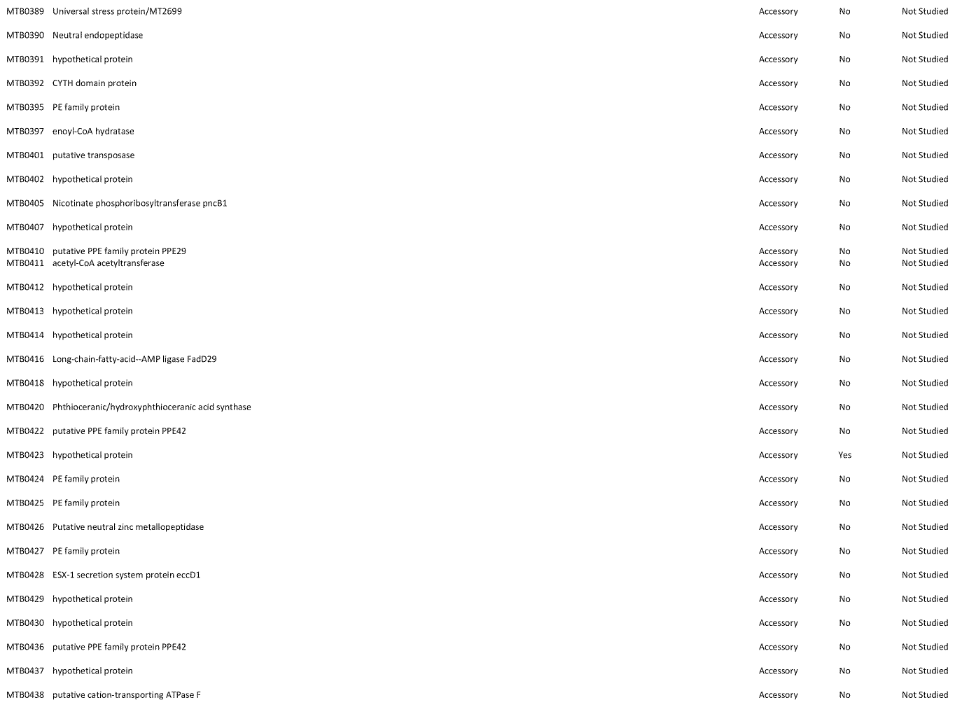

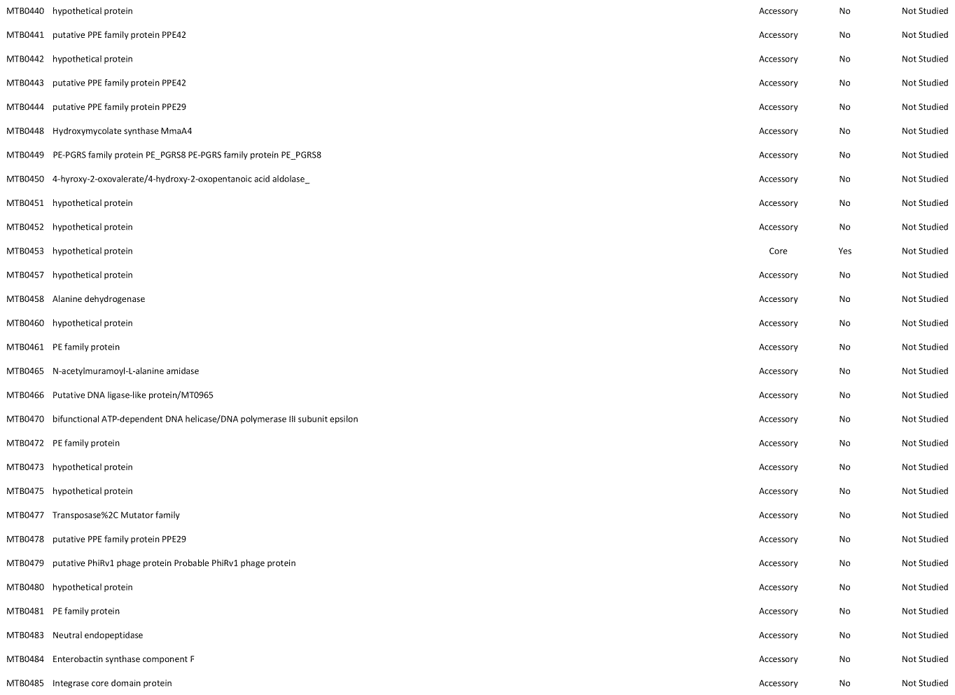

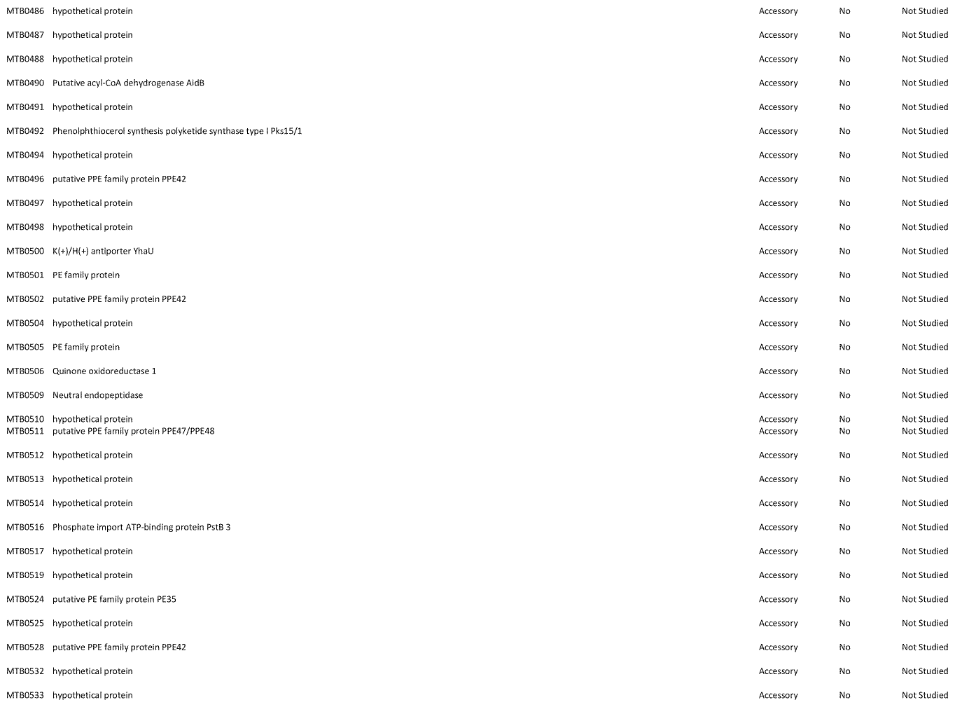

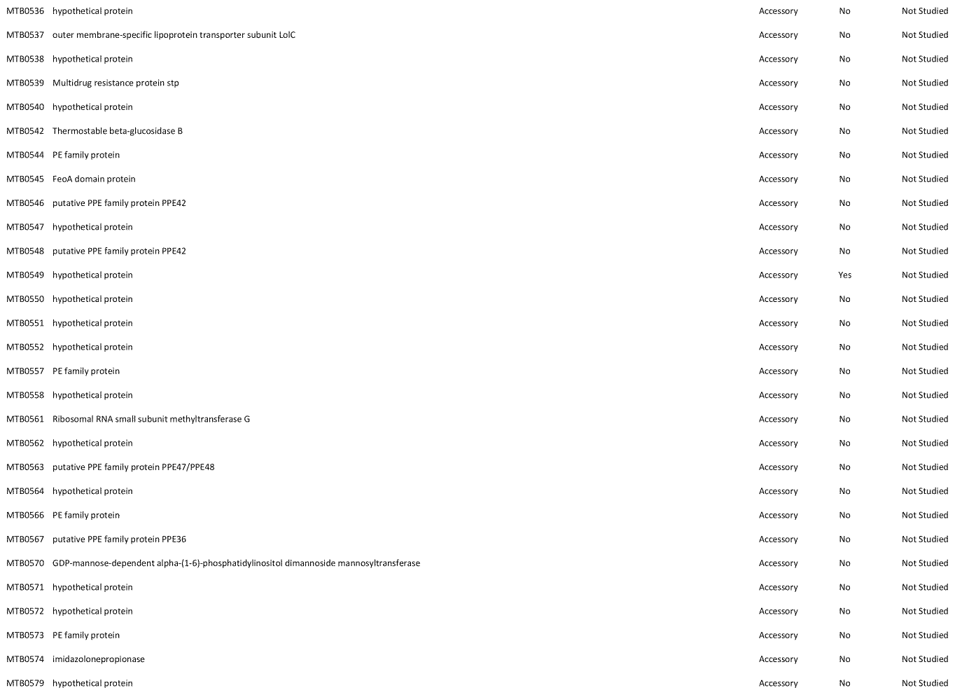

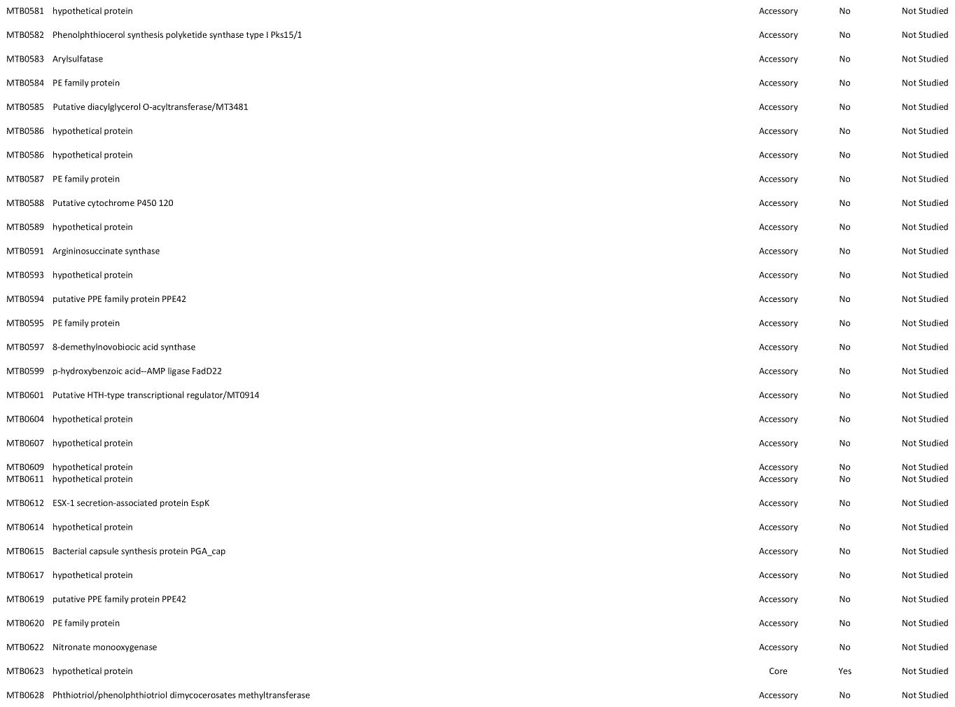

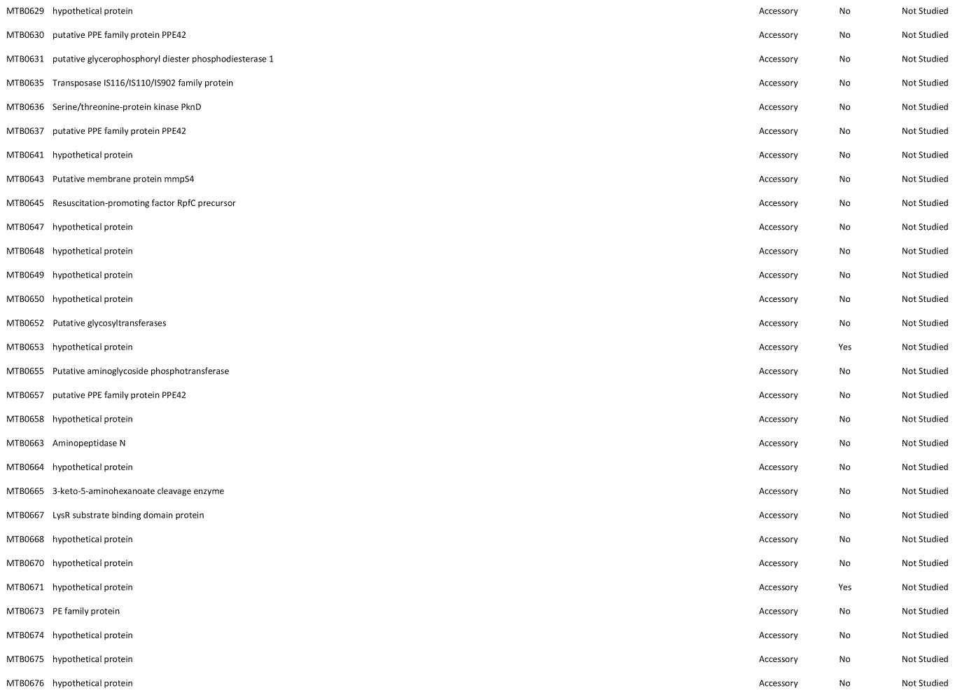

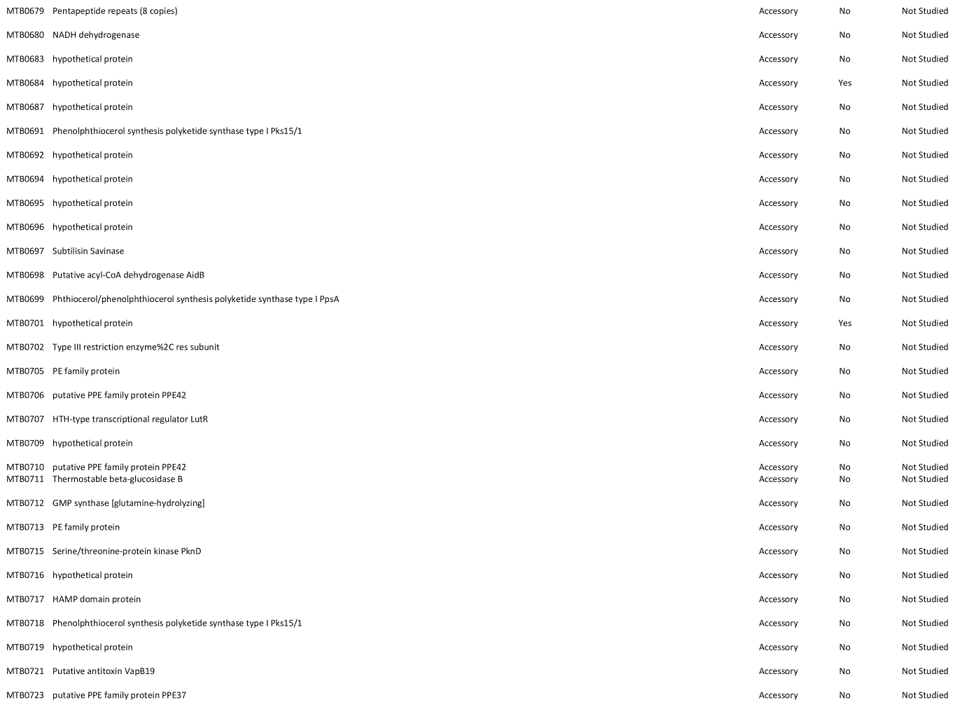

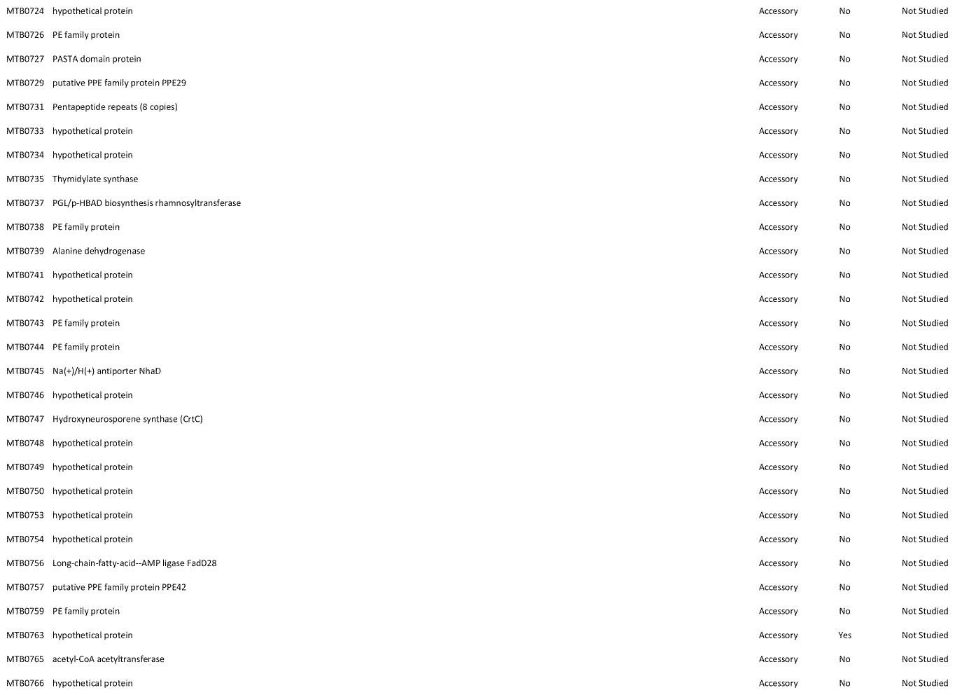

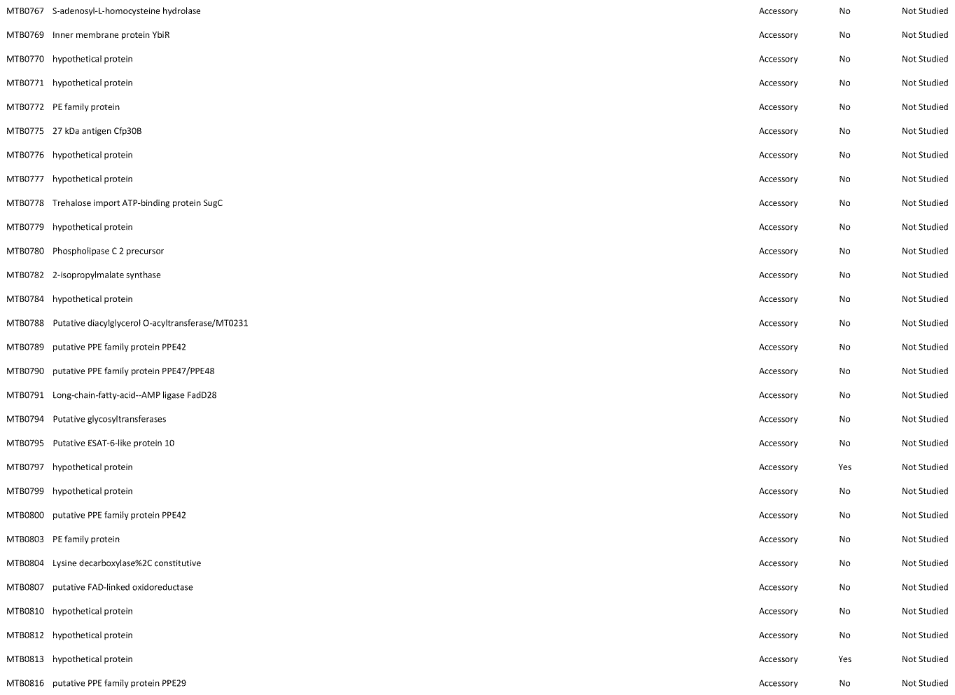

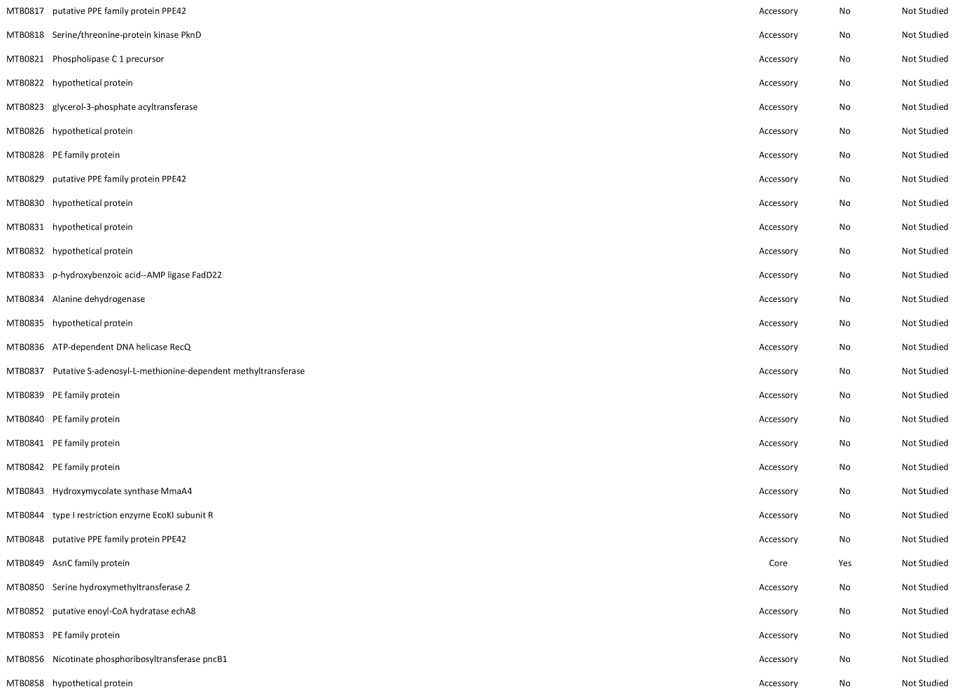

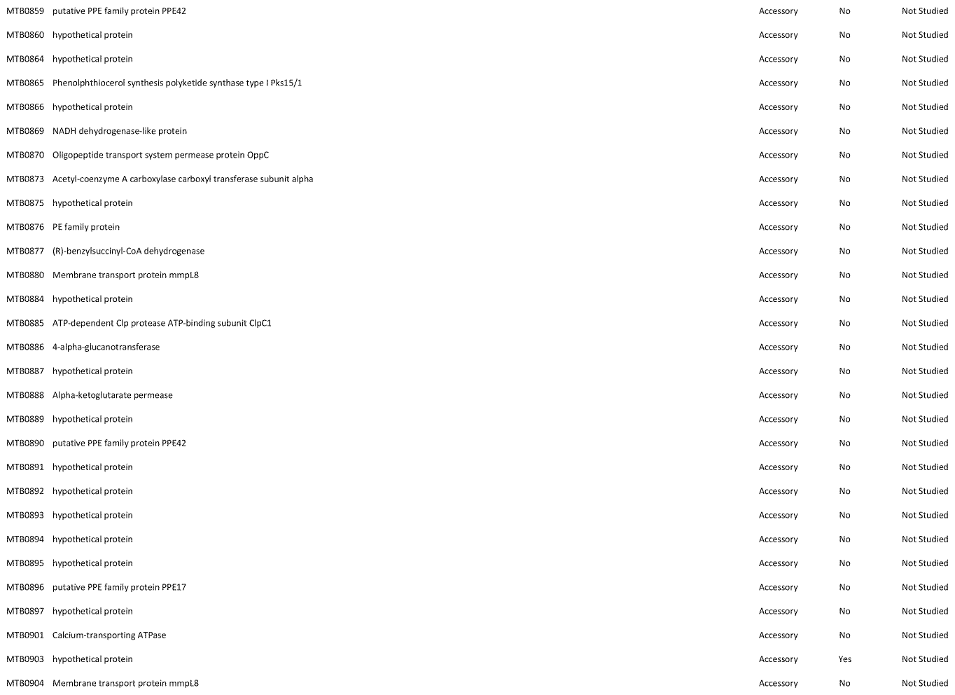

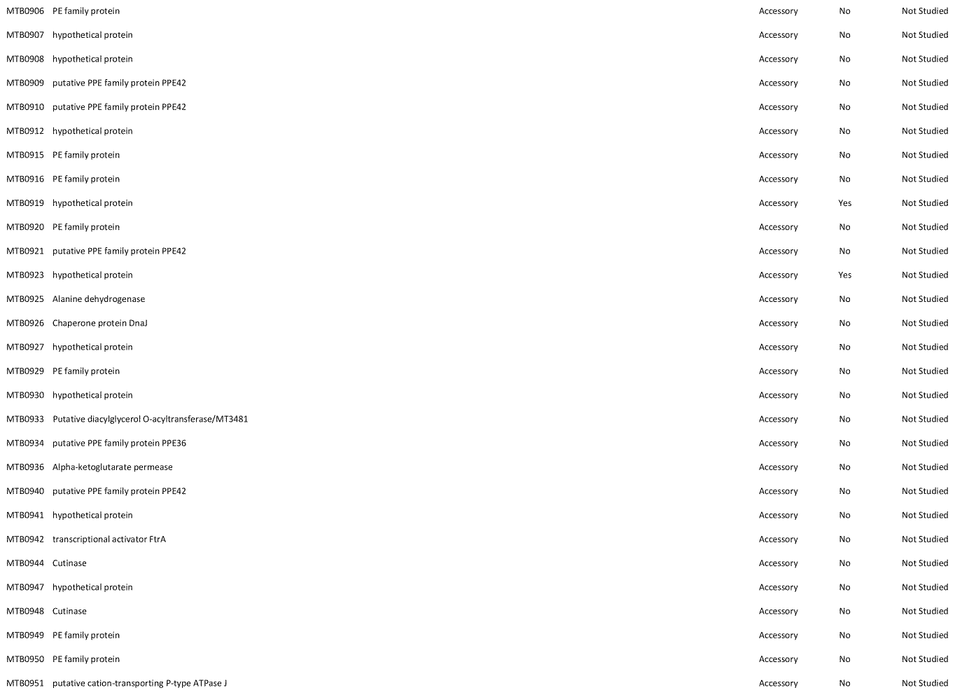

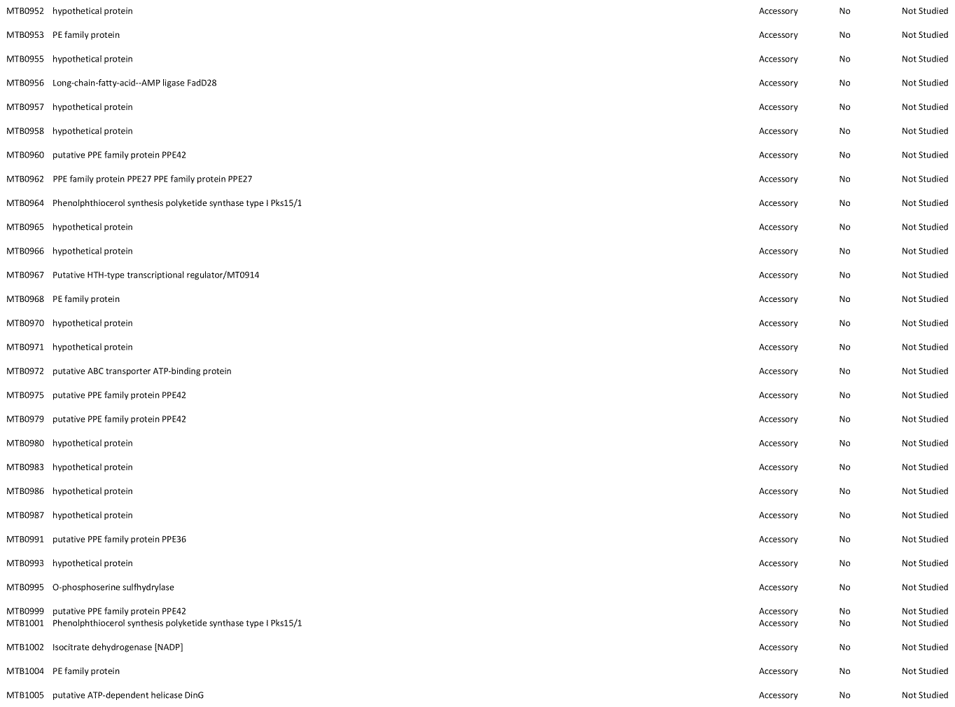

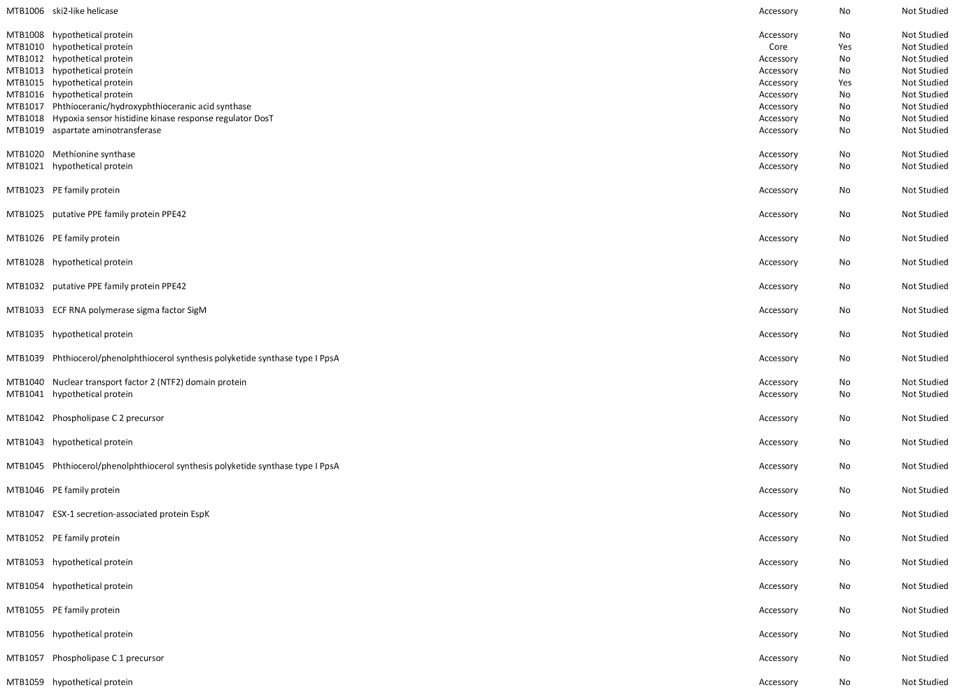

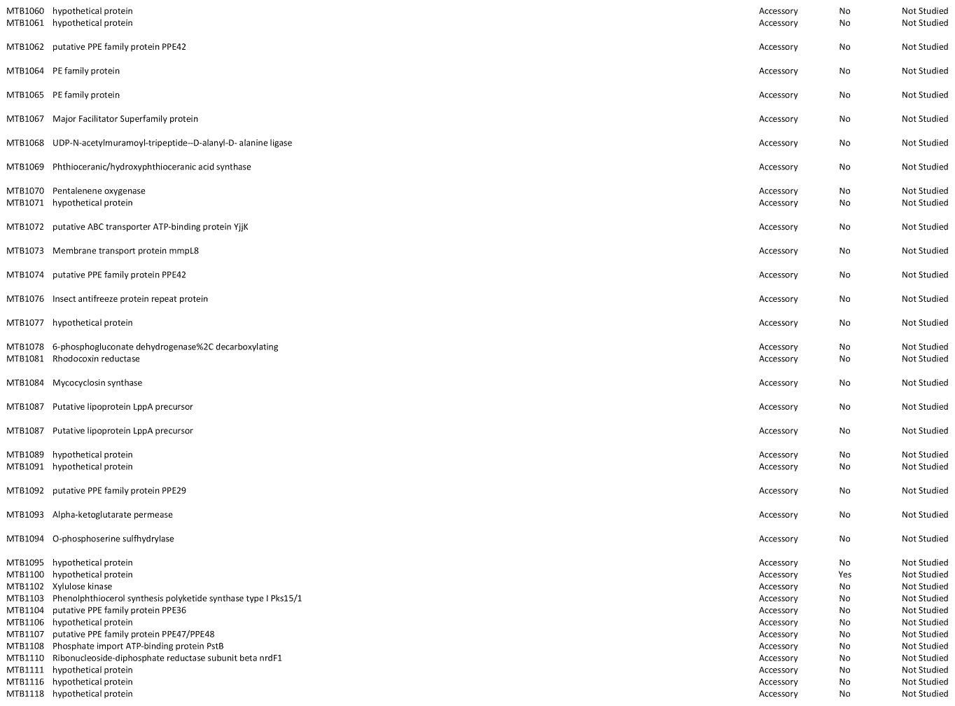

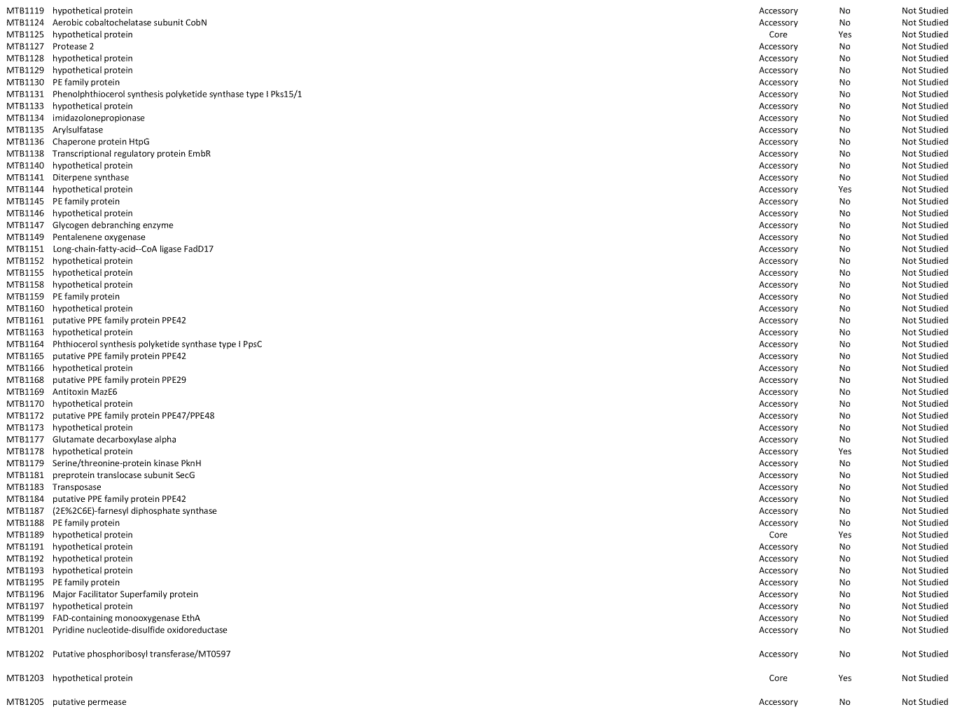

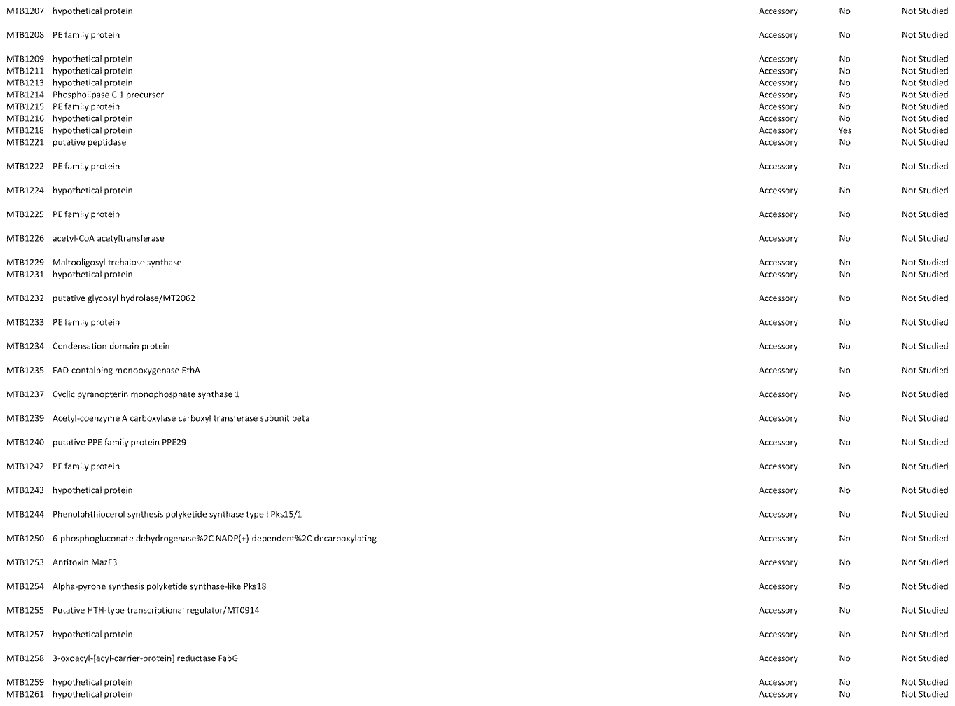

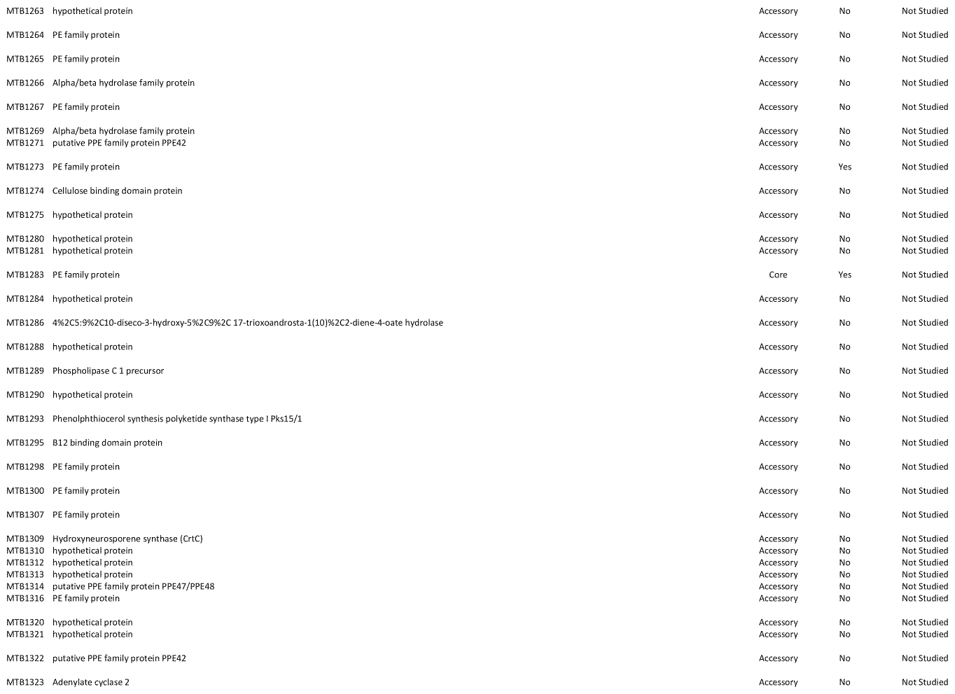

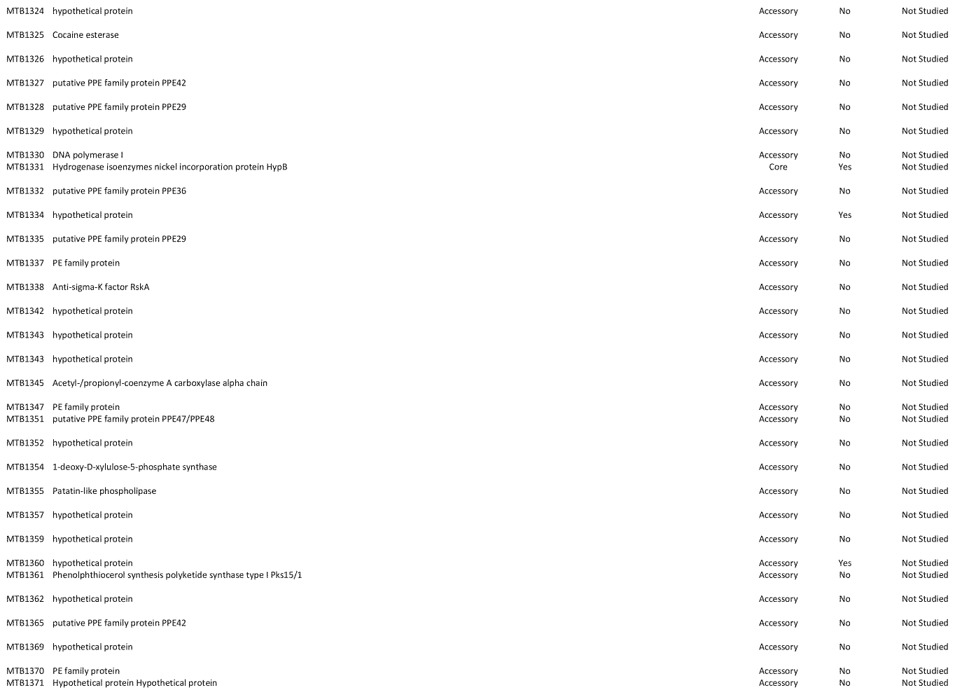

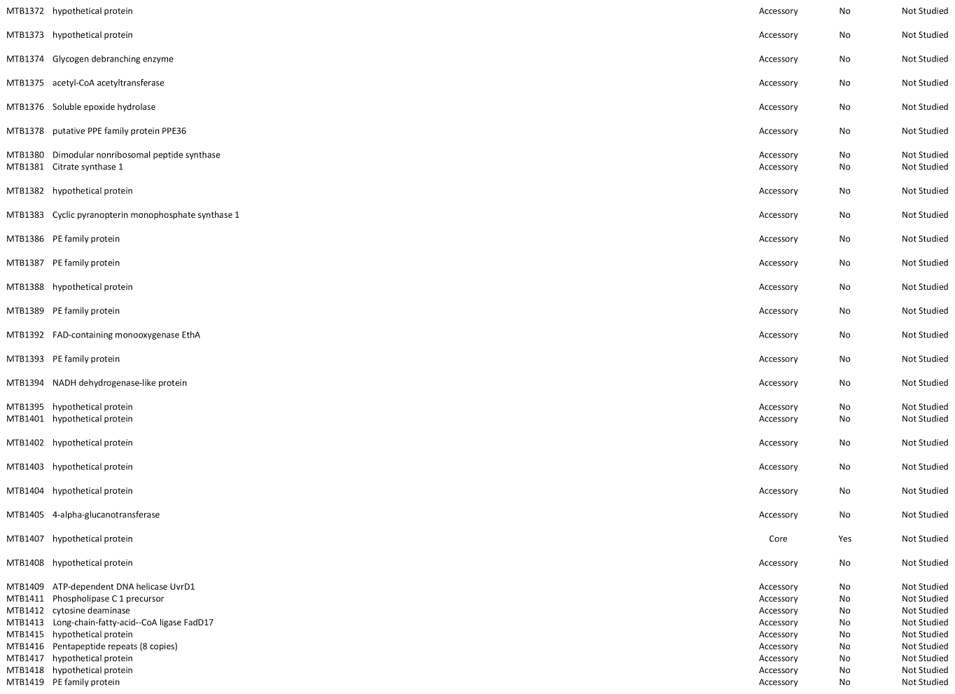

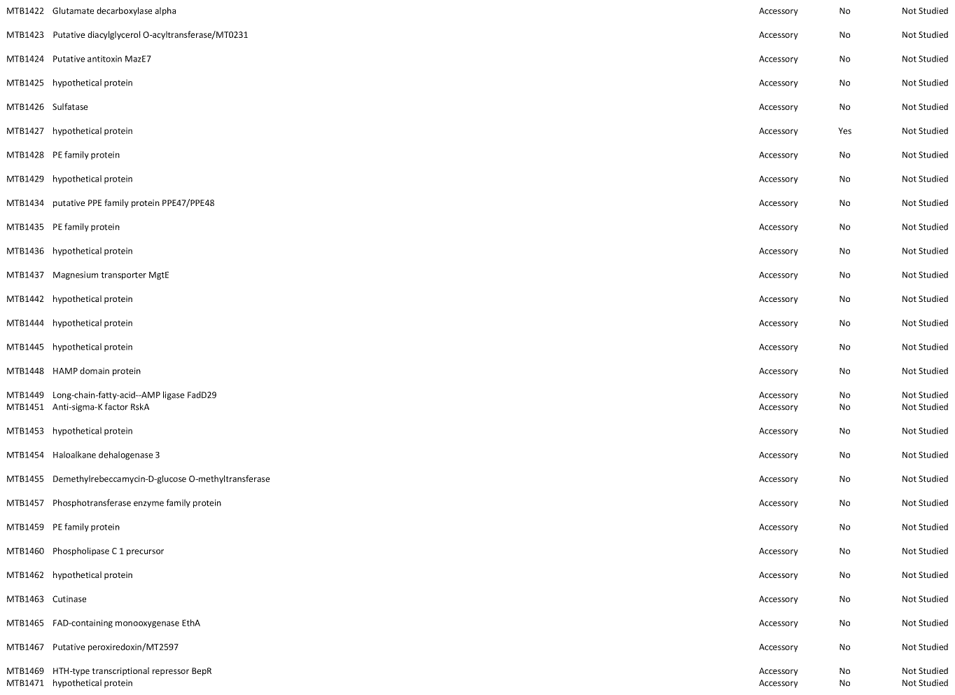

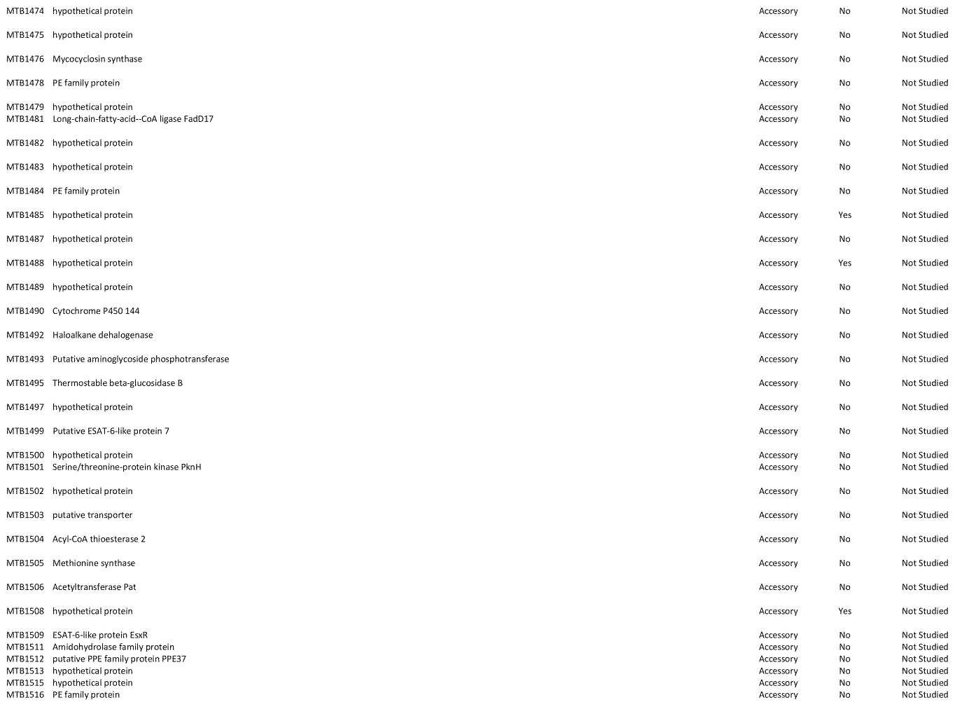

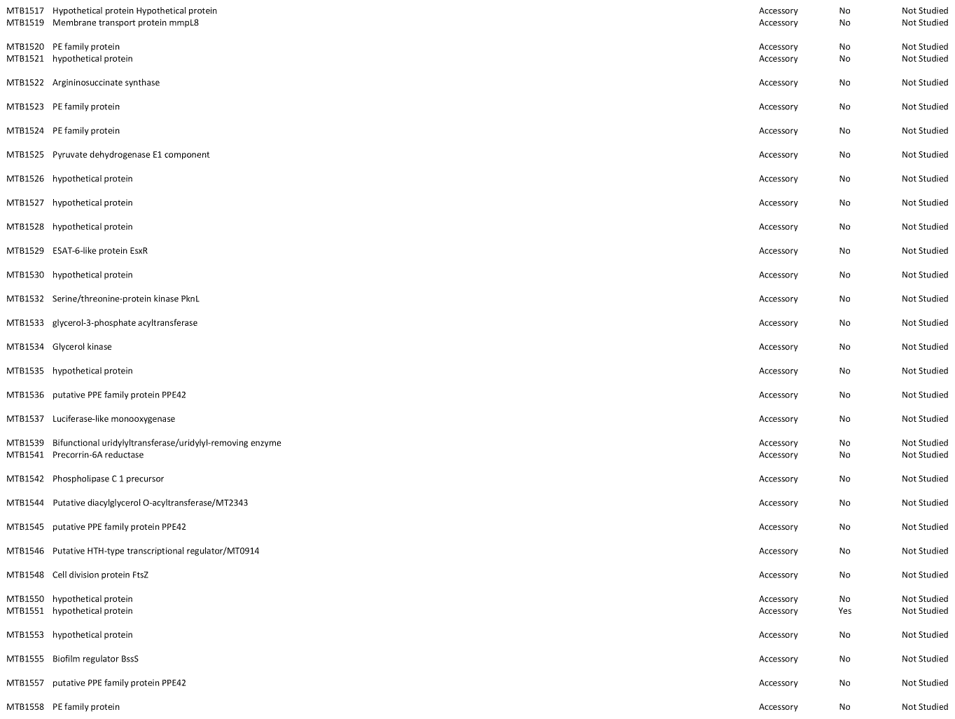

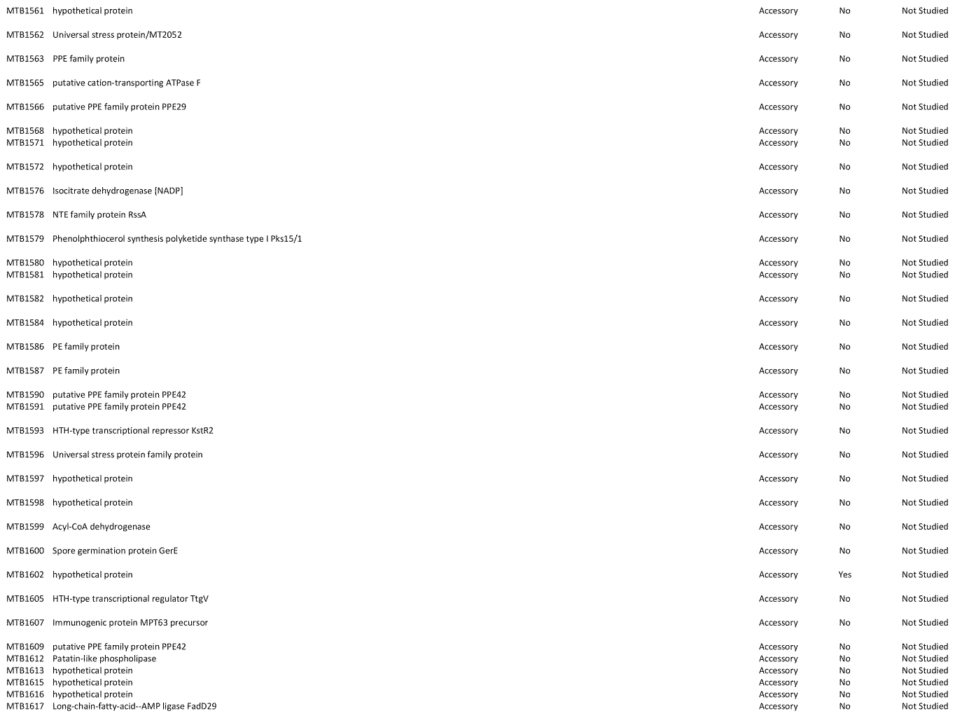

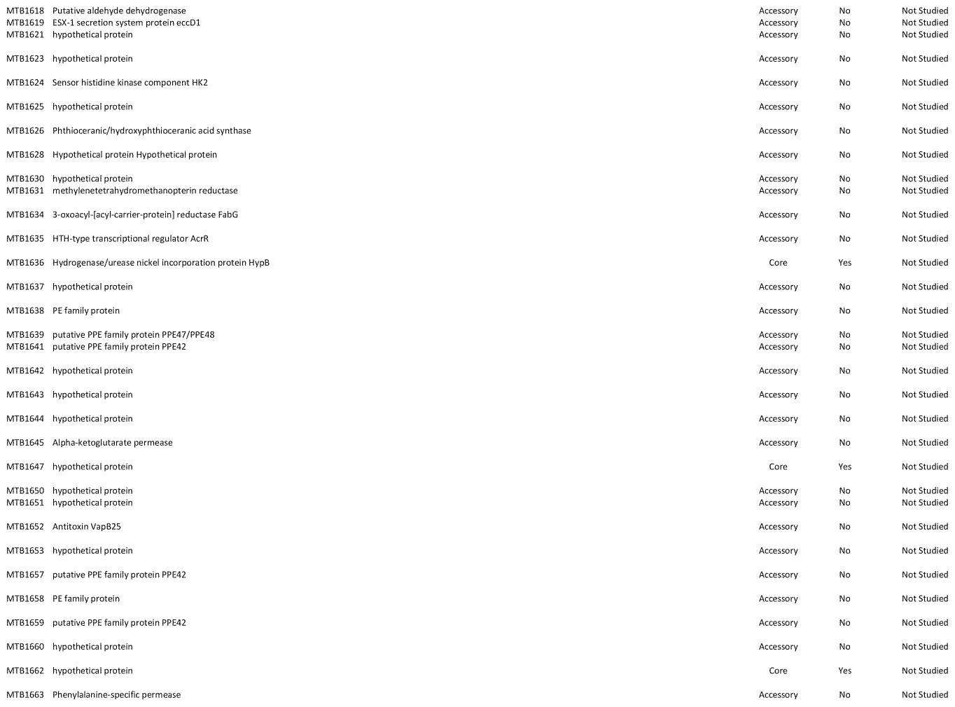

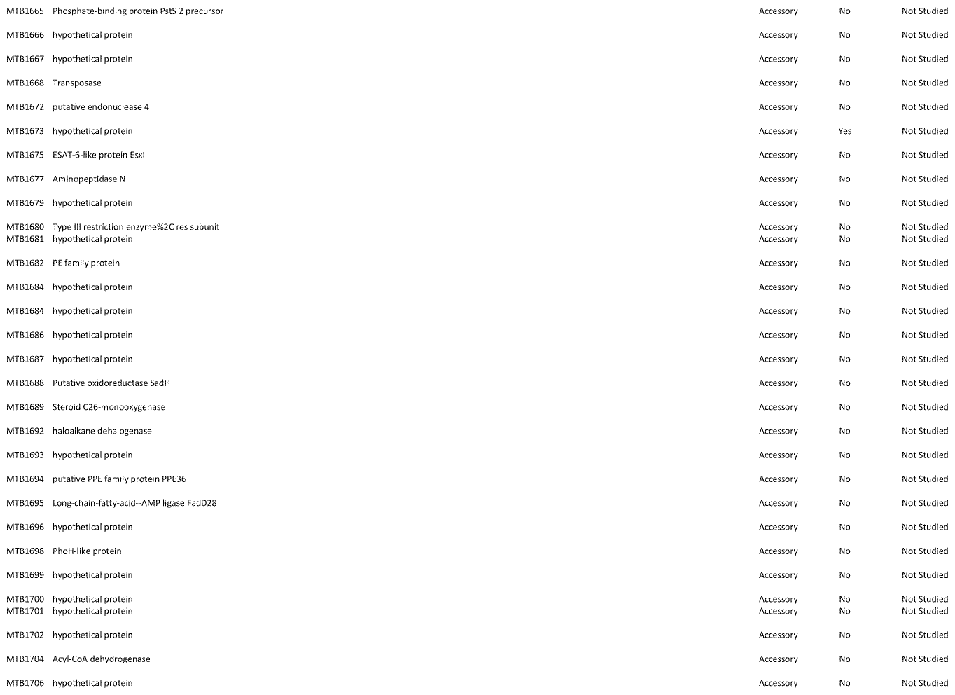

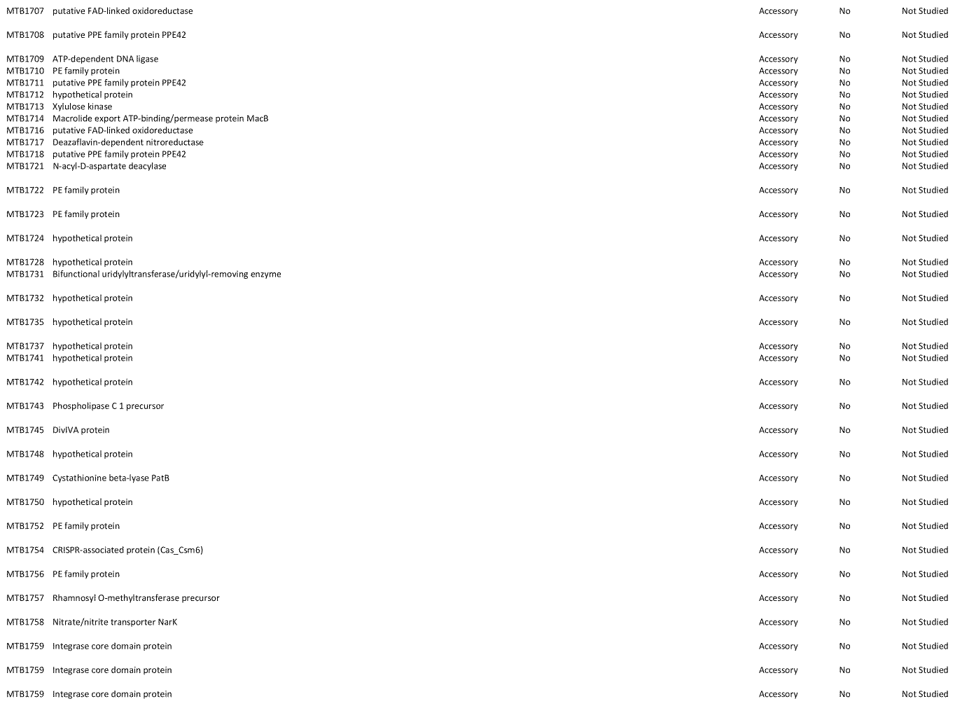

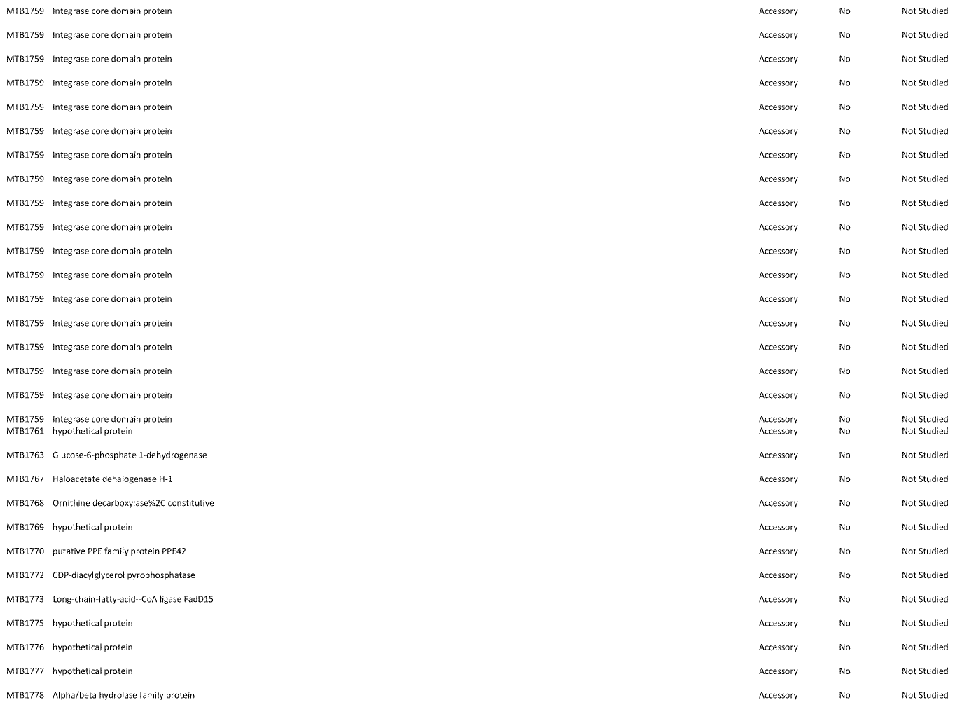

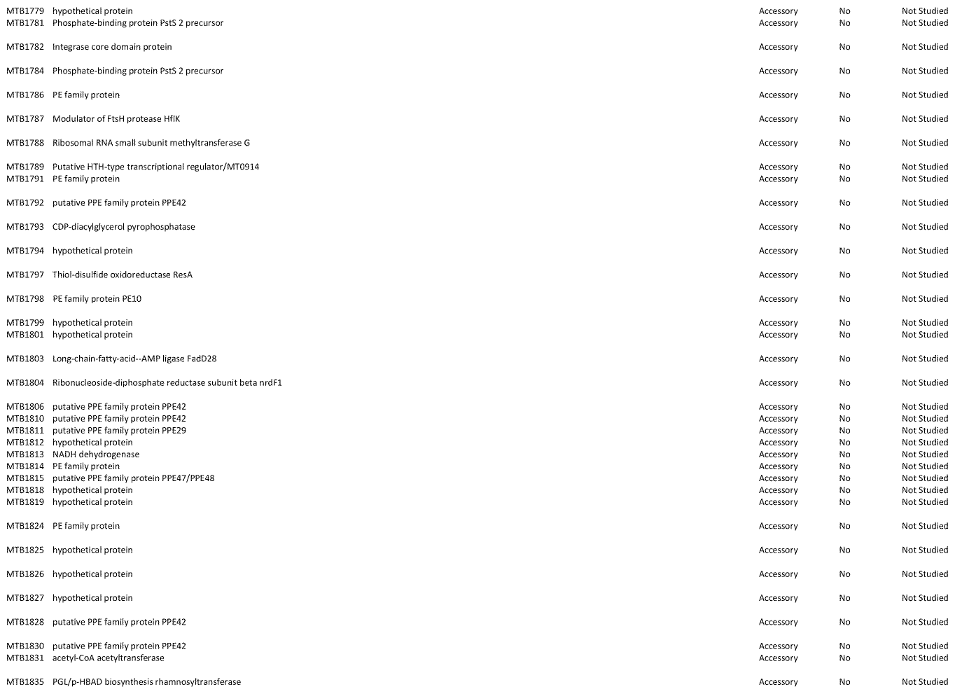

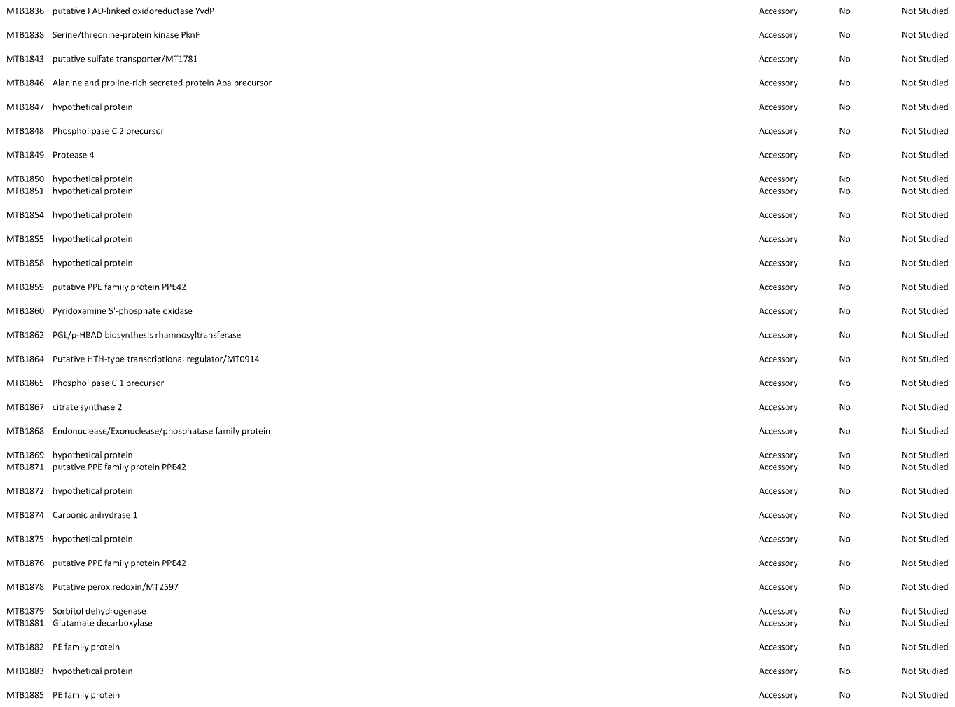

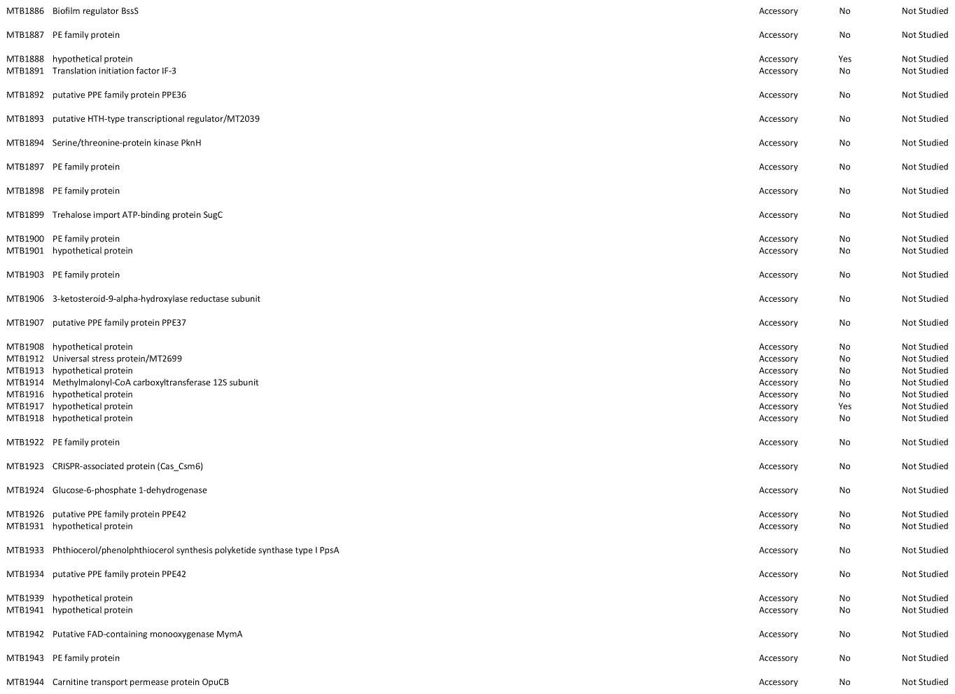

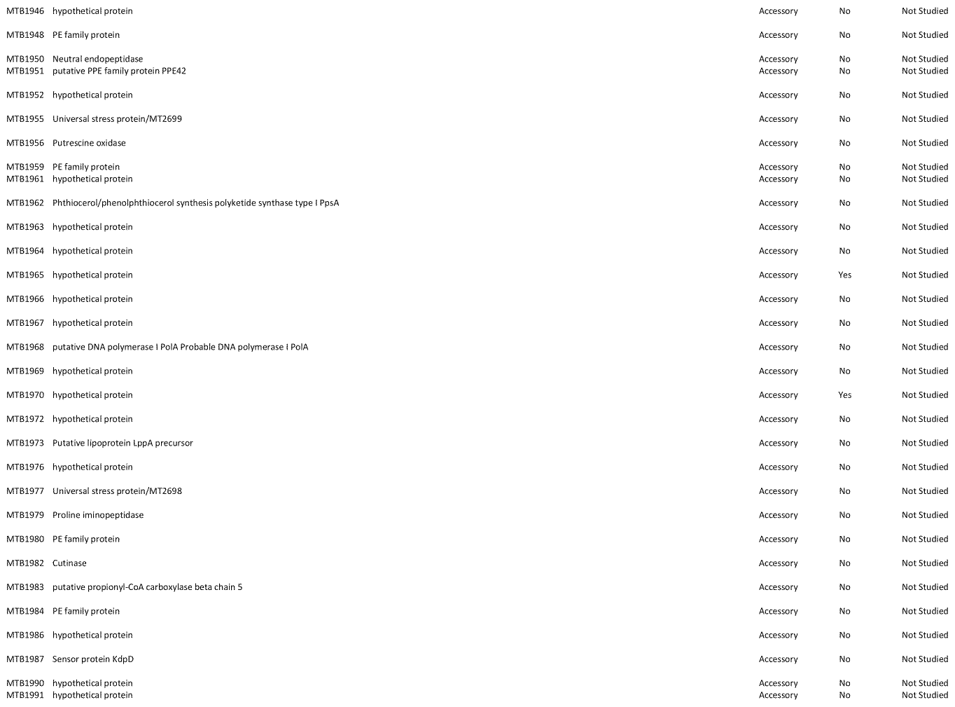

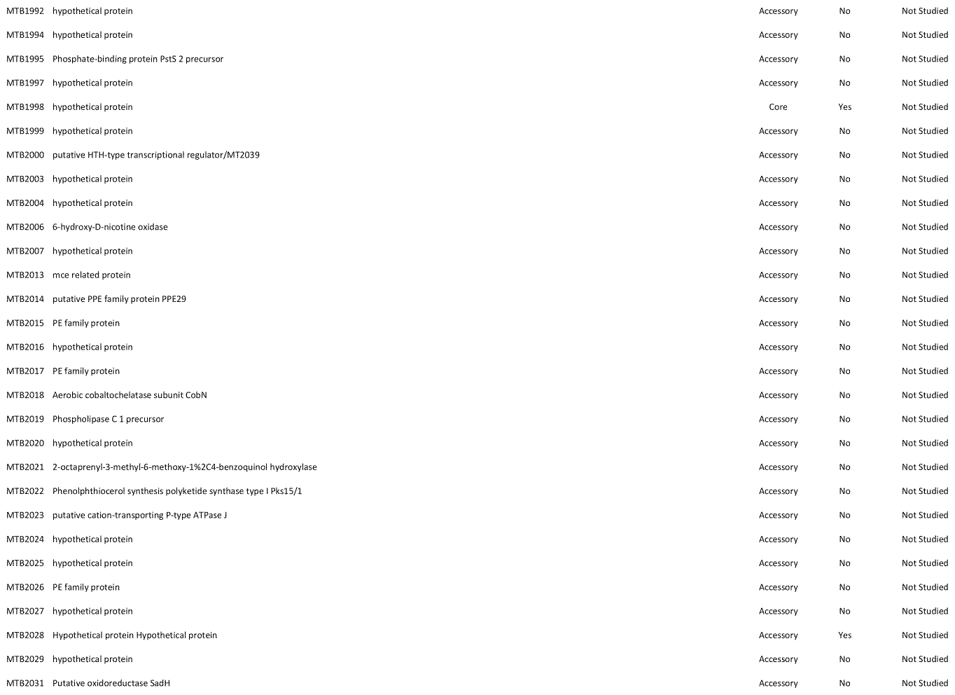

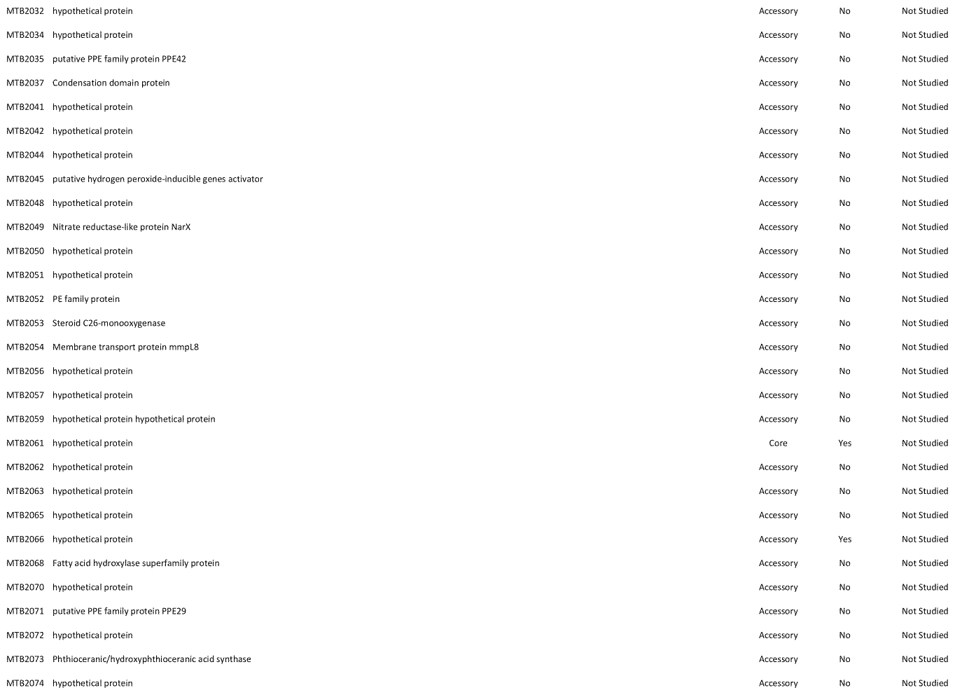

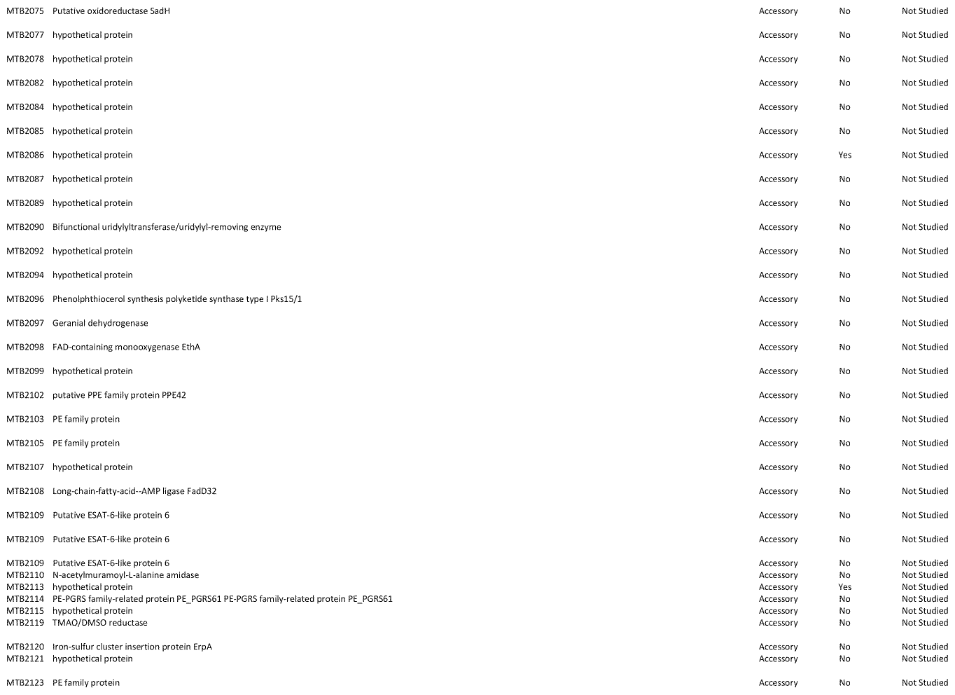

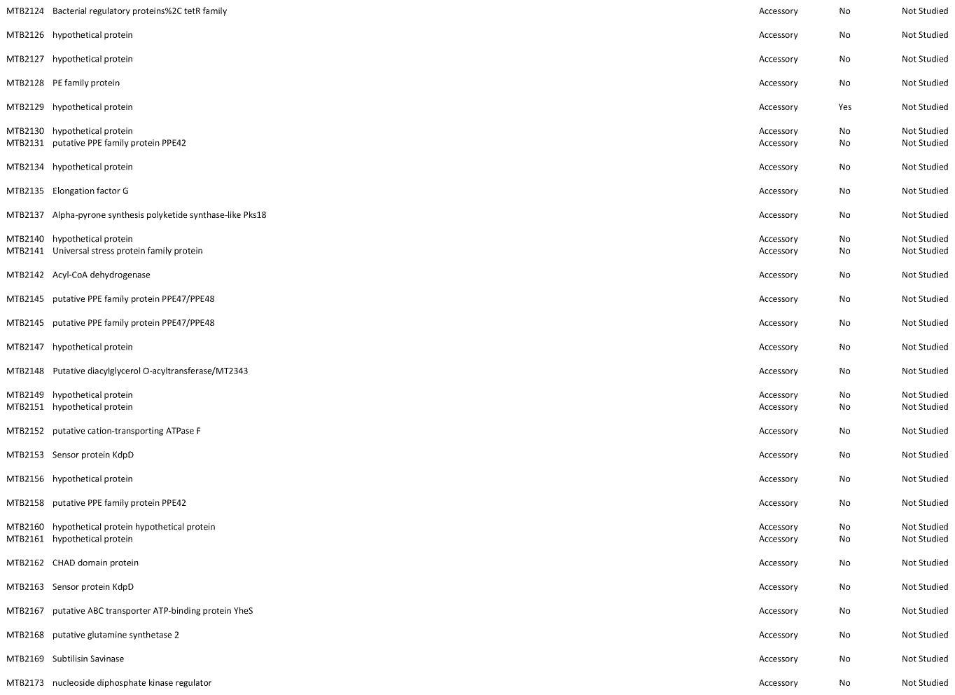

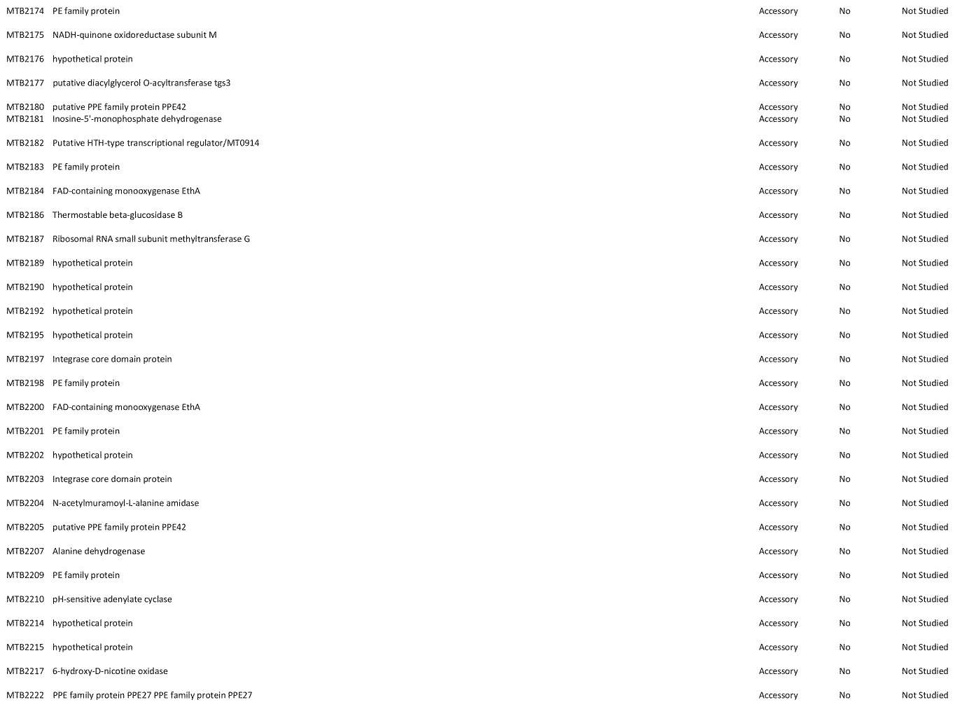

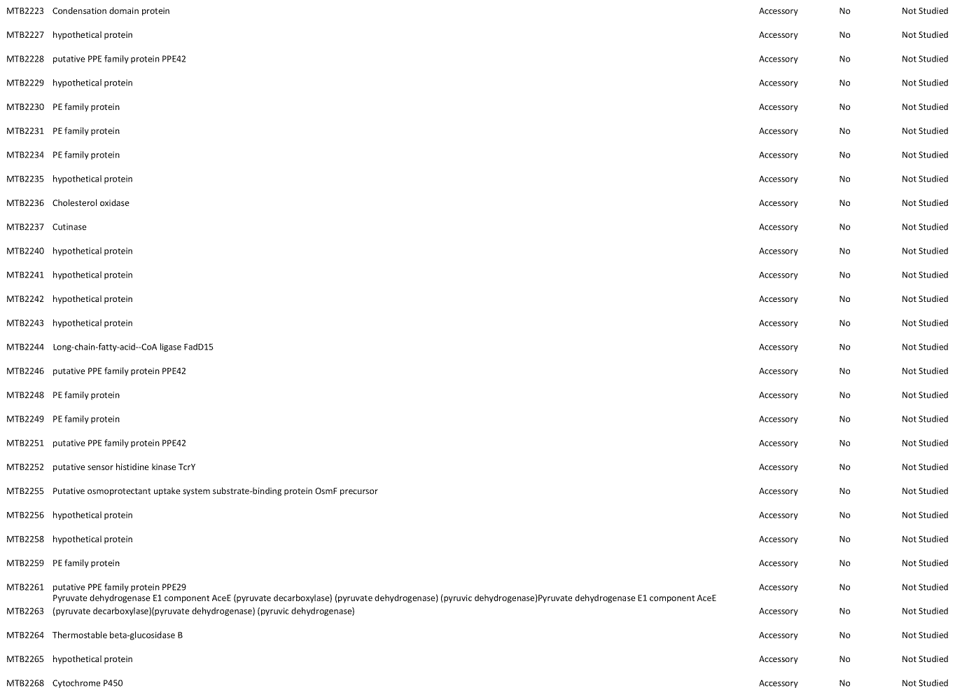

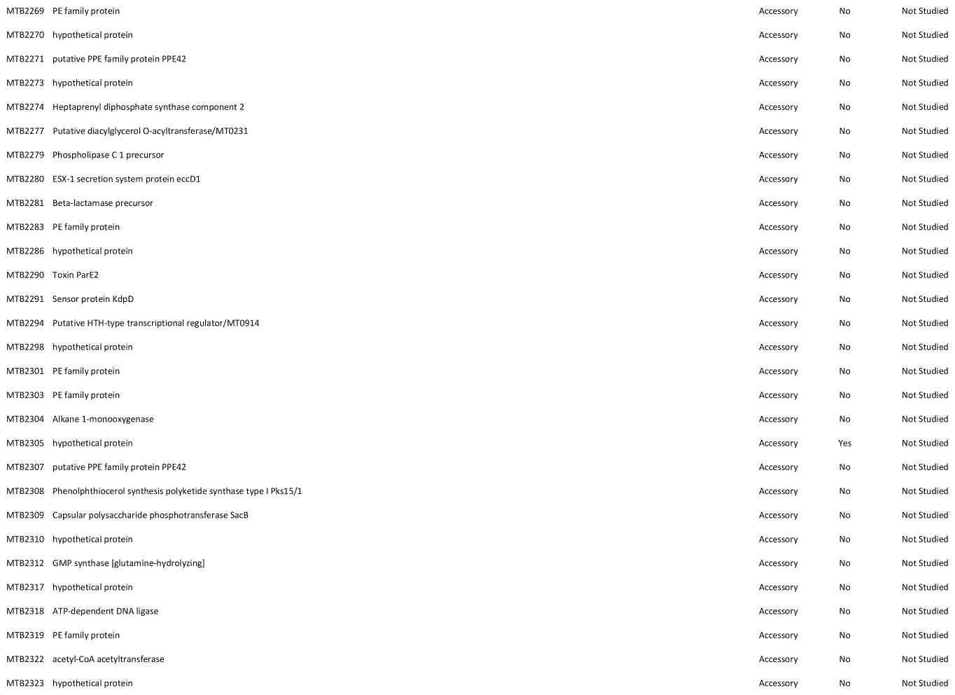

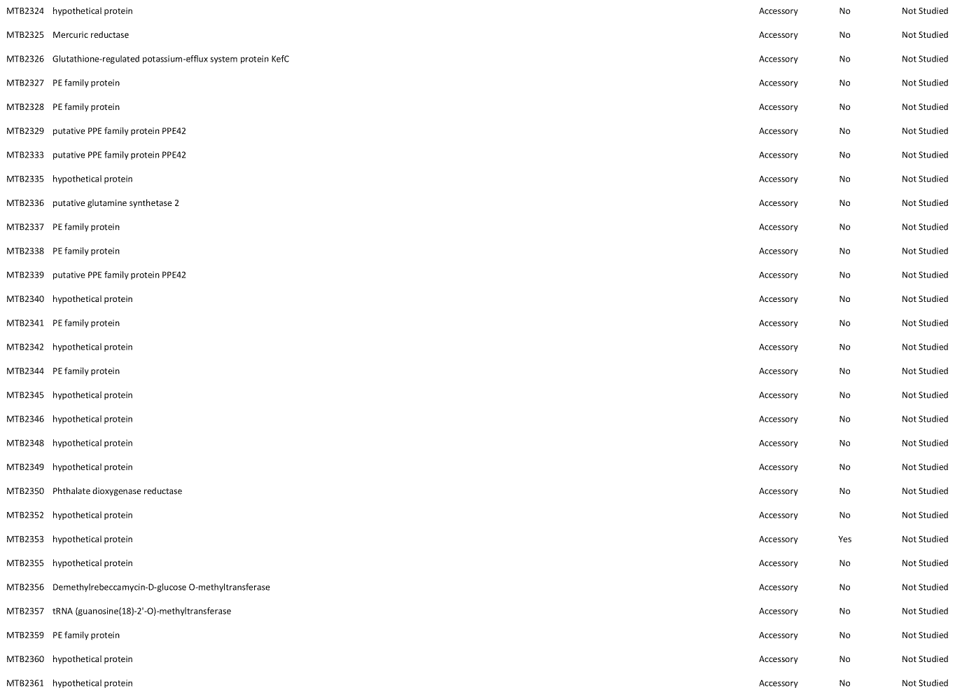

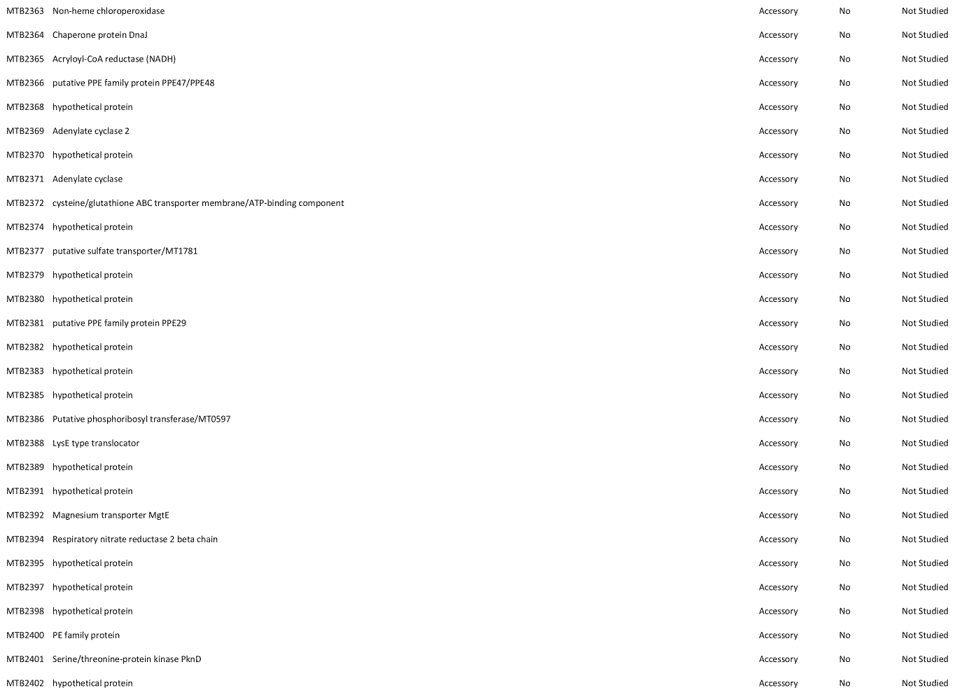

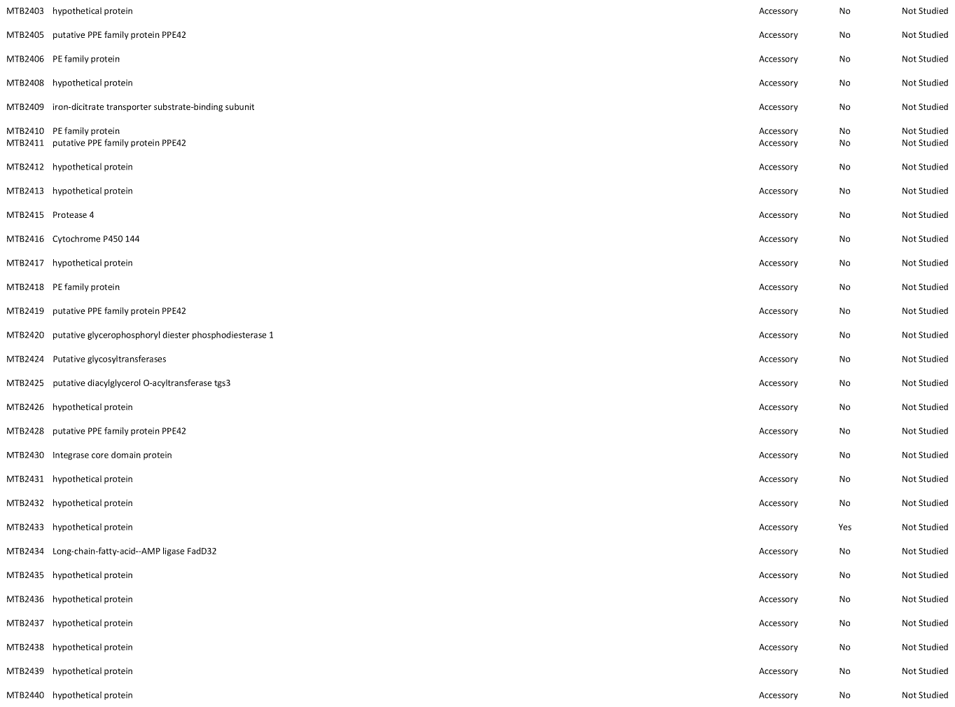

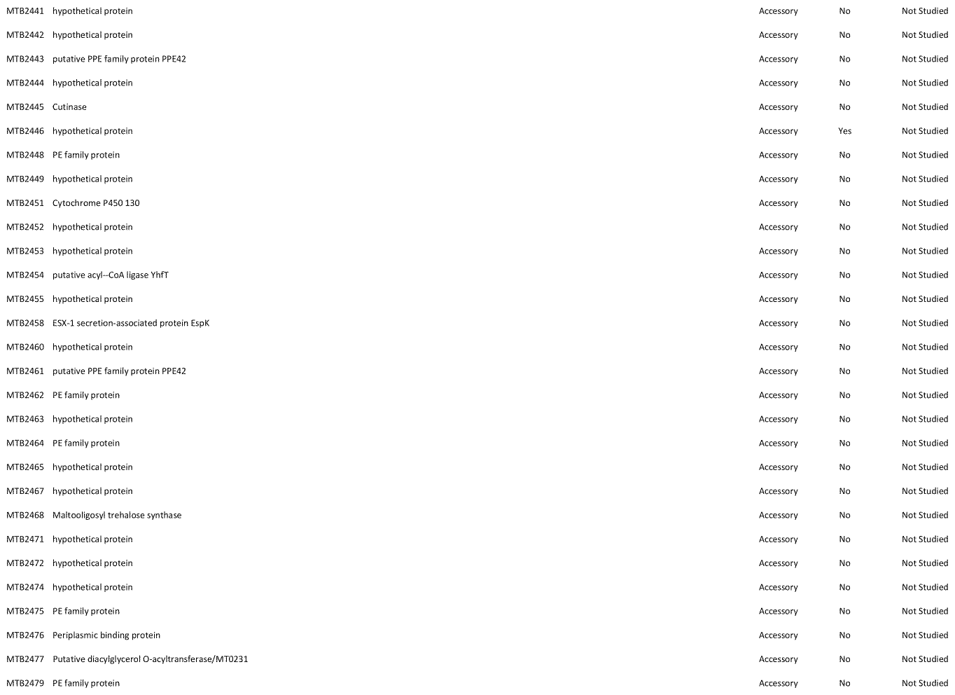

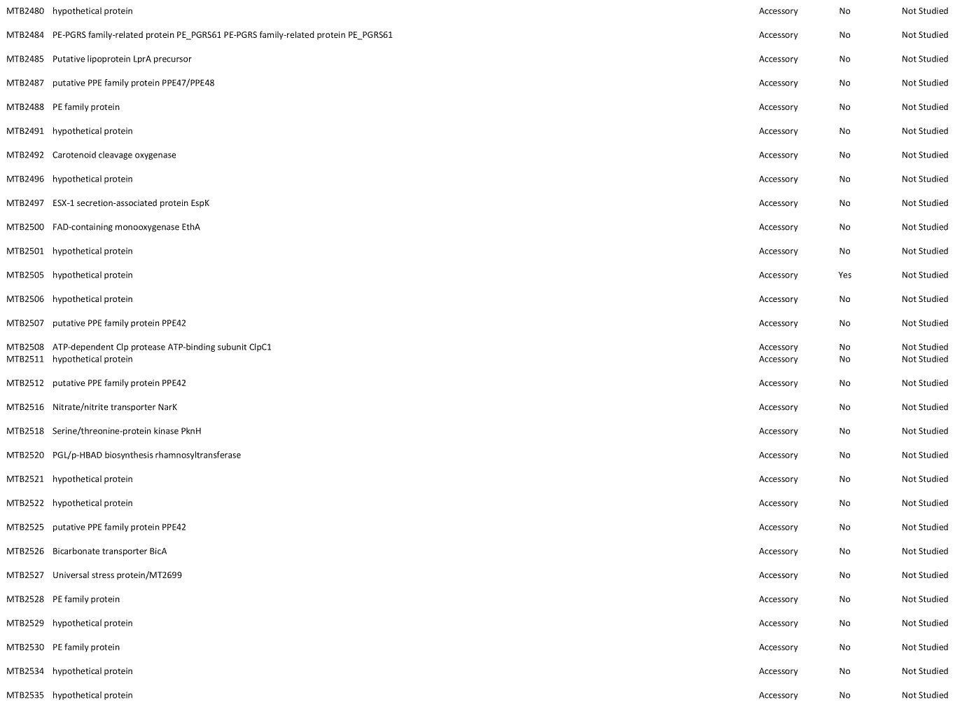

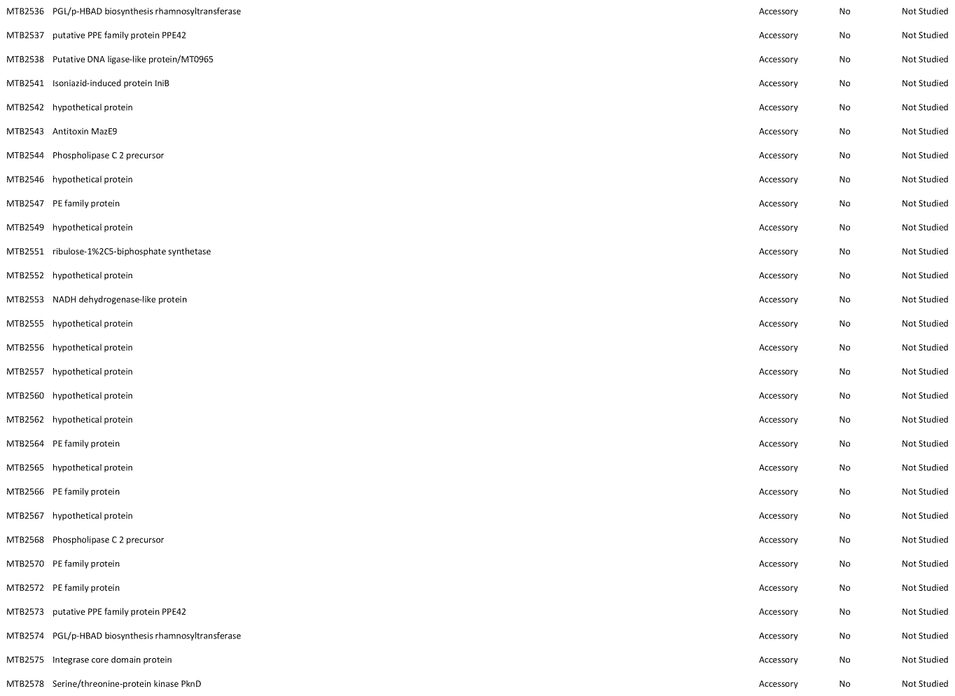

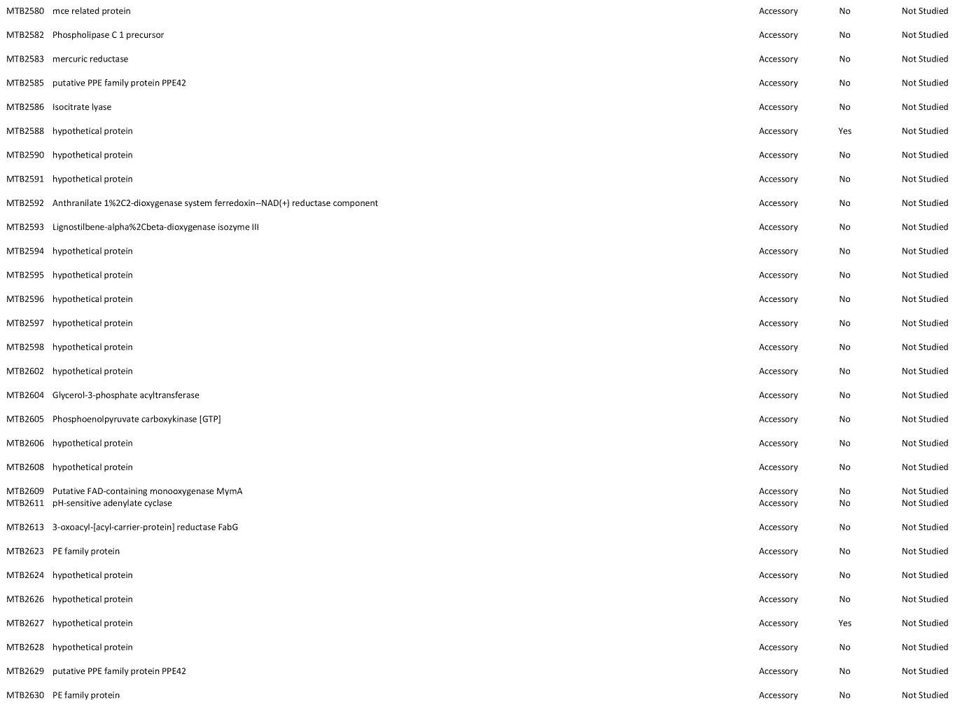

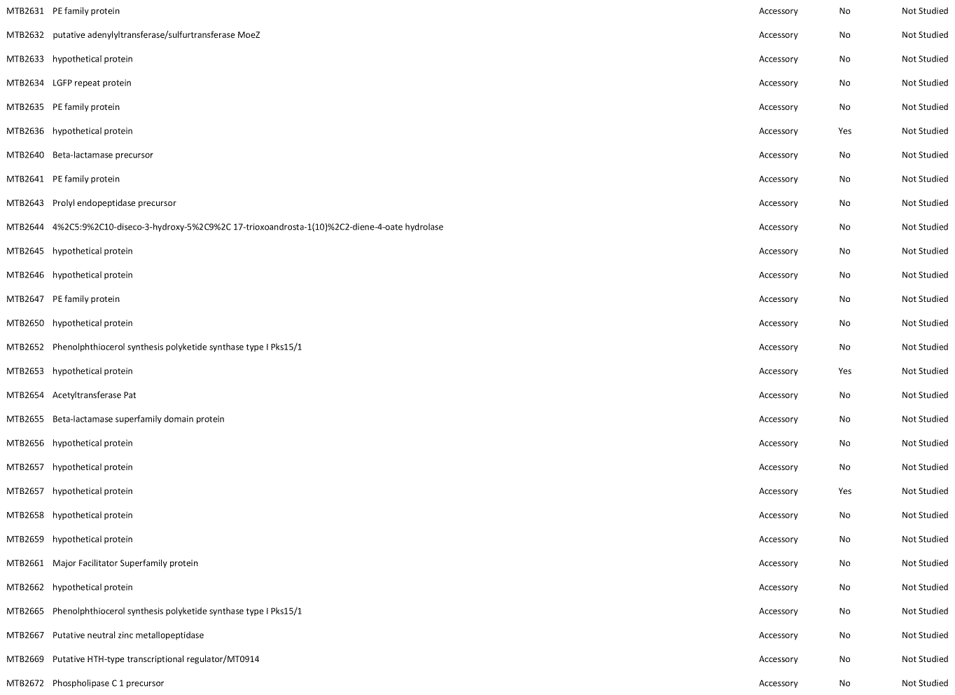

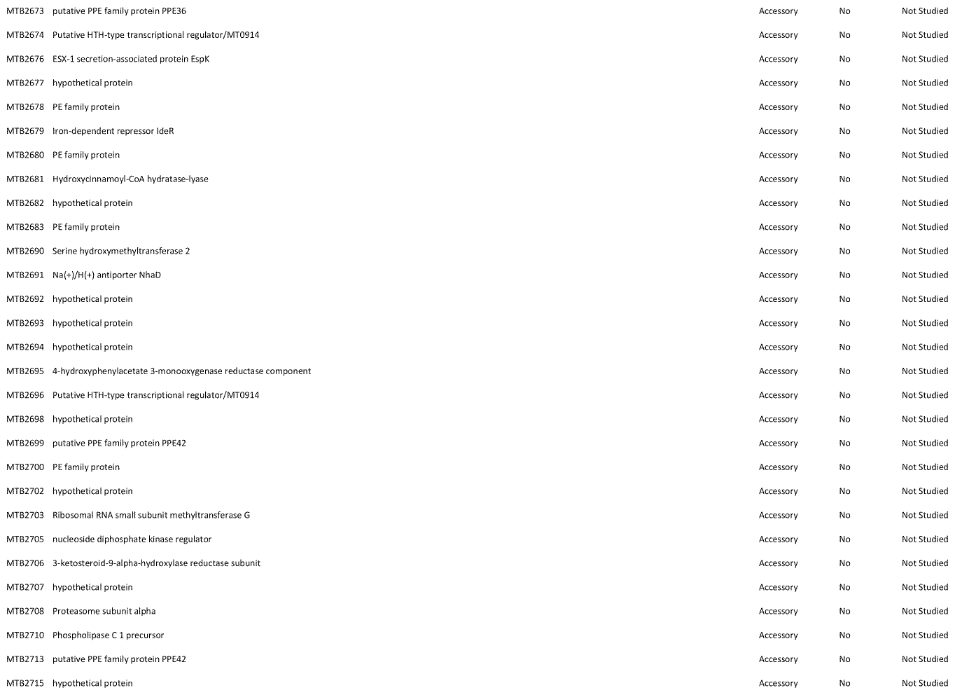

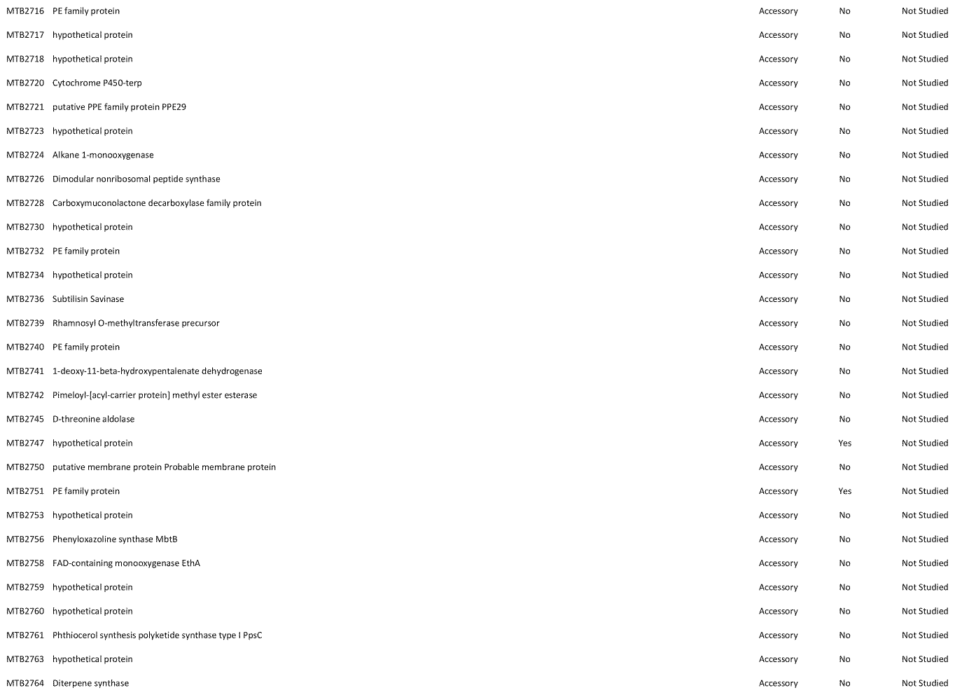

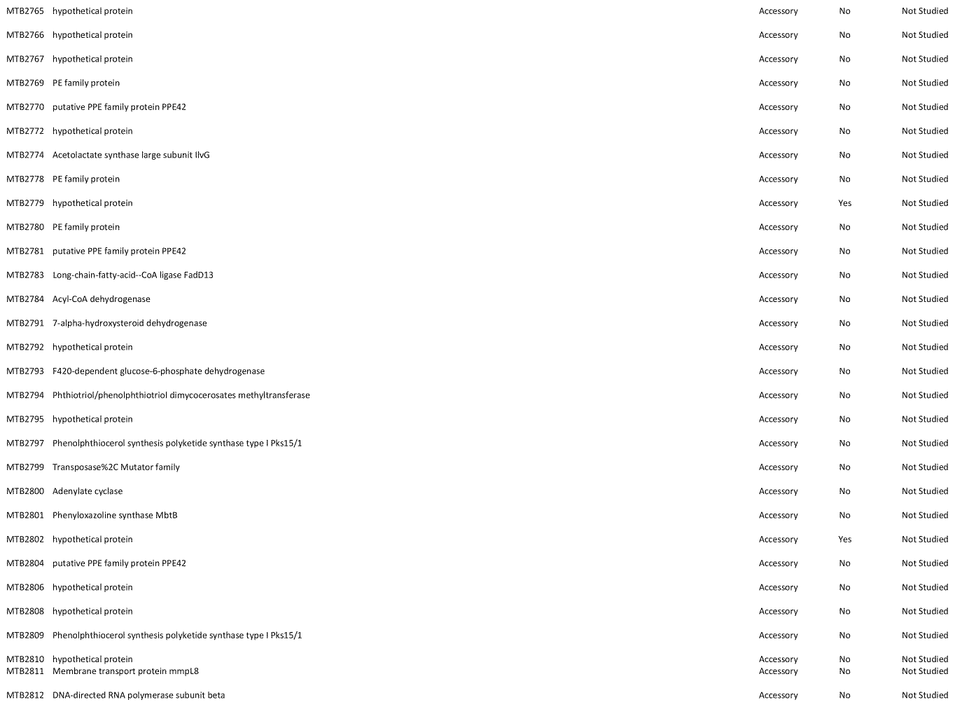

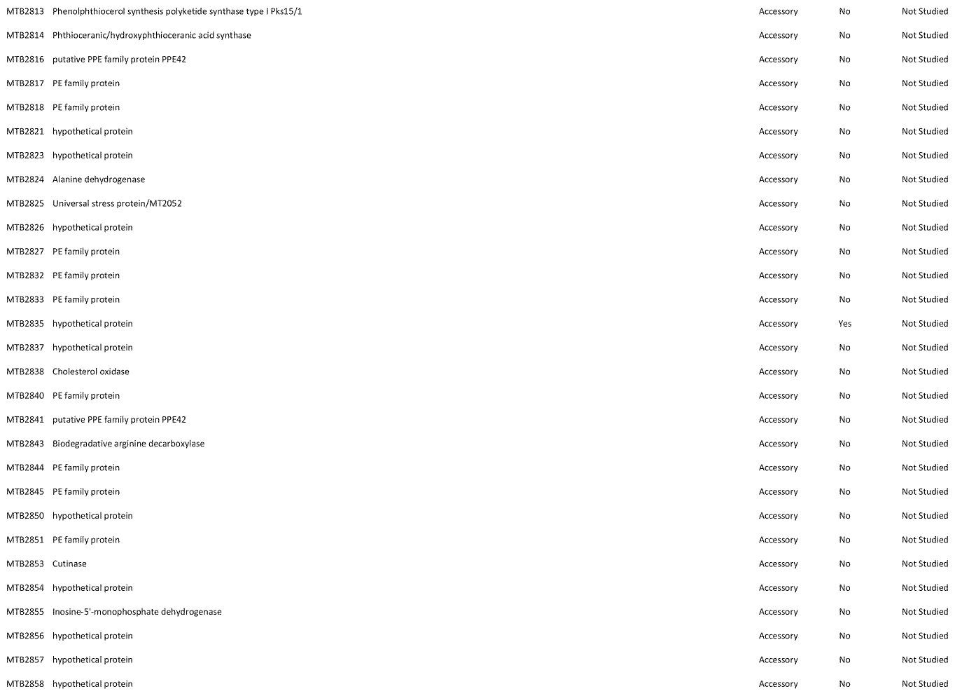

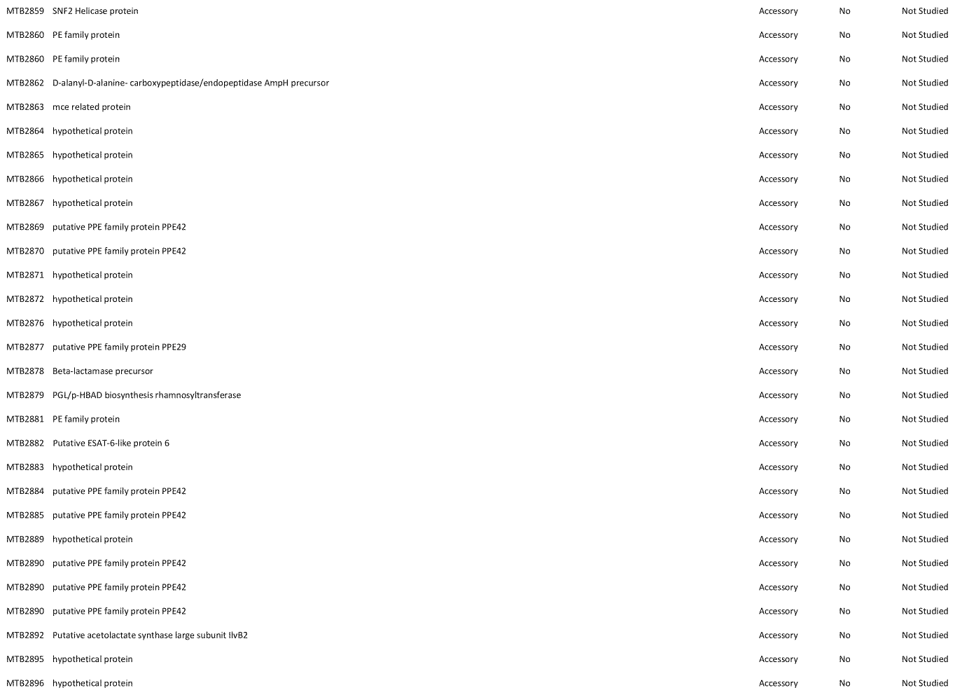

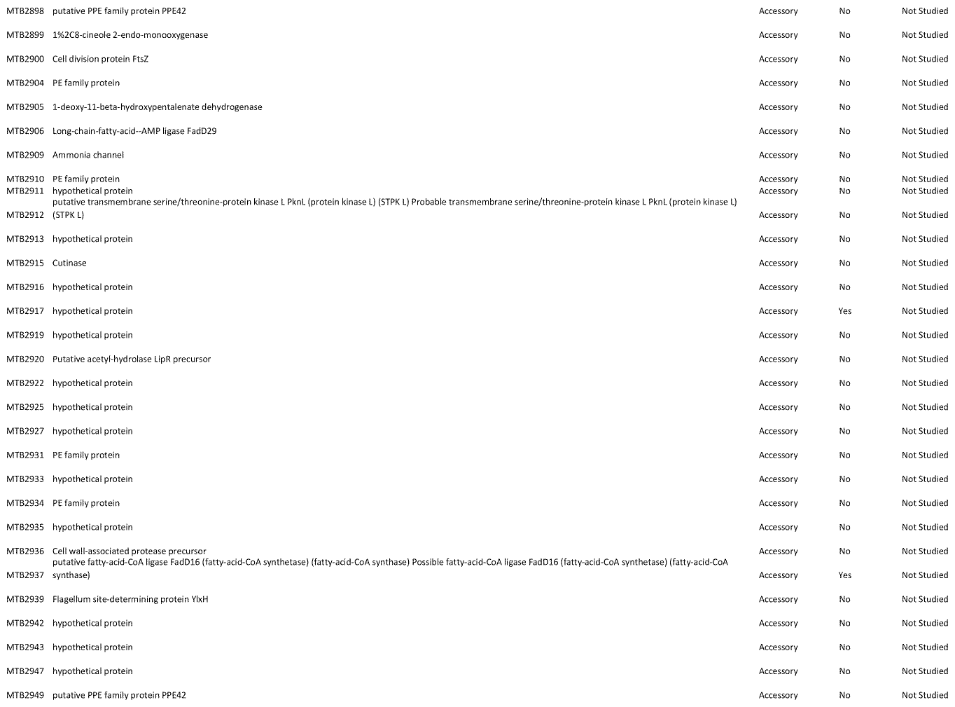

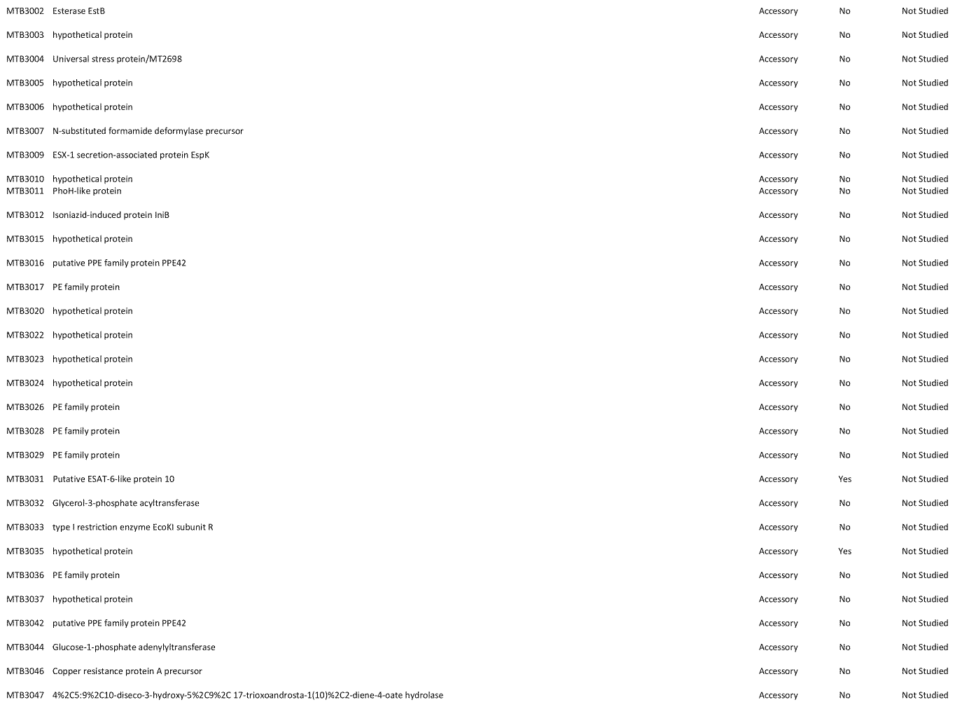

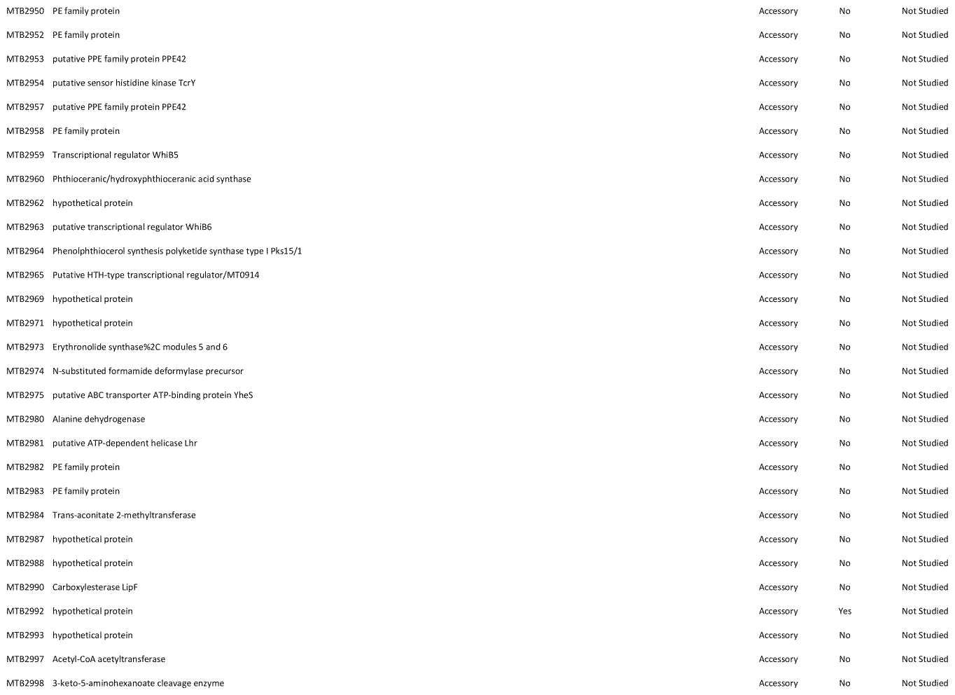

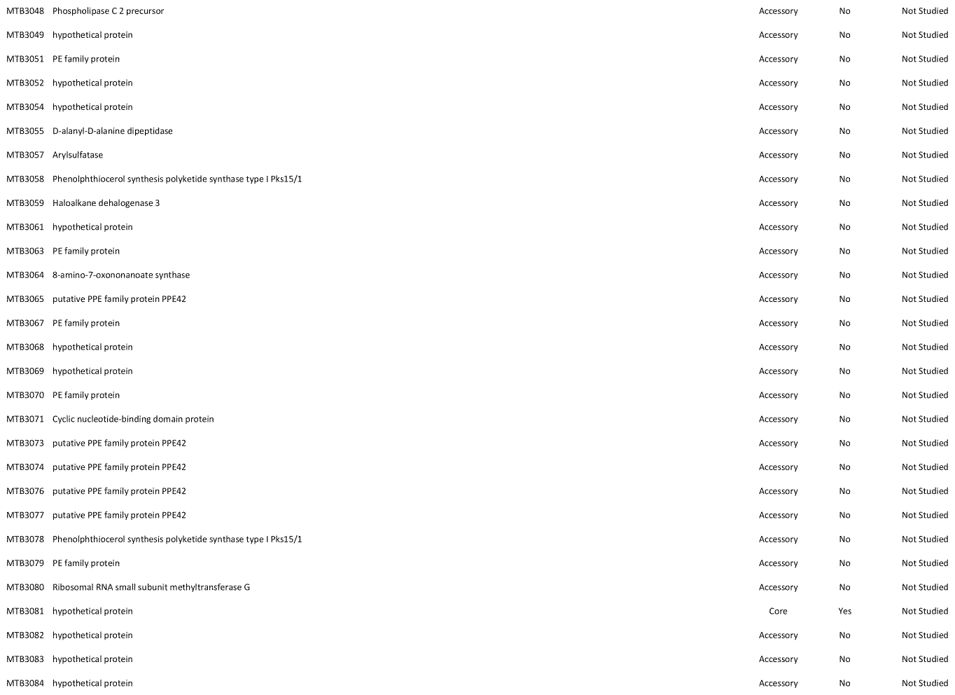

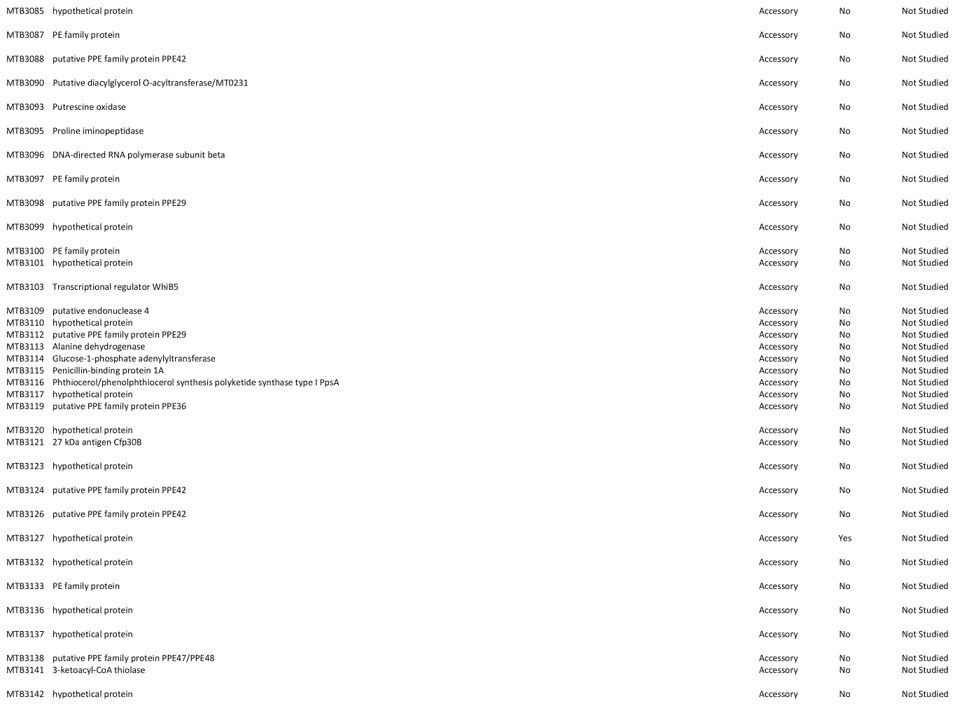

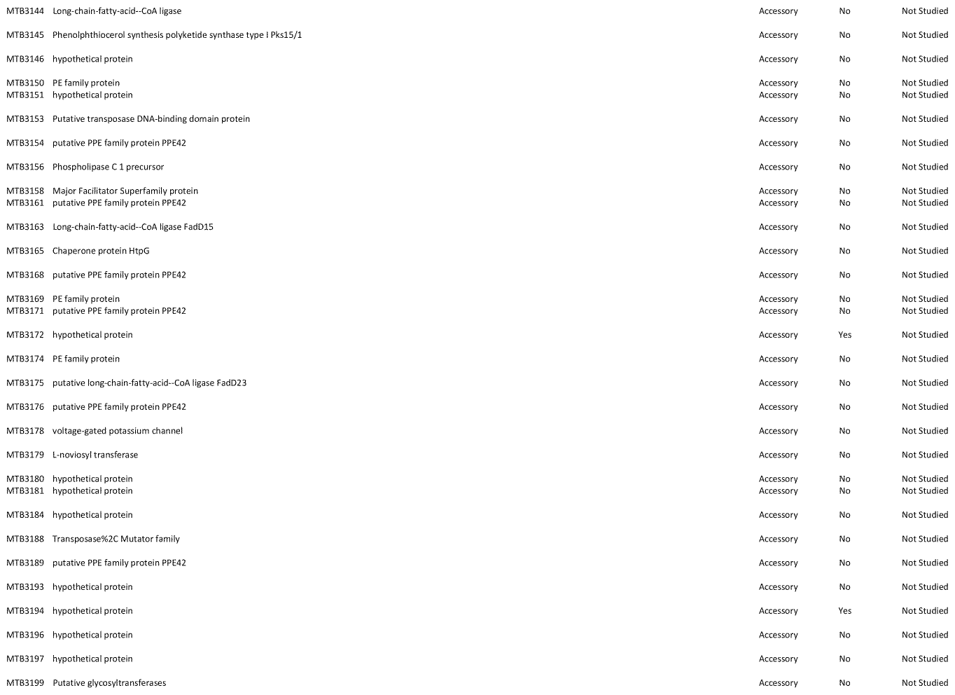

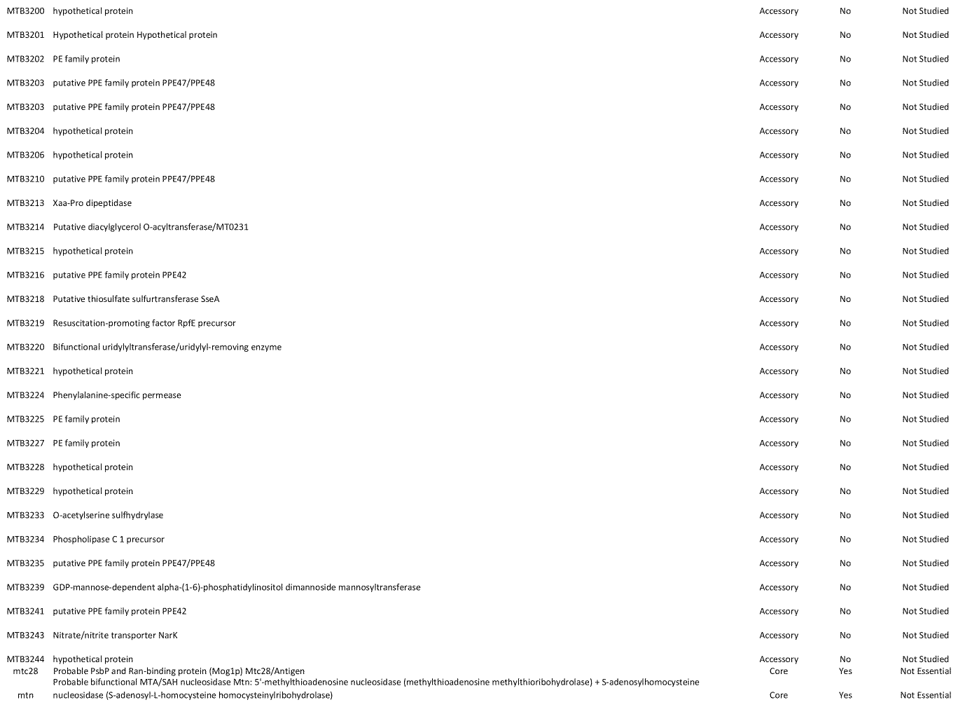

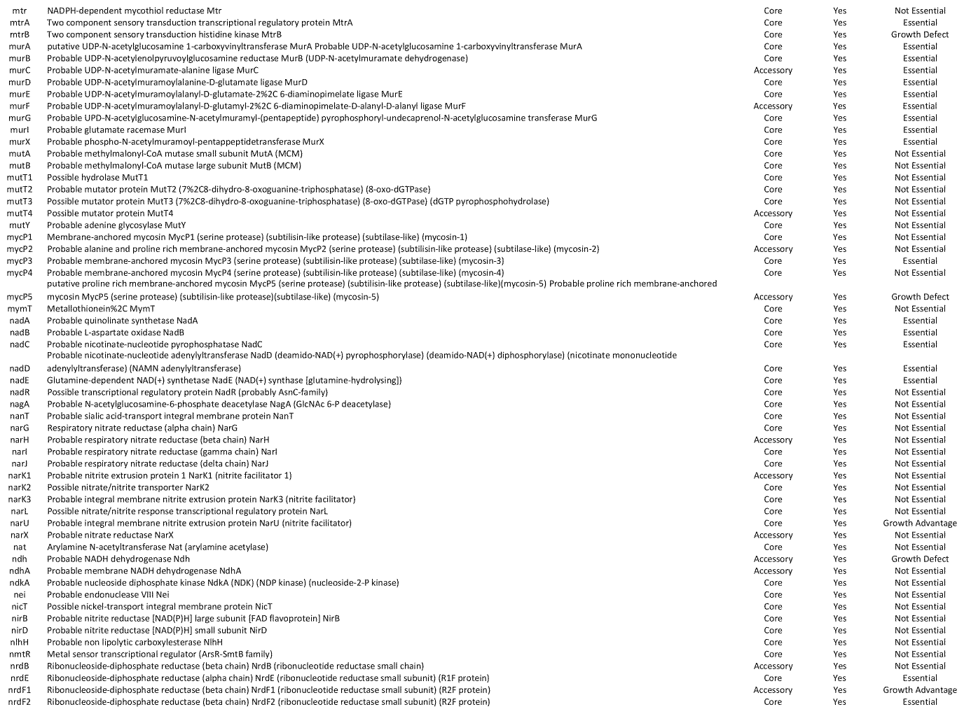

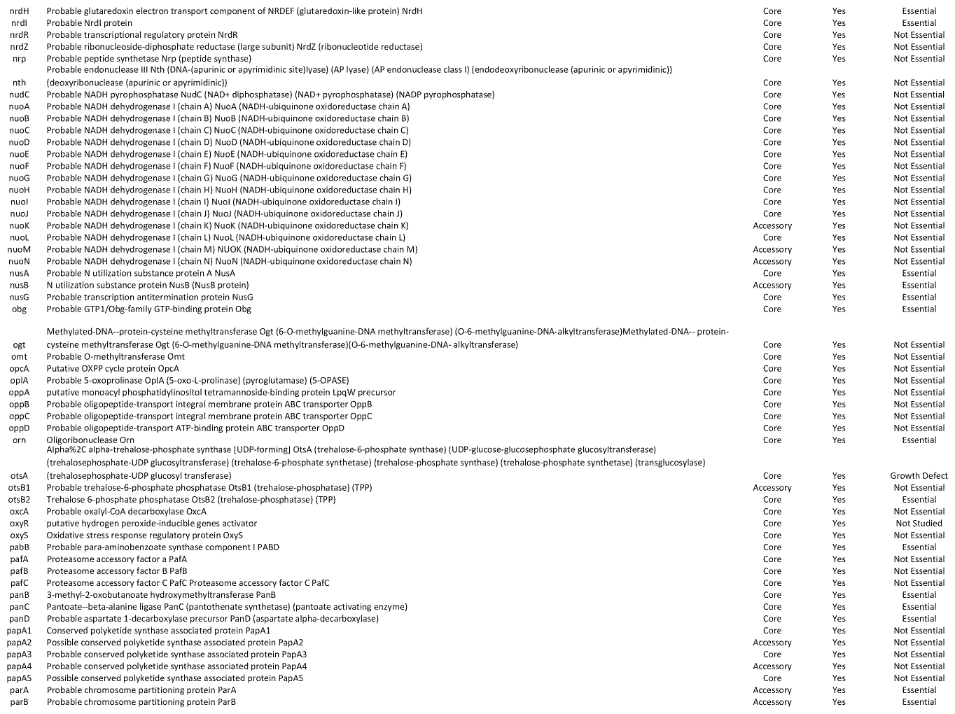

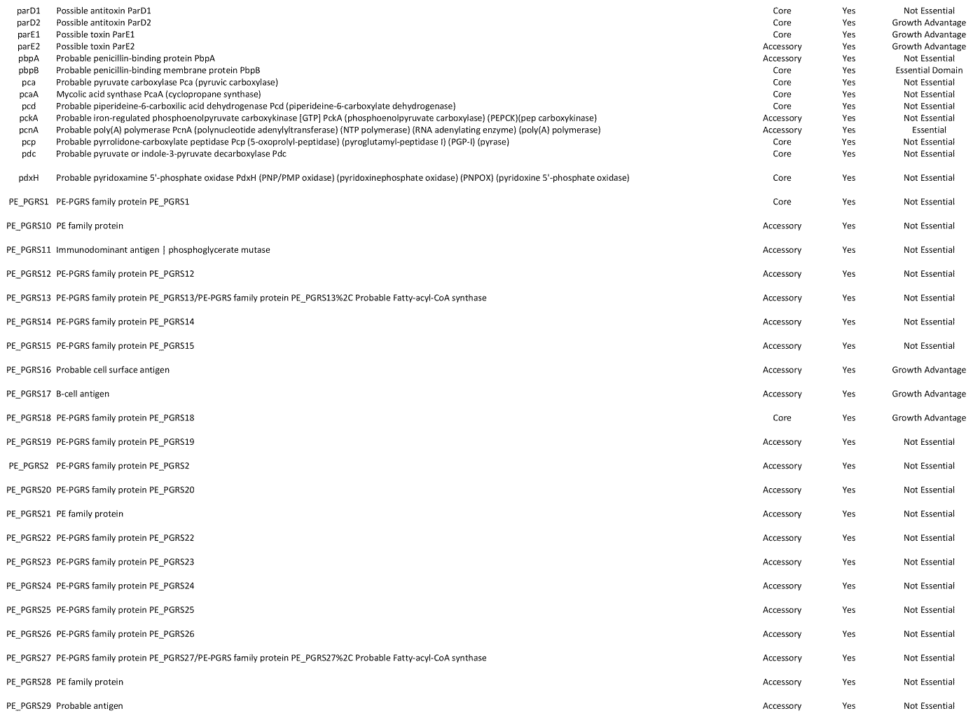

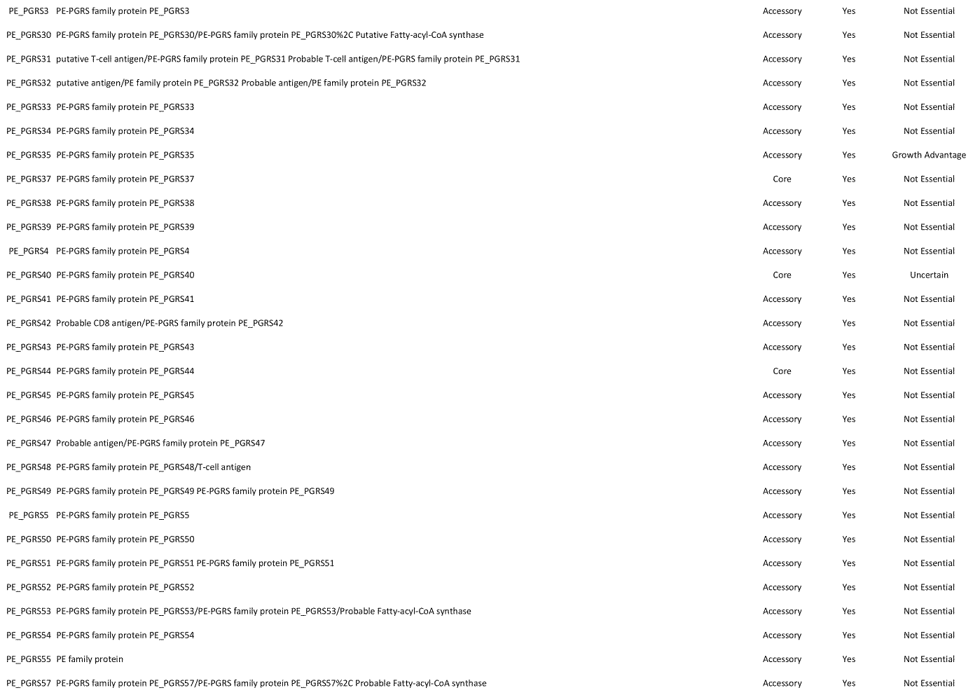

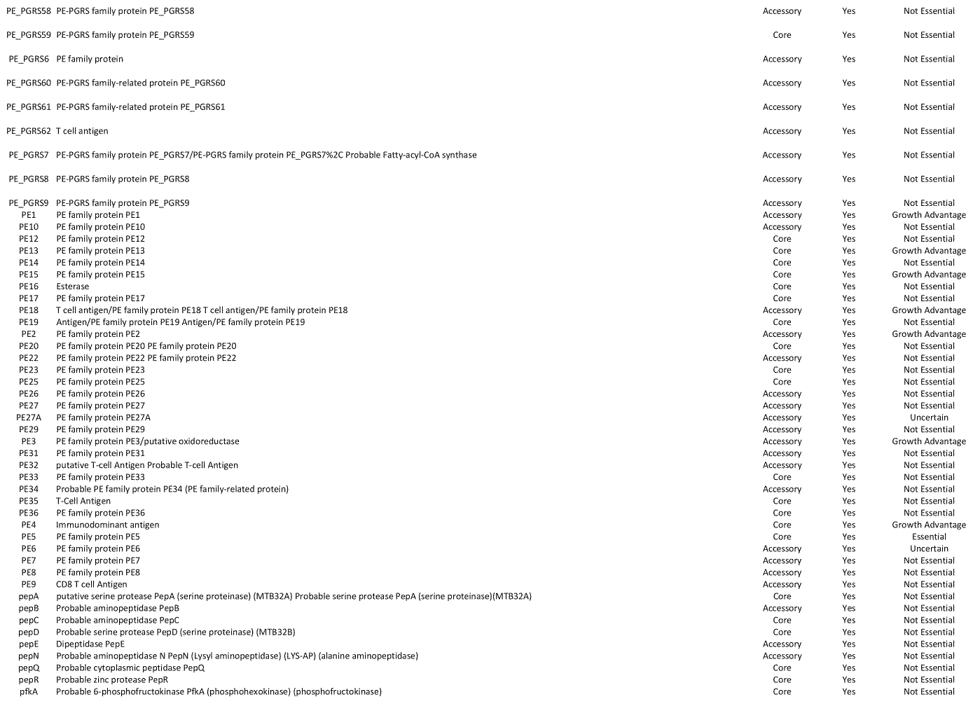

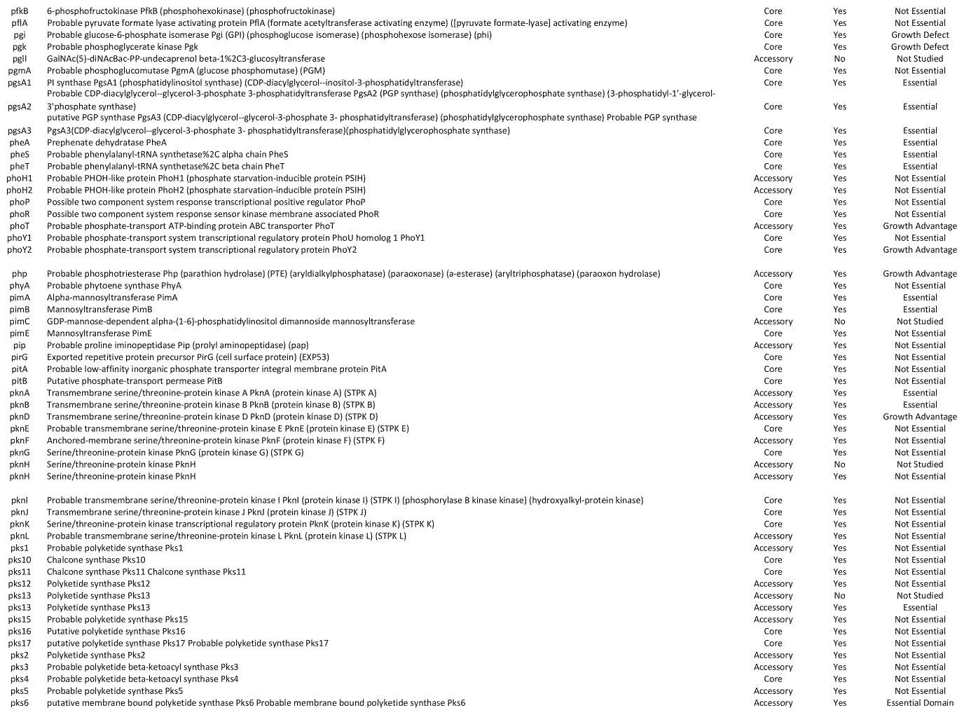

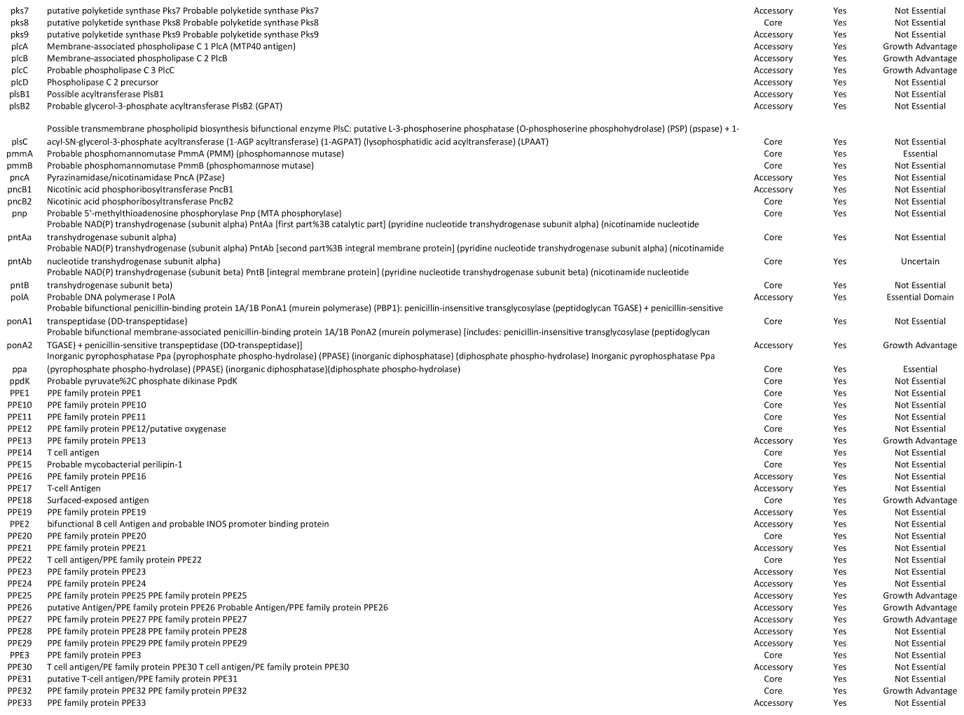

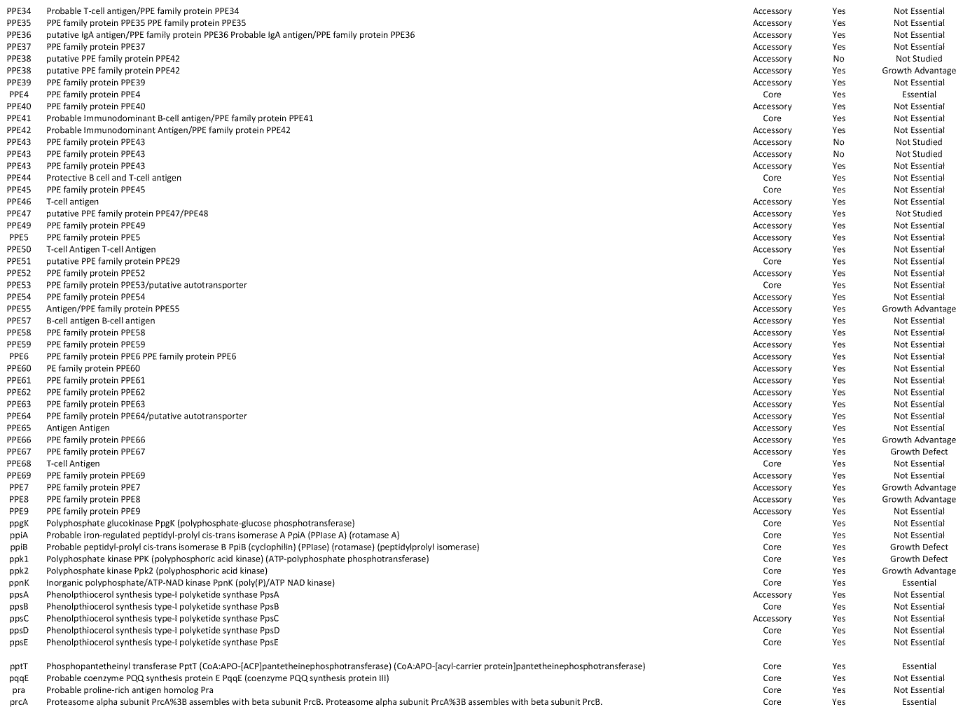

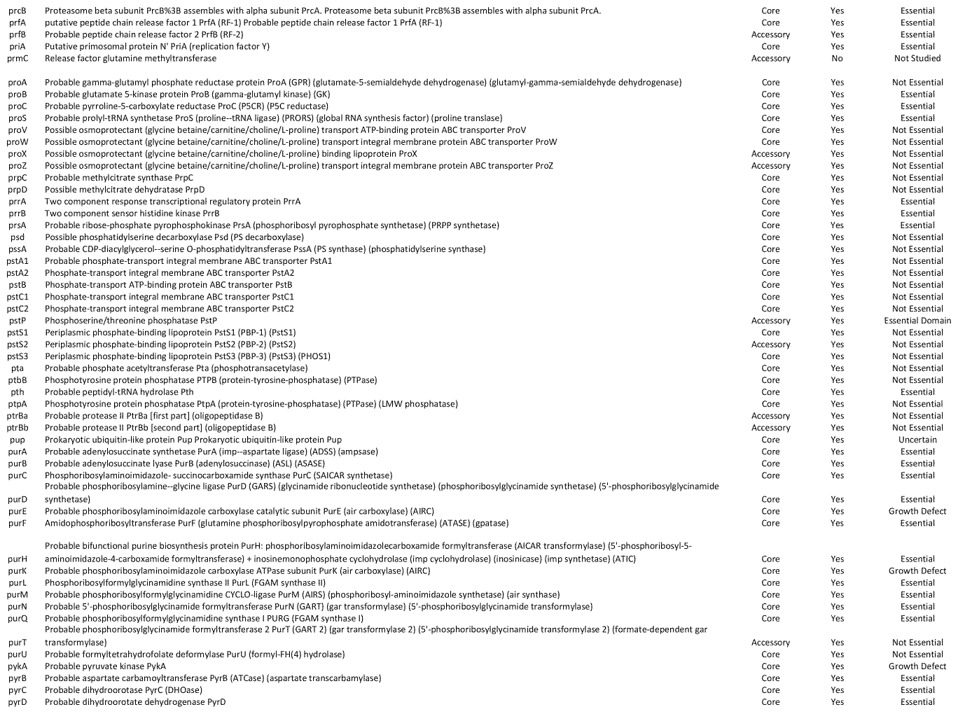

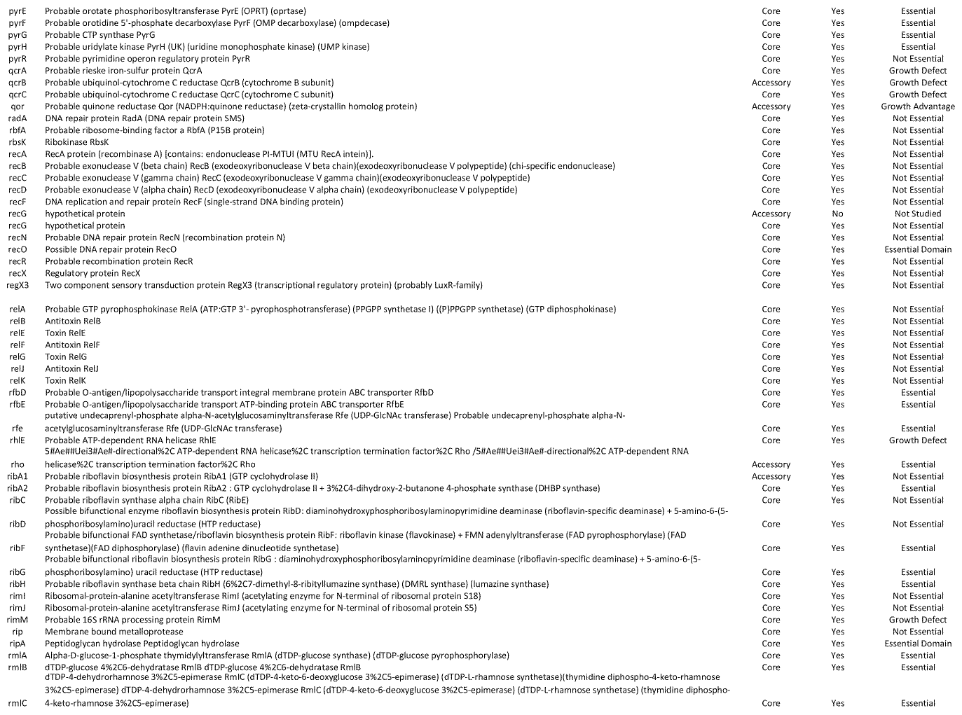

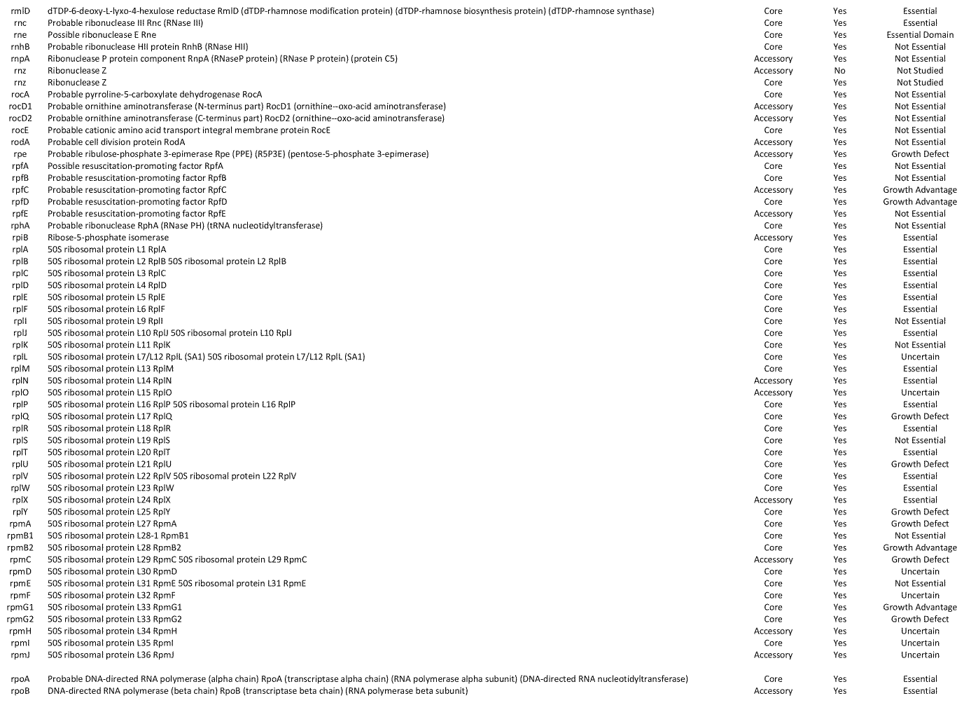

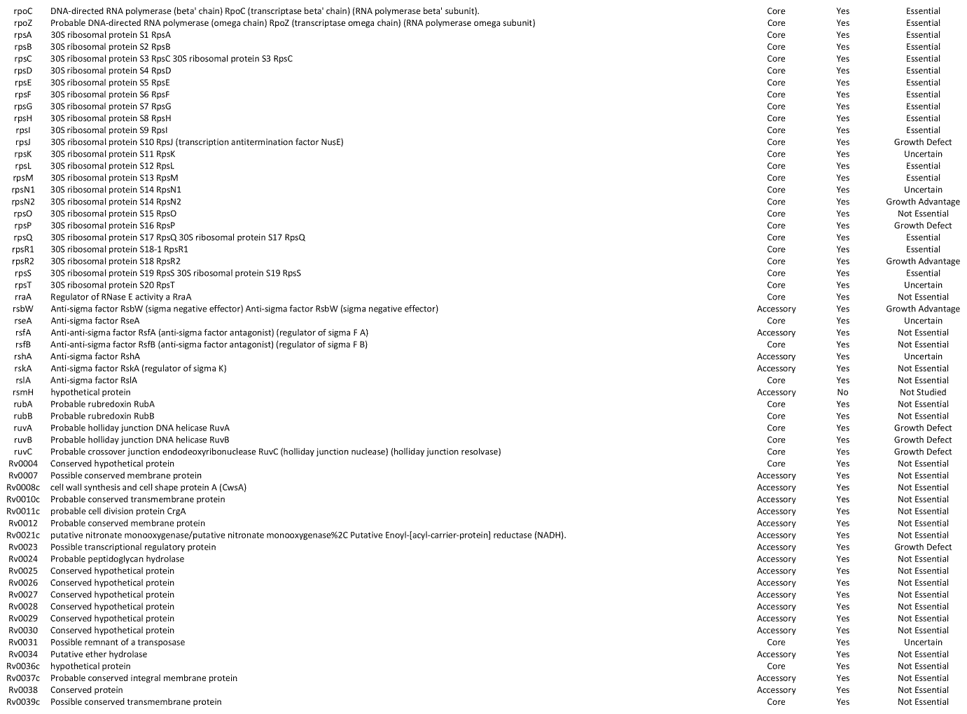

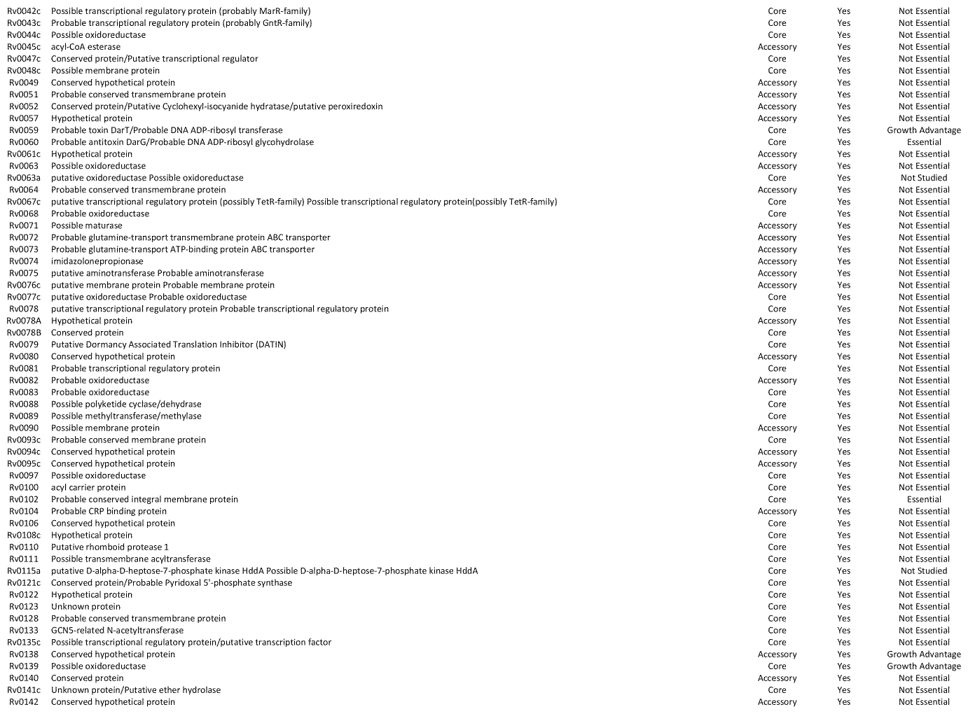

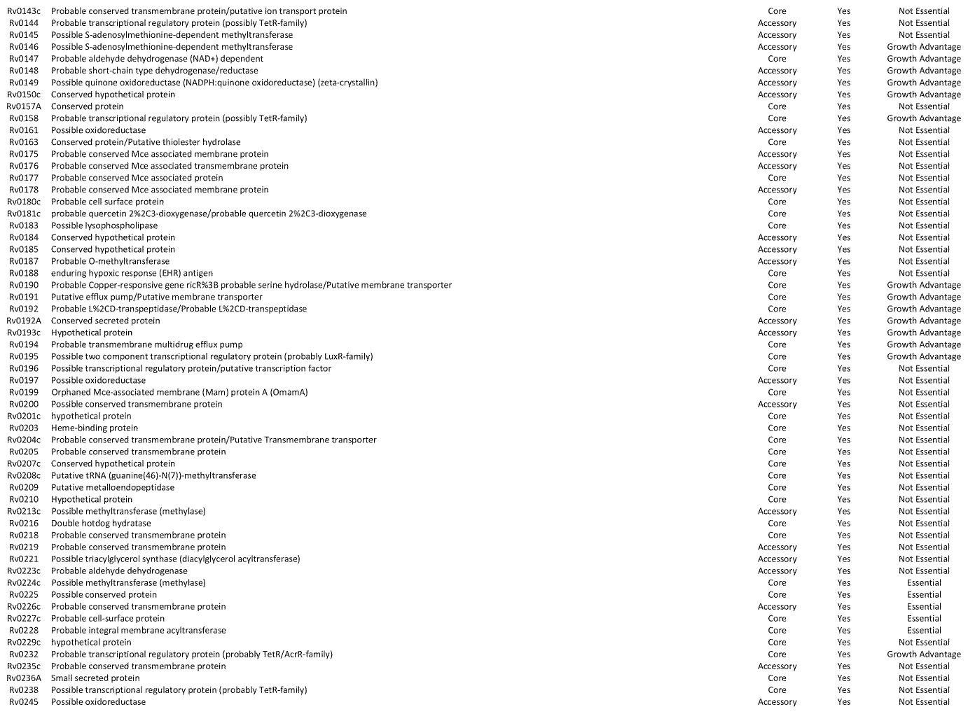

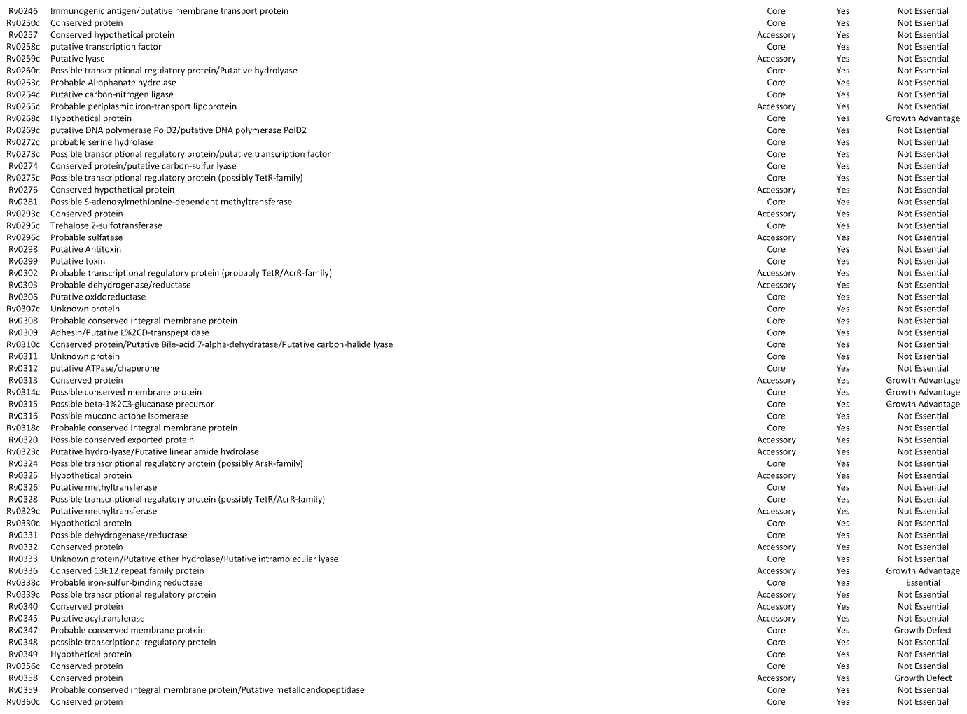

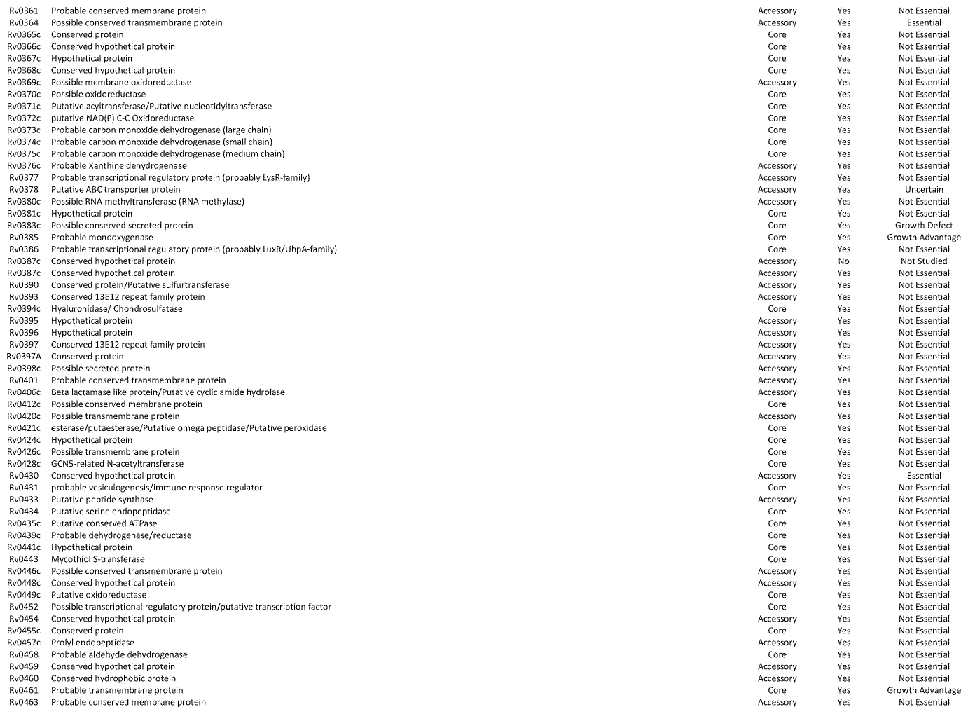

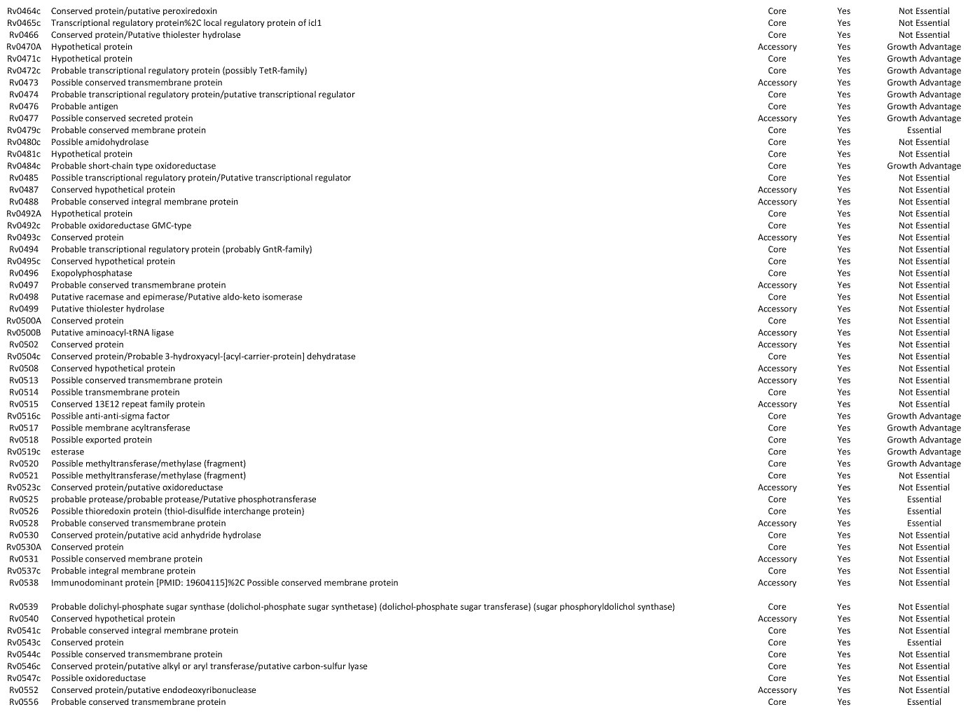

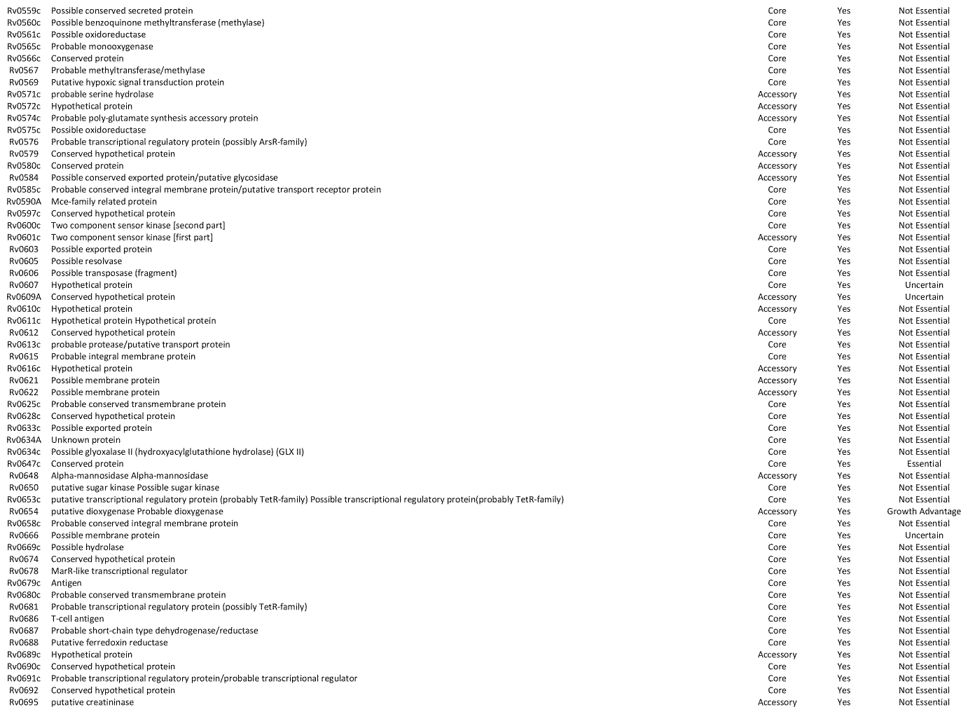

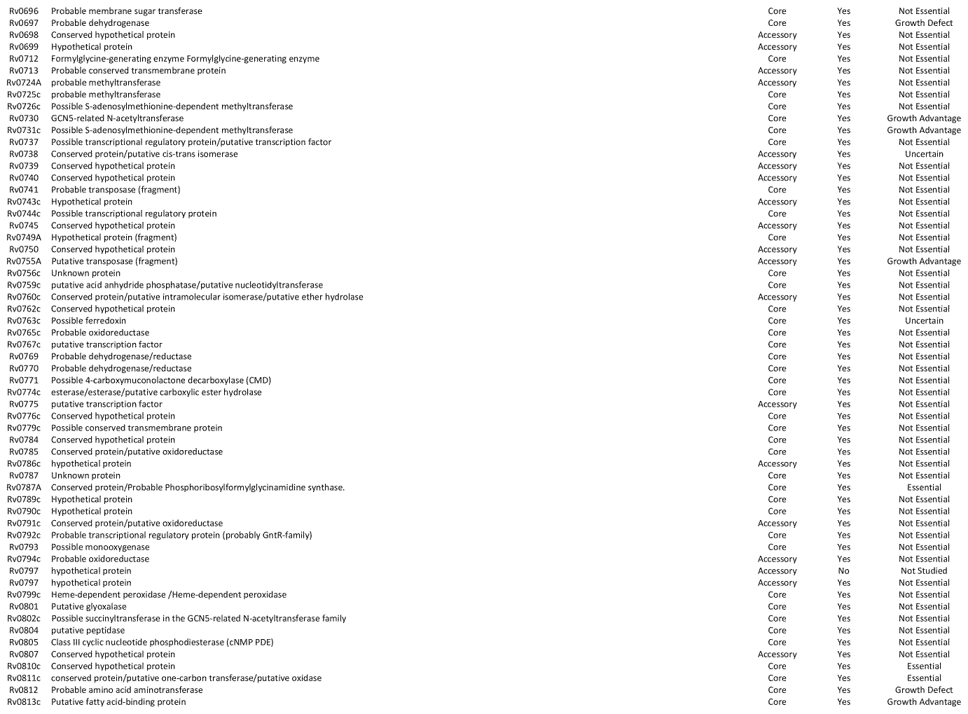

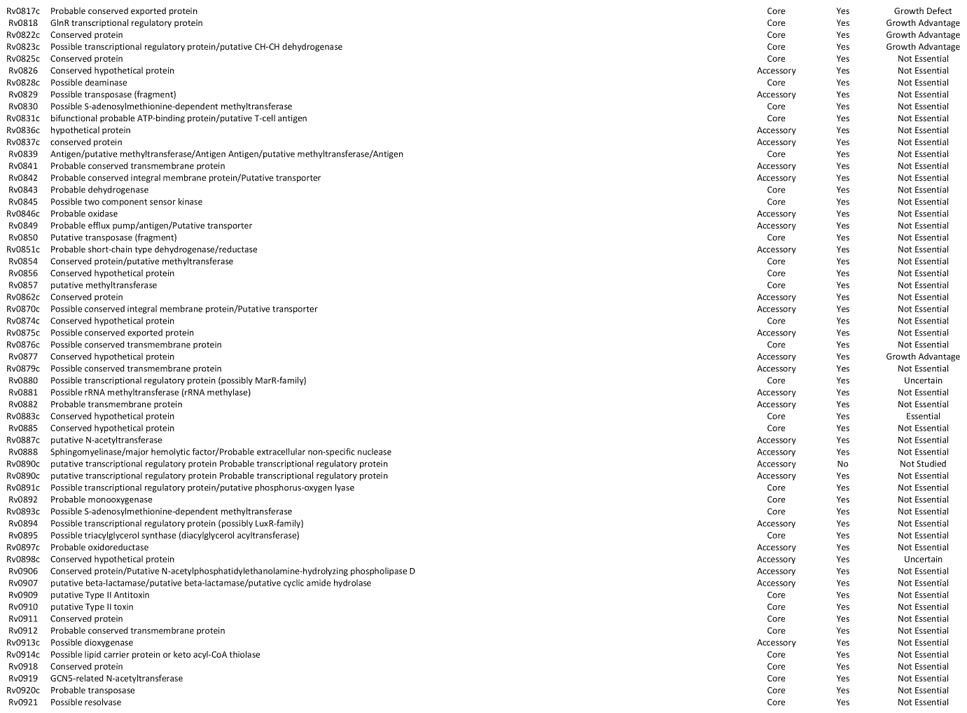

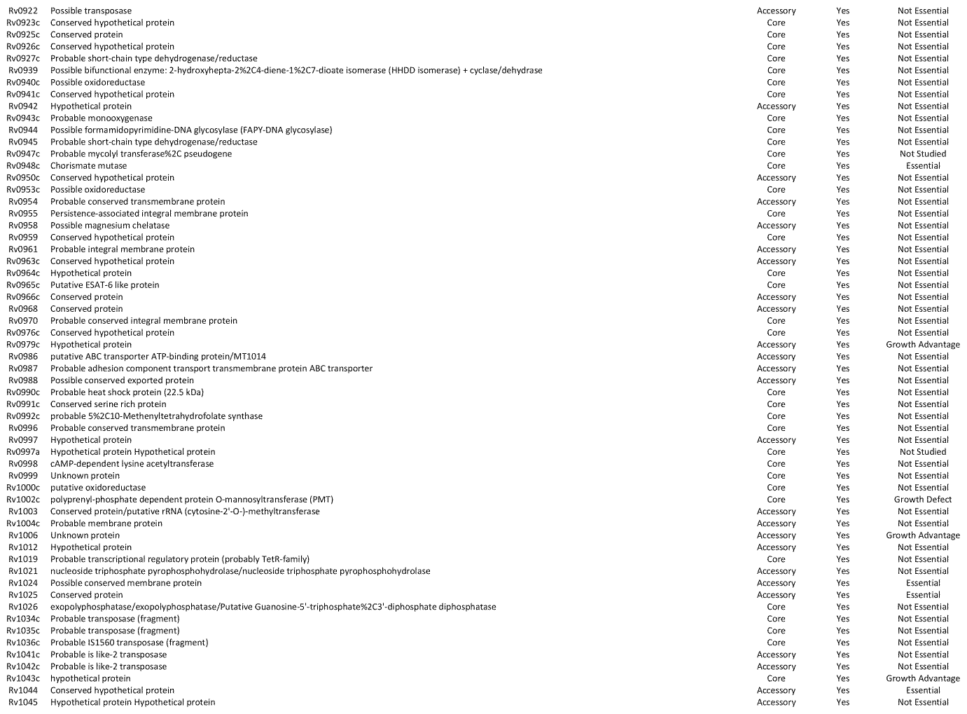

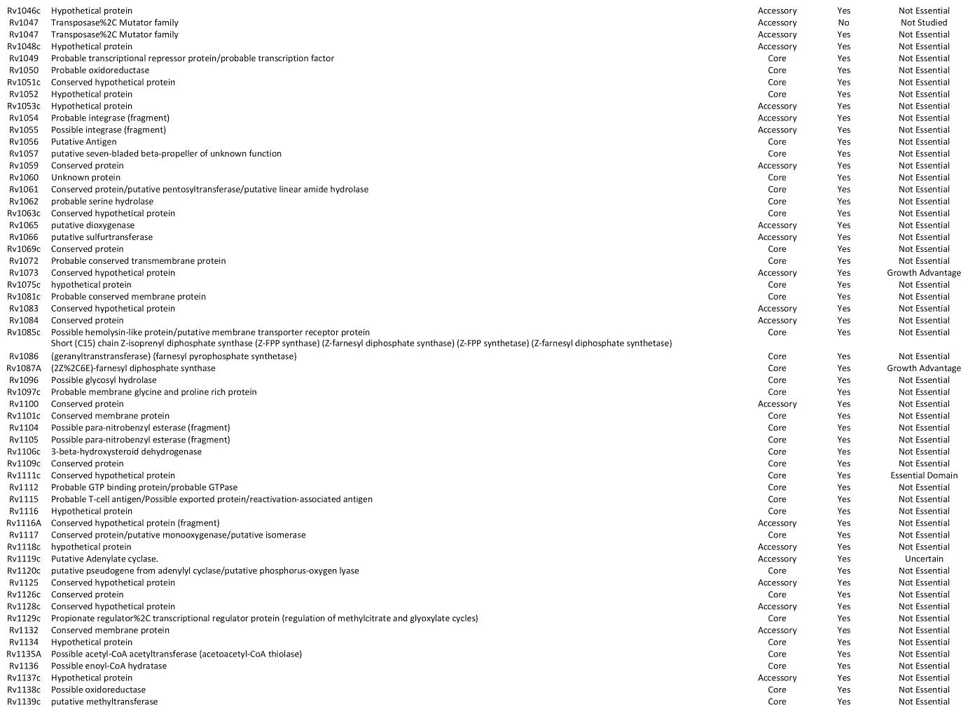

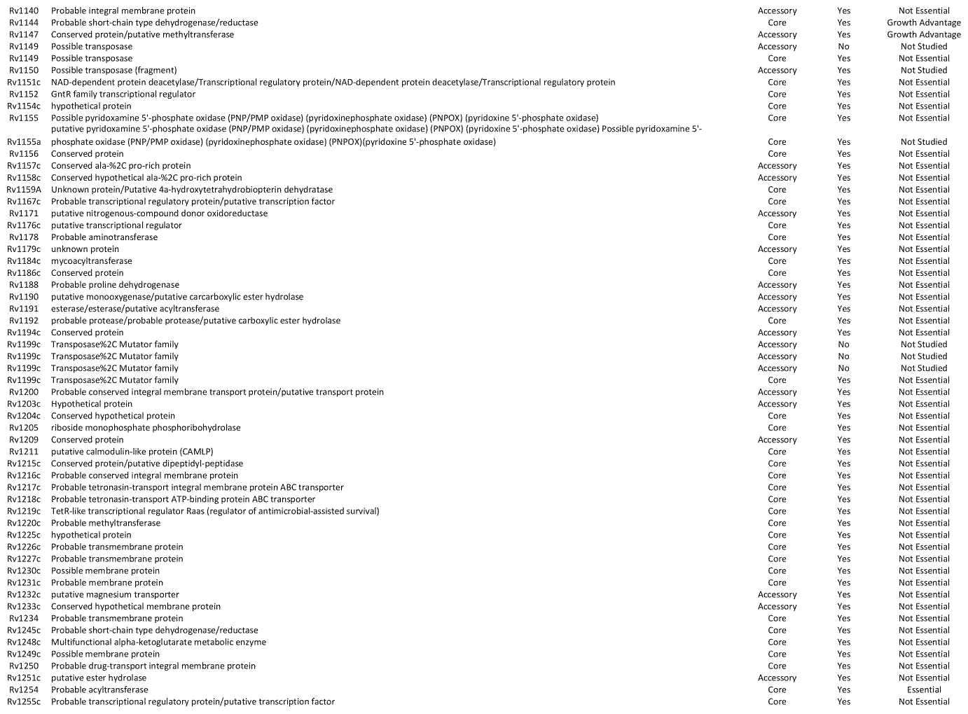

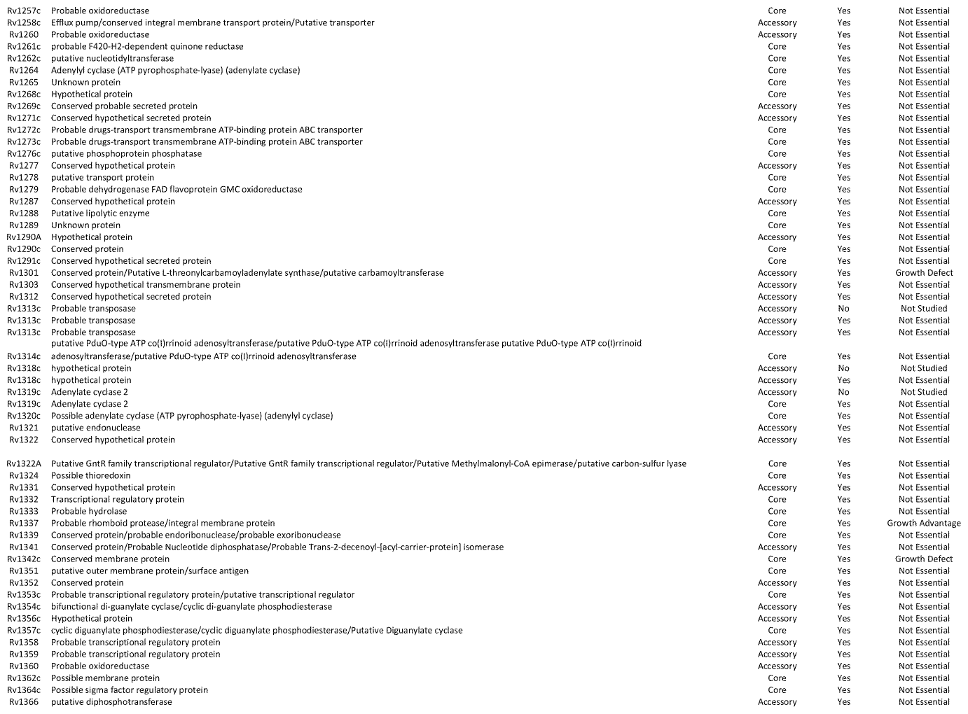

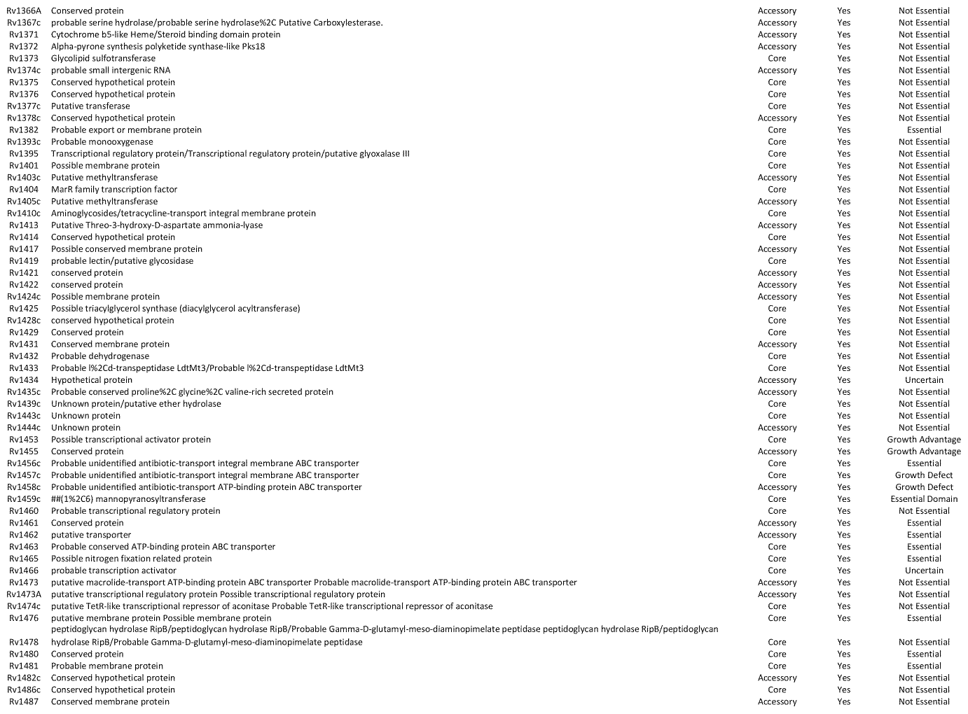

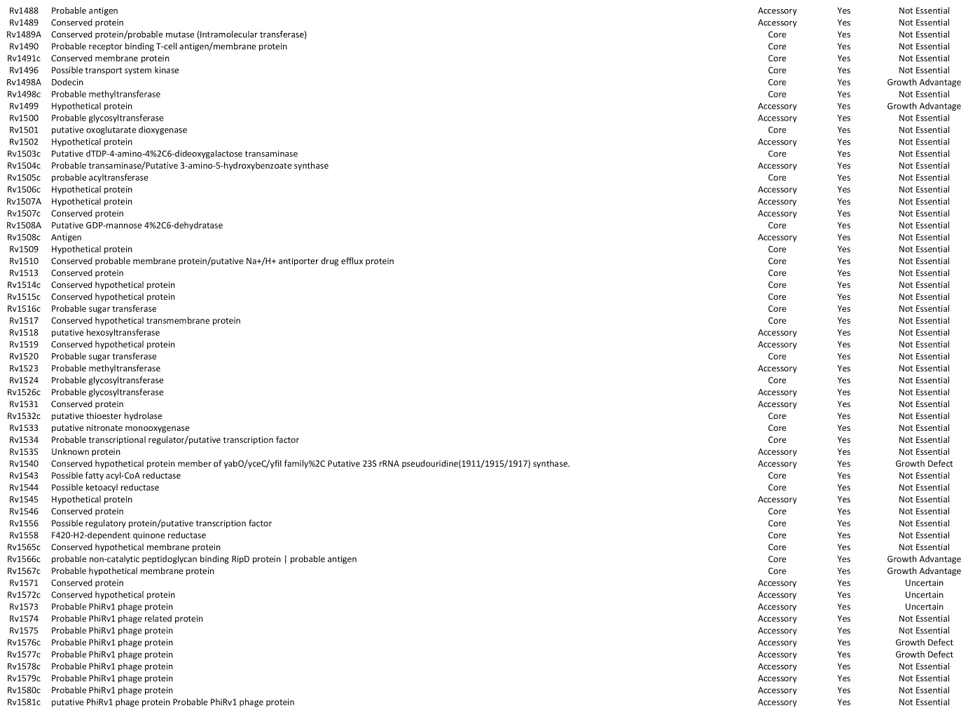

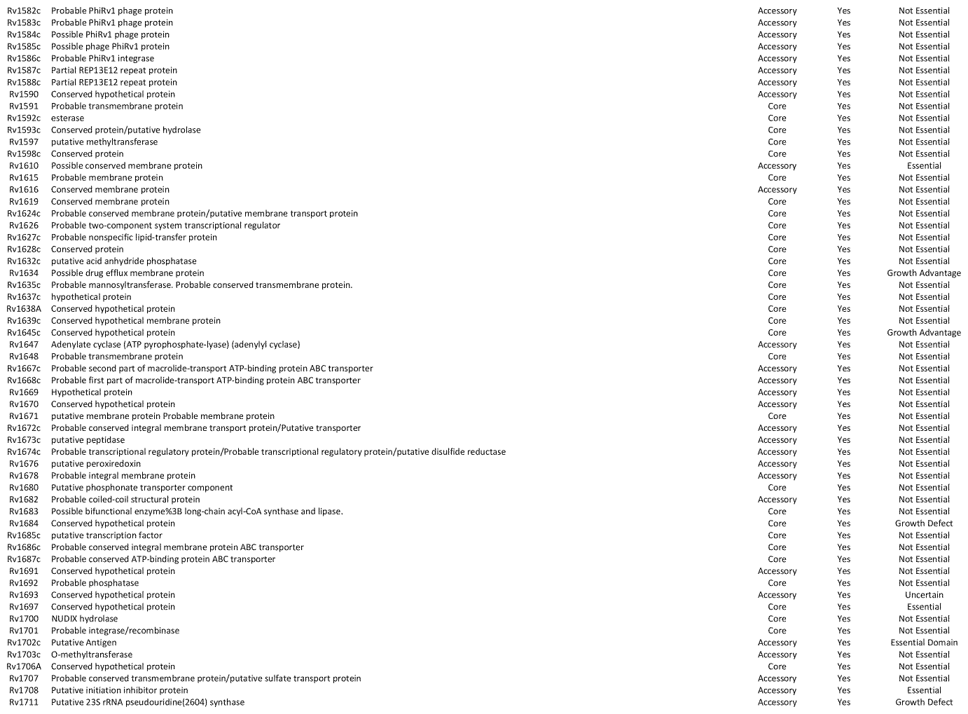

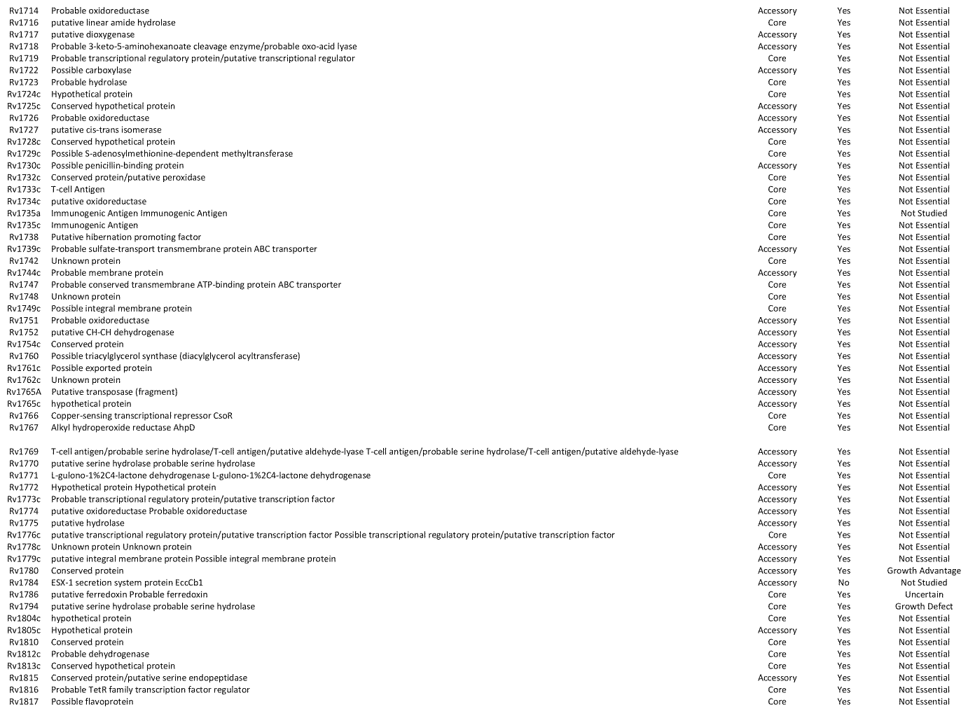

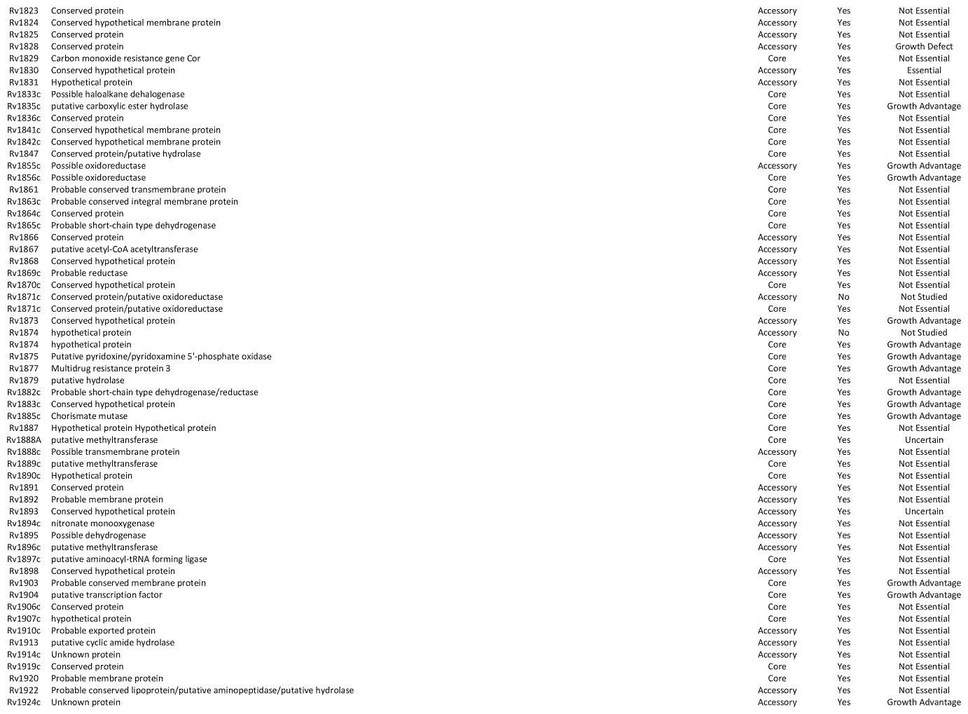

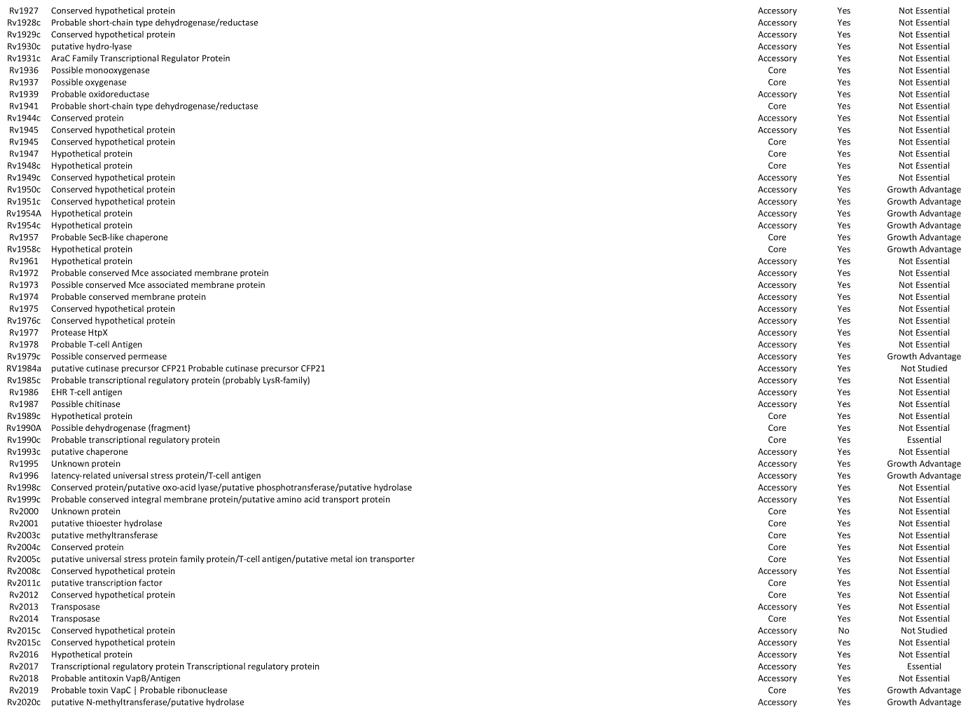

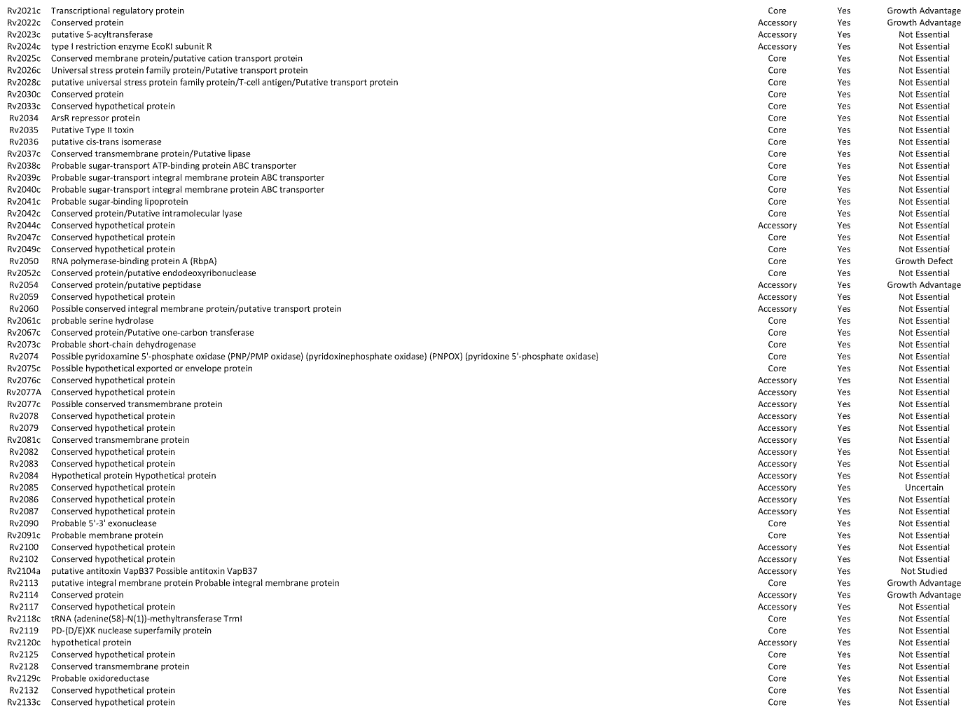

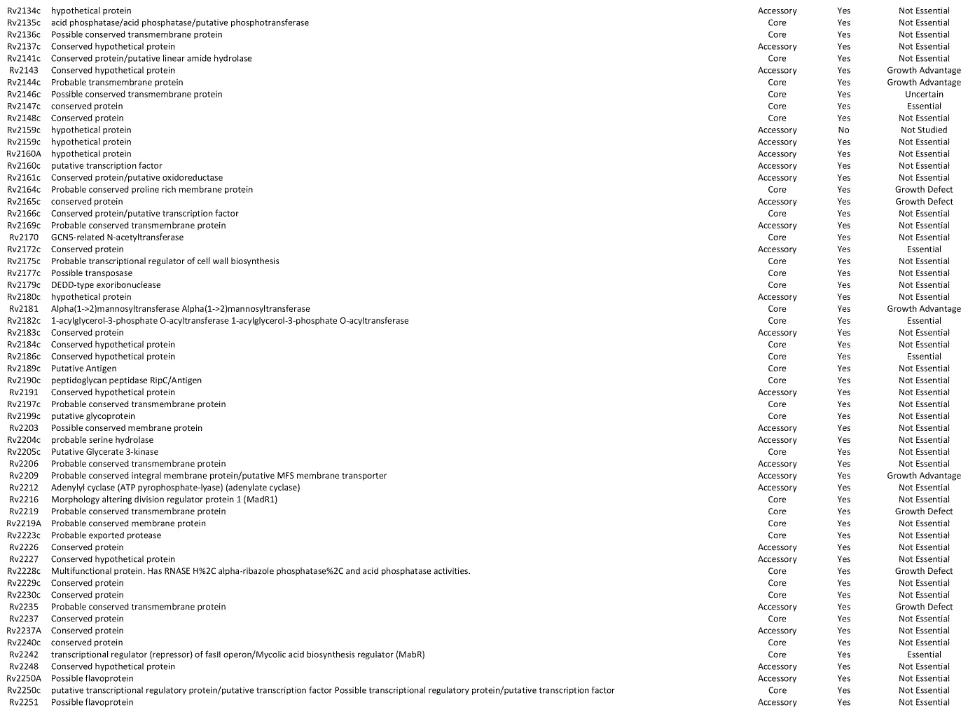

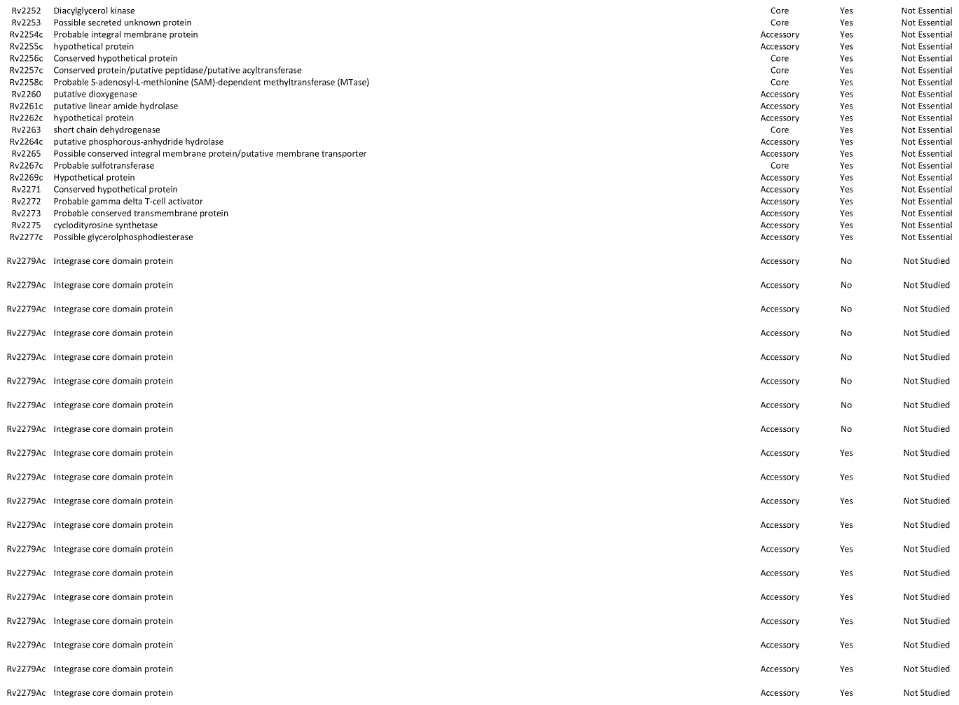

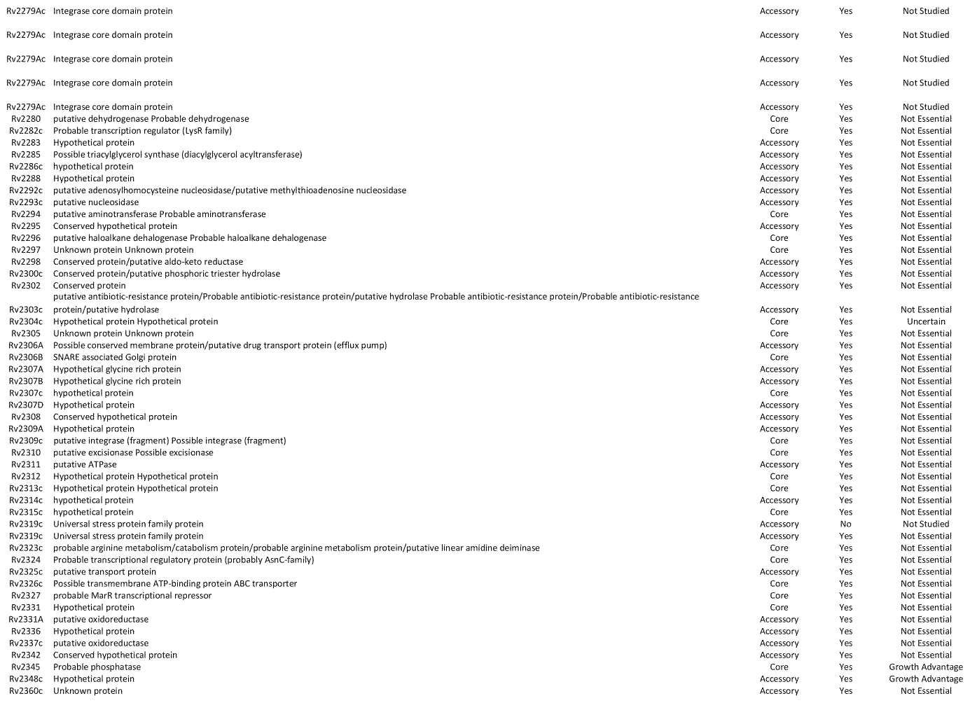

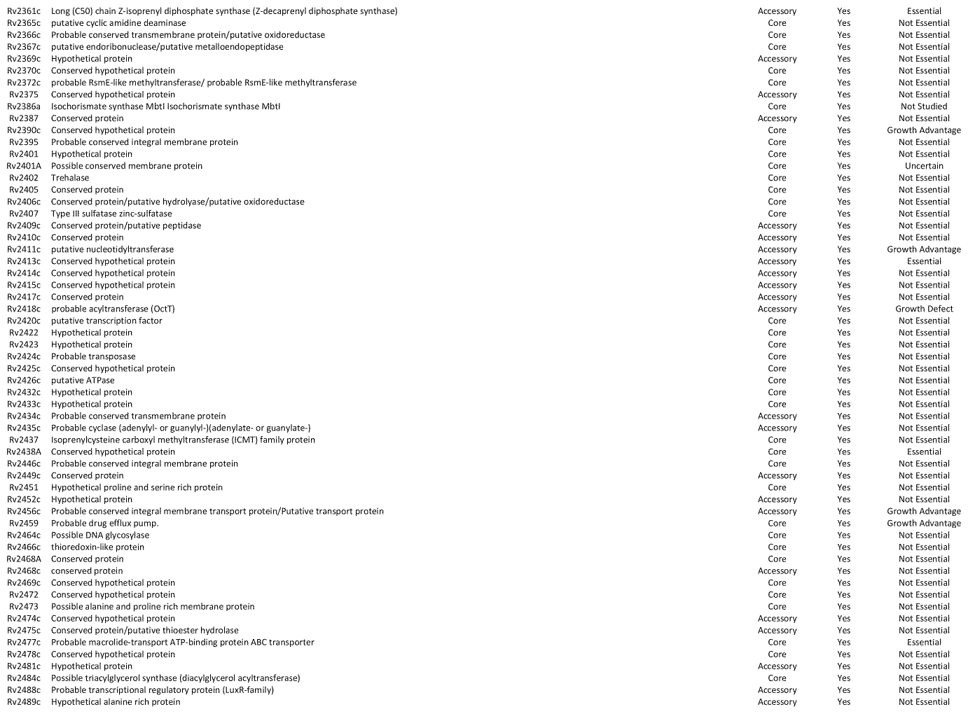

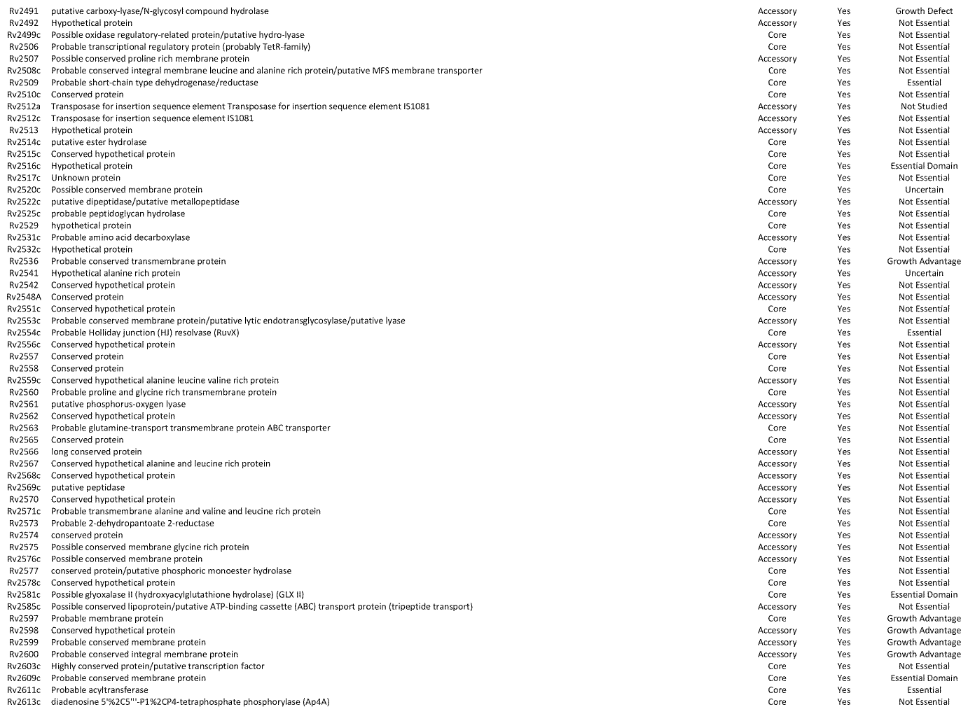

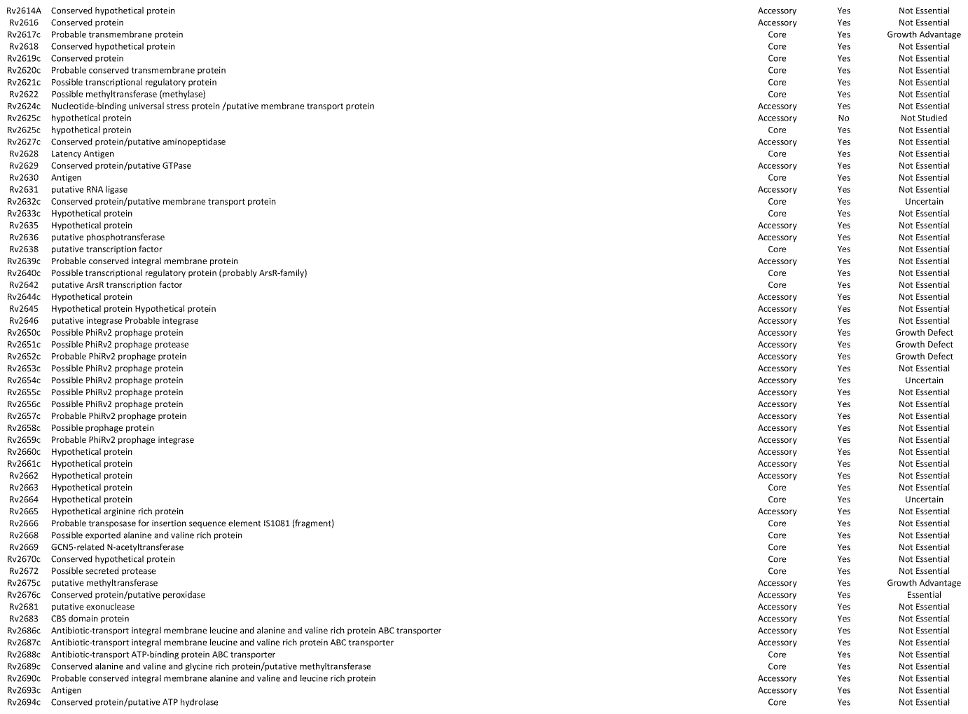

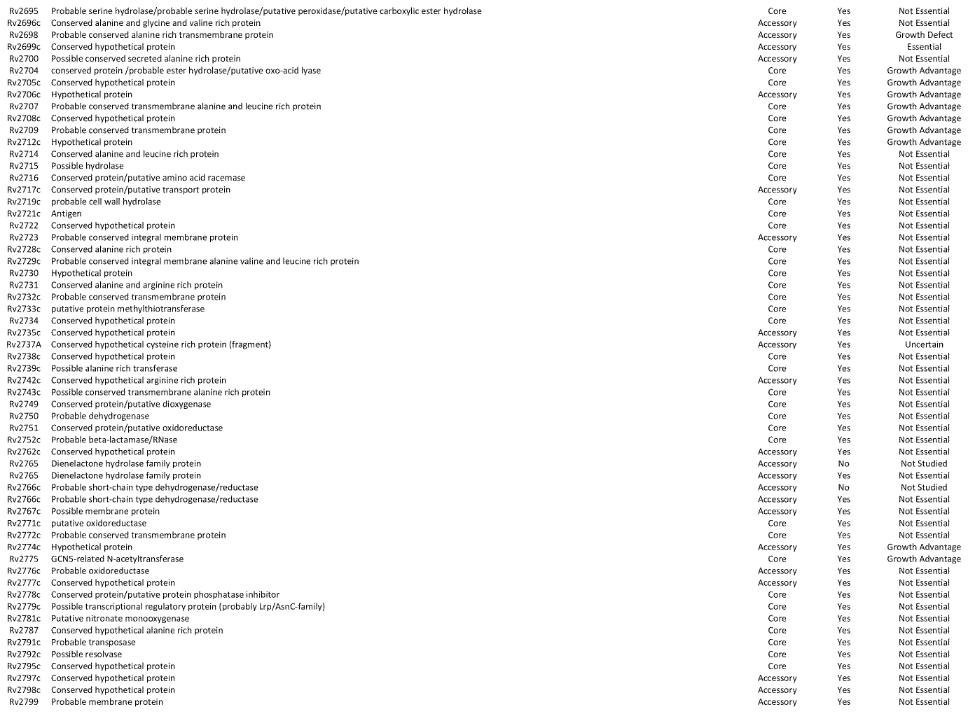

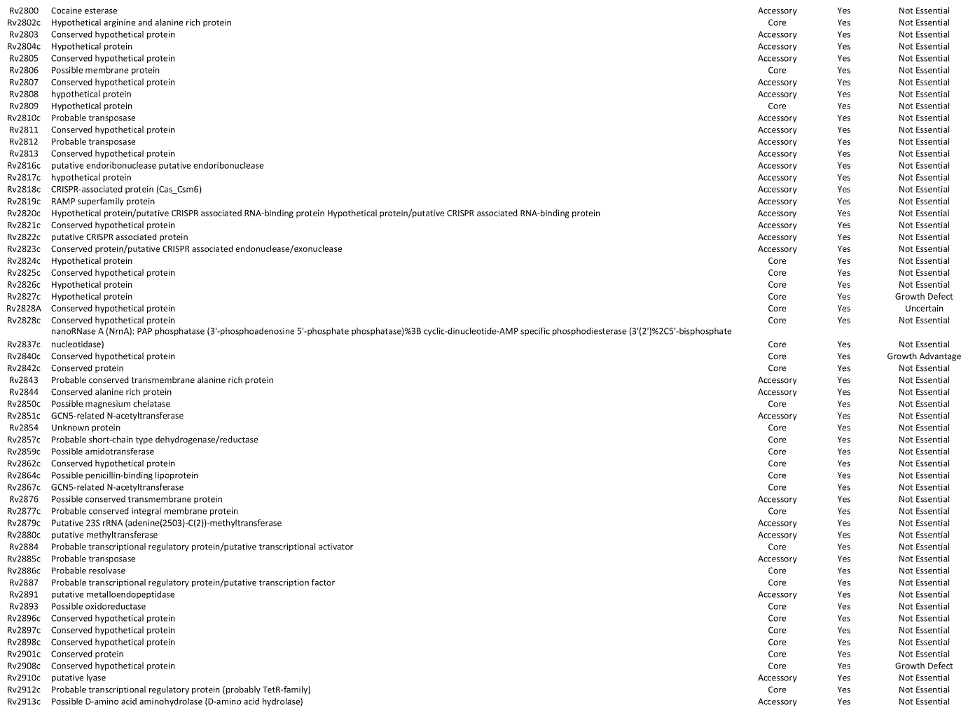

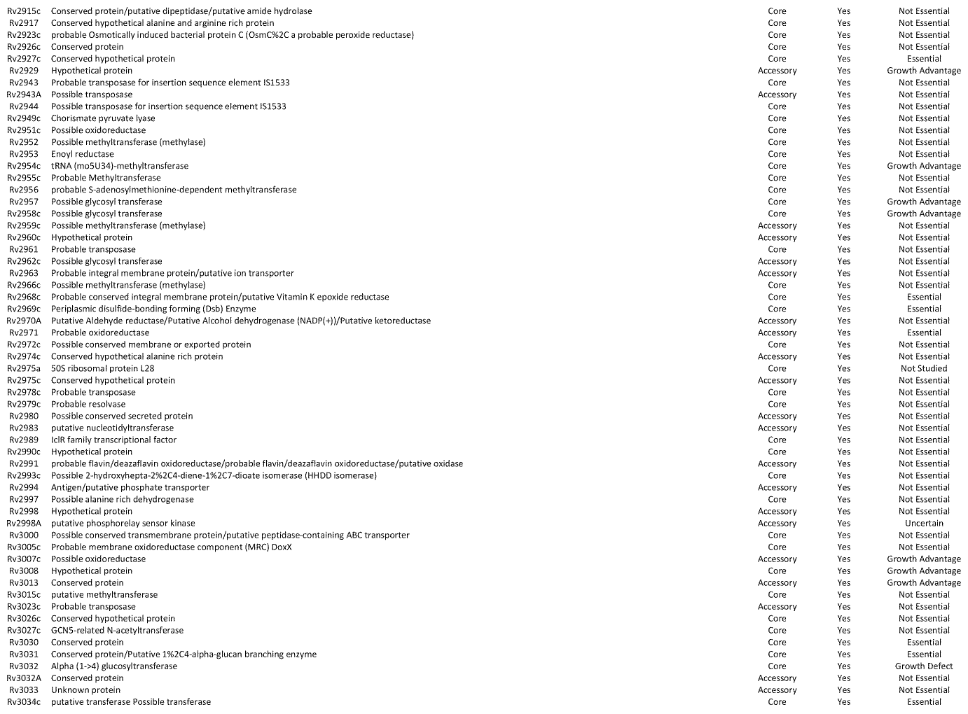

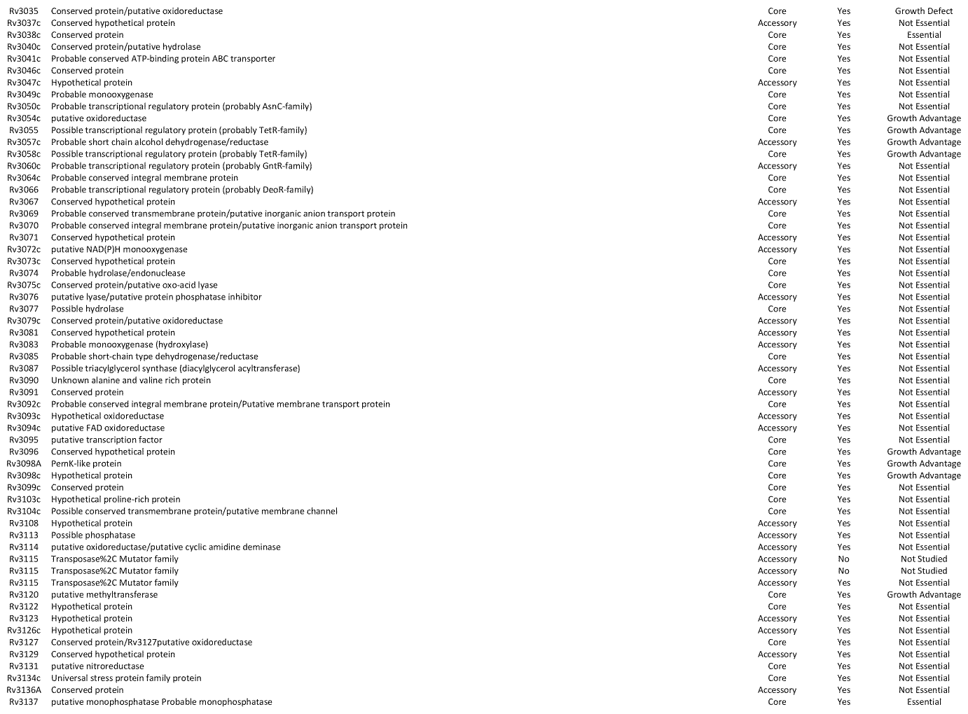

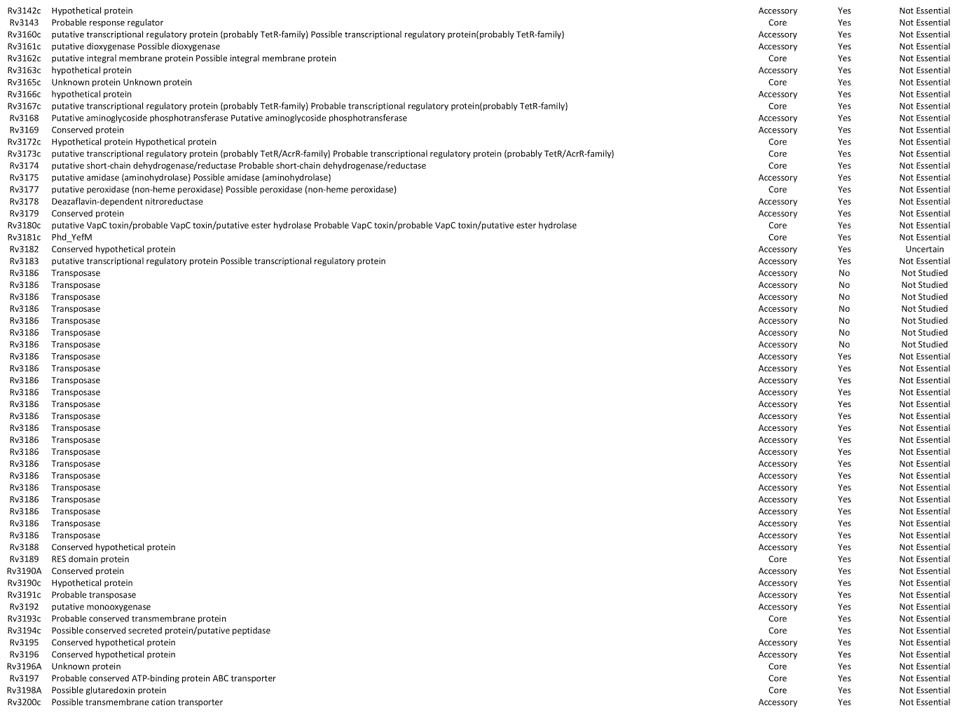

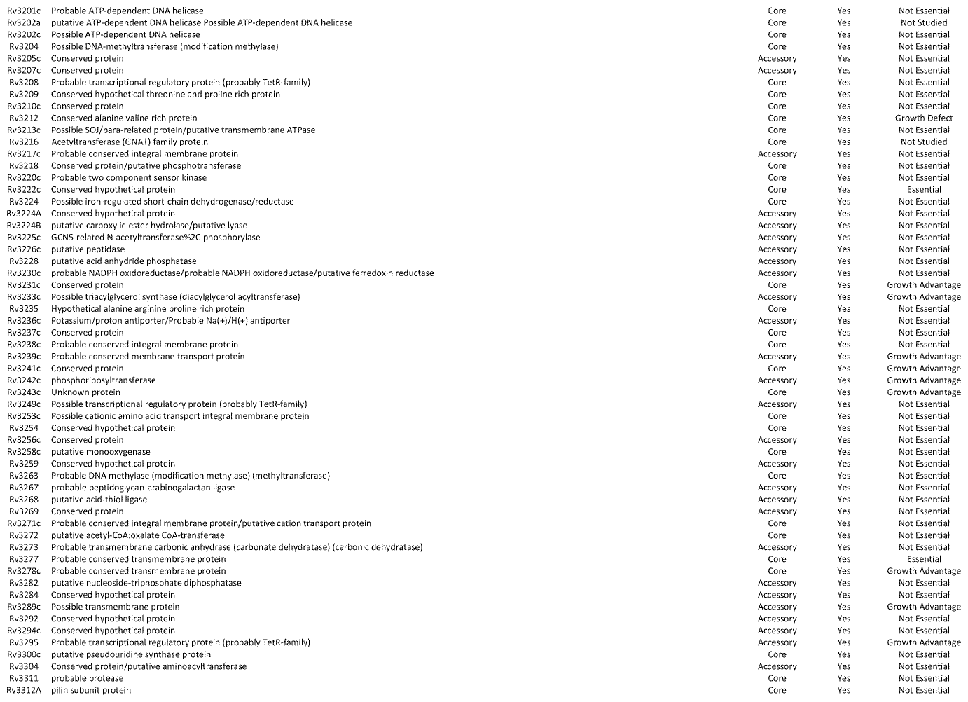

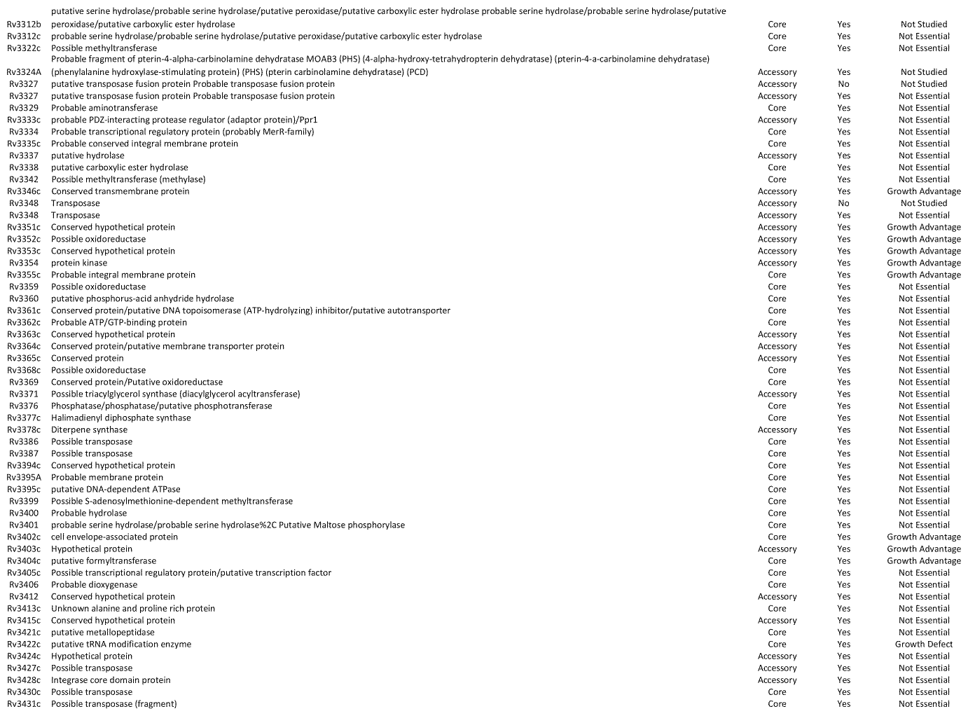

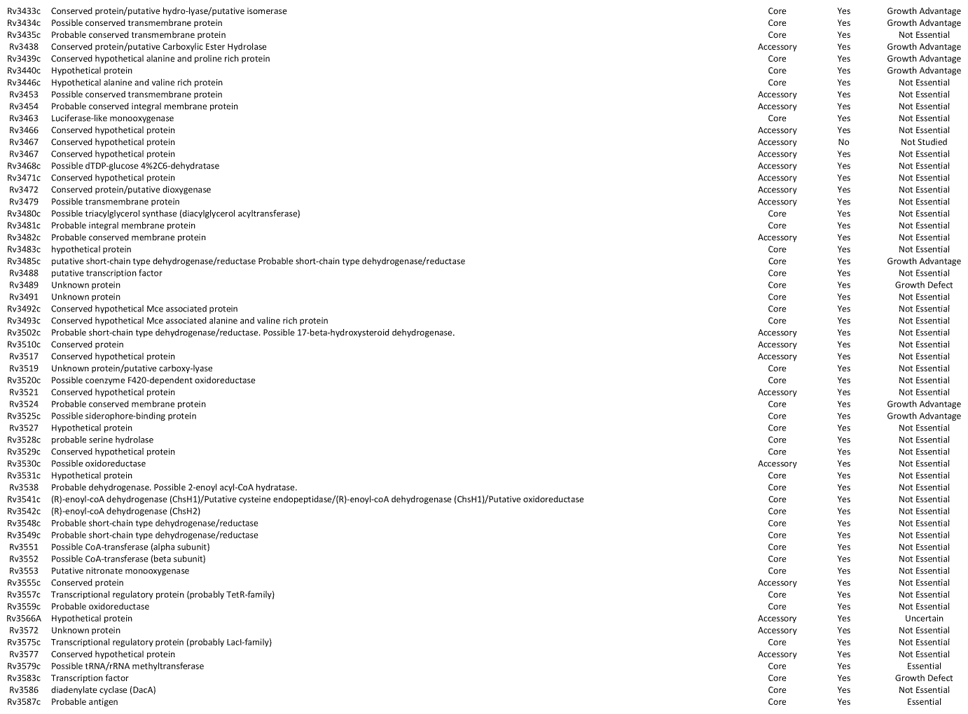

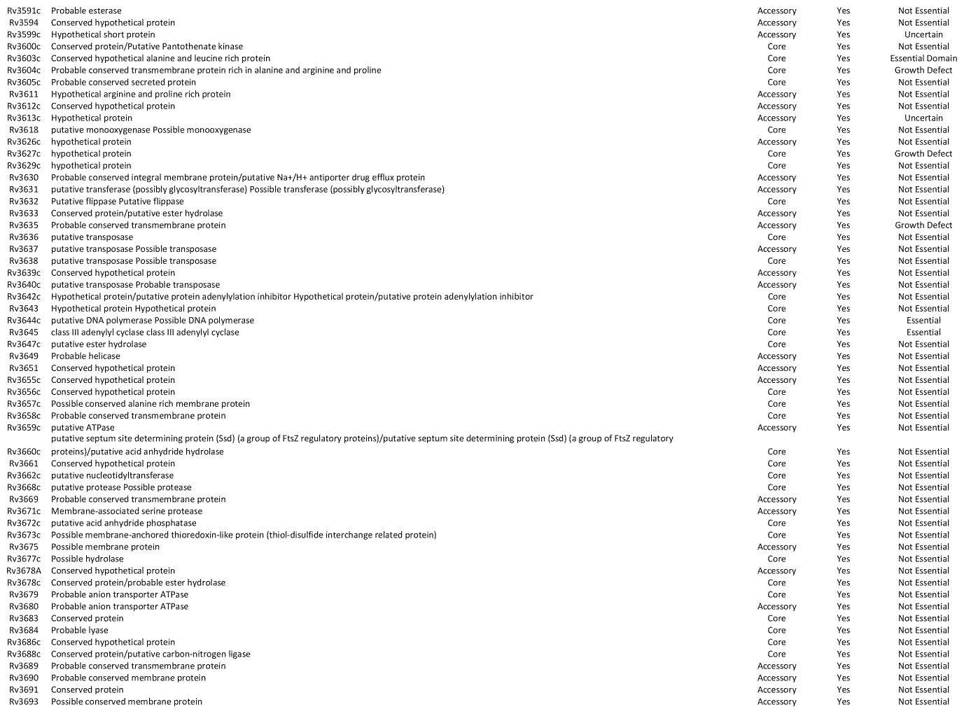

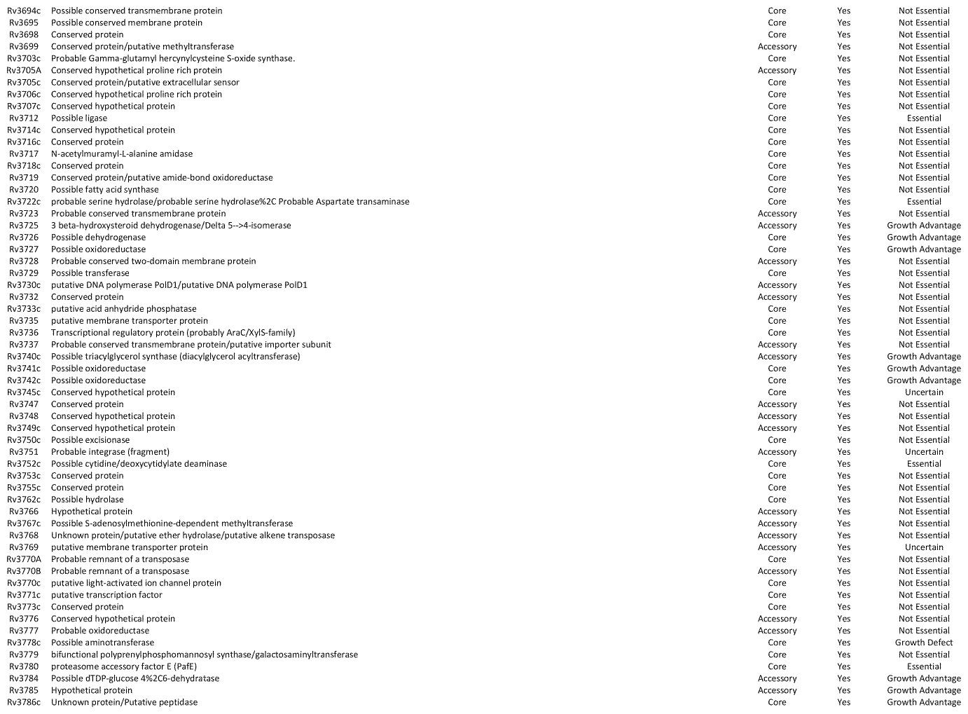

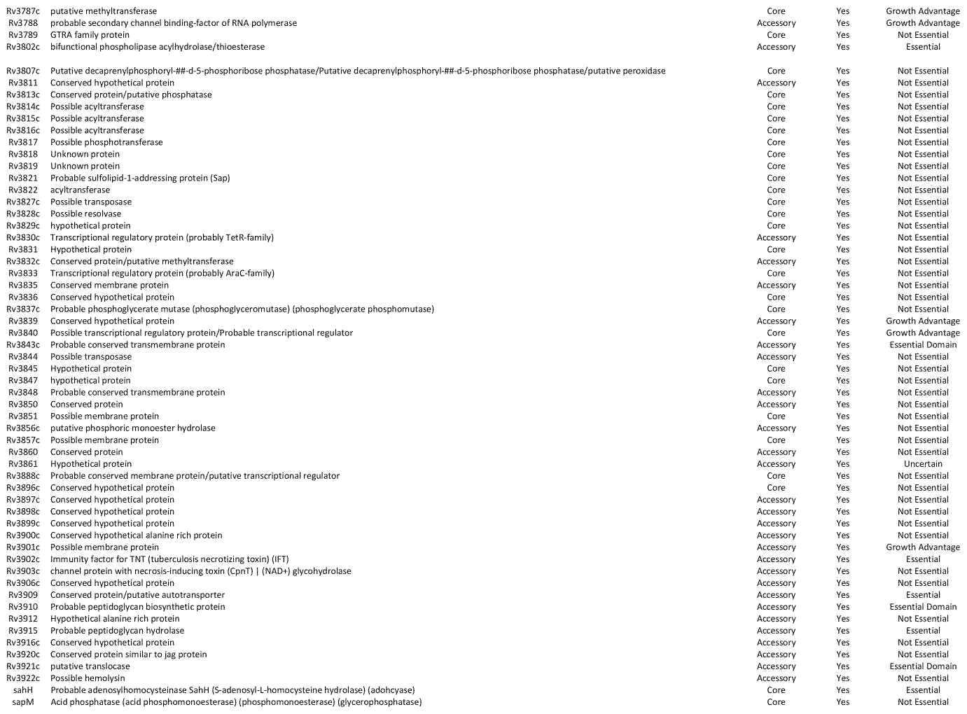

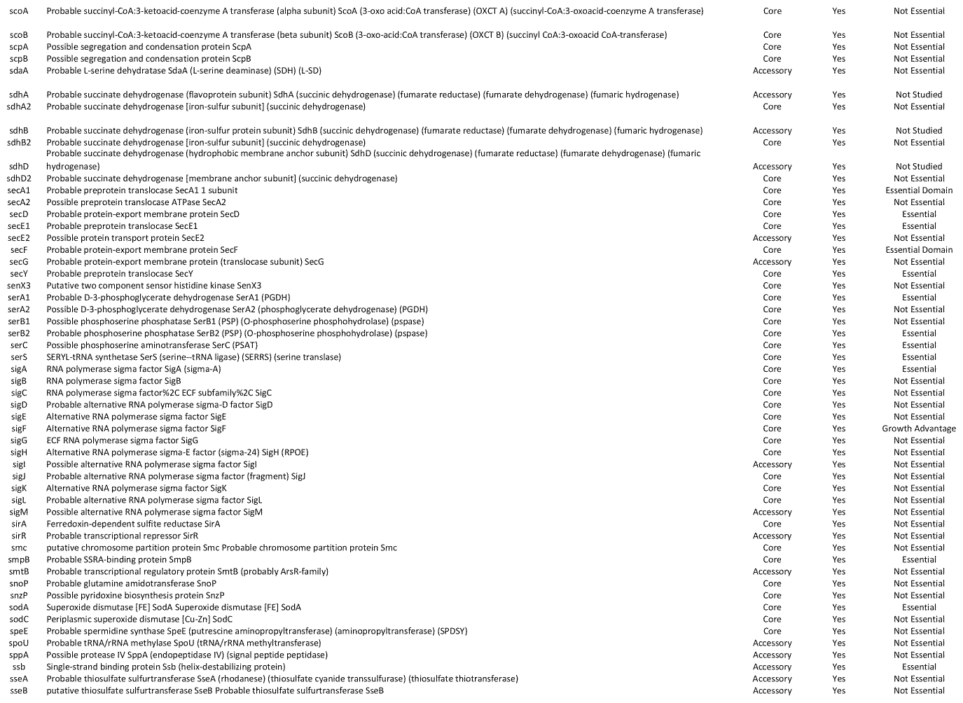

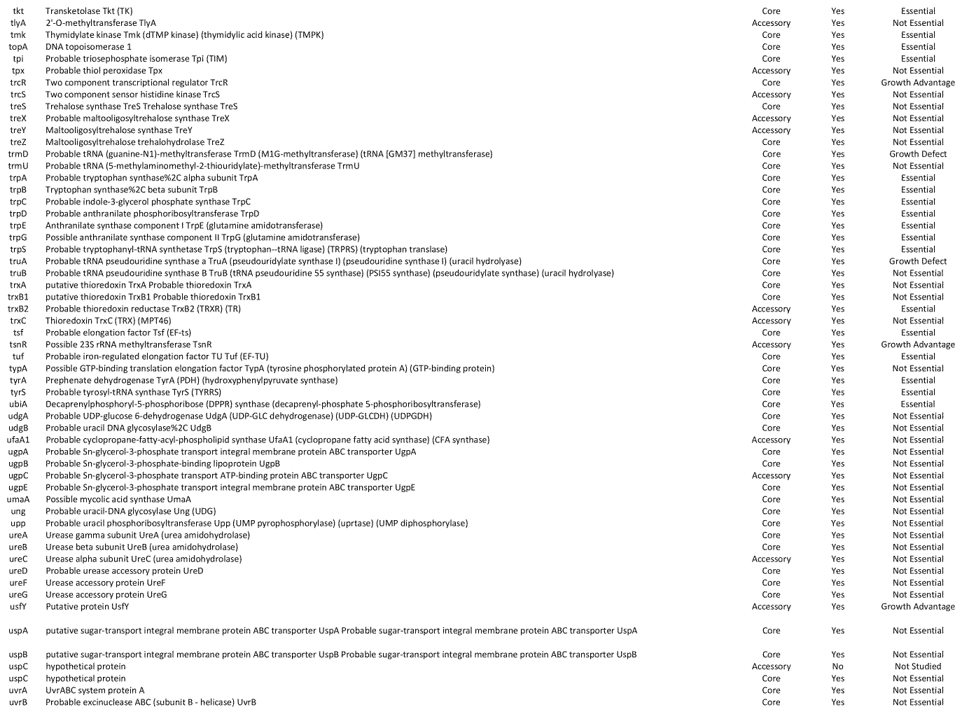

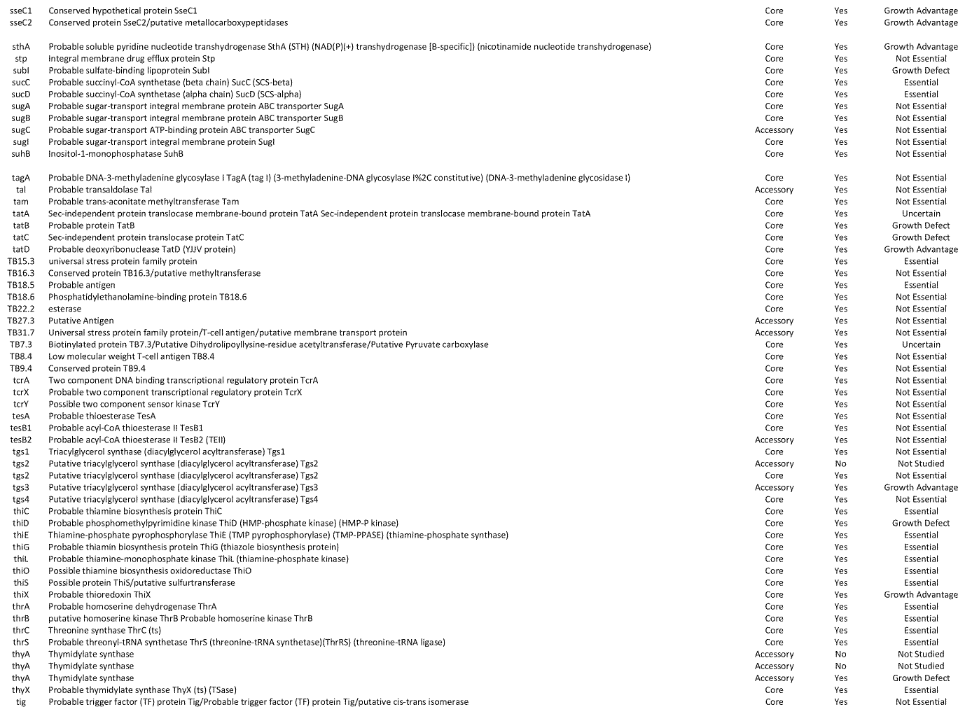

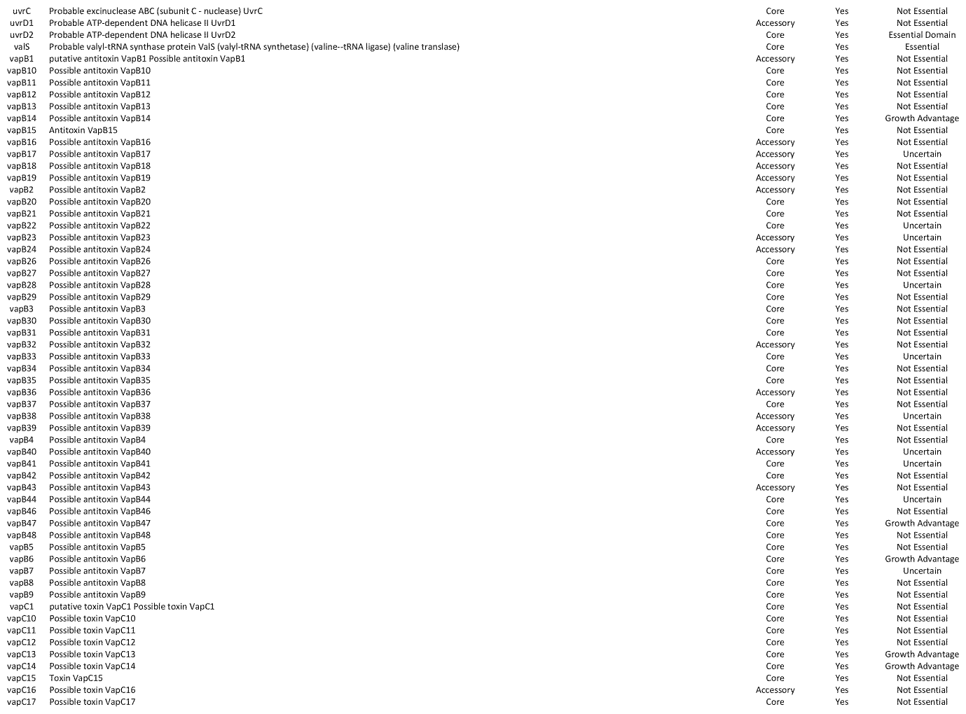

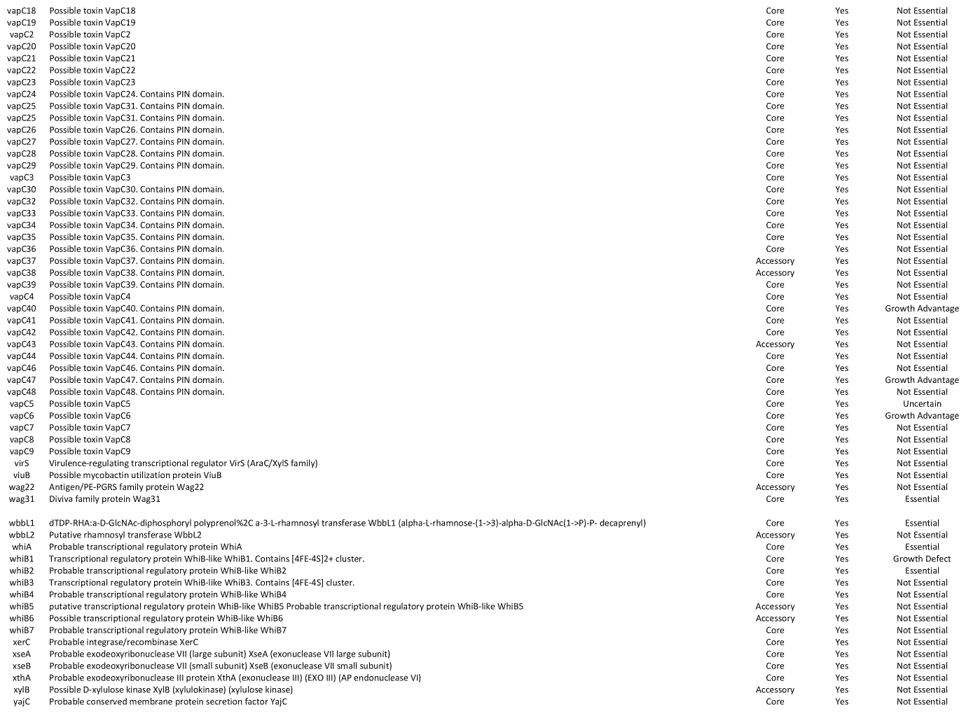

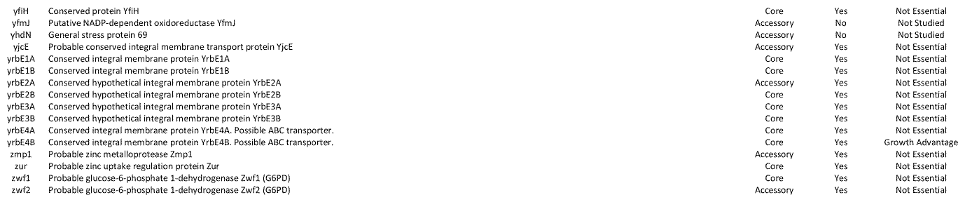

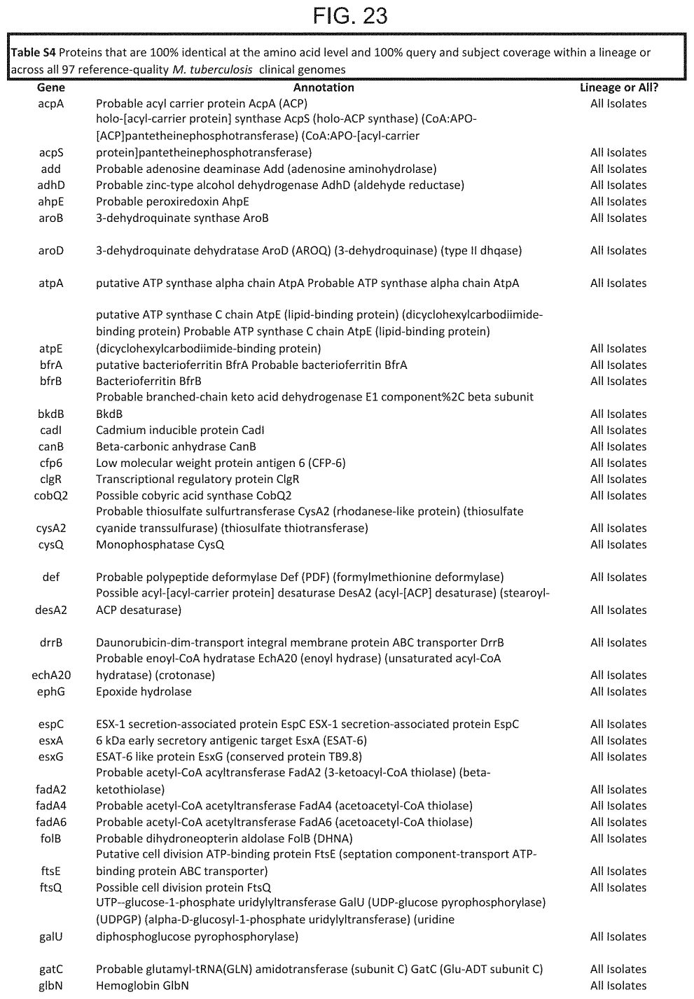

M. tuberculosis has been reported to have an open pangenome³⁶ (additional genomes would be needed to identify all genes in the species³⁷), which indicates an expanding and divergent accessory genome (CDSs present in less than 99% of the population). To see if finished assemblies corroborate this, we constructed a pangenome using Roary³⁸ from annotations of the 97 de novo assembled PacBio-only genomes, H37Rv, and H37Ra. The resulting pangenome (n=6160 CDSs) revealed an accessory genome (n=3604 CDSs) enriched for evasion of host defenses and a core genome (conserved genes, n=2556 CDSs) enriched for housekeeping functions specific to the species (FIG. 1, Table S3, or FIG. 22). Importantly, the core was significantly enriched for essential genes³⁹ (X² statistic 392.27, df was 1 with p-value 2.66×10⁻⁸⁷, Table S3, or FIG. 22). A search for 100% identical CDSs across 97 clinical genomes (H37Rv and H37Ra were excluded) revealed 366 genes with 100% amino acid sequence identity and subject and query coverage (determined by CDHIT, Table S4, or FIG. 23).

Hyperconserved Proteins as Prudent Drug Targets for Infections by M. tuberculosis

The M. tuberculosis genome sequence was published 22 years ago¹ and cited by over 8,500 works, yet no drug informed by M. tuberculosis genomic analyses has been put to market. While target essentiality has been a popular criterion for target discovery, gene essentiality studies on virulent type strain H37Rv are typically done in artificial media. Others have noted that a more relevant measure of essentiality may be conservation of genes over evolutionary time, since it implies essentiality in the native environment².

Conservation over long evolutionary time periods has been used previously as an alternative measure of essentiality of M. tuberculosis, but focused on metabolic genes³, and used Illumina sequencing, which, while largely reliable, has areas of systematic bias that preclude identification of invariable genes in many portions of the genome. Third generation sequencing technologies bring new fervor to this endeavor, however, by permitting unbiased, comprehensive genomic characterization of M. tuberculosis strains, including PE/PPE genes, which have eluded full genomic characterization during the predominance of short-read sequencing. Here, we have analyzed the proteomes of 97 finished-grade de novo assembled M. tuberculosis clinical genomes and identified invariable protein sequences within them. Next. we evaluate these hyperconserved proteins according to their essentiality, chromosomal distribution, and functional composition. We then integrate these findings with data collected in situ and ex vivo to develop a list of targets particularly robust against resistance development and with high potential for efficacy during infection.

Replisome Conflicts with Transcriptional Machinery Biases Protein Conservation Across the MTBC.

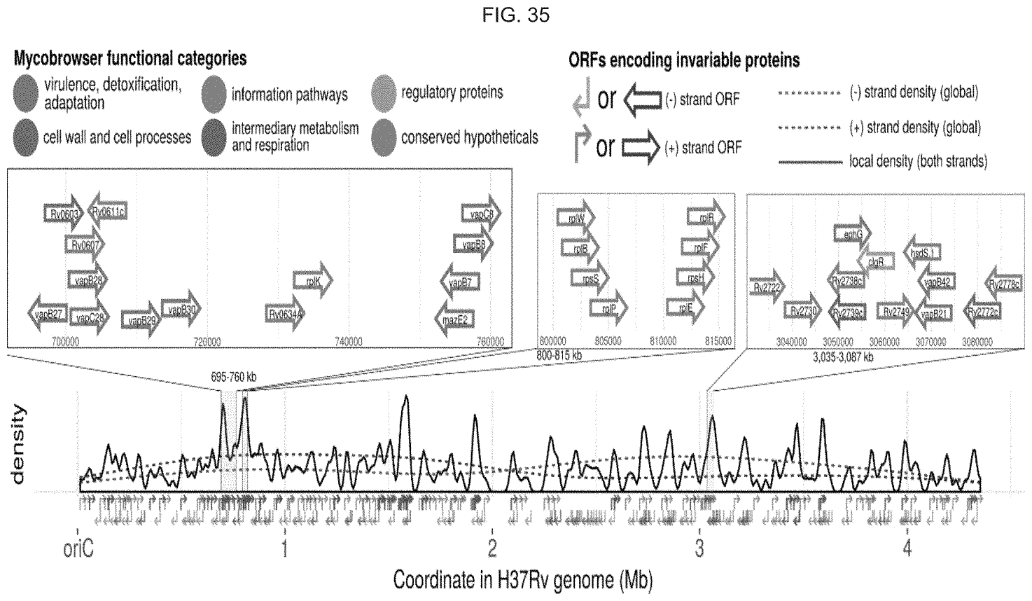

First, we analyzed how invariably conserved proteins distribute across the M. tuberculosis chromosome. Head-on collisions between the replication and transcriptional machinery bias genes on the lagging strand toward higher mutability in prokaryotes and specifically in M. tuberculosis⁴. We asked whether this bias penetrated over the long time-course between the common ancestors of these diverse strains to affect identical gene distribution. Identical gene density is higher on the leading strand than on the lagging strand (FIG. 35D, dashed lines). After normalizing for the total number of ORFs on each side of each replisome, however, only the left chromosome arm (oriC to half the genome length) retained the bias toward identical genes on the leading strand (58/194, p=0.0131, odds ratio=1.51, 95% CI: 1.08-2.50).

We reasoned that lagging strands genes that are hyperconserved despite their biased mutability must be under strong purifying selection and thus likely essential for pathogenesis. Eight operons meet this criterion (Table Y) and successfully recover targets with in vivo essentiality or promise for developing vaccine and therapeutics. The eight operons include multidrug efflux pumps (p55 and mmr) and an operon currently in trials as a vaccine candidate and drug target (Rv1410c-lprG). The Rv1410c-lprG operon has demonstrated in vivo essentiality⁵ and regulates import of triacylglycerol, a key carbon source during infection and energy reservoir during dormancy⁶. The operon is also essential for drug export via the P55 efflux pump⁷ and for evading immunity through inhibiting phagosome-lysosome fusion⁸. Encouragingly, small molecule inhibitors efficacious against LprG were recently identified⁹. Our results add to the evidence for this operon as a promising therapeutic target.

TABLE Y

| operon | gene | function |
|---|---|---|
| Rv0249c-Rv0250c | sdhD2 | |
| Rv0249c-Rv0250c | Rv0250c | |
| Rv1410c-Rv1411c | Rv1410c | “p27-p55 operon” multidrug |
| | | efflux pump |
| Rv1410c-Rv1411c | lprG | multifunctional lipoprotein |
| Rv2641-Rv2642 | cadI | Cadmium inducible protein CadI |
| Rv2641-Rv2642 | Rv2642 | metalloregulatory ArsR family |
| | | transcription factor |
| Rv3065-Rv3066 | mmr | multi-drug efflux pump |
| Rv3065-Rv3066 | Rv3066 | Transcriptional regulator |
| Rv3208A-Rv3209 | Rv3208A | |
| Rv3208A-Rv3209 | Rv3209 | unknown |
| Rv3218-whiB1 | Rv3218 | |
| Rv3218-whiB1 | whiB1 | whiB transcriptional regulator |
| Rv3488-Rv3489 | Rv3488 | Transcriptional regulator |
| Rv3488-Rv3489 | Rv3489 | |
| Rv3851-hns | Rv3851 | unknown |
| Rv3851-hns | hns | disputed |

CadI-Rv2642, a cadmium-inducible gene and an ArsR-family transcription factor that regulates its transcription, form part of the “arsenic detoxification operon” which, unexpectedly, were the only consistently downregulated genes across all attenuated MTB¹⁰, adding to the mounting evidence that metal ion homeostasis factors critically into M. tuberculosis pathogenesis¹¹. Transcriptional regulators are significantly overrepresented in the operons (p=0.008, odds ratio=6.03, 95% CI: 1.40-20.1) and regulate their co-operonic protein in several cases. These include Rv3066, which regulates mmr, a multidrug efflux protein underpinning intrinsic resistance to multiple drugs, and whiB1, an important coordinator of the transcriptional response to hypoxia.

Identical genes distribute into regional (FIG. 1D) and strand-specific (FIG XE) peaks. We highlight three of these clusters. Cluster A is a dual-strand 65 kb bicluster of fourteen mostly non-essential proteins that includes a remarkably well-conserved set of eight toxin-antitoxin (TA) proteins, including two complete pairs (vapBC28 and vapBC8). TA proteins are poorly conserved in other species but abundant (more so than any intracellular pathogen, in fact) and well-conserved in M. tuberculosis. Their primary function is inhibiting protein translation through RNase activity but they have been linked to persistence in E. coli, though this link has recently come under scrutiny. Cluster B resembles a more canonical hyperconserved cluster: eight essential ribosomal proteins co-oriented and clustered densely (15 kb) on the leading strand. Cluster C is the exact opposite of B. It comprises a dual-stranded set of twelve non-essential genes, predominately encoding proteins with uncharacterized function. ClgR is a transcriptional regulator essential for replication in macrophage and controls multiple proteases and chaperones¹². Toxin and antitoxin proteins and a DNA methyltransferase specificity subunit hsdS.1 suggest further regulatory roles, although the hyperconservation of HsdS.1 is curious, considering its MTase has loss-of-function mutations in over one-third of the isolates in this study. HsdM knockout alters transcription of dormancy (Rv1813c), efflux (RaaS), and TA (VapB28) proteins, suggesting a potential role in dormancy for the cluster.

### Metal Ion Processing and Self-Regulation Predominate the Functional Composition of Hyperconserved Proteins.

Second, we assessed the functional distribution of invariable M. tuberculosis proteins. The As expected, considering their fundamental role in life, 505 (rplBEFKPRTUW and rpmAEF) and 30S ribosomal proteins (rpsBFHKPS/N2/R1/R2) were abundant among invariable genes. TA genes were not restricted to the clusters highlighted in FIG. 35; 24 were identical across all isolates, see Table Z.

TABLE Z

| Function | Genes |
|---|---|
| Ribosomal proteins | rplBEFKPRTUW, rpmAEF, rpsBFHKPS, |
| | N2, R1, R2, |
| Toxins/antitoxins | mazE2, relBGJ, vapB7, 8, 10-13, |
| | 21, 22, 27-30, 37, 42, vapC8, 15, |
| | 17, 28, 33, 41, 47 |
| Two-component regulatory | mtrA, prrA, regX3 |
| systems | |
| Iron regulation & utilization | bfrAB, glbN, ideR, mhuD, mmps4-5, |
| | mbtDLN, ppiA, Rv0763, sufT, whiB1, |
| | whiB3, |
| initiation & elongation | greA, infA, tsf, tuf |
| factors | |
| copper resistance | mymT, mctB, ricR |
| molybdenum | moaE2, moaC2, mog |

The importance of iron M. tuberculosis pathogenesis showed in their conservation. Genes encoding proteins mediating Iron acquisition (mbtDLN and mmpS4/5), storage (brfAB), transcriptional regulation (ideR), and FeS clusters (sufT, whiB1, whiB3) are invariably conserved. As were genes encoding proteins for other metals, including copper resistance (mymT, mctB, and ricR) and molybdenum processing (moaC2/E2 and mog) proteins.

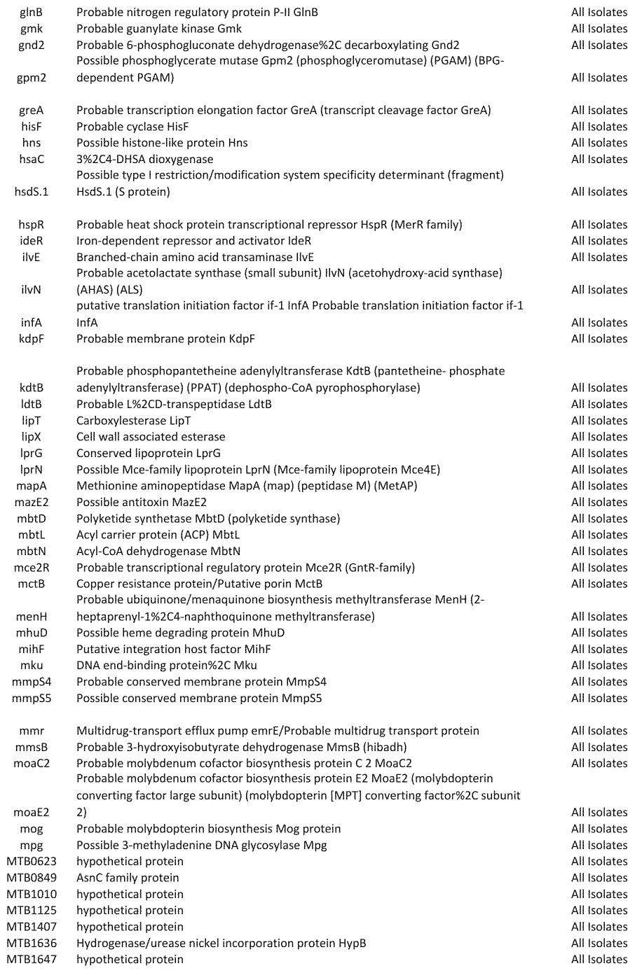

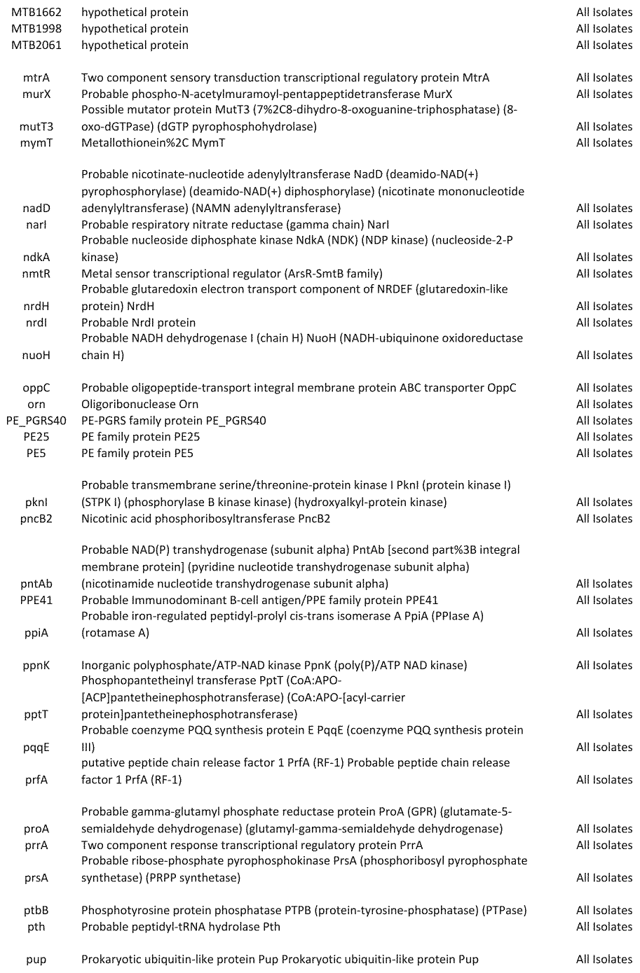

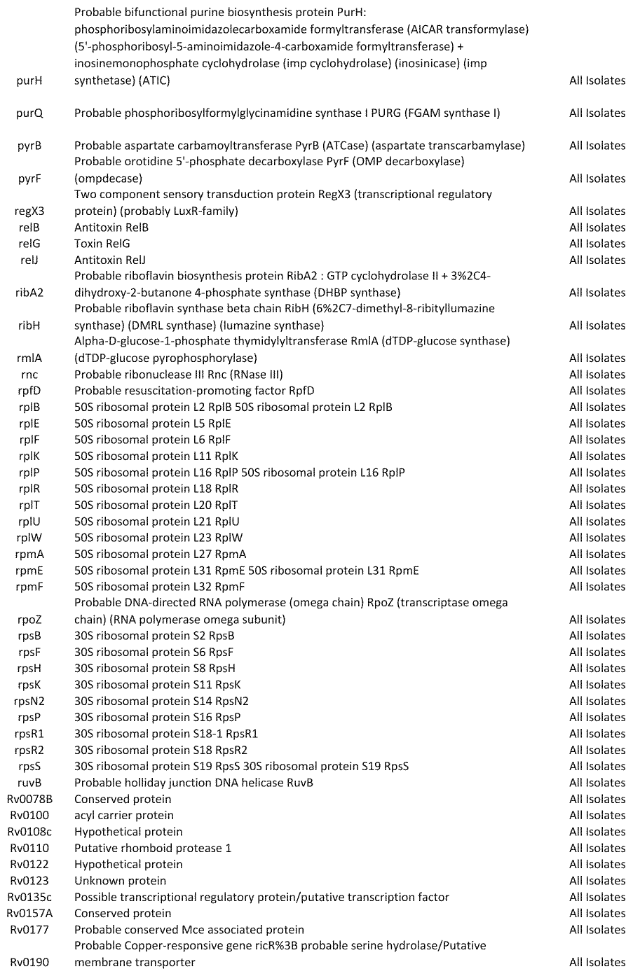

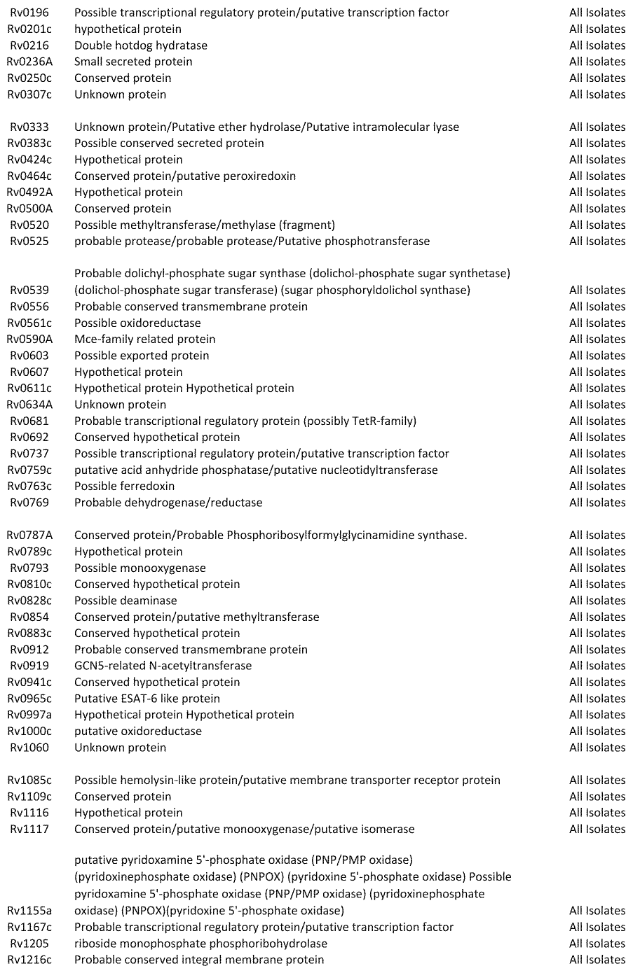

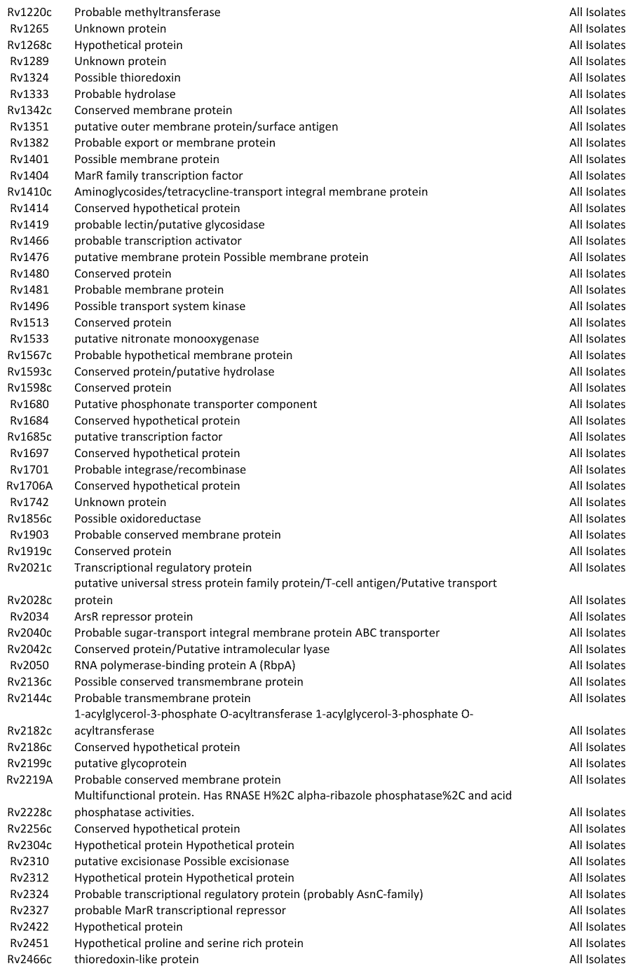

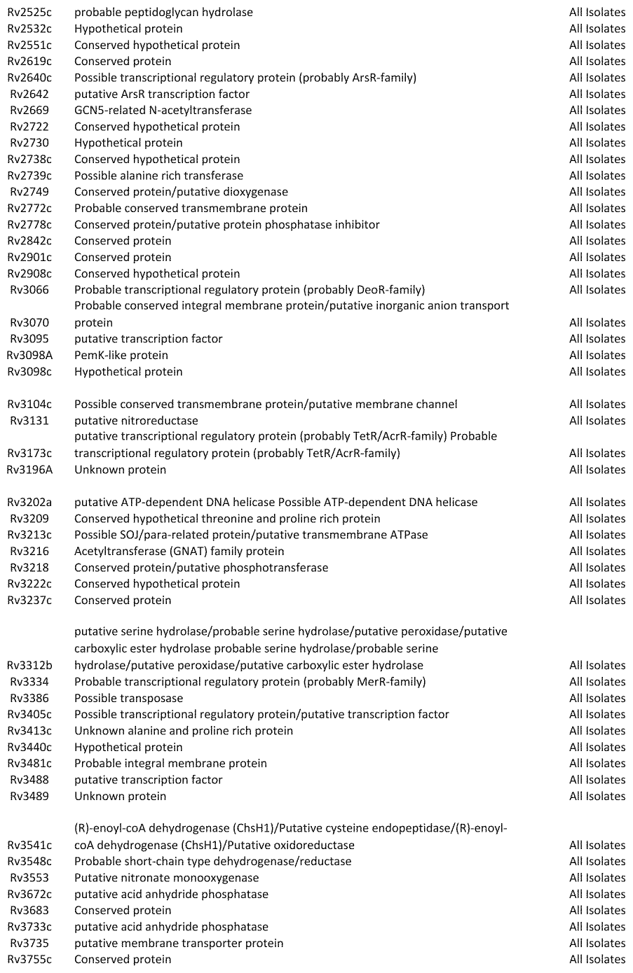

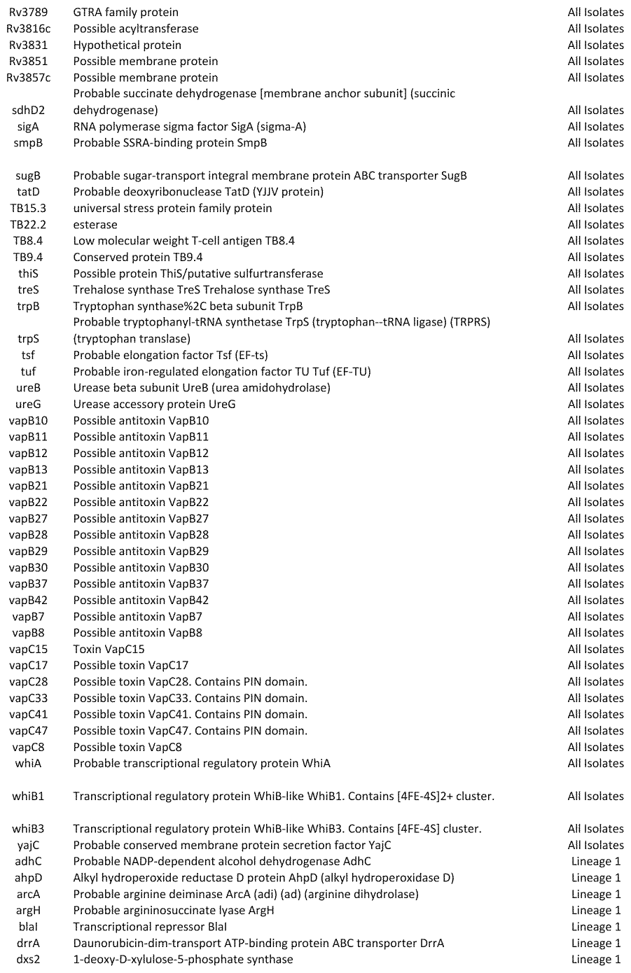

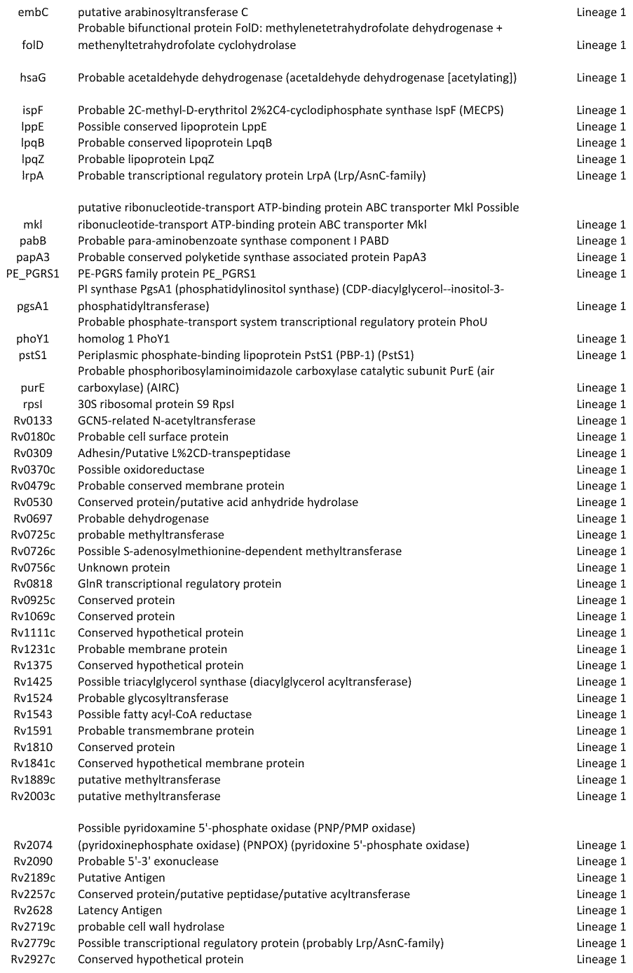

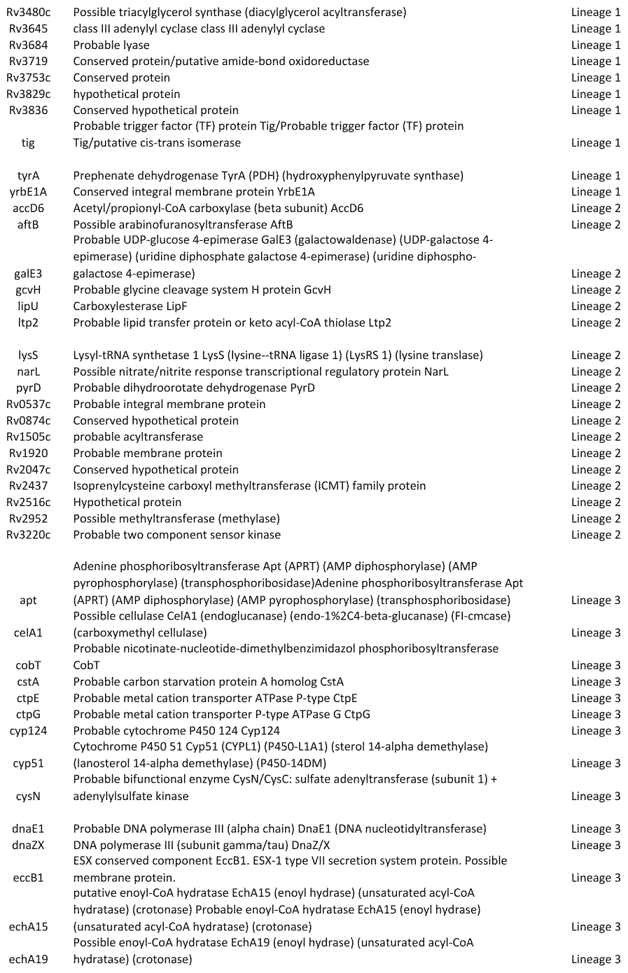

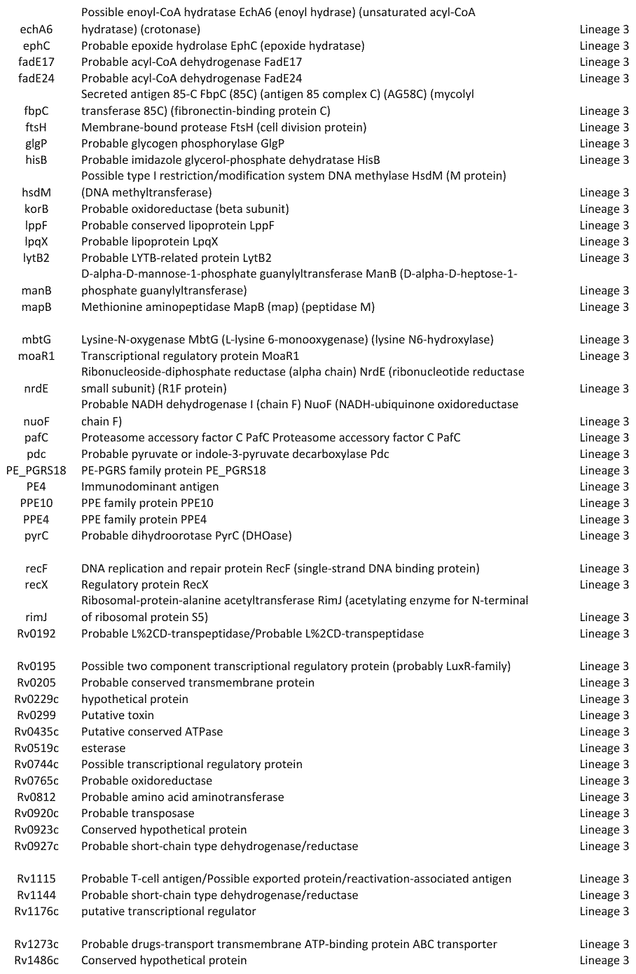

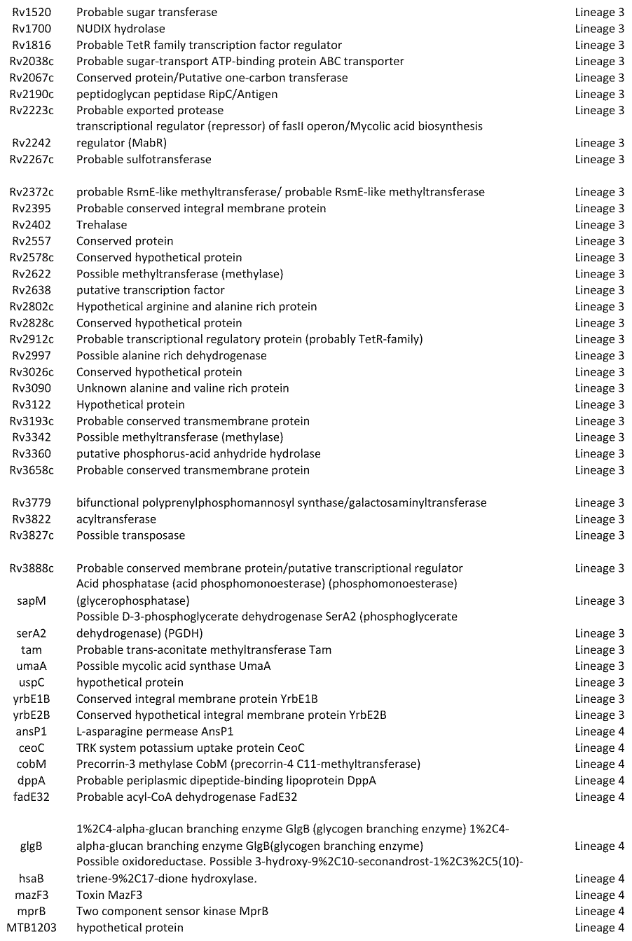

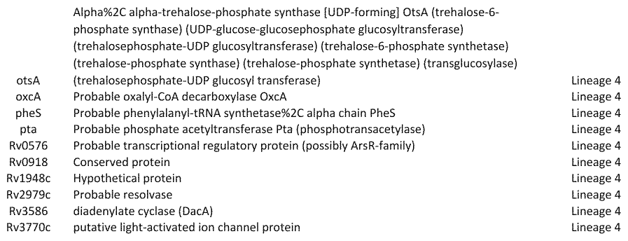

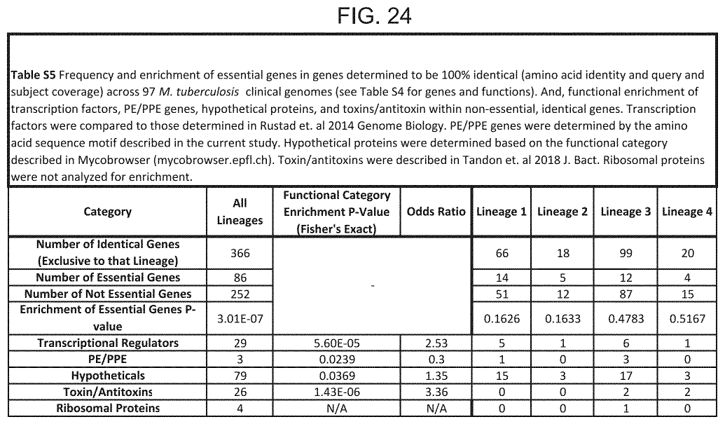

These identical genes were also significantly enriched for essential genes (determined in H37Rv in an in vitro system), but not within each lineage (Table S5, or FIG. 24). To identify what systems were over-represented among non-essential but identical proteins, we performed enrichment analyses for several protein classes notable in adaptation (transcription factors) or abundant yet poorly understood in M. tuberculosis (toxin/antitoxins, PE/PPE, and conserved hypotheticals, Table S5, or FIG. 24). Hypothetical proteins were over-represented underscoring the lack of knowledge about M. tuberculosis proteins with key functions in vivo. Even more starkly over-represented were transcription factors and toxin-antitoxin proteins, suggesting modes of transcriptional and translational regulation key to survival within-host are not essential in vitro. Toxins are implicated in dormancy induction through global translational inhibition in Enterobacteria⁴⁰, and suggested to play the same role in M. tuberculosis⁴¹. Therefore, their survival benefit to M. tuberculosis may lie in inhibiting growth at key points in response to certain conditions. This would further explain their absence from essentiality as determined by typical methods, which define essentiality by growth rate³⁹ rather than survival. In contrast, PE/PPE were significantly underrepresented, though it is not clear whether this reflects antigenicity across PE/PPE family members, intrinsic propensity for variation, or a consequence of their hypervariable regions.

The over-representation of regulatory, virulence, and hypothetical proteins aligns with the functional composition of the enduring hypoxic response stimulon¹⁹, supporting the notion that these genes fill conserved roles required for enduring the stressors imposed by host immunity.

Integrative analysis with in situ expression data highlights non-essential transcriptional factors as priority drug targets. To distill targets most likely accessible throughout infection out of this unexpectedly large trove of invariably conserved non-essential genes, we integrated invariable proteins with the genes comprising the “core transcriptome” of M. tuberculosis active and inactive macrophage²⁰ (Table Z). The genes in this intersection were remarkably overrepresented for non-essential transcription factors (4/11, p=0.019, odds ratio=10.34, 95% CI: 2.20-41.1). Yet this remains an underestimate of the degree of their enrichment; Four of the seven genes not annotated as TFs in the Rustad dataset, have since characterized had transcriptional regulatory roles characterized in M. tuberculosis (abmR²¹, Rv2327²², Rv2028c, and sufT) or in homologs in other species.

Like RpoB, the target of first-line antitubercular therapeutic rifampin, HspR (protein folding & stability) and PrfA (protein syntheses) mediate fundamental processes, making them intriguing therapeutic candidates. However, prfA has a SNP specific to sublineage 4.2.2.1 (a sublineage absent from our isolates). The hspR gene developed mutations in a PafE (which degrades HspR) mutant²³, but it is unclear whether this variation would occur naturalistically during infection or if its permissibility is restricted to the artificial experimental environment.

Several hits in Table Z are known players in drug resistance. Rv2327 is upregulated upon BDQ exposure²⁴, and its M. smegmatis homolog is a redox-sensing MarR-type repressor that confers resistance to rifampicin, erythromycin, and hypochlorite stress in M. smegmatis through regulation of a multidrug efflux pump in response to oxidative stress²². These reports suggest a potential mechanism of tolerance to BDQ, or perhaps multiple drugs that could be targeted for synergistic effects. Other hits mediate immune subversion of host defenses or are involved in stress response. These are interesting targets for combinatorial therapy with more direct drugs with metabolic targets.

Transcription factor abmR is required for regulation of central carbon metabolism genes by sRNA Mcr11²⁵ and appears to mediate adherence and invasion of host cells²¹. These roles in metabolic adaptation and infectivity make abmR an appealing therapeutic target. One intriguing direction would be targeting AbmR as a prophylactic measure, given its potential essentiality for transmission.

Hyperconserved PE/PPE proteins in Mycobacterium tuberculosis. Next, we examined invariable PE/PPE genes. The lack of systematic bias in SMRT-sequencing permits bona fide de novo analysis of PE/PPE gene composition, which has been difficult to resolve with short read sequencing technologies²⁶. To our knowledge, this is first report of their conservation across a global set of finished genomes. While they are starkly under-represented among invariable proteins (5/366, p=0.00239, odds ratio=0.291), four of the five invariable PE/PPE proteins have clear important roles, substantiating the validity of our screen for inferring phenotypic significance during infection. pe25 and ppe41 are co-operonic and together encode products to form the PE25/PPE41 heterodimer. PE25/PPE41 is secreted out of M. tuberculosis and acts as a highly immunogenic immune effector″, inducing macrophage death by necrosis²⁸, though the precise mechanisms are not yet teased out. Also known as pe11, lipX encodes a functional lipase, LipX, that influences the lipid composition of the mycobacterial membrane and propensity for biofilm formation²⁹. PE5 lies within the esx-3 region of the genome and is secreted with PPE4 by the ESX-3 secretion system. Once secreted, PE5 scavenges iron and suppresses host-generated nitric oxide stress. Its expression is upregulated more than two-fold following exposure to multiple stressors, including the recently repurposed TB antimicrobial Clofazamine³⁰.

PE_PGRS40, on the other hand, is functionally uncharacterized. At only 60 AA it is extremely short (compared to 176-1,901 AA of other PE_PGRS genes³¹) and reported to have 0 T-cell epitopes, unlike others in the PE_PGRS family, which have up to 52³¹.

The rigid conservation of clear functions of LipX, and PE25, and PPE41 suggests their functions are conserved mechanisms of host subversion and stress response, rather than population-specific adaptations or mechanisms to generate variability. This is consistent with recent reports of PE/PPE genes as mediating specific functions like acquiring iron³² and acting as a selective porin for nutrient uptake³³. Given the recent discovery that some, and likely many, PE/PPE genes act as selective nutrient gates³³, PE/PPE proteins stable across the phylogeny would imply mechanisms of interfacing with the host milieu that are conserved across humanity. Disrupting such processes thus make attractive therapeutic candidates.

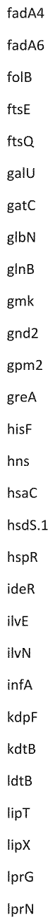

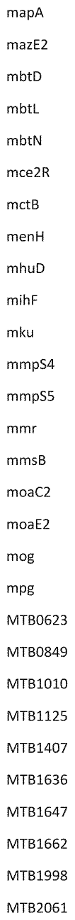

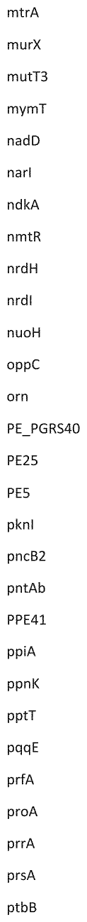

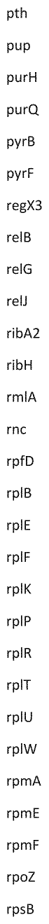

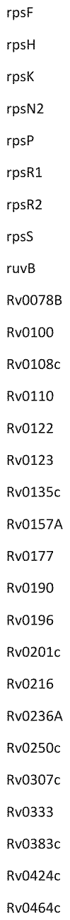

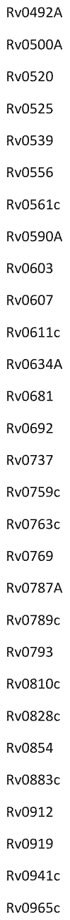

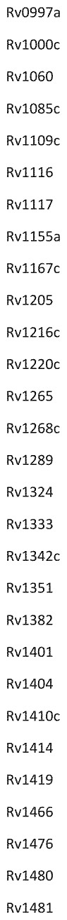

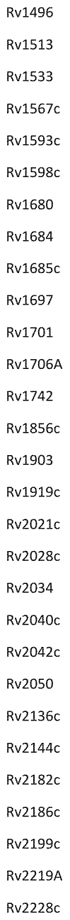

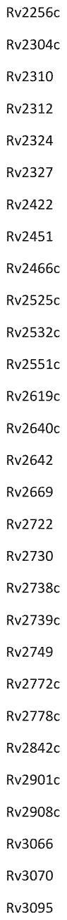

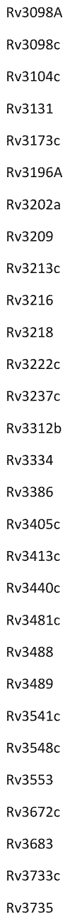

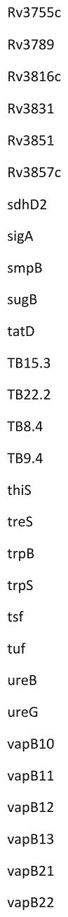

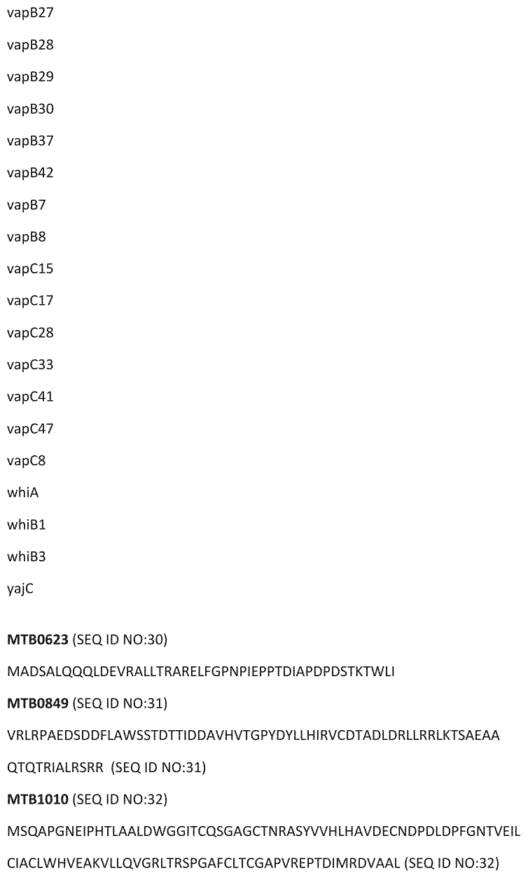

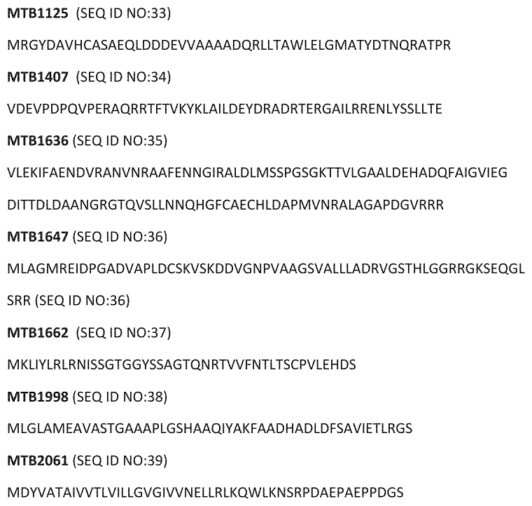

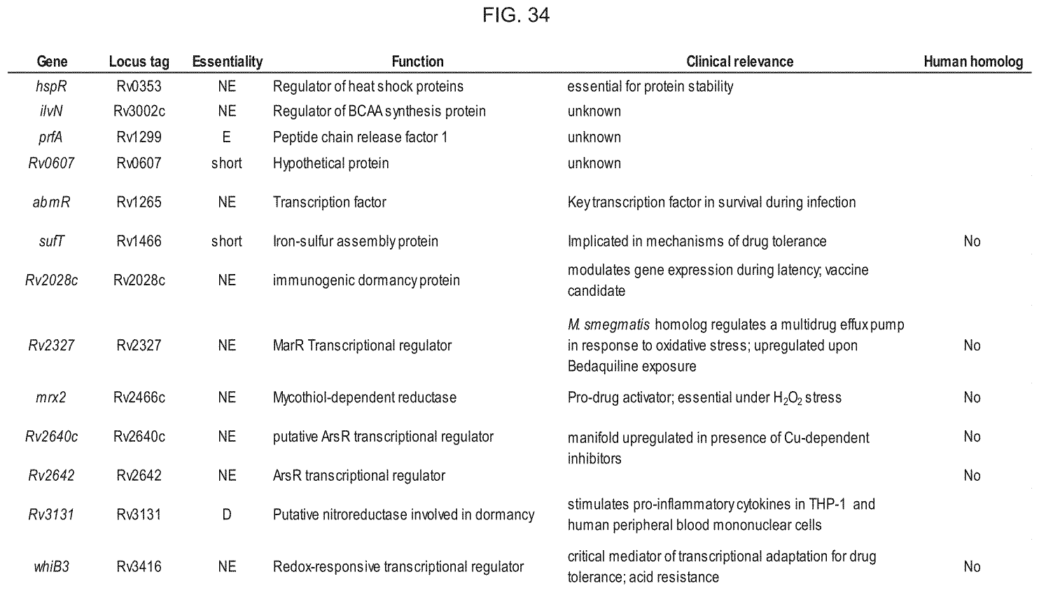

Through interrogating finished genomes for hyperconservation, and filtering invariant proteins according to their (i) resilience to mutagenic replication-transcription conflict and (ii) consistent transcription in situ, we capture 7 targets therapeutic or vaccine targets under active development and highlight 22 genes as new high-priority targets (Table Z, above, and the Table in FIG. 34), and emphasize the essentiality of transcriptional and translational regulatory proteins for pathogenesis. We also provide the first analysis of PE/PPE conservation on a global set of finished genomes, highlighting five PE/PPE genes as globally conserved.

The 11 target proteins distilled in Table Z provide a short list of conserved gene targets expressed during infection. The criteria implemented in our approach was rather strict; proteins need not be entirely invariable to make good drug targets. The overlap of known genes mediating M. tuberculosis stress response, drug tolerance, and pathogenesis recovered by this approach is encouraging, and suggests the less well-known genes may play similar roles, yet undiscovered.

Gene Loss (Pseudogenization) is More Prevalent than Gene Gain (Duplication)
Gene loss, measured by prevalence of pseudogenes, is proposed to be the primary mechanism to generate diversity in M. tuberculosis (Bolotin et al. 2015). We sought to confirm this observation by using a homology-based approach which has been described previously (Carlier et al. 2013; Danneels et al. 2018). On average, there were 82 pseudogene regions per genome (min=75, max=94), which contributed to 3.37% of the genome (bp) (min=2.81%, max=4.09%), while gene duplications contributed only 0.135% of the genome (bp) (min=0.036, max=0.619). Based on these statistics and previous reports (Singh and Cole 2011; Bolotin et al. 2015), we postulated that there would be more genome contraction (pseudogenization) than amplification (duplication). To account for lineage being a confounding factor, an ANOVA test was performed based on the proportional differences between pseudogene length and duplication length (lineage 7 was excluded from statistical analyses as only one genome represented the lineage). Thep-value was 0.236, indicating that genome contraction vs amplification is not more pronounced in specific lineages. A paired Student's t-test was then used to determine if there was significantly more genome contraction than amplification per lineage. We observed significantly more genome contraction than amplification across all lineages (Table 1).

TABLE 1
Difference of percentage of sequence length considered
between pseudogene and duplication using a paired Student's
t-test in 97 M. tuberculosis clinical isolate genomes, M. tuberculosis
H37Rv (NC_000962.3), and M. tuberculosis H37Ra
(CP016972.1)

| | Mean Difference | Standard Deviation | |
|---|---|---|---|
| Lineage | (%) | (%) | p-value |
| Lineage 1 | 3.45 | 0.175 | 8.75e−25 |
| Lineage 2 | 3.01 | 0.212 | 4.03e−43 |
| Lineage 3 | 3.21 | 0.211 | 1.53e−9 |
| Lineage 4 | 3.34 | 0.258 | 5.58e−35 |

A GO enrichment was performed on the functional homologs of pseudogenes. This resulted in several functions related to host evasion (Table 2). Loss of evasion mechanisms is likely related to the ever-adapting landscape of antigens.

TABLE 2
Gene ontology (GO) enrichment of pseudogenes' functional
homologs within a pangenome of 97 clinical M. tuberculosis genomes
and two reference strains, M. tuberculosis H37Rv and H37Ra.
Functions with significant enrichment were included if the
p-value was less than or equal to 0.01. BP: Biological Process,
CC: Cellular Component, MF: Molecular Function.

| | | | Overrepre- |
|---|---|---|---|
| Ontol- | | | sented |
| ogy | GO ID | Function | p-value |
| BP | GO:0051832 | evasion or tolerance of | 0.000702499 |
| | | defenses of other organism | |
| BP | GO:0030682 | evasion or tolerance of | 0.001173383 |
| | | host defenses | |
| BP | GO:0006952 | defense response | 0.001339001 |
| BP | GO:0042783 | evasion of host immune | 0.006612928 |
| | | response | |
| BP | GO:0006950 | response to stress | 0.006633834 |
| BP | GO:0045934 | negative regulation of | 0.006770709 |
| | | nucleobase-containing | |
| | | compound metabolic process | |
| BP | GO:0006857 | oligopeptide transport | 0.006862877 |
| BP | GO:0052173 | response to defenses of other | 0.009795324 |
| | | organism | |
| BP | GO:0052200 | response to host defenses | 0.009795324 |
| BP | GO:0052564 | response to immune response | 0.009795324 |
| | | of other organism involved | |
| | | in symbiotic interaction | |
| BP | GO:0052572 | response to host immune | 0.009795324 |
| | | response | |
| CC | GO:0030312 | external encapsulating | 0.001119691 |
| | | structure | |
| CC | GO:0005618 | cell wall | 0.001220004 |
| CC | GO:0005623 | cell | 0.001225644 |
| CC | GO:0034081 | polyketide synthase complex | 0.002268105 |
| MF | GO:0008375 | acetylglucosaminyltransferase | 0.003883265 |
| | | activity | |
| MF | GO:0031177 | phosphopantetheine binding | 0.005791198 |
| MF | GO:0016616 | oxidoreductase activity, | 0.006527358 |
| | | acting on the CH—OH | |
| | | group of donors, NAD or | |
| | | NADP as acceptor | |

The M. tuberculosis Pangenome Contains Over 1000 Genes not Present in H37Rv
Comparing the size of the pangenome (6160) to the size of the H37Rv proteome (4099 CDSs re-annotated by AnnoTUB), we identified 2061 genes that were not observed in H37Rv (Table S3). The 2061 genes were enriched for avoidance of host defenses and negative regulation of host response (FIG. 2).
We postulated that the genes not in H37Rv may be diverged homologs of genes in H37Rv. A hierarchy was developed to categorize these genes into mutually exclusive groups. The order of the hierarchy was: RvD1-6/TbD1, in CDC1551, transposases, duplications, pseudogenes, contained frameshift mutations, truncations, extensions, and partial alignments (Table 3).

The first category listed (RvD1-6/TbD1) represents previously described regions within the H37Rv genome that are deleted but present in other members of the M. tuberculosis complex (Zheng et al. 2008; Brosch et al. 1999). A BLASTP query in the 2061 genes not in H37Rv to each of these RvD1-6/TbD1 regions revealed 44 genes with at least 99% amino acid sequence identity (Table S3).

Second, a BLASTP search of the remaining 2017 genes to the CDC1551 proteome identified 120 homologs not in H37Rv. This included pbp-1 (mt0930), which encodes a penicillin-binding protein 4. In the current dataset, four genomes had pbp-1 in addition to the H37Rv penicillin-binding protein homologs, ponA1 and ponA2. This protein has been reported to be secreted in two proteomics studies of M. tuberculosis CDC1551 (Sinha et al. 2005; Non et al. 2006). Although penicillin-binding protein 4 homologs have been linked to penicillin resistance in several pathogenic bacteria (Zawadzka-Skomial et al. 2006; Stefanova et al. 2004; Finan et al. 2001), the role of this protein has yet to be elucidated in M. tuberculosis.

TABLE 3
Categories representing divergence of genes discovered in
97 clinical M. tuberculosis genomes but not found in the
primary M. tuberculosis reference strain and their frequency
within each category. InDel: Insertion or Deletion:

| | Number of CDSs not in H37Rv |
|---|---|
| Category | within the category |
| Within TbD1 or RvD1-6 Regions | 44 |
| Aligned with gene in M. tuberculosis | 120 |
| CDC1551 | |
| Transposases | 20 |
| Duplications | 37 |
| Pseudogenes | 280 |
| Contains Single Base InDel or InDel in | 259 |
| Homopolymer | |
| Truncations | 887 |
| Extensions | 56 |
| Partial Alignment | 322 |
| No Category | 36 |

After classifying genes not in H37Rv within the RvD1-6/TbD1 and CDC1551 categories, the remaining genes were subsequently categorized. Truncations and pseudogenes were excluded from further analysis of the categories because they were merely shortened or mutated genes present in H37Rv, which reduced the number of genes not in H37Rv to 730 (11.9% of the pangenome).

These results indicate that across M. tuberculosis strains, there still appears to be a large number of differences in genetic content. Therefore, the number of genes not in H37Rv were identified and summed for each genome and a proportion was calculated based on the total number of CDSs per genome. There was an average of 1.5% of genes not present in H37Rv per genome (median=1.6%, minimum=0.71%, maximum=2.1%, standard deviation=0.33%). Although this aligns with previous reports of the highly clonal nature of M. tuberculosis strains, the percentages were still higher than these reports (0.4% of genes in M. tuberculosis CDC1551 not present in H37Rv (Fleischmann et al. 2002)).

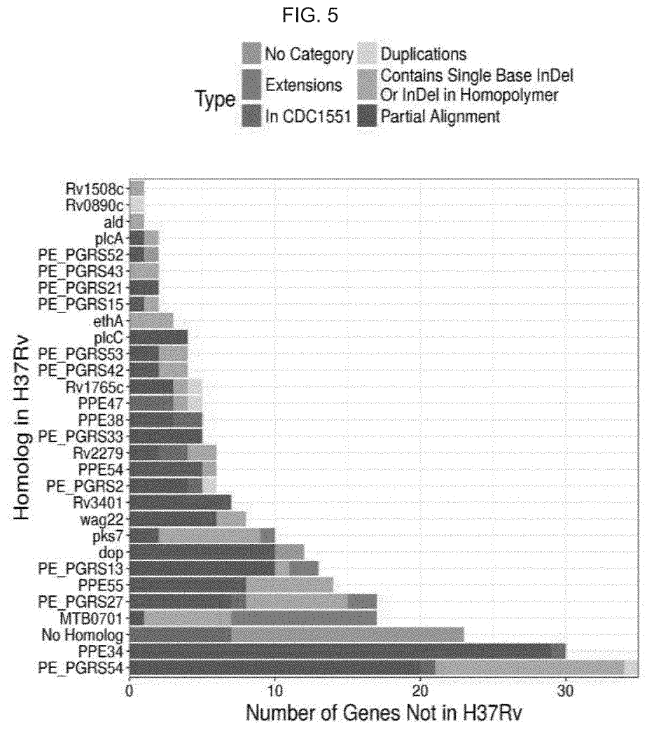

To identify a possible homolog in H37Rv, we aligned (blastn (Altschul et al. 1990)) all 730 to the nucleotide proteome of H37Rv. H37Rv homologs were identified based on lowest Evalue among all blastn hits (maximum Evalue of 0.01). Genes not meeting criteria for any category were categorized as having no homolog in H37Rv (did not align to any H37Rv gene or had an Evalue above the 0.01 threshold). Of the 730 genes, 705 aligned to a homolog in H37Rv. Additionally, 192 were considered PE/PPE genes (26.3%). Partial alignments represented the most PE/PPE genes not present in H37Rv compared to other categories (FIG. 5). This indicates possible gene conversion, which supports previous reports that describe recombination hotspots in these families (Phelan et al. 2016).

Gene Duplication is the Primary Mechanism of Gene Gain in M. tuberculosis

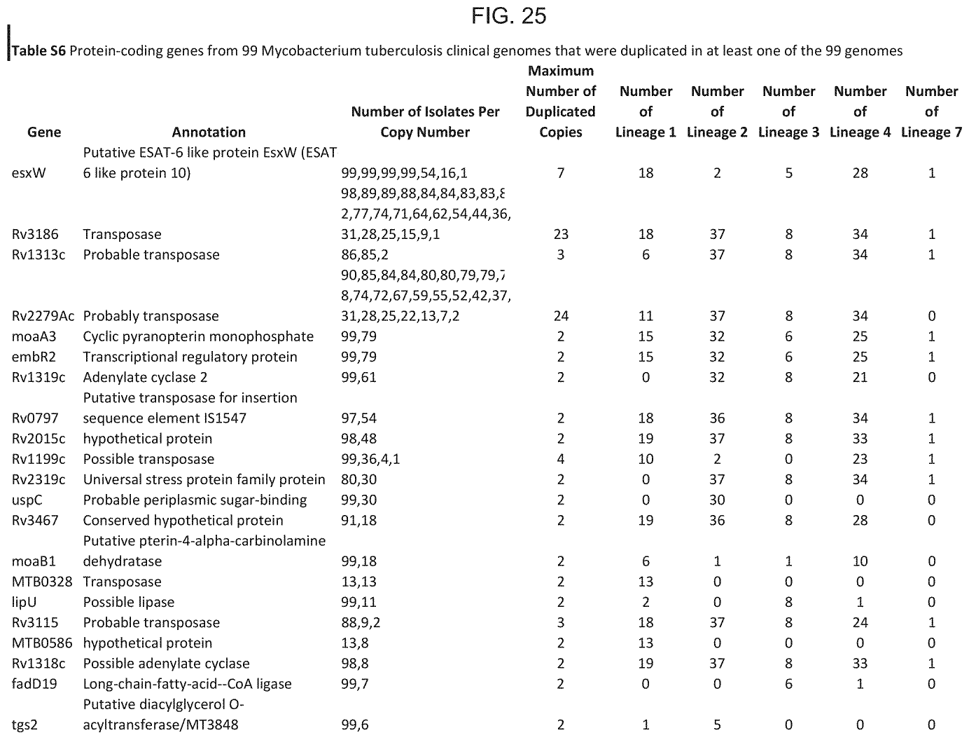

To identify duplicated genes, we used CDHIT⁴² to find clusters of more than one sequence in the proteome of each isolate. We also queried for genes annotated as the same gene name from AnnoTUB annotations within all genomes. Sixty-three unique genes were duplicated at least once within the current dataset (Table S6, or FIG. 25). These duplicated genes were enriched for cAMP catabolic process and nucleotide catabolic process (GO enrichment, FIG. 1). Of the 60, only eight were PE/PPE proteins (one PE, seven PPE), each from a different genome.

After excluding transposases (n=10), embR2, moaA3, and rv1319c were the top three most frequent gene duplications. All three duplications events have been described to occur in M. tuberculosis CDC1551 and lineage 4 strains (Azhikina et al. 2006; Stavrum et al. 2008; Gautam et al. 2017), the duplications moaA3 and rv1319c (mt1360) have been reported additionally in lineage 2 strains (Stavrum et al. 2008).

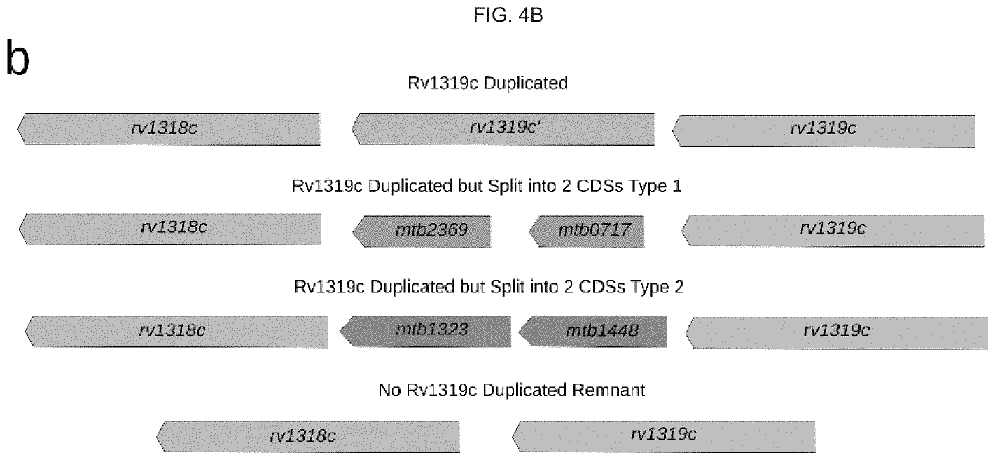

The single homolog in H37Rv (rv1319c) was described previously to be a result of a deletion-fusion event where the ancestor had two copies as demonstrated in CDC1551 (mt1360 and mt1361) but were fused into a single CDS (Fleischmann et al. 2002). M. tuberculosis Haarlem strain and M. bovis also demonstrated this fused CDS (Fleischmann et al. 2002). However, our investigation revealed duplications of rv1319c existed across the phylogeny, with the exception of all lineage 1 strains and the single lineage 7 strain (FIG. 4a). The duplication likely occurred in the M. tuberculosis progenitor and was lost in the lineage 1 and 7 ancestor (FIG. 4a). Furthermore, we observed two clusters, one within lineage 2 and one within lineage 4, that contained two CDSs where the mt1360 homolog existed, suggesting the ancestor of these clades had a fully functional copy but had frameshift mutations, resulting in a premature stop codon (FIG. 4b). This resulted in two CDSs where other strains had a single CDS (homolog of mt1360). Finally, because of the few clades that demonstrated a loss-of-function mutation in the redundant copy, the configuration in H37Rv was likely a result of loss-of-function mutations then subsequent loss of the redundant copy, not a deletion then subsequent fusion of mt1360 and mt1361 to create rv1319c.

Seventy-nine strains had a previously reported duplication event involving moaA3 and embR2 (Fang et al. 1999), known as RvD5 (Brosch et al. 1999). This region contains moaX-moaB3-moaA3-embR, similar to M. bovis and M. tuberculosis CDC1551 (Sarojini et al. 2005). The remaining 20 genomes did not have moaA3 or embR2: two strains matched H37Rv in this region (moaX-moaB3-rv3325-rv3326-rv3327) and the remaining had 2-4 copies of IS elements in various orientations. The twenty strains represented lineages 1, 2, 3, and 4, indicating that this variation is not lineage-specific. Since the moa duplication was postulated to occur in the ancestor of tubercle baccilli (Levillain et al. 2017), the duplication of embR2 may have also been duplicated in the ancestor. A BLAST search of embR2 in M. canettii strains in the BLAST nr database returned an identical embR2, indicating M. canettii also has two homologs of embR. Since both M. bovis and M. canettii strains have RvD5 intact (moaX-moaB3-moaA3-embR2), the expanse of IS elements in the 20 M. tuberculosis clinical strains likely occurred independently. This was not unexpected as this region is a known “hotspot” for IS element activity (IS6110 preferential locus [ipl]) (Fang and Forbes 1997; Ho et al. 2000).

FIG. 4 illustrates an evolutionary analysis of the duplicated Rv1319c gene (encodes an adenylyl cyclase) in 99 M. tuberculosis genomes. a) SNP maximum likelihood phylogenetic tree with the clades colored by the duplication status (has a duplicate (pink), has a remnant [two Types] (blue/green), or does not have a remnant or duplicate (gray)). The outer rim colors represent Lineage determined by MIRU-VNTR/spoligotype. Outgroups for the tree were M. bovis and M. canettii. b) A schematic of the synteny within the four types of gene duplication/remnant. rv1319c′ represents the copied version of the original rv1319c. Rows 2 and 3 represent different CDSs created by different frameshifts. The last row represents the lineage 1 and lineage 4 isolates with no duplicate or remnant CDS(s) (matches the gene order in H37Rv).

Gene Conversion Alters B Cell Epitopes in M. tuberculosis PE_PGRS Proteins

Partial alignments represented the most genes not present in H37Rv (FIG. 2c). Furthermore, PE/PPE genes were most frequent within partial alignments compared to other categories (FIG. 2c). Together with the previous observation of recombination hotspots in the PE/PPE¹¹, we investigated potential gene conversion in both protein families with a software designed to identify recombination events and significant breakpoints, Recombination Detection Program 4 (RDP4)⁴⁵. To do this, we first annotated all PE/PPE genes in the pangenome.

FIG. 5 illustrates the homolog in M. tuberculosis H37Rv based on an all-against-all pairwise nucleotide alignment for all genes identified to not exist in H37Rv from a pangenome of 97 M. tuberculosis genomes and categorized into several groups based on alignment statistics. Homologs represented by more than seven genes are present in the plot.

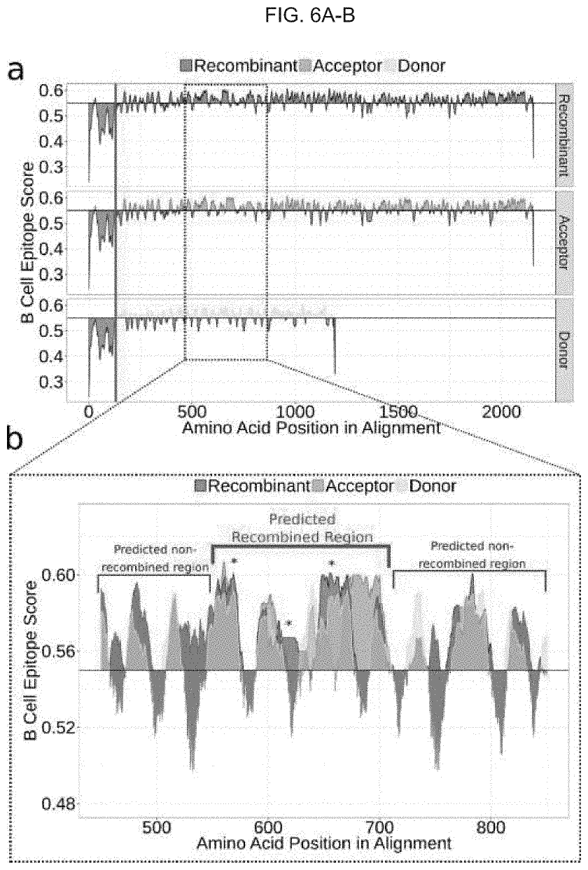

FIG. 6 illustrates B cell epitope scores predicted by Bepipred 2.0 across the alignment of PE_PGRS genes involved in a predicted gene conversion event predicted by RDP4 in a M. tuberculosis lineage 4 isolate. A) B cell epitope scores along the alignment of the putative acceptor (PE_PGRS54), putative donor (PE_PGRS57) and recombinant (MTB1316). Horizontal line at 0.55 represents threshold. Grey areas represent regions of the gene with epitope scores below the 0.55 threshold, indicating no predicted epitopes. Red vertical line represents the end of the conserved PE domain (˜110 amino acids) and start of variable PGRS domain. b) B cell epitope scores of the region within the alignment from part A where RDP4 detected gene conversion. Red bracket represents the epitope scores in the recombinant region that was replaced by the donor gene, blue brackets represent the scores of the alignment in the non-recombined region. Asterisks represent differential epitope peaks in the recombinant, acceptor and donor that may have resulted from the predicted gene conversion event.

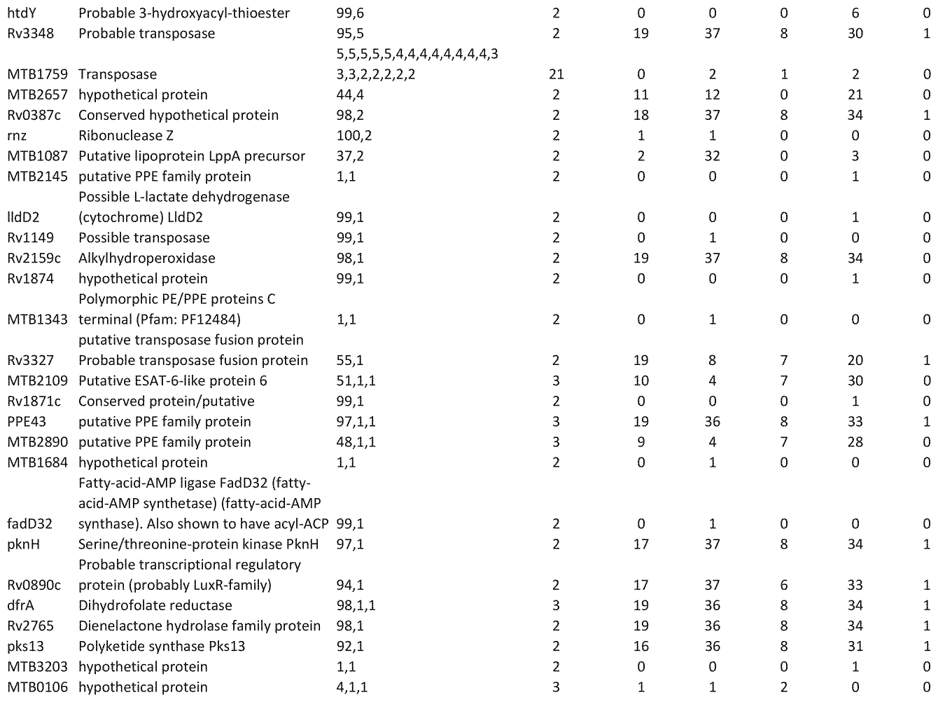

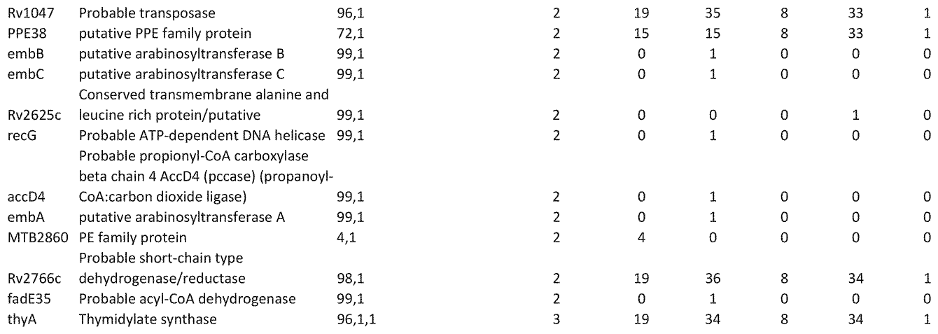

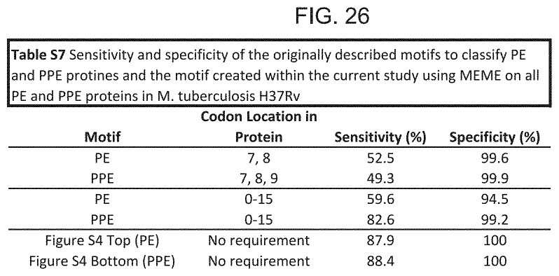

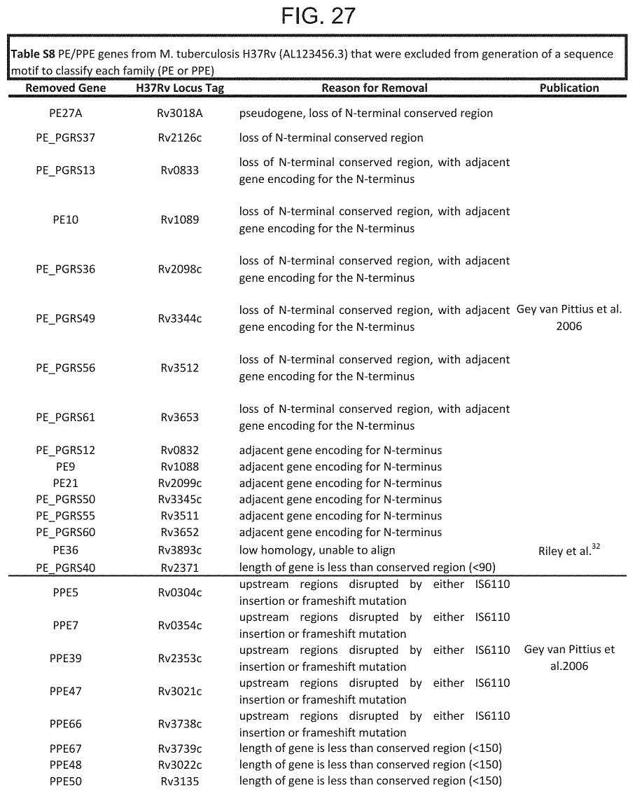

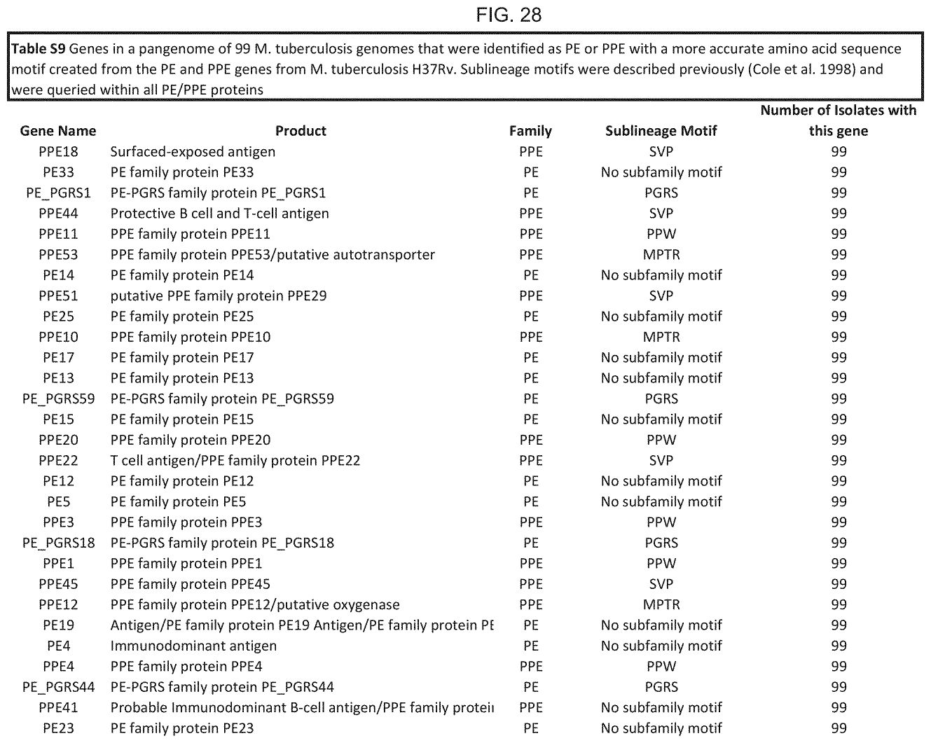

We initially attempted to use the originally described PE and PPE motifs¹³, but these motifs detected PE/PPE genes in the H37Rv proteome with low sensitivity and specificity (Table S7, or FIG. 26). To improve on this sensitivity and to identify PE/PPE genes in clinical strains that were not transferred from the H37Rv annotation, we identified a new amino acid sequence motif for each family. The PE motif spanned 56 amino acids of the conserved region, while the PPE motif spanned 84 (FIG. 5 and Data Files 1 and 2). These motifs yielded a much higher sensitivity and specificity in identifying PE/PPE genes (Table S7, or FIG. 26). The PE/PPE genes that were not detected with these motifs (false negatives) were among the set of PE/PPE genes that lack the protein structure expected for these families (Table S8, or FIG. 27). The new PE and PPE motifs identified 247 PE (74% of all PE) and 118 PPE genes (66% of all PPE) that were not annotated in H37Rv (Table S9, or FIG. 28), resulting in 333 unique PE and 177 unique PPE genes in the pangenome. Conserved PE/PPE genes have been proposed as effective vaccine candidates¹¹. A search for such genes returned 38 core PE/PPE genes (4 PE_PGRS, 15 PE, and 19 PPE). A functional enrichment of 10 PPE genes that had GO terms revealed host interaction functions (FIG. 11), important characteristics for vaccine candidates.

FIG. 11: Gene ontology enrichment for 10 PPE proteins that were considered core genes in a pangenome analysis of 99 M. tuberculosis genomes.

While these large numbers of PE/PPE genes within the accessory genome suggests active diversification within these families, their frequency in individual isolates varied within a tight range (142-162, median of 154). Furthermore, there was an average of 25 PE/PPE genes not present in H37Rv per genome (range 4-39, median of 25). These numbers suggest that extensive gene amplification is not occurring in these protein families even with the high amount of sequence divergence. This apparent limited amplification, together with the enrichment of PE/PPE genes not in H37Rv within partial alignments (FIG. 2c) further supports recombination occurring in the PE/PPE genes.

We investigated this hypothesis further and focused on gene conversion, which has been previously suggested to occur in PE_PGRS genes¹². Gene conversion is a biased homologous recombination mechanism that results in intragenomic donation of genetic information⁴⁶. Detecting gene conversion requires existence of non-converted versions of the two genes originally involved in the event (FIG. 6). We chose Mycobacterium bovis BCG Pasteur to represent non-converted PE/PPE genes (Genbank accession AM408590.1). PE/PPE proteins and subfamilies were classified in the same manner as the M. tuberculosis clinical genomes. Gene conversion occurs at the intrachromosomal lever; thus, we randomly chose three representative isolates from lineages 1, 2, 3, and 4 (lineage 7 was represented by a single isolate in this study) to query for gene conversion. All newly annotated genes (i.e. not transferred from H37Rv) within each subfamily (PE_PGRS, PE non-PGRS, PPE_SVP, PPE_PPW, PPE_MPTR, PPE no motif) were concatenated with corresponding subfamily genes in M. bovis BCG for each isolate, totaling 78 comparisons. RDP4 detected gene conversion only in the PE_PGRS subfamily (FIG. 12).

FIG. 12: RDP4 output for the gene conversion event discovered in the lineage 4 M. tuberuclosis isolate, SEA06535, and the corresponding neighbor-joining trees created from the acceptor sequence alignment (left) and donor sequence alignment (right). DSB: double-stranded break.

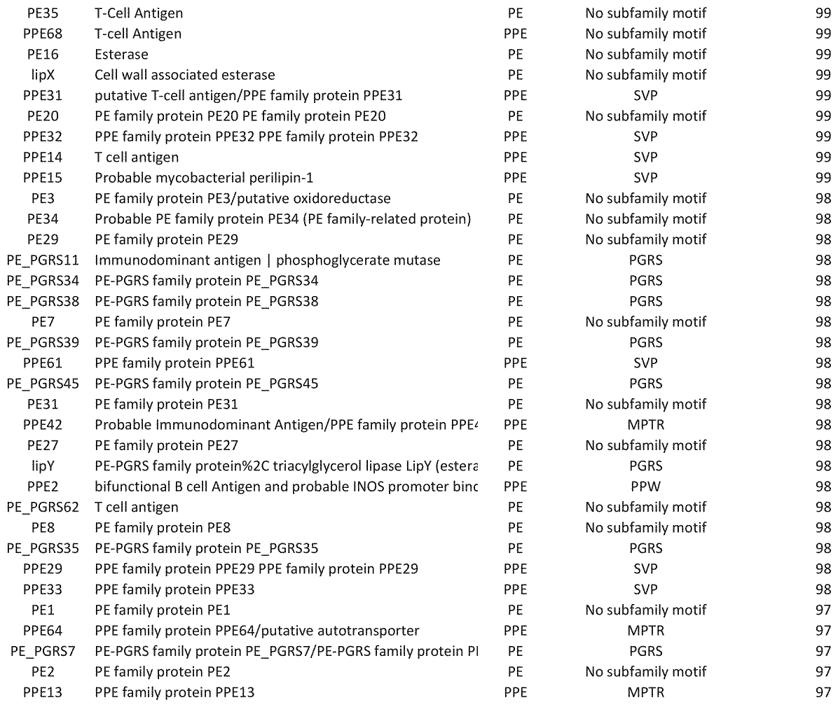

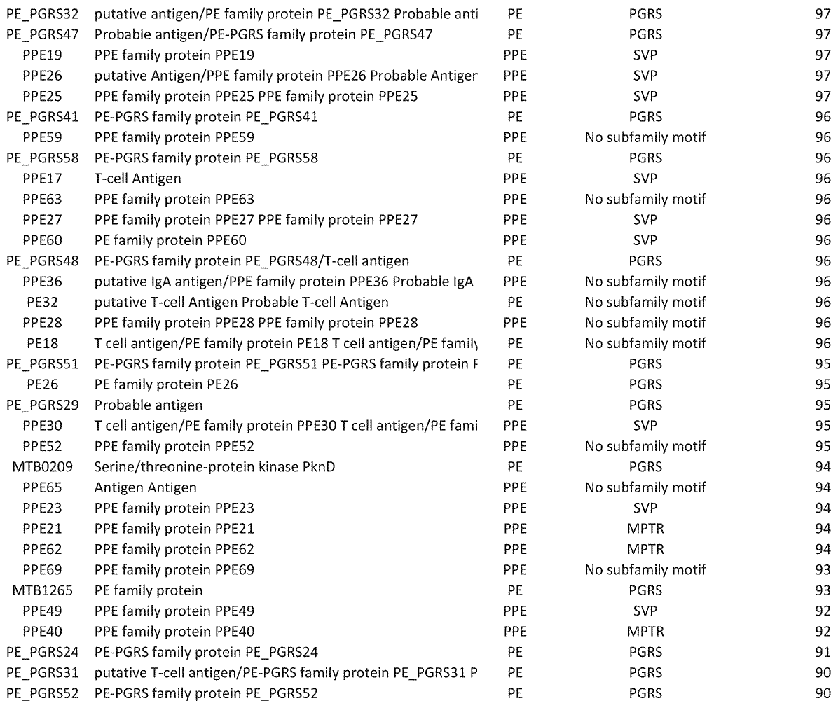

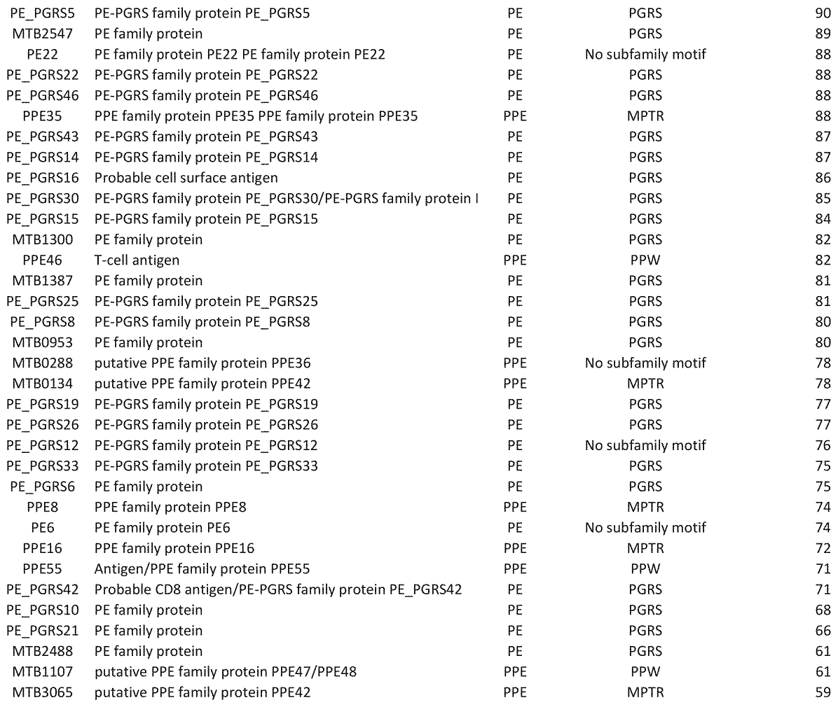

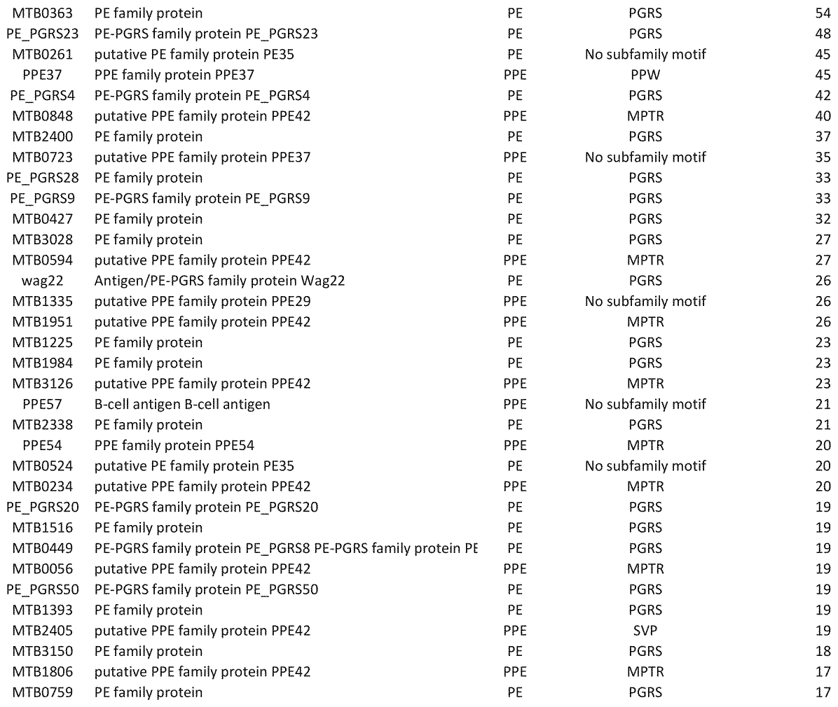

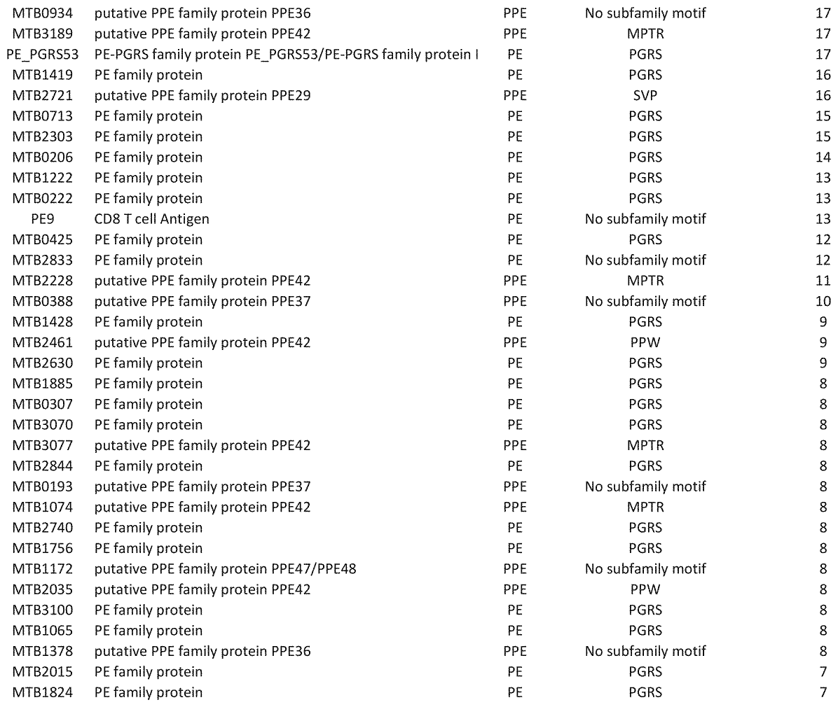

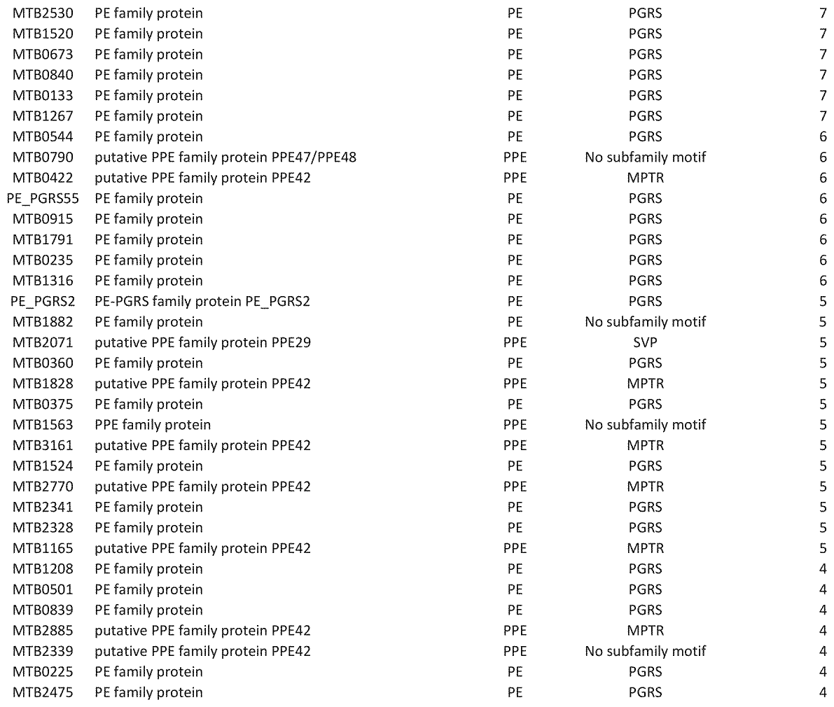

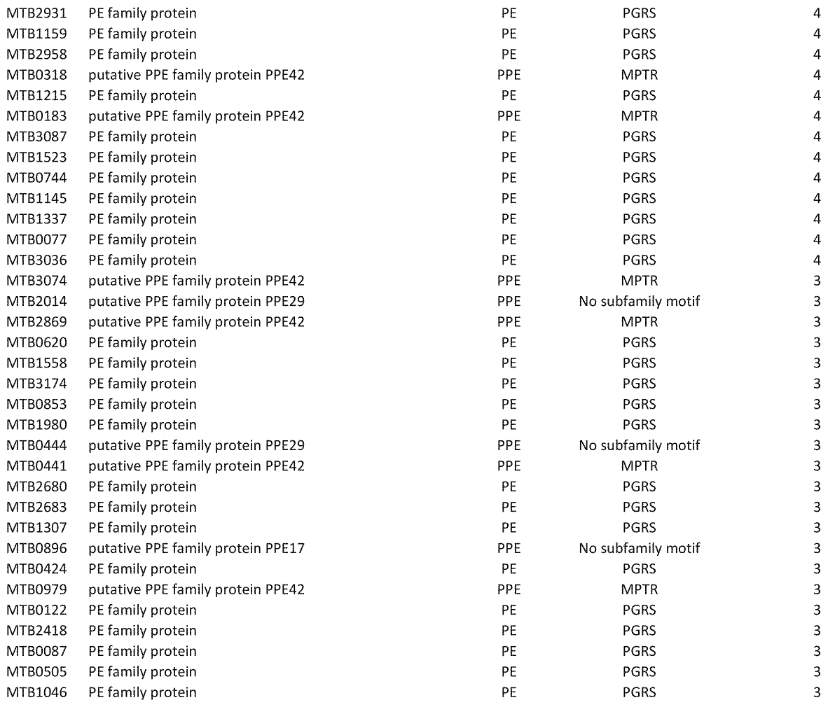

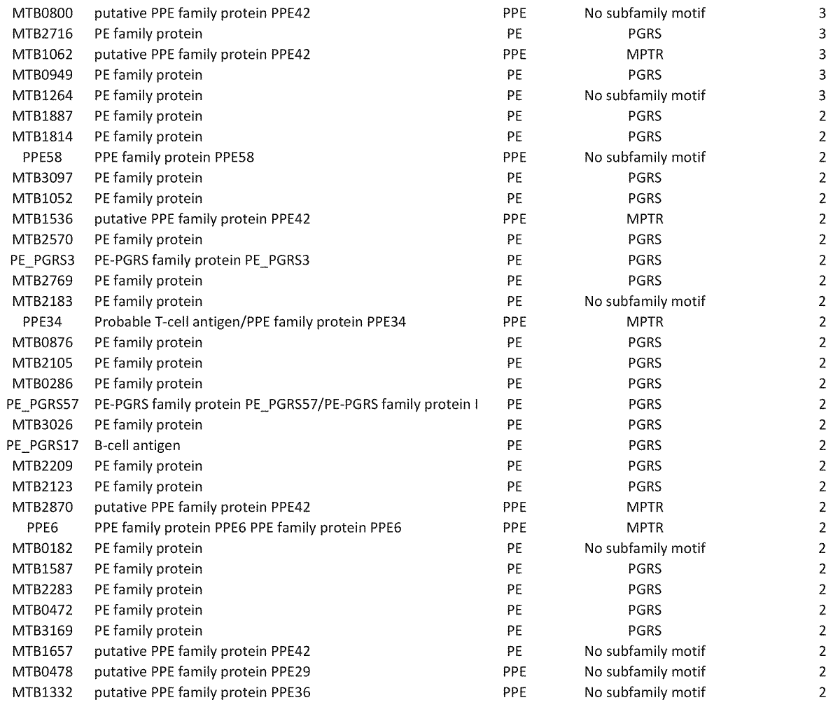

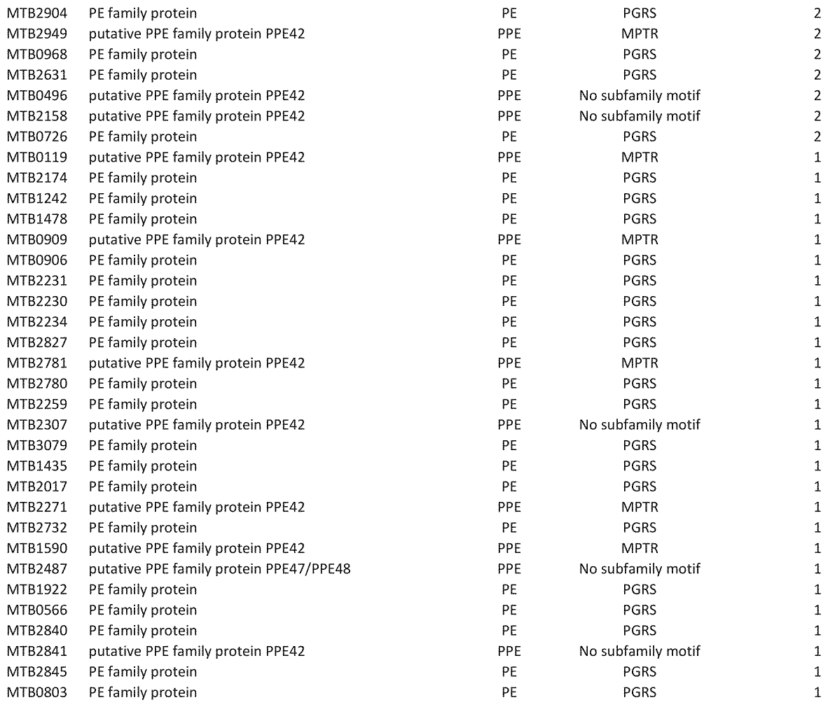

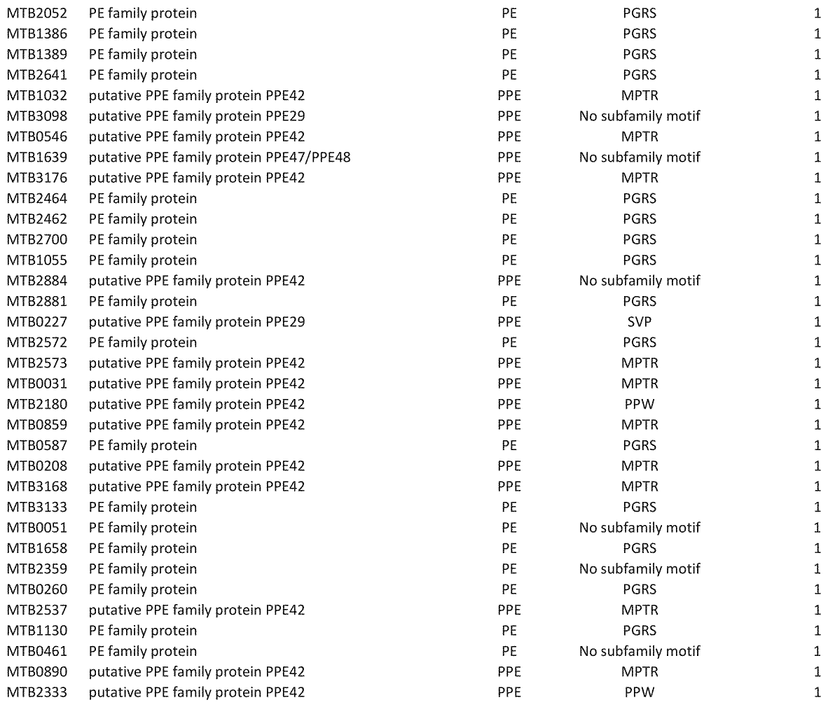

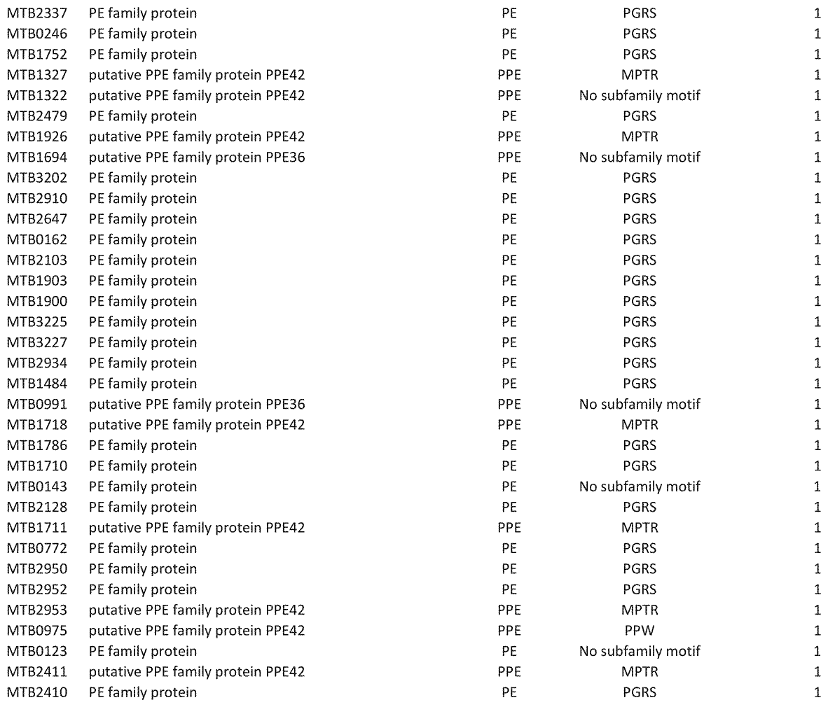

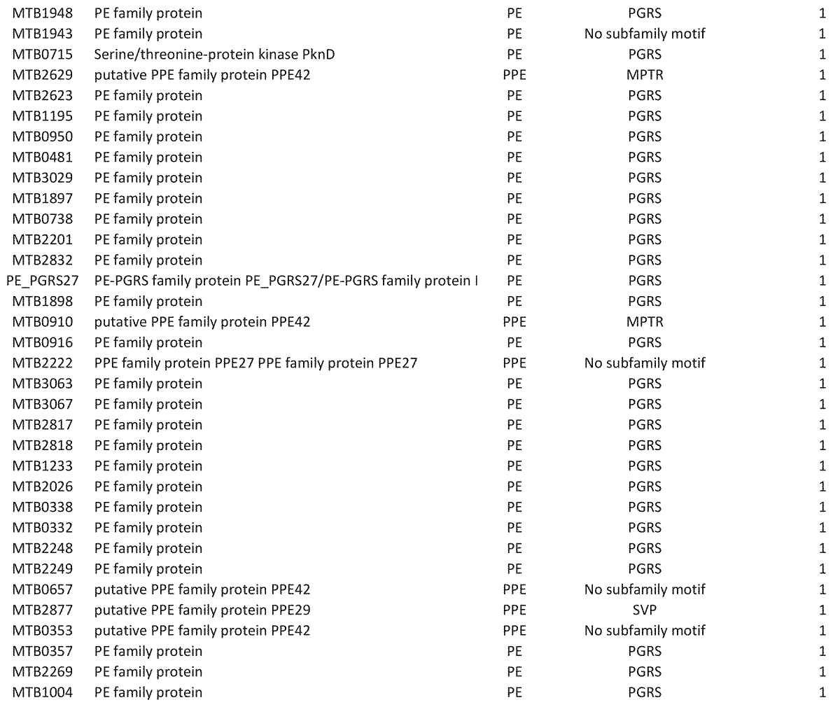

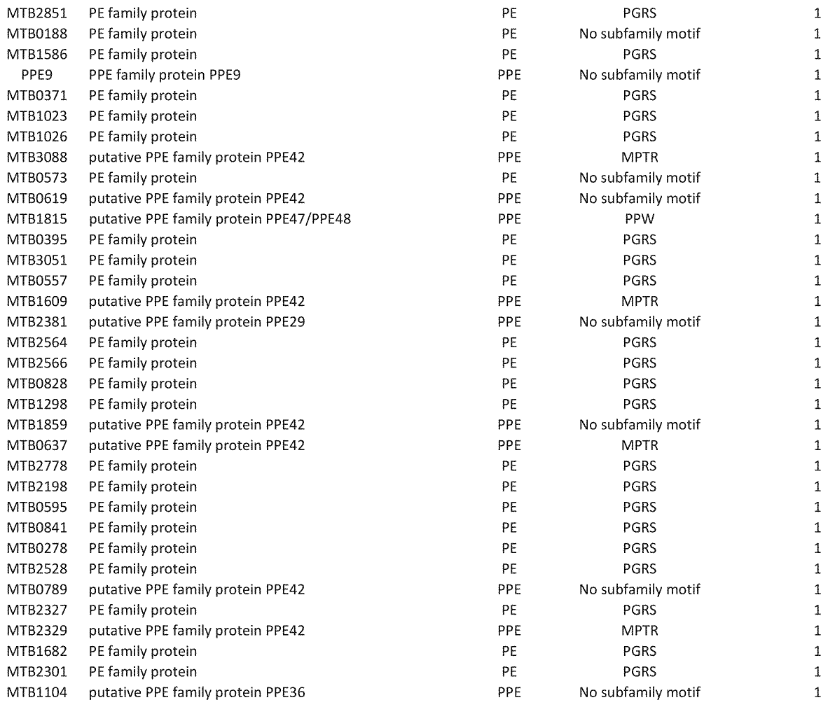

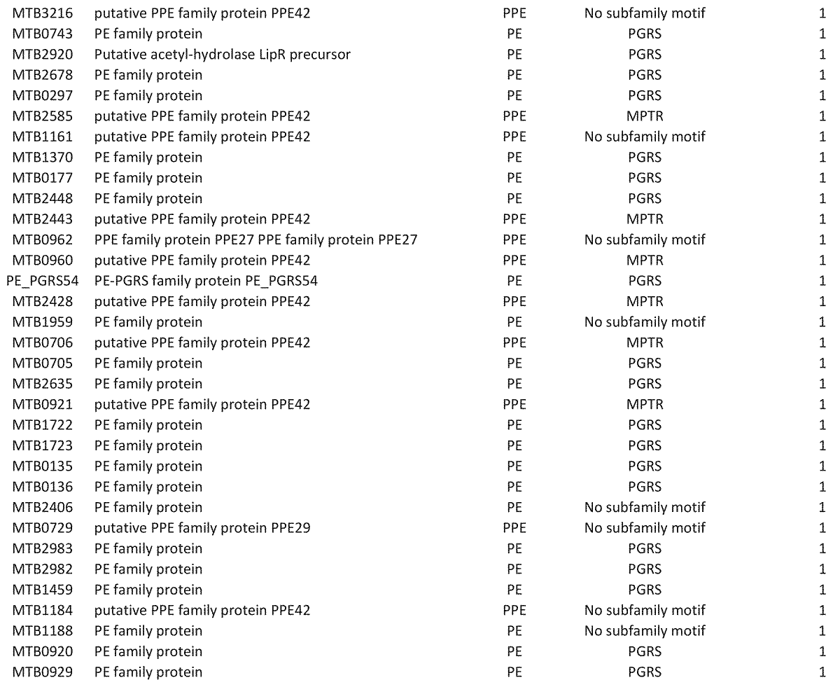

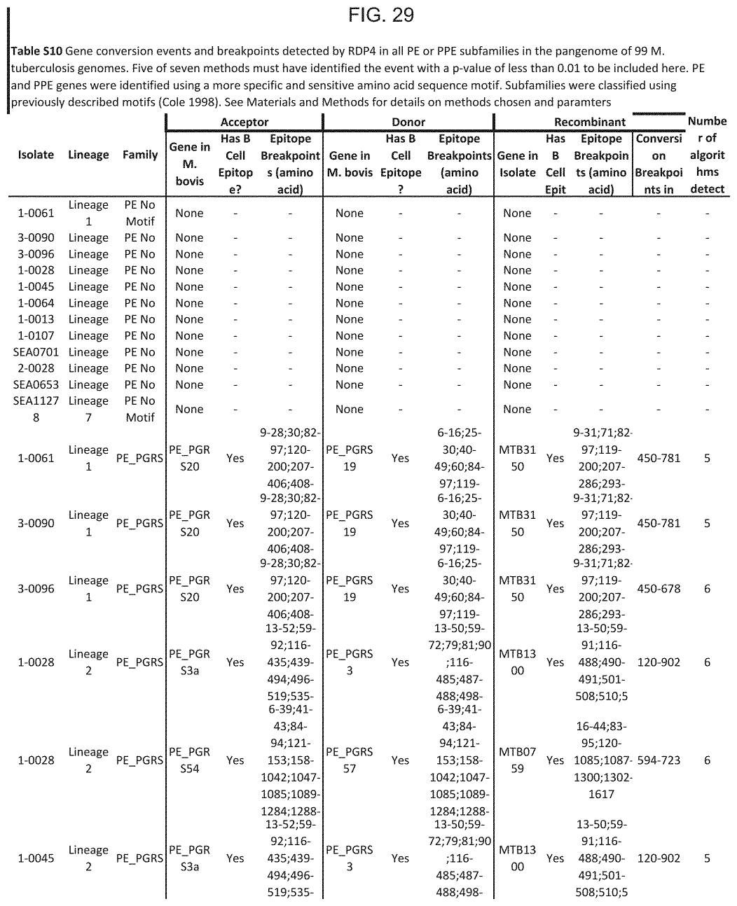

One gene conversion event was discovered in lineage 1, 3, 4, and 7; two events were identified in lineage 2 (Table S10, or FIG. 29). For isolates not tested for gene conversion, a syntenic analysis queried for the genes in the same syntenic region as those found in the representative set of isolates. The event in lineage 1 and the event involving putative acceptor M. bovis BCG PE_PGRS3a and putative donor PE_PGRS3 in lineage 2 did not have many different recombinants; however, many different recombinants were observed in lineages 2, 3, and 4 (FIG. 13).

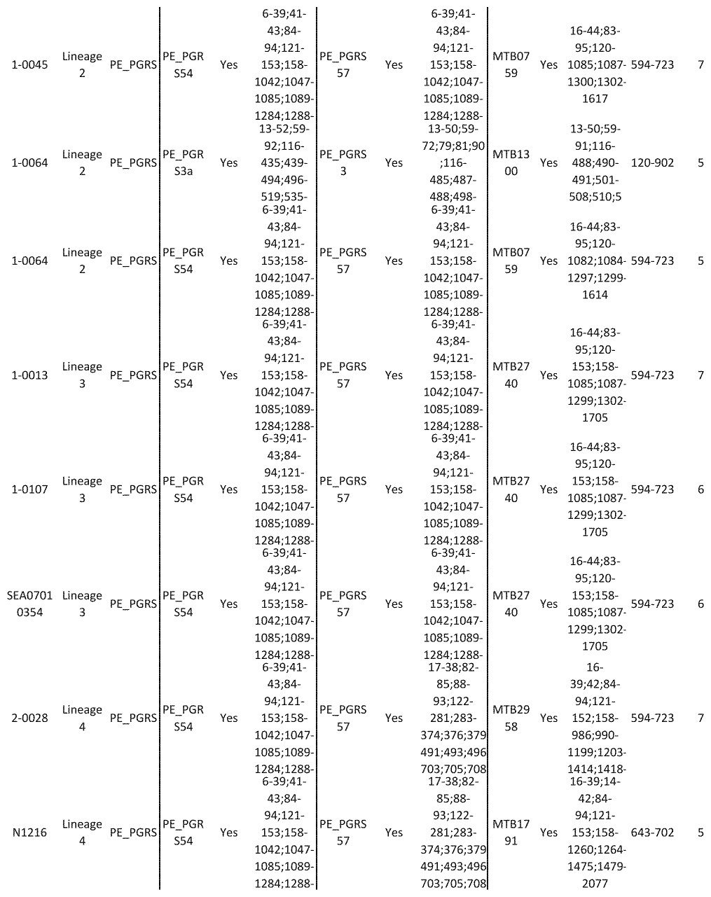

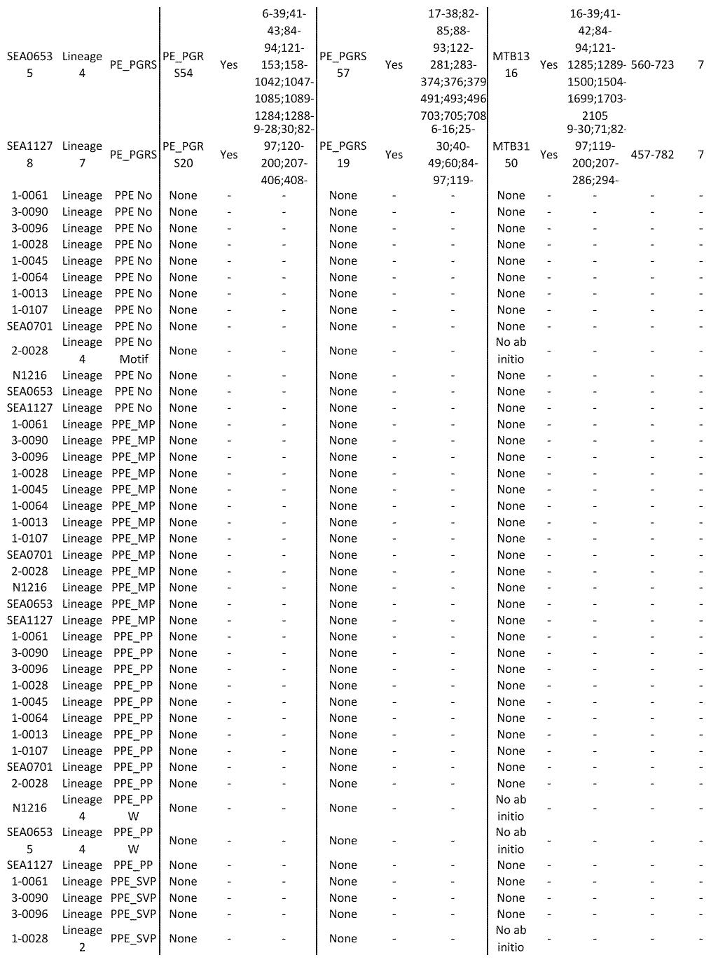

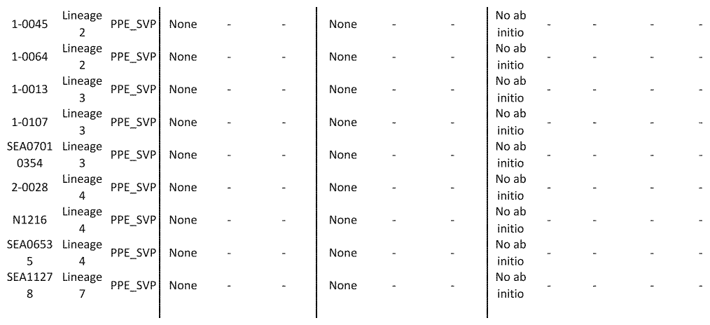

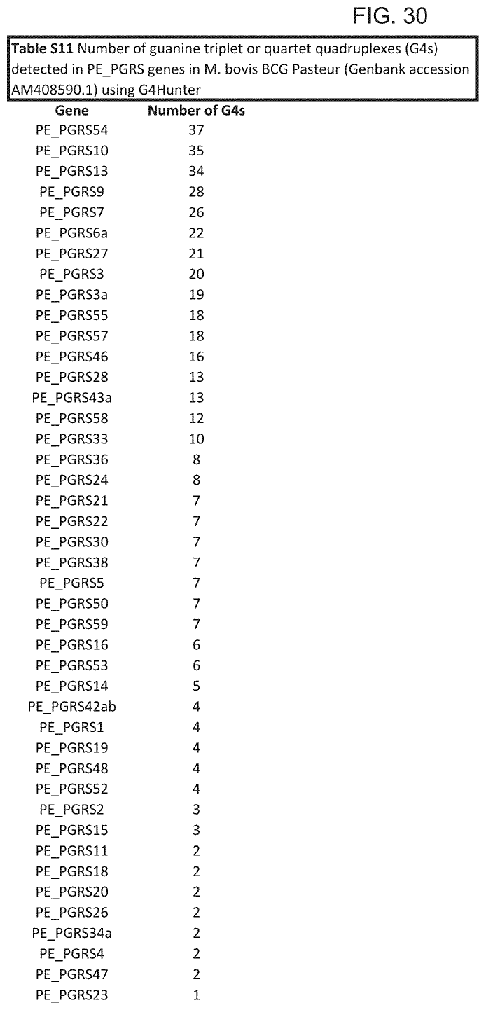

FIG. 13: Recombinant products identified in all isolates mapped to a SNP phylogenetic tree resulting from probable gene conversion events within each lineage. Conversion events determined by RDP4 for each isolate representing each lineage were queried in isolates not analyzed by RDP4 based on synteny (Table S11, or FIG. 30). Recombinants produced by a) PE_PGRS20 acceptor, PE_PGRS19 donor in lineage 1; b) PE_PGRS3a acceptor, PE_PGRS3 donor in lineage 2; c) PE_PGRS54 acceptor, PE_PGRS57 donor in lineage 2; d) PE_PGRS54 acceptor, PE_PGRS57 donor for lineage 3; and e) PE_PGRS54 acceptor, PE_PGRS57 donor for lineage 4. Lineage 7 was represented by a single isolate and is not present in this figure. See Table S11, or FIG. 30, for gene conversion events in lineage 7.

The representative isolates from all lineages 2, 3, and 4 had predicted conversion events where M. bovis BCG PE_PGRS54 was the putative acceptor and M. bovis BCG PE_PGRS57 was the putative donor (Table S10, or FIG. 29).

This consistent observation of the same parental genes converting but at different breakpoints suggests increased opportunity for recombination in different areas of the gene. A non-B DNA structure—G triplet/quartet quadruplexes (G4s)—could be acting as signals for DNA strand nicking⁴⁸, which is the first step required for gene conversion. This is supported by significantly more G4s in PE_PGRS and frequent G4s observed in PE_PGRS54 (Table S11, or FIG. 30). The frequent G4s provide several opportunities for DNA nicking, which could explain the different recombination breakpoints (Table S10, or FIG. 29). G4s may provide the opportunity for gene conversion in PE_PGRS genes, generating antigenic variation.

PE_PGRS genes are known antigens that contain GC-rich, repetitive motifs but lack T cell epitopes within the PGRS domain¹⁷, which we support (FIG. 14A-B). Conversely, antigens with repetitive sequences tend to elicit a T-cell-independent response¹⁹, indicating a B-cell-specific recognition. With this in mind, B cell epitopes were queried and identified in 97% of PE_PGRS genes (Table S12). These B cell epitopes primarily resided within the PGRS domain (FIGS. 7a and b), which suggests the PGRS domain interacts with antibodies.

FIG. 14A-B: Frequency of T cell epitopes across PE_PGRS proteins in 97 M. tuberculosis clinical isolates' genomes and two reference genomes, H37Rv (NC000962.3) and H37Ra (CP016972.1) for a) MHC class I and b) MHC class II. Vertical dotted line represents the end of the conserved PE domain (left to right).

Noting that gene conversion events affected PGRS regions, we hypothesized that the resulting recombinants were selected for because they altered B cell epitopes, affecting antibody recognition and creating antigenic variation. To test this hypothesis, recombination breakpoints within each gene conversion event were compared to B cell epitope scores in each acceptor, donor, and recombinant. Importantly, the regions surrounding the recombined region within the acceptor and resulting recombinant aligned almost exactly (blue dotted boxes in FIGS. 7c and d). Notably, gene conversion breakpoints associated with altered B cell epitope scores (FIGS. 7c and d). These results suggest a potential mechanism to create antigenic variation, thereby evading antibody recognition in M. tuberculosis.

### DISCUSSION

We sought to elucidate genetic diversity in M. tuberculosis clinical strains beyond small chromosomal changes, which are thought to be the main contributors of diversification in the species⁴⁹. Yet, genetic events such as duplications and recombination events contribute to genetic diversity far more than small changes. Duplications described in the M. tuberculosis W/Beijing sublineage contribute to the constitutive overexpression of the dormancy regulon, increasing virulence (Domenech et al. 2010). Tandem gene duplications described in Vibrio species provide a protein dosage effect, which elevates respiration under anaerobic conditions⁵⁰. Meanwhile, recombination via large segmental gene conversion has been shown to drive antigenic variation in pathogens like Neisseria gonorrhoeae⁵¹, Borrelia species⁵², and African Trypanosoma species⁵³. However, due to the widespread use of short-read sequencing on M. tuberculsosis genomes, these events have been obscured by the systematic biases that exist with this type of sequencing. The most commonly used method to process short reads—reference alignment—precludes detection of new genetic content not present in the reference. Another bias—GC amplification—can obscure recombination hotspots, as these are most commonly found in GC rich regions⁵⁴.

By de novo assembling long reads produced by PacBio, which have none of the systematic biases described above, we generated the largest number of reference-quality M. tuberculosis clinical isolate genomes reported to-date. If strict QC measures are not implemented, random sequencing errors (single-base indels⁵⁵) propagate into downstream analyses, which can introduce inaccurate gene coordinates in genome annotation. To mitigate this effect of erroneous single-base indels, we developed a comprehensive quality control protocol, carefully verified every genome, and confirmed the consensus sequence. However, these strict criteria reduced the sample size for analysis by almost 50%. Those that were excluded contained a higher rate of error (FIG. 7A). Proper loading of the DNA onto the sequencer (PacificBiosciences 2014), avoiding freezing and thawing of the DNA (Klingström et al. 2018), and reducing culture time for DNA extraction (Andreu and Gibert 2008) can reduce these errors and improve assembly accuracy. These strict quality control measures produced 97 polished consensus and circular genomes.

These genomes allowed annotation of genetic content absent from the most studied reference strain, H37Rv. Nevertheless, this reference strain has the most experimentally-verified CDS functions and coordinates among all M. tuberculosis reference strains⁵⁶. Therefore, a hybrid annotation approach (AnnoTUB) was developed to preserve annotations from this well-studied reference while carefully annotating new genetic content ab initio. This resulted in a tight range of CDS number across the population (FIG. 9).

FIG. 9 graphically illustrates a genome count density for number of CDSs transferred from the Rapid Annotation Transfer Tool (RATT), which is a subset of the density for Annotate TUBerculosis (AnnoTUB), a custom annotation pipeline designed to annotate M. tuberculosis genomes.

Owing to systematic exclusion of these genes from short-read sequencing studies, this work is the first to assemble PE/PPE genes and study their conservation across a global collection of clinical isolates. A pangenome analysis compiled all annotations and revealed an unexpected abundance of absolutely identical genes across all 97 clinical genomes. These genes were enriched for essential genes but over 200 were considered not essential, which is unsurprising for an intracellular pathogen. Because the dataset referenced to analyze essentiality was performed in an in vitro environment³⁹, it's possible these non-essential but identical genes are essential in vivo. To confirm their essentiality in vivo, transposon insertion mutants grown in an in vivo model would be needed. Several unexpected non-essential genes were several toxin-antitoxins (TAs), which typically mutate at high frequencies and are often lost through pseudogenization (Soo et al. 2014). Observing these identical TAs across all 97 strains indicates that a few genes within this family are not mutating or being lost. Although any of the identical proteins could be potential drug targets, toxin-antitoxin proteins are particularly attractive as they lack human homologs⁵⁷, are active during dormancy⁴¹, and may be essential for its induction through inhibiting protein synthesis⁴¹. Outside of identical genes, the pangenome also uncovered an unprecedented number of genes not present in H37Rv (FIG. 2b), including gene duplications.

Gene duplications represent an important source of gene gain in M. tuberculosis due to lack of HGT in the species (Boritsch et al. 2016). Gene duplication is hypothesized to be the safest way to acquire adaptive mutations rather than increasing mutation and recombination rates genome-wide (Francino 2005). However, once a duplication event has occurred, they are expected to be lost through genetic drift, random mutation, or recombination (Lynch and Conery 2000; Lynch and Force 2000). Retention of extra gene copies would likely be a recent evolutionary event or a selective force maintains both or all copies that originated from the ancestor, restricting the freedom to diverge (Bergthorsson et al. 2007). One such duplication in M. tuberculosis (moaA3-embR2), which represents the RvD5 region (deletion of this region in H37Rv) (Zheng et al. 2008), has been reported in several clinical strains (Sekar et al. 2009; Fang et al. 1999; Sarojini et al. 2005) including M. tuberculosis CDC1551 (Gautam et al. 2017), M. bovis (Sarojini et al. 2005), and M. canettii (Levillain et al. 2017). This region was created via an inverted gene duplication of moa1 operon and subsequent fusion of moaE1 and moaD1 (moaX), within the ancestor of tubercle (TB) bacilli (Levillain et al. 2017). embR2 may have also been an inverted duplication event within the ancestor of TB as M. bovis and M. canettii have embR2 adjacent to moaA3. This region appears to be conserved as the majority of strains in the present study had the ancestral moaA3-embR2 region intact. Another frequent gene duplication was rv1319c. In H37Rv, this region contains three adenylyl cyclases (rv1318c-rv1319c-rv1320c) but in CDC1551 there are four adenylyl cyclases (mt1359-mt1360-mt1361-mt1362). mt1359, mt1361, and mt1362 are considered homologs of rv1318c, rv1319c, and rv1320c, respectively, with mt1360 having close homology to mt1361/rv1319c (Fleischmann et al. 2002). It was previously suggested that rv1319c was created by a fusion between mt1360 and mt1361 and a subsequent deletion of mt1360 (Fleischmann et al. 2002). However, the orientation of three cyclases was more likely a result of the duplicated homolog (mt1360) being lost, not a deletion-fusion event. This hypothesis is supported by two clades in lineage 2 and lineage 4 in which the ancestors of the clades seemed to have had a functional mt1360 homolog but had accumulated mutations that introduced premature stop codons (FIG. 4). This likely resulted in a loss-of-function within the mt1360 homolog. In both events (moaA3-embR2 and rv1319c/mt1360), the majority of strains likely maintained the redundant copy for a selective benefit under environmental stressors. Although the phenotypic effects of the moaA3 and rv1319c/mt1360 duplications have yet to be identified, the redundancy of embR2 has been studied. Molle et al. (Molle et al. 2008) observed EmbR2 binds to PknH, like EmbR, but cannot be phosphorylated by PknH. The authors hypothesize that the EmbR2/PknH interaction may inhibit PknH activation and increase virulence in strains that have EmbR2. Because these duplications were conserved in the majority of clinical strains, knockouts and complementation in strains that have these regions conserved, such as M. tuberculosis CDC1551, may elucidate their phenotypic effects during infection. Although duplication has been reported to occur in vitro, the study observed such duplications after thousands of passages over a span of 13 years (Brosch et al. 2007). The strains included in the present study did not undergo multiple passages in culture prior to sequencing. Furthermore, the most prevalent duplications are likely not the result of spontaneous duplication.

When a gene is duplicated, one of the two copies is freed from selective constraints and can accumulate neutral mutations⁶. However, these mutations can often be deleterious and result in a loss-of-function, producing a pseudogene. Gene loss is expected to occur more frequently than gene gain because of the expected genome contraction of intracellular organisms⁶⁰. Gene loss has been reported to be the primary source of diversity in M. tuberculosis but this conclusion could not be substantiated, as duplications were excluded from the analysis⁷. Our analyses demonstrated a slightly higher rate of gene loss measured by pseudogene frequency, but frequent gene gain indicates gene loss is not the sole source of diversity in this pathogen.

Recombination is another important source of genetic diversity, generating antigenic variation in many pathogens²¹. PE/PPE proteins are thought to play a role in evasion of immune responses⁶¹ and have been suggested to be undergoing gene conversion¹², indicating a potential mechanism of antigenic variation. We confirm this hypothesis, though gene conversion was observed only in the PE_PGRS subfamily in the hypervariable PGRS domain (FIG. 12). This consistent observation of gene conversion could be due to the high GC-content within these genes as recombination hotspots are common in GC-rich regions⁵⁴. Furthermore, G-rich regions often form a non-B DNA structure, G-quadruplexes⁶², which have been associated with frequent recombination resulting in antigenic variation in other pathogens^{63,64}. The G4 structures are postulated to be signals for DNA nicking creating a double-stranded breaks⁶³, the first step required for subsequent recombination to occur (FIG. 6). However, a recent study in N. gonorrhoeae has demonstrated that G4s do not generate double-stranded breaks but may induce a nick or attract other factors to initiate recombination⁶⁵. Comparison of variants across serially isolated samples from the same M. tuberculosis-infected patient would be needed to confirm gene conversion as a mechanism of generating antigenic variation. The association of G4s to these gene conversion events would also need to be confirmed using similar experimental procedures described previously^{63,65-68}.

The consistent recombination in PE_PGRS genes indicates a benefit for survival in the human host, possibly linked to immune evasion due to their putative function as antigens³². Hypervariable regions of antigens are expected to mutate to evade recognition by immune cells⁶⁹. However, a previous report¹⁷ and the data in the present study (FIG. S8) discovered conserved T cell epitopes (MHC class I and class II) primarily in the PE domain rather than the PGRS domain, indicating the PE domain is involved in T cell recognition, (FIG. S8). Baena and Porcelli hypothesized that proteins containing MHC class I and class II epitopes distract CD4⁺ and CD8⁺ T cells⁷⁰. Such a decoy antigen has been described in M. tuberculosis, TB10.4, which was shown to elicit a CD8⁺ T cell response; but these primed CD8⁺ T cells could not recognize an M. tuberculosis-infected macrophage⁷¹. The conserved nature of the T cell epitopes suggests the PE domain of PE_PGRS proteins may also serve as a decoy antigen to distract CD4⁺ and CD8⁺ T cells.

In contrast, B cell epitopes were predicted within the PGRS domain indicating a potential interaction with B cells. In pathogens like N. gonorrhoeae, antigens undergo gene conversion to create new combinations of B cell epitopes, significantly affecting the binding affinity of existing antibodies. This mechanism requires a donor sequence that is often a pseudogene, which always invades the acceptor sequence through homologous recombination, resulting in a novel chimera of acceptor and donor epitopes (Deitsch et al. 2009; Palmer et al. 2016). This chimera requires the immune system to create new antibodies with high specificity to the mutated epitopes. We hypothesize the pgrs domain of pe_pgrs genes are undergoing a similar mechanism of recombination to create chimeras of the donor and acceptor sequences to promote immune evasion. This mechanism has been experimentally verified in several species (Palmer et al. 2016) but would need to be confirmed in M. tuberculosis with immune assays. Additionally, secretion is a required aspect of antigens to interact with immune cells. Several reports have noted that PE_PGRS proteins are exported to the surface of the cell (Banu et al. 2002; Brennan et al. 2001), while others have noted certain lineages lack the ability to secrete PE_PGRS proteins (Ates et al. 2018). Secretion of PE_PGRS proteins across all M. tuberculosis lineages would need to be confirmed.

Although care was taken in producing reference-quality genomes and annotation, results presented here and in other studies (Bolotin et al. 2015; Yang et al. 2018b) are affected by sample size and population structure. Additionally, M. africanum is known to cause human tuberculosis but was not included in the current study. A similar study on PacBio-sequence M. africanum genomes may elucidate clinically relevant genetic content. Diverse samples representing all M. tuberculosis lineages proportionally as well as isolated from several geographic regions would further support the conclusions made here. Additionally, M. bovis BCG may not contain all non-converted PE/PPE genes which can increase false negatives in a gene conversion detection analysis. A reference-quality, annotated genome from an ancestral strain closely related to M. tuberculosis would more accurately represent non-converted PE/PPE genes. Finally, accuracy in detecting linear B cell epitopes is low compared to detection of T cell epitopes. To identify more true positives, a higher threshold was chosen to identify more confident epitope locations, but this also increased false negatives. Experimental verification of antibody recognition to the inferred epitopes in PE_PGRS with predicted gene conversion would be needed to confirm the findings presented here.

This distraction and the predicted dearth of B cell epitopes observed in the PGRS domain (FIG. 7) suggests this domain elicits a T-cell-independent response. The T-cell-independent response does not activate helper T cells, failing to create memory B cells, affinity maturation, or immunoglobulin class switching⁷². Rather, these B cells generate low affinity antibodies (IgM) that have low specificity and high avidity but cannot retain lasting memory¹⁹. However, these B cells may produce M. tuberculosis-specific antibodies in later stages of the disease since granulomas have germinal center-like properties⁷³. This creates higher affinity antibodies, though not as high as those produced in true germinal centers like lymph nodes¹⁹. The slightly higher specificity of these antibodies pressures the bacteria to initiate evasion mechanisms, which may be caused by gene conversion within PE_PGRS genes. Although care was taken in producing reference-quality genomes and annotation, results presented here and in other studies^{7,36} are affected by sample size and population structure. Diverse samples representing all M. tuberculosis lineages proportionally as well as isolated from several geographic regions would further support the conclusions made here. Additionally, M. bovis BCG may not contain all non-converted PE/PPE genes which can increase false negatives in a gene conversion detection analysis. A reference-quality, annotated genome from an ancestral strain closely related to M. tuberculosis would more accurately represent non-converted PE/PPE genes.

Finally, accuracy in detecting linear B cell epitopes is low compared to detection of T cell epitopes. To identify more true positives, a higher threshold was chosen to identify more confident epitope locations, but this also increased false negatives. Experimental verification of antibody recognition to the inferred epitopes in PE_PGRS with predicted gene conversion would be needed to confirm the findings presented here.

This study demonstrates that relying solely on small variations compared to M. tuberculosis H37Rv limits resolution when studying genetic diversity within the species. We show that by producing reference-quality genomes and carefully annotating each genome, the genetic diversity of M. tuberculosis is evolving and expanding through gene gain, gene loss, and gene conversion. Overall, this study opens the door to understanding the remarkable genetic diversity of this deadly pathogen.

### Methods

### Sample Selection

One hundred and seventy-eight genomes were either re-sequenced due to low coverage in a previous dataset (n=113)⁷⁴, chosen from the strain collections at the World Health Organization Supranational References Laboratories in Stockholm, Sweden and Antwerp, Belgium based on their drug susceptibility profile and discordant genotype (n=41), or downloaded from the Sequence Read Archive (n=24).

Isolate selection for the Global Consortium for Drug-resistant tuberculosis Diagnostics (GCDD) population was described previously (Hillery et al. 2014). We selected 113 isolates from the GCDD to re-sequence on Pacific Biosciences polymerase 6-chemistry 4 (P6C4) that had the lowest coverage genome-wide in a previous sequencing run (Bioproject PRJNA353873). We chose an additional 41 isolates from World Health Organization Supranational References Laboratories in Stockholm, Sweden and Antwerp, Belgium to sequence based on their drug susceptibility profile and a corresponding discordant susceptibility genotyping result (i.e. resistant to amikacin but did not observe rrs A1401G on the MTBDRsl platform); or, they were chosen because mono-resistance was detected. Each strain was isolated from a single patient.

Additionally, publicly available PacBio sequences were queried within the SRA (Leinonen et al. 2011) database. FASTQ formatted files do not contain the required quality scores to run any SMRTAnalysis protocol. We therefore downloaded the HDF5 (. bax.h5) files, which contain all necessary sequencing quality metrics including the interpulse duration (IPD) for subsequent methylation analysis. The HDF5 files were downloaded using wget for the SRA accession numbers within the following Bioproject accession numbers: PRJEB21888, PRJBA270004, and PRJEB8783. Knockout strains and non-tuberculosis Mycobacteria were excluded from this study. This resulted in 19 genomes from public datasets. Two reference strains were additionally included: M. tuberculosis H37Rv (NC000962.3) and M. tuberculosis H37Ra (CP016972.1).

### Growth, DNA Extraction, and Sequencing

For isolates re-sequenced from the GCDD, DNA was extracted as described previously (Elghraoui et al. 2017). Briefly, cells were cultured on Middlebrook 7H11 agar plates and incubated until growth of a full lawn. Extraction was performed using Genomic-tips (Qiagen Inc., Hilden, Germany) following manufacturer's protocol. B1/RNase buffer solution was introduced to the culture, which was then homogenized by vortex mixing and inactivated at 80° C. for 15 minutes. Lysozyme was added and incubated for 30 minutes at 37° C. then proteinase K was added and incubated at 37° C. for 60 minutes. Wide-bore pipet tips were used to recover large DNA fragments and DNA purity and concentration were analyzed on Nanodrop 1000 Spectrophotometer (ThermoFischer Scientific, San Diego, Calif., USA).

Sequencing was also performed as previously described (Elghraoui et al. 2017). Briefly, each clinical strain was sequenced on the PacBio RS II platform with P6C4 and a 20 kb insert library preparation using a single SMRT cell per strain.

DNA extraction and sequencing for the publicly available HDF5 files were described previously (Berney et al. 2015; Zhu et al. 2015; Phelan et al. 2018).

Genome Assembly with Quality Control

FIG. 15 represents a unique assembly pipeline designed in-house, which can be used on any circular, haploid genome; and FIG. 15 illustrates a de novo assembly pipeline designed and utilized to assemble 179 M. tuberculosis clinical strains. This pipeline is not designed exclusively for M. tuberculosis and can be used on any haploid, circular genome. Details of each step are as follows:

Assembly: For isolates that were sequenced on multiple SMRT cells (for example multiple SRA entries sharing the same isolate name), all SMRT cell sequencing runs were combined. Hierarchical Genome Assembly Process (Chin et al. 2013) (HGAP) version 2 (RS_HGAP_Assembly.2 protocol) was used to assemble raw reads from HDF5 formatted files using default parameters. If HGAP2 was unable to assemble the genome (for example multiple contigs or no contig could be circularized [see #0]) or a misassembly was detected (see #0), the assembler Canu (Koren et al. 2017) (v1.6) was used with default parameters with the exception of the -pacbio-raw flag. Canu (Koren et al. 2017) was confirmed to be more accurate in assembly than several assemblers and was, therefore, the assembler of choice for isolates sequenced in 2017 (the publication year of Canu). Any genome that failed assembly using Canu was excluded from this study.

Circularization: For isolates sequenced prior to 2017, a custom Perl script was written to reset the genome start to the origin of replication with dnaA as the first gene (Brzostek et al. 2009); minimus2 from AMOS (http://amos.sourceforge.net) was then used to circularize each genome using default parameters, as described previously (Elghraoui et al. 2017). However, minimus2 could not successfully circularize all genomes due to multiple contigs or no detection of dnaA. For these cases, circulator (Hunt et al. 2015) was used to circularize using the ‘all’ command and the --genes_fa flag set to the H37Rv dnaA sequence to reset the genome order, default parameters were used otherwise. All isolates sequenced in 2017 (the publication year of circlator) or later were circularized with circlator. Any genome that failed to circularize after this step was excluded from this study.

Consensus error correction/polishing: Consensus error correction is an important step in producing a final consensus sequence of high-accuracy, particularly since HGAP2 and Canu are not sensitive enough to call consensus in repetitive regions. Error correction (or “polishing”) was performed as previously described (Elghraoui et al. 2017). Briefly, the circularized sequence was polished using BLASR+Quiver (RS_Resequencing protocol) in SMRTAnalysis (v2.3) to achieve a complete consensus sequence; three rounds were determined to be sufficient to attain a consensus sequence. The maximum coverage parameter in Quiver was set to 1000, default parameters were used otherwise. If a consensus sequence could not be achieved after three rounds of polishing, it was excluded from this study.

Assembly Quality Control: In order to validate the assembly, each genome was analyzed for potential breaks using PBHoney (English et al. 2014) and errors in consensus calling (see #3 above). We considered regions as “breaks” in PBHoney when the variant was supported by at least 10% of the total coverage at the location in the genome. Assemblies that failed this step (had breaks with at least 10% support) were excluded from further analysis in this study. Verifying the final consensus sequence was included in QC since iterating over polishing rounds is not a convergent process for repetitive genomes). A threshold of four variants in the final error correction run was chosen based on preliminary analyses indicating a high rate of single-base deletions (Error! Reference source not found.7) and significant breaks in the annotation when more than four variants were detected (see Sequence QC through Detection of Unique Genes below).

### Genome Annotation (AnnoTUB)

A custom annotation pipeline was created and designed specifically to annotate M. tuberculosis genomes. This annotation pipeline (AnnoTUB) first transfers annotations from a well-characterized reference, H37Rv, where sequence homology is high; next, performs ab initio annotation on regions where the reference annotation could not be transferred; followed by merging these two annotation methods; identifies orthologous annotations where the transfer and ab initio methods could not infer function, and, lastly, identifies PE and PPE genes. The characteristics of the M. tuberculosis genome allow for specific assumptions to be made (for example high genome conservation) which are discussed in detail below. The full pipeline of AnnoTUB is presented in FIG. 16.

Annotation Transfer: The first step in the annotation pipeline was transferring genes from a well-characterized reference. We prioritized this transfer step over ab initio methods because the reference annotation contains a larger number of experimentally verified functions, as compared to databases referenced by ab initio software (see Ab initio Annotation). In addition to a required high amino acid sequence identity, synteny is considered during transfer, which is absent in ab initio algorithms. Because of the high sequence conservation across M. tuberculosis strains, resulting in a 98.8% transfer of CDSs in H37Rv to M. tuberculosis F11 in a previous report (Otto et al. 2011), we assumed that the majority of the reference annotation will transfer to each clinical isolate's genome, with the exception of a few corner cases (see Merge section).

We transferred an in-house curated M. tuberculosis H37Rv annotation to all genomes included in this study. The custom curation was part of a comprehensive study of all hypothetical and ambiguously annotated proteins in H37Rv that included literature curation and function inferred from shared protein structure (Modlin et al. 2018). The Rapid Annotation Transfer Tool (RATT) (Otto et al. 2011) (v18) was used to perform the transfer. Although RATT allows for the configuration of custom start codons, start codons were not reset due to a lack of prioritization of the configured codons; we therefore relied on experimentally and computationally predicted start codons in the H37Rv annotation. The codons TGA, TAA, and TAG were configured as stop codons. We set the transfer type to be Strain (Transfer between strains), which has been tested on transferring annotations in H37Rv to clinical M. tuberculosis genomes (Otto et al. 2011). Within this setting, a 95% nucleotide sequence identity was implemented.

Ab initio Annotation: Although M. tuberculosis has high sequence conservation across strains (Otto et al. 2011), there are areas of the genome that continually change due to recombination, duplication, or spontaneous mutation (Phelan et al. 2016). In these regions, RATT will not be able to transfer annotations since the sequence identity has altered significantly or the sequence does not exist in the reference. When these regions were encountered, we relied on Prokka (Seemann 2014) (v2.6) to annotate. Prokka utilizes the gene prediction algorithm, Prodigal (Hyatt et al. 2010), to identify candidate gene coordinates and annotates these genes, prioritizing accuracy to minimize over-annotation, then references several databases to infer function based on sequence homology, domain search, and motif discovery. We ran Prokka twice on each genome; the two executions of Prokka differed by a single parameter, the --proteins flag with the custom H37Rv annotation as the argument (“Prokka-reference”). This flag prioritizes the user-defined input/database in annotating CDSs, then queries RefSeq, UniProt, and InterPro (for domains); however, we observed several corner cases (see below in

Merge Transfer and Ab Initio Annotations-Identifying partial annotations). We performed another run of Prokka without the --proteins argument, allowing Prokka to first query RefSeq for all CDSs (see below

Merge-Annotations with Prokka-no-reference). For both executions of Prokka, common parameters were as follows: --genus was set to Mycobacterium, --kingdom was set to bacteria, --rfam was flagged, --rnammer was flagged, --gram was set to pos, --usegenus was flagged, --centre was set to C, and --locus_tag was set to the isolate ID.

Merge Transfer and Ab Initio Annotations: We created a custom software, annomerge (implemented in Python), that merges the output of RATT (Otto et al. 2011) and Prokka (Seemann 2014) into a Genbank format file. annomerge accepts annotations in a hierarchical manner: first, taking confident annotations from the output of RATT; second, identifying confident annotations from Prokka-reference; third, filling in remaining annotations from Prokka-no-reference, with strict criteria (see below).

The following describes how corner cases were identified and resolved:

Non-contiguous CDSs: If a CDS annotation from RATT spanned non-contiguous regions (typically caused by a frameshift or ribosomal slippage in the coding region (Tatusova et al. 2016; National Center for Biotechnology Information) and represented as join's in the output), this annotation was not accepted and potential annotations of this region were queried for in Prokka.

Resolving Overlapping Annotations between RATT and Prokka-reference: In cases where a CDS overlaps between RATT and Prokka-reference, we presumed that either Prodigal did not set the start codon correctly or, because we did not allow RATT to reset the start codons, it's possible the gene starts with a rare codon and may be misannotated. If the two annotations did not share the same open-reading frame (i.e. did not share a start or stop codon), both RATT and Prokka annotations were accepted.

However, in cases where the frame was the same (typically shared the same stop codon), we compared the nucleotide sequence of the promoter region (assuming a leaderless transcript, 40 bp upstream of the gene start) from Prokka-reference coordinates to the sequence of the same region in H37Rv (AL123456.3) using blastn with default parameters. If a mutation was observed in the promoter region in the clinical isolate, the gene coordinates obtained from the Prokka-reference annotation were maintained, otherwise, the gene start was modified to match the relative start of the gene in H37Rv and the gene product annotation was transferred.

Verification of gene coordinates: Prodigal, executed in Prokka, is an unsupervised machine learning algorithm that uses the input genome sequence to learn gene structure properties such as codon statistics, ribosome binding site (RBS) motif usage, start codon frequencies (only ATG, GTG and TTG) and predicts gene coordinates in this genome (Hyatt et al. 2010). Although utilization of genome-specific metrics such as codon statistics make Prodigal an unbiased tool for gene predictions, some implicit biases may occur due to restriction of start codons to ATG, GTG and TTG. If a rare codon is the start, Prodigal would propagate until one of the three start codons was identified. This bias can be corroborated with the results of gene coordinate prediction of 62 experimentally verified genes in Mycobacterium tuberculosis H37Rv using Prodigal, where all 62 genes were identified with 58 having the correct start coordinate, indicating that the start site prediction by Prodigal has an accuracy of 93.6% for the set of 62 experimentally verified genes (Hyatt et al. 2010).

Because Prodigal was trained on a specific set of prokaryotic genomes and contains general rules about gene structure in prokaryotes (Hyatt et al. 2010), we executed Prodigal on the H37Rv genome and compared the gene coordinates to those predicted in the H37Rv annotation available on NCBI Protein database (AL123456.3). This resulted in an 88.09% concordance, with 480 genes having incorrect coordinates predicted by Prodigal. The incorrect gene coordinate annotations for the 480 genes were then used to recalibrate start codons for these genes in the clinical isolates.

When any of these 480 genes were encountered in a genome, the length of each gene was compared to the same gene in H37Rv. If the lengths were different, the nucleotide sequence upstream (40 bp) of this gene (the start was considered the same, relative start as that in H37Rv) was compared to the same upstream region in H37Rv to check for any mutations that may have caused Prodigal to choose a different start coordinate. If a mutation in the upstream region of the gene was found, the gene coordinates predicted by Prokka were accepted; otherwise, the coordinates were recalibrated to match that of H37Rv.

Identifying partial annotations: A partial sequence was classified as such if the alignment between the query (isolate) and subject (database hit in RefSeq or UniProt) satisfied the identity threshold (>95%) but was below the coverage threshold (<95%, subject or query coverage). Often, due to the use of an E-value rather than identity and alignment coverage, Prokka will annotate a CDS with the same functional information as the subject sequence even though the query coverage was extremely low. However, preliminary analyses indicated that these cases were often caused by a mutation, abolishing a stop codon, which extends the protein length; or, the mutation introduced a stop codon, prematurely, thereby truncating the protein. We assumed that in either case, the new sequence encodes an entirely different protein. If a CDS annotated by Prokka-reference was a partial sequence from a gene in H37Rv then the CDS, but not the gene name, was accepted and a downstream clustering or orthology-based method (mentioned below) was used to assign functional annotation.

CDSs with only domain annotation: If a CDS was annotated by Prokka (reference or no-reference) solely based on domain information (i.e. using InterPro alone), then a downstream clustering method (discussed in Clustering) was used to assign functional annotation for this CDS instead of direct transfer of functional annotation from Prokka. The InterPro results were maintained as a note.

Annotations with Prokka-no-reference: During the incorporation of CDSs from Prokka-no-reference, CDS functional annotations categorized as hypothetical protein that were not annotated with a gene name or Rv locus tag were excluded. These CDSs were not annotated as they were putative protein-coding genes and evidence of the proteins translated from these coding sequences have not been reported in the Mycobacterium genus yet(Seemann 2014). This was to ensure reduction of false positive annotations in this pipeline.

Annotation of non-CDS genomic elements: In addition to protein-coding genes, other genes and genomic features were incorporated as part of the annotation. Although these non-CDS genomic elements were incorporated into the annotation of clinical isolate for completeness, they were not used in any analyses in this study. Genomic features such as transfer RNAs (tRNAs), ribosomal RNAs (rRNAs), and signal peptides were annotated in each genome. Prokka executes RNAmmer(Lagesen et al. 2007) (v1.2) to identify and annotate rRNAs. tRNAs can be annotated by both RATT and Prokka; RATT uses tRNAscan-SE (Lowe and Eddy 1997) and Prokka uses ARAGORN(Laslett and Canback 2004) (v1.2) for the annotation of tRNAs. However, because ARAGORN is reported to have higher sensitivity compared to tRNAscan-SE (Laslett and Canback 2004), only tRNAs annotated by Prokka were accepted. Signal peptides were also accepted from Prokka.

### Clustering:

To identify genes that were not assigned a locus tag or gene name (annotated by Prokka) and identify which genes were the same across isolates, we clustered sequences first using CD-HIT (Li and Godzik 2006; Fu et al. 2012) then the Markov Cluster (MCL) (Enright et al. 2002) algorithm.

CD-HIT sorted a given set of sequences (here all CDSs present in the population) in descending length and the longest sequence was chosen as the representative sequence for the first cluster. Each sequence was then compared to this representative and considered part of the same cluster based on a 99% to 98% amino acid sequence identity and 100% coverage. If the query sequence did not meet these thresholds, it was considered the representative sequence of a new cluster. This process continued for all protein sequences in all the clinical isolates. The identity threshold was then reduced by 0.005% and the process began again based on the previous set of clusters, which created a cluster of clusters that was input into an all-against-all BLAST.

An all-against-all blastp (Altschul et al. 1990) alignment was implemented to create a matrix. The matrix represents a connection graph with sequences or CDSs as nodes and sequence identity as edges (greater than 95% identity and query and subject coverage). Weights were calculated by taking the logarithmic value (base 10) of the E-value from the blastp alignment between those two nodes or CDSs.

The matrix was then passed into MCL, which performs iterative rounds of matrix multiplication (expansion) and inflation (contraction) until there was no net change. To determine when there was no net change, the inflation parameter, which determines the granularity or tightness of the clusters, was set to 1.5 in our clustering step. The lower the inflation threshold the tighter the clusters (range of 1 to 10). This tighter threshold was chosen based on the assumed high genome conservation across M. tuberculosis strains.

### Assigning Gene Names

Based on the clusters created above, we isolated clusters that contained unnamed sequences (i.e. CDSs with randomly assigned locus tags), which resulted in three types of clusters: i) clusters containing only unnamed sequences, ii) clusters with unnamed sequences and named sequences with the same name, and iii) clusters with unnamed sequences and named sequences with different names. Here, named sequences were those with an Rv locus tag or a fully characterized gene name; unnamed sequences were those that Prokka identified as a CDS but was unable to identify a gene name (see

Genome Annotation (AnnoTUB)—

Annotation Transfer)

Steps to assign unnamed sequences for each case were as follows:

1. Clusters containing only unnamed sequences: The representative sequence of this cluster was compared to all CDSs in H37Rv (AL123456.3) using blastp to see if the unnamed sequence was homologous to a gene in H37Rv. Thresholds for this consideration were at least 95% amino acid sequence identity and 95% query and subject coverage. If no hit was identified the H37Rv proteome, the cluster was assigned a new gene name with the syntax ‘MTBxxxx’, where the ‘x’ indicates a digit, starting at MTB0001 assigned in order of the clusters output from MCL.

2. Clusters with unnamed sequences and named sequences with the same name: All unnamed sequences in these types of clusters were assigned the gene name given to the representative sequence of this cluster, if the representative was annotated with an Rv locus tag or a gene name. If the representative sequence was not annotated with a gene name or Rv locus, the first named sequence was identified within the cluster and all other unnamed sequences, including the representative sequence, were annotated with this gene name.

3. Clusters with unnamed sequences and named sequences with different names: Each unnamed sequence was compared to all named sequences in the cluster using blastp to determine which named gene the unnamed sequence was most identical to using a 95% threshold of identity and coverage. The unnamed sequences were annotated with a gene name based on the best hit to a named sequence. If the thresholds were not met, the gene was assigned a ‘MTB’ gene name.

All clusters that only contained gene names (either multiple or just one and no unnamed sequences), were not modified or processed further.

Annotations were updated with the newly assigned gene names using a custom Python script.

### Orthologous Annotation

Thresholds used in previous steps were designed to be strict to offer confidence in the annotation. All thresholds were decided based on RATT and Prokka defaults. However, this leaves approximately 3300 genes without any functional annotation across 99 genomes. We therefore sought to annotate these unannotated genes using orthology via the eggNOG-Mapper software (Huerta-Cepas et al. 2017), which parses the eggNOG database based on an input multisequence FASTA file. This software utilizes two annotation mapping algorithms: DIAMOND and HMMER. The DIAMOND algorithm performs a BLASTP across all the protein sequences in the eggNOG database to identify the best seed ortholog match for the query sequence. HMMER on the other hand, parses all Hidden Markov Models (HMMs) of orthologous groups in the eggNOG database and identifies the best match. Once the orthologous group is identified, phmmer is used to identify the best sequence hit within the orthologous group. In both instances, the seed ortholog, which is the best sequence hit to the query, is identified. This seed ortholog is then used to infer fine-grained orthologs, based on the pre-computed Cluster of Orthologous Group (COG) this seed ortholog belongs to in the eggNOG 4.5 database (Huerta-Cepas et al. 2017). An E-value threshold of 10⁻⁶ was set to avoid transfer of functional annotation from distant orthologs. Functional annotation was transferred from orthologs that were taxonomically closest to the query (this parameter was automatically adjusted for each query by eggNOG-mapper), minimizing false/mis-annotations.

We chose to first process all unannotated genes (identified in the

Clustering: step) using the DIAMOND algorithm since this algorithm is more time-efficient and has a higher sensitivity as HMMER when the source organism of the query proteins is well-represented in the eggNOG database (Powell et al. 2012). We set the E-value threshold to 10⁻⁶ (the default in Prokka). Genes that could not be annotated with the DIAMOND algorithm were passed into the HMMER algorithm. The annotation of the seed ortholog was then picked as the best annotation. Additional functional information such as experimentally verified Gene Ontology (GO) terms and KEGG pathways were transferred from the orthologs that were taxonomically closest to the query.

The gene feature was updated with the gene name predicted by eggNOG apart from PE/PPE genes. Genes annotated by eggNOG-mapper as a PE or PPE family proteins were not updated because these genes more likely aligned across the conserved region, not by the variable region, which is what defines different PE/PPE genes within each family (Cole et al. 1998). PE/PPE gene assignments is described in-text. (Identifying PE and PPE Genes). Seqret v.6.6.0.0 (http://emboss.sourceforge.net/apps/release/6.6/emboss/apps/seqret.html) was used to convert Genbank format to GFF3 for input into Roary (see below Error! Reference source not found.).

AnnoTUB and all corresponding modules are available at https://gitlab.com/LPCDRP/annotub.

### Sequence QC Through Detection of Unique Genes

Although we implemented a strict quality control protocol post-assembly, we sought to further QC each assembly by: 1) annotating each genome that passed assembly and consensus QC; 2) execute a pangenome analysis (see Error! Reference source not found. below for details); 3) tabulate the number of unique genes per genome. Error! Reference source not found. a demonstrates an outlier with a higher than average number of unique genes as compared to their closest relative (determined by SNP Hamming distance). We postulated that these outliers may have a high amount of single-base deletions. We, therefore, calculated the deletion-to-insertion ratio and each outlier had two times higher the amount of single-base deletions than insertions. This outlier was excluded due to potential sequencing errors (FIG. 17A-B).

FIG. 17A-B: Scatter density plots of the number of unique genes determined by a pangenome analysis as a function of the minimum Hamming SNP distance of each isolate for a) 98 genomes that passed assembly quality control (the red point indicates an outlier genome with a high number of unique genes that was more than its closest relative in the data set); and b) 97 genomes with the removal of the outlier from a.

### Pangenome

To create pan, core, and accessory genomes, we used the software Roary (Page et al. 2015). We separated isolates into five populations: all isolates in the population, Euro-American isolates, East Asian isolates, East African-Indian isolates, and Indo-Oceanic isolates. The ‘all’ isolates population provided a global perspective of genes in clinical isolates while the lineage separation allowed analysis of lineage-specific effects, genome-wide. The pangenome is defined as the unique union of all genes in the given population; the core genome is considered the compendium of genes that are represented in 99% of the population; and the accessory genome is the remaining genes that did not meet the core genome threshold but existed in more than one genome. A 99% threshold for the core genome was chosen since the population consists of the same species of a clonal bacteria, which is also recommended by Roary. We split inparalogs to identify copies of genes that may be part of the accessory genome; we flagged the −e option to create a core alignment using PRANK; default parameters were used otherwise (apart from the custom edits described below).

Custom Edits to Roary: A few modifications were implemented to the Roary source code to account for the clonality and high sequence similarity of M. tuberculosis genomes. Specific details to the modifications were as follows:

More Strict Thresholds in blastp Parallel All-Against-All: We modified the criteria within the parallel all-against-all blastp execution to be stricter when identifying aligned pairs. Our criteria were: at least 95% identity, query coverage, and subject coverage to pass. This was implemented to match thresholds used within AnnoTUB and provide matching results in the cluster output.

“Collapsing” Clusters that Share Gene Names: We favored the annotation of a given CDS over the clustering generated by Roary. This translates to iterating over the output of MCL (after splitting paralogs) and identifying clusters that were annotated as the same gene name (except for PE/PPE genes annotated by eggNOG, see

Genome Annotation (AnnoTUB)—

Orthologous Annotation). Clusters that were not considered inparalogs were collapsed if they shared a gene name.

### Gene Essentiality of the Core Genomes

We utilized the H37Rv gene classified as essential or otherwise from DeJesus et al (DeJesus et al. 2017). This study had five classifications for essentiality in H37Rv: essential (ES), essential domain (ESD), growth defect (GD), growth advantage (GA), and non-essential (NE). We classified ES and ESD as essential and GD, GA, and NE as non-essential. We developed a custom Python script to compare each lineage's core genome to the list of essential and non-essential genes, assuming essential genes are more likely conserved across strains.

### Genes not in H37Rv

Classifying genes not in H37Rv: We created four categories to classify these genes:

1. Within RvD1-6/TbD1
a. Amino acid sequences from genes not in H37Rv were aligned to genes within RvD1-6 using blastp. Table X below represents the Genbank accession numbers and associated publications that reported the RvD and TbD1 sequences.

2. In M. tuberculosis CDC1551
a. Amino acid sequences from genes not in H37Rv were aligned to the proteome of CDC1551 (Genbank accession AE000516.2) and hits were accepted if the amino acid sequence identity and subject and query coverage were at least 95%.

3. Transposases
a. A gene was considered a transposase if the annotated product was Transposase

4. Duplications
a. See Detecting Gene Duplications in the in-text Methods section

5. Pseudogenes
a. See Identifying Pseudogenes in-text Methods section

6. Genes that containing a single-base indel or contained an indel in a homopolymer region
a. This was identified by performing a BLASTN alignment to all CDSs in H37Rv and a threshold of at least 95% nucleotide identity was required. A single-base indel must exist in the alignment or an indel must exist in a homopolymer (for example AA, CC, GG, TT)

7. Gene truncations
a. This was identified by performing a BLASTN alignment to all CDSs in H37Rv and a threshold of at least 95% nucleotide identity was required. The alignment length was required to be shorter than the subject (best matched gene in H37Rv). Furthermore, Evalue was required to be no higher than 0.01.

8. Gene extensions
a. This was identified by performing a BLASTN alignment to all CDSs in H37Rv and a threshold of at least 95% nucleotide identity was required. The query (gene no in H37Rv) was required to be longer than the subject (best matched gene in H37Rv) by at least one nucleotide. This translates to a query coverage of greater than 100%. Furthermore, Evalue was required to be no higher than 0.01.

9. Partial alignments
a. This was identified by performing a BLASTN alignment to all CDSs in H37Rv and a threshold of at least 0.01 Evalue. The alignment was required to be less than both the subject (gene in H37Rv) and query (gene in H37Rv)

TABLE X
Accession numbers and corresponding reference for all
deletions identified in M. tuberculosis H37Rv with
respect to other members of the M. tuberculosis complex
and M. tuberculosis CDC1551 strain (RvD, TbD1)

| Genbank Accession | Reference |
|---|---|
| AJ426486 | Brosch et al PNAS (2002) |
| Y18605.1 | Gordon et al Mol. Microbiol. (1999) |
| Y18606.1 | Gordon et al Mol. Microbiol. (1999) |
| AE000516.2 (MT1812, MT1813) | Fleischmann et al J. Bacteriol. (2002) |
| AE000516.2 (MT2423) | Fleischmann et al J. Bacteriol. (2002) |
| AE000516.2 (MT3427, MT3428) | Fleischmann et al J. Bacteriol. (2002) |
| AE000516.2 (MT2420, MT2421) | Zheng et al PLoS One (2008) |

### Identifying Most Homologous Gene in H37Rv

To identify the closest H37Rv gene relative for all genes in the not in H37Rv gene list, we performed a pairwise all-against-all BLASTN for all genes not in H37Rv against all genes in H37Rv. We chose the closest gene relative based on the following criteria. Of all BLASTN hits, choose the hit with
With no higher than 0.1 Evalue, and
Must have the lowest Evalue of all BLASTN alignments
   All code generated for the pangenome analysis (including the Python script created to collapse the Roary clusters) is available at https://gitlab.com/LPCDRP/pangenome.

### Assembly

The HGAP2⁷⁵ protocol from SMRT Analysis v2. 3™ was used to assemble each genome (isolates sequenced on multiple SMRT cells were combined). If HGAP2 could not produce a quality genome, Canu⁷⁶ was used to attempt to resolve the genome into a single contig. Due to the similarity of the algorithms between the two assemblers and a previously reported minimal difference in consensus sequences of E. coli K12 between the two assemblers⁷⁷, we did not expect many of these genomes to be resolved by Canu. Each genome was circularized with either minimus2 (http://amos.sourceforge.net) (if HGAP2 was the assembler) or circlator⁷⁸ (if canu was the assembler). Consensus polishing was performed over three rounds of alignment and variant calling to obtain a final consensus sequence. Several quality control measures were implemented: successful circularization, accuracy of the final consensus sequence, and/or misassemblies detected by PBHoney⁷⁹. Detailed parameters and thresholds are discussed in the Supplemental Information.

### Annotation

A custom annotation pipeline, Annotate TUBerculosis (AnnoTUB) (https://gitlab.com/LPCDRP/annotub), was designed to annotate M. tuberculosis genomes. To reduce false positive annotations, we relied on an initial annotation transfer step, performed by the Rapid Annotation Transfer Tool (RATT) (Welcome Trust Sanger Institute), which has been previously validated in M. tuberculosis genomes³³. Because H37Rv has the most well-characterized functional CDSs as well as experimentally confirmed gene coordinates, we used H37Rv as the reference annotation to transfer. We used RATT's Strain flag, which implements a 95% identity to identify conserved genomic regions. Large stretches of unannotated regions were observed; therefore, we attempted to fill these regions with an ab initio approach, implemented by Prokka first using a reference database (H37Rv) and then prioritizing public databases such as UniProt and InterPro/PFAM. An Evalue threshold was set to 10⁻⁶, the default in Prokka. We further verified any annotation to a gene in H37Rv using a 95% amino acid identity and subject and query coverage. For any predicted CDSs lacking functional annotation, we queried EggNOG⁸⁰ (European Molecular Biology Laboratory (EMBL)) using the eggnog-mapper⁸¹ for orthologous genes, requiring H37Rv to be the seed orthologue. We first utilized the DIAMOND method using an Evalue threshold of 10⁻⁶ to match the threshold set in Prokka (Victorian Bioinformatics Consortium); we then queried any remaining CDSs lacking functional annotation using the HMM method. A custom Python script (https://gitlab.com/LPCDRP/annomerge) merged all of these annotations into a single annotation file for each genome, incorporating information in a hierarchical manner: i) transferred CDSs from H37Rv, ii) ab initio annotations, iii) EggNOG orthologues.

rRNAs were annotated using Prokka's implementation of HMMER⁸². tRNAs were annotated using Prokka's implementation of ARAGORN⁸³.

### Assembly Quality Scores

We further sought to score each assembly based on a previously defined set of criteria used to quantify assembly quality³¹, including sequence quality score, rRNA score, tRNA score, and Pfam-A essentiality score. Because the first three scores were 1.0 across all genomes, calculating an overall quality score using deviations in variance was not applicable. Instead, all four scores were averaged for each genome.

### Lineage Determination

For isolates that were re-sequenced, lineage information was obtained by inserting the MIRU-VNTR and spoligotype patterns determined previously⁸⁴ into TBInsight⁸⁵. For all other genomes, a custom script, MiruHero (https://gitlab.com/LPCDRP/miru-hero), determined lineage.

### Generating Phylogenetic Trees

First, SNPs were called using dnadiff (Marcais et al. 2018) (v1.3) with M. tuberculosis H37Rv (NC_000962.3) as the reference and called SNPs and small indels with default parameters. A custom Perl script converted the dnadiff SNP output into a VCF v4.0 file and the Variant Effect Predictor (McLaren et al. 2016) (v87) determined the consequence of each variant.

Next, a multisequence FASTA file was created by concatenating the SNP calls present in the VCF of each genome. If a SNP was not called in a genome, the reference base was inserted. This resulted in 20549 SNPs.

Finally, the SNP FASTA was uploaded to the CIPRES Science Gateway (Miller et al. 2010). RAxML (Stamatakis 2014) v8.2.10 created a maximum likelihood phylogenetic tree using the GTR+GAMMA substitution model, 100 bootstraps, and ascertainment bias correction with Lewis. The SNP tree was rooted using Mycobacterium bovis (AM408590.1) and Mycobacterium canetti (NC_019951.1) as outgroups.

Tree visualization and analysis were performed with the Interactive Tree of Life (iTOL) (Letunic and Bork 2016).

### Detecting Gene Duplications

Gene duplications were identified using two methods: i) sequence homology clustering and ii) annotated as the same gene name from AnnoTUB. The first was performed by executing CDHIT on each proteome using a 95% amino acid identity and overall coverage to cluster sequences. Any cluster with more than one gene was considered a duplication event. For the second, gene names were compared across each genome and if there were more than one CDS with the same gene name, the name and copy number was noted.

To search for embR2 in M. canettii, EmbR2 from M. tuberculosis CDC1551 was queried across all M. canettii strains available in the nr database using BLASTP with default parameters.

Gene Ontology (GO) enrichment analysis

All known protein coding genes in H37Rv (GenBank accession AL123456.3) and all genes annotated by AnnoTUB were compiled in multisequence amino acid FASTA file. This file was input into the eggNOG-Mapper (Huerta-Cepas et al. 2017) software to retrieve experimentally verified GO terms for each protein coding gene. GO annotations associated with a protein sequence have evidence codes as additional information (Gene Ontology Consortium 2017). The evidence code corresponds to varying degrees of confidence and the source for inference of function of the protein. The evidence for GO terms range from the most confident source of annotation, ‘inferred from experiment’, to the least confident ‘inferred from electronic annotation’. For this study, only GO terms denoting functions inferred from experimental methods (including, direct enzymatic assays, mutant phenotype, gene expression pattern) were included from eggNOG-Mapper (Huerta-Cepas et al. 2018, 2017) for each protein-coding gene. Additionally, a custom Python script was used to retrieve GO term predictions based on structural homology, as part of the study of all proteins of unknown function in H37Rv (Modlin et al. 2018). A dataset of these GO terms, either known to be experimentally verified for the genes, or annotated based on structural homology, was used as the custom annotation for GO enrichment analysis.

GO term for candidate set of genes was performed using a custom R script using the GOfuncR package (Grote 2018) (https://github.com/sgrote/GOfuncR). For all candidate sets, the pangenome was used as the background dataset to determine enrichment.

### Pangenome

Pan, core, and accessory genomes were created using Roary³⁸. Roary was executed on all isolates. Details on parameters for Roary are described in the Supplemental Information.

Identical genes were isolated from the core genome using CDHIT⁸⁶. First, all protein alleles for core genes were compiled across all isolates. Next, CDHIT was executed for each gene requiring 100% amino acid sequence identity, 100% query coverage, and 100% subject coverage (100% identical). A gene was considered identical if and only if all alleles were 100% identical in at least all 97 M. tuberculosis clinical genomes. A gene was not excluded if H37Rv and/or H37Ra were also 100% identical.

To determine enrichment of essential genes, genes were compared to the essential genes described in DeJesus et al³⁹. Genes listed as Essential or Growth Defect were considered essential; genes listed as Not Essential or Growth Advantage were considered not essential. Enrichment of essential genes was determined with a Fisher's Exact test with a p-value of less than 0.05 to be considered significant. Functional categories were assigned to genes that were identical but considered not essential including transcription factors⁸⁷, hypothetical proteins (described as “conserved hypothetical s” in Mycobrowser⁸⁸), toxin/antitoxins⁸⁹, and ribosomal proteins (if the annotation had the term “ribosomal” or “ribosome”). A functional enrichment was performed for functional categories transcription factors, hypothetical proteins, and toxin/antitoxins using a Fisher's Exact test. A p-value of less than 0.05 was considered significant.

### Finding and Classifying Genes not in H37Rv

Briefly, the pangenome was parsed for genes that did not exist in H37Rv. Criteria for the categories (duplications, transposases, frameshifted, truncations, extensions, and pseudogenes) are described in full in the Supplemental Information. The closest gene relative in H37Rv was considered the best matching based on lowest E-value and E-value was less than 0.1. Full details are described in the Supplemental Information.

### Identifying Pseudogenes

Pseudogene classification was based on methods by previous authors (Danneels et al. 2018; Carlier et al. 2013). Briefly, intergenic regions between annotated CDS features were queried against the pangenome protein database using the NCBI blastx+ program (Altschul et al. 1990) (v2.6.0) with default parameters. A pseudogene was identified in an intergenic region if it met the following criteria: i) the pairwise alignment had an E-value less than or equal to 10E-6; ii) the alignment query coverage and identity was greater than or equal to 50%; iii) putative pseudogenes shared synteny with the functional homolog in at least one clinical isolate; iv) the putative pseudogene had a frameshift or premature stop codon that affected more than 20% of the gene (using the tfasty (Pearson 2000) program (v36) with default parameters).

Conversely, for annotated CDSs, a blastp (v2.6.0) pairwise all-against-all of the pangenome was executed. If a gene aligned to multiple CDSs, the best hit was chosen based on lowest E-value. An annotated CDS was classified as a pseudogene if i) aligned less than 80% and greater than 10% the length of the respective homolog in the pangenome ii) shared amino acid identity of at least 50%, iii) the alignment Evalue less than or equal to 10E-6, iv) the query length must be less than the subject length, v) the putative pseudogene had a frameshift or premature stop codon that affected more than 20% of the gene (using the fastx (Pearson 2000) program (v36) with default parameters)(Carlier et al. 2013). Finally, similar to a previous study (Bolotin et al. 2015), pseudogenes must be present in less than 75% of the study population, known as the “near core” genome.

Statistical tests were then performed to determine if there was more genome contraction (pseudogenes) than amplification (duplications). To account for lineage being a confounding factor, for each genome, the nucleotide lengths of all classified pseudogenes and, separately, duplications were summed and a proportion to the genome size was calculated. The difference between the proportion of pseudogene length and duplication length was calculated per genome and differences were grouped based on lineage. An ANOVA test was performed to determine if genome contraction vs amplification was more pronounced in specific lineages. These same calculations were performed for the frequency of pseudogenes and duplications and the proportion to the number of CDSs per genome was calculated.

A paired t-test was then used to determine if there was significantly more pseudogene than amplification (by length and frequency) for each lineage. Both the ANOVA and paired t-test were calculated using the scipy (Virtanen et al. 2019). A p-value of less than 0.05 was considered significant.

### Identification of PE/PPE Genes

We sought to identify a more sensitive motif than the originally described “PE” at codons 8 and 9, and “PPE” at codons 7, 8, and 9, by searching for the longest motifs that were present within the PE and PPE proteins in H37Rv. All PE/PPE protein sequences from H37Rv were compiled. Several PE/PPE protein sequences were excluded from the creation of the motif (Table S8, or FIG. 27). The first set were excluded for various reasons reported previously (Gey van Pittius et al. 2006; Riley et al. 2008). Additionally, we postulated that the motifs for each family would exist within the conserved domain of the proteins (within the first 110 amino acids of PE and 180 amino acids for PPE (Cole et al. 1998; Gey van Pittius et al. 2006)). As such, proteins with a length less than the conserved domain were also excluded (Table S8, or FIG. 27). This final sequence list resulted in 83 PE sequences and 61 PPE sequences (derived from GenBank accession AL123456.3).

For the PE motif discovery using MEME (Bailey et al. 2015) (v5.0.5), -minw was set to 40, -maxw was set to 70, and -nsites was set to 83. For the PPE motif discovery using MEME, -minw was set to 70, the -maxw was set to 180, and the -nsites was set to 61. This resulted in one PE motif that was 56 amino acids long, with an E-value of 7.2e-2536; and one PPE motif that was 84 amino acids long with an E-value of 8.6e-2422 (FIG. 10A-B, Data Files 1 and 2 contain the position weight matrices for both position weight matrices for PE and PPE, respectively).

FIG. 10A-B illustrate logo plots for amino acid sequence motifs for a) PE; and, b) and PPE generated from 84 PE and 61 PPE amino acid sequences from M. tuberculosis H37Rv (Genbank accession AL123456.3).

To determine a threshold of high confidence when searching for these motifs in the clinical strains, we queried for these motifs using Finding Individual Motif Occurrences (FIMO) in the H37Rv proteome (AL123456.3). We then iteratively calculated the false positive rate and the false negative rate in a custom R script to find a corresponding p-value threshold. We remained conservative by favoring a higher false negative rate. For PE, a threshold of 2.14e-16 yielded a false positive rate of 0.0% and a false negative rate of 6.06%. For PPE, a threshold of 1.21e-33 yielded a false positive rate of 0.0% and a false negative rate of 7.25%. The genes that were called false negative were among the sequences excluded from the creation of the motif (Table S8, or FIG. 27). Importantly, the sensitivities and specificities were higher than the original PE and PPE motifs in detecting true PE and PPE proteins (Table S7, or FIG. 26).

To identify all possible PE/PPE genes in the 99 genomes included in this study, we queried for our created motifs in all pangenome protein sequences, using the determined p-value threshold for each family.

Sublineage motifs were reported previously (Gey van Pittius et al. 2006) and were converted into regular expressions. A custom Python script iterated across all PE or PPE amino acid sequences and identified which sublineage motifs existed in the sequence. The entire protein sequence was queried since the sublineage motifs exist in the variable region of these genes.

All code developed to analyze the PE/PPE gene families is available at https://gitlab.com/LPCDRP/pe-ppe.

### Gene Conversion in PE/PPE Genes

To detect gene conversion, non-converted versions of the genes must be present in the analysis. PE/PPE genes were identified using the same method described above in M. bovis BCG Pasteur (Genbank accession AM408590.1) and subfamily motifs were also identified as described in the Supplemental Information. Three isolates were randomly chosen to represent each lineage, with the exception of lineage 7 in which only one isolate represented this lineage in the study. Ab initio predicted subfamily genes were isolated for each representative isolate and concatenated with all genes within the respective subfamily from M. bovis BCG Pasteur, creating a multisequence FASTA for each comparison (n=78).

Each multi-sequence FASTA was aligned with CLUSTALO⁹⁴ (v1.2.1) using default parameters, and then uploaded into RDP4⁴⁵. The following methods were chosen to evaluate potential recombination events: RDP⁹⁵, GENECONV⁹⁶, Chimaera⁹⁷, MaxChi⁹⁸, Bootscan⁹⁹, SiScan¹⁰⁰, and 3SEQ¹⁰¹. Default parameters were used with the following exceptions: highest acceptable P-value was set to 0.01; GENECONV g-scale was set to 2; Bootscan was set to “Use Neighbor-Joining trees” with the cutoff percentage set to 90, and the substitution model was set to “Kimura Model”; MaxChi was set to “Variable window size”. To confidently call a recombination event and reduce false positives, at least five methods were required to detect the event. Additionally, RDP4 creates two neighbor-joining trees to assess whether the recombinant sequences were correctly identified. The first tree was built using the portion of sequences outside of the recombinant region. In this tree, the putative acceptor gene and the putative recombinant gene should share homology and exist in the same clade separate from other input sequences (FIG. 12). The second tree features the portion of sequences within the recombinant region, where the putative donor gene and the putative recombinant gene share homology, therefore existing in the same clade (FIG. 12).

Each predicted recombinant event that passed our thresholds was assessed in this manner, and subsequently considered to be correctly identified. Recombination breakpoints were manually corrected to MaxChi-identified statistically optimal positions, as recommended by the developer of RDP4. All recombination events meeting these criteria were recorded (Table S10, or FIG. 29).

After identifying conversion events in the representative isolate set, we sought to identify genes in the same syntenic region in isolates not directly tested for conversion. To this end, the genomic location of the gene directly after the converted gene was identified. As the location of these genes was highly conserved across the genomes, we searched for the location of this conserved gene and identified the gene directly before it. This was performed for all remaining isolates within each lineage in which gene conversion was found.

### Identifying G4s

G4Hunter¹⁰⁴ was used to identify guanine triplet or quartet quadruplexes (G4s) (https://github.com/AnimaTardeb/G4Hunter). The score threshold was set to 1.5 and the window was set to 20 based on previous accuracy calculations¹⁰⁴. A two-proportion Chi-square test¹⁰⁵ was used to determine if the number of G4s in a given subfamily of PE or PPE was significantly greater than other subfamilies.

### Epitope Detection

T Cells

T cell epitope prediction was replicated as previously described¹⁷. Briefly, the Immune Epitope Database (IEDB)¹⁰⁶ command-line epitope prediction program using the NetMHCpan¹⁰⁷ (v4.0) and NetMHCIIpan¹⁰⁸ (v3.1) methods were used for predicting CD8⁺ and CD4⁺ T cell, respectively. For MHC class I prediction, the HLA-A and HLA-B 9-peptide long alleles described previously were used¹⁷. For MHC class II prediction, the HLA-DR alleles described previously were used¹⁷. Threshold cutoff values corresponding to IC50 values of <50 nM for high affinity binding and <500 nM for intermediate affinity binding were used for both MHC class II and class I predictions. All PE_PGRS protein sequences (n=282) were included in this analysis.

B cells

Enrichment of linear B-cell epitopes was queried for within all PE_PGRS proteins using iBCE-EL¹⁰⁹ with default parameters. Proteins with enrichment scores meeting or exceeding a score of 0.35 or higher were considered enriched for B cell epitopes. Linear B cell epitopes queried within PE_PGRS proteins that had gene conversion and were enriched for B cell epitopes determined by iBCE-EL using Bepipred 2.0¹¹⁰ in IEDB¹⁰⁶ with default parameters. A threshold of 0.55 was chosen to increase specificity, reducing false positives, though sensitivity was sacrificed.

Complete genomes for 97 M. tuberculosis clinical strains are deposited under BioProject accessions PRJNA555636 and PRJEB8783.

CODE AVAILABILITY: Custom code including AnnoTUB and associated pangenome analyses are located at https://gitlab.com/LPCDRP/

### REFERENCES

Akhter Y, Ehebauer M T, Mukhopadhyay S, Hasnain S E. 2012. The PE/PPE multigene family codes for virulence factors and is a possible source of mycobacterial antigenic variation: Perhaps more? Biochimie 94: 110-116.
Altschul S F, Gish W, Miller W, Myers E W, Lipman D J. 1990. Basic local alignment search tool. J Mol Biol.
Andreatta M, Karosiene E, Rasmussen M, Stryhn A, Buus S, Nielsen M. 2015. Accurate pan-specific prediction of peptide-MHC class II binding affinity with improved binding core identification. Immunogenetics 67: 641-650.
Andreu N, Gibert I. 2008. Cell population heterogeneity in Mycobacterium tuberculosis H37Rv. Tuberculosis (Edinb) 88: 553-9.
Ates L S, Sayes F, Frigui W, Ummels R, Damen M P M, Bottai D, Behr M A, van Heijst JWJ, Bitter W, Majlessi L, et al. 2018. RD5-mediated lack of PE_PGRS and PPE-MPTR export in BCG vaccine strains results in strong reduction of antigenic repertoire but little impact on protection. PLoS Pathog 14: 1-29.
Azhikina T, Gvozdevsky N, Botvinnik A, Fushan A, Shemyakin I, Stepanshina V, Lipin M, Barry C, Sverdlov E. 2006. A genome-wide sequence-independent comparative analysis of insertion-deletion polymorphisms in multiple Mycobacterium tuberculosis strains. Res Microbiol 157: 282-290.
Baena A, Porcelli S A. 2009. Evasion and subversion of antigen presentation by Mycobacterium tuberculosis: REVIEW ARTICLE. Tissue Antigens 74: 189-204.
Bailey T L, Johnson J, Grant C E, Noble W S. 2015. The MEME Suite. Nucleic Acids Res 43: W39-W49.
Banu S, Honoré N, Saint-Joanis B, Philpott D, Prévost MC, Cole S T. 2002. Are the PE-PGRS proteins of Mycobacterium tuberculosis variable surface antigens? Mol Microbiol 44: 9-19.
Bedrat A, Lacroix L, Mergny J-L. 2016. Re-evaluation of G-quadruplex propensity with G4Hunter. Nucleic Acids Res 44: 1746-1759.
Bergthorsson U, Andersson D I, Roth J R. 2007. Ohno's dilemma: Evolution of new genes under continuous selection. Proc Natl Acad Sci USA 104: 17004-17009.
Berlin K, Koren S, Chin C S, Drake J P, Landolin J M, Phillippy A M. 2015. Assembling large genomes with single-molecule sequencing and locality-sensitive hashing. Nat Biotechnol 33: 623-630.
Berney M, Berney-Meyer L, Wong K-W, Chen B, Chen M, Kim J, Wang J, Harris D,
Parkhill J, Chan J, et al. 2015. Essential roles of methionine and S-adenosylmethionine in the autarkic lifestyle of Mycobacterium tuberculosis. Proc Natl Acad Sci 112: 10008-10013. http://www.pnas.org/lookup/doi/10.1073/pnas.1513033112.
Bolotin E, Hershberg R, B F, K O, I T. 2015. Gene Loss Dominates As a Source of Genetic Variation within Clonal Pathogenic Bacterial Species. Genome Biol Evol 7: 2173-2187.
Boritsch E C, Khanna V, Pawlik A, Honore N, Navas V H, Ma L, Bouchier C, Seemann T, Supply P, Stinear T P, et al. 2016. Key experimental evidence of chromosomal DNA transfer among selected tuberculosis-causing mycobacteria. Proc Natl Acad Sci 113: 9876-9881. http://www.pnas.org/lookup/doi/10.1073/pnas.1604921113.
Brennan M J, Delogu G, Chen Y, Bardarov S, Kriakov J, Alavi M, Jacobs J. 2001. Evidence that mycobacterial PE_PGRS proteins are cell surface constituents that influence interactions with other cells. Infect Immun.
Brosch R, Gordon S V., Gamier T, Eiglmeier K, Frigui W, Valenti P, Dos Santos S, Duthoy S, Lacroix C, Garcia-Pelayo C, et al. 2007. Genome plasticity of BCG and impact on vaccine efficacy. Proc Natl Acad Sci USA.
Brosch R, Gordon S V, Marmiesse M, Brodin P, Buchrieser C, Eiglmeier K, Gamier T, Gutierrez C, Hewinson G, Kremer K, et al. 2002. A new evolutionary scenario for the Mycobacterium tuberculosis complex. Proc Natl Acad Sci USA 99: 3684-9. http://www.ncbi.nlm.nih.gov/pubmed/11891304 (Accessed Oct. 14, 2019).
Brosch R, Philipp W J, Stavropoulos E, Colston M J, Cole S T, Gordon S V. 1999. Genomic analysis reveals variation between Mycobacterium tuberculosis H37Rv and the attenuated M. tuberculosis H37Ra strain. Infect Immun.
Brown W C, Brayton K A, Styer C M, Palmer G H. 2003. The hypervariable region of Anaplasma marginale major surface protein 2 (MSP2) contains multiple immunodominant CD4+T lymphocyte epitopes that elicit variant-specific proliferative and IFN-gamma responses in MSP2 vaccinates. J Immunol 170: 3790-8.
Cahoon L A, Seifert H S. 2009a. An alternative DNA structure is necessary for pilin antigenic variation in Neisseria gonorrhoeae. Science 325: 764-7.
Cahoon L A, Seifert H S. 2009b. An Alternative DNA Structure Is Necessary for Pilin Antigenic Variation in Neisseria gonorrhoeae. Science (80-) 325: 764-767.
Cahoon L A, Seifert H S. 2013. Transcription of a cis-acting, Noncoding, Small RNA Is Required for Pilin Antigenic Variation in Neisseria gonorrhoeae ed. X. Nassif. PLoS Pathog 9: e1003074.
Carlier A L, Omasits U, Ahrens C H, Eberl L. 2013. Proteomics analysis of psychotria leaf nodule symbiosis: Improved genome annotation and metabolic predictions. Mol Plant-Microbe Interact 26: 1325-1333.
Chaitra M G, Shaila M S, Nayak R. 2008. Characterization of T-cell immunogenicity of two PE/PPE proteins of Mycobacterium tuberculosis. J Med Microbiol 57: 1079-86.
Chen J-M, Férec C, Cooper D N. 2010. Gene Conversion in Human Genetic Disease. Genes (Basel) 1: 550-563.
Chin C-S, Alexander D H, Marks P, Klammer A a, Drake J, Heiner C, Clum A, Copeland A, Huddleston J, Eichler E E, et al. 2013. Nonhybrid, finished microbial genome assemblies from long-read SMRT sequencing data. Nat Methods 10: 563-569.
Chiner-Oms Á, Sánchez-Busó L, Corander J, Gagneux S, Harris S R, Young D, González-Candelas F, Comas I. 2019. Genomic determinants of speciation and spread of the Mycobacterium tuberculosis complex. Sci Adv 5: eaaw3307.
Cole S T, Brosch R, Parkhill J, Gamier T, Churcher C, Harris D, Gordon S V, Eiglmeier K, Gas S, Barry C E, et al. 1998. Deciphering the biology of Mycobacterium tuberculosis from the complete genome sequence. Nature 393: 537-44.
Comas I, Hailu E, Kiros T, Bekele S, Mekonnen W, Gumi B, Tschopp R, Ameni G, Hewinson R G, Robertson B D, et al. 2015. Population Genomics of Mycobacterium tuberculosis in Ethiopia Contradicts the Virgin Soil Hypothesis for Human Tuberculosis in Sub-Saharan Africa. Curr Biol.
Copin R, Coscollá M, Seiffert S N, Bothamley G, Sutherland J, Mbayo G, Gagneux S, Ernst J D. 2014. Sequence diversity in the pepgrs genes of Mycobacterium tuberculosis is independent of human T cell recognition. MBio 5: e00960-13.
Danneels B, Pinto-Carbó M, Carlier A. 2018. Patterns of nucleotide deletion and insertion inferred from bacterial pseudogenes. Genome Biol Evol 10: 1792-1802.
Deitsch K W, Lukehart S A, Stringer J R. 2009. Common strategies for antigenic variation by bacterial, fungal and protozoan pathogens. Nat Rev Microbiol 7: 493-503.
DeJesus M A, Gerrick E R, Xu W, Park S W, Long J E, Boutte C C, Rubin E J,
Schnappinger D, Ehrt S, Fortune S M, et al. 2017. Comprehensive Essentiality Analysis of the Mycobacterium tuberculosis Genome via Saturating Transposon Mutagenesis. MBio 8: e02133-16. http://www.ncbi.nlm.nih.gov/pubmed/28096490 (Accessed Jan. 15, 2018).
Domenech P, Kolly G S, Leon-Solis L, Fallow A, Reed M B. 2010. Massive gene duplication event among clinical isolates of the Mycobacterium tuberculosis W/Beijing family. J Bacteriol 192: 4562-70. http://www.ncbi.nlm.nih.gov/pubmed/20639330 (Accessed Feb. 18, 2019).
Eddy S R. 2011. Accelerated Profile HMM Searches. PLoS Comput Biol 7: e1002195. http://www.ncbi.nlm.nih.gov/pubmed/22039361 (Accessed Jan. 3, 2019).
Elghraoui A, Modlin S J, Valafar F. 2017. SMRT genome assembly corrects reference errors, resolving the genetic basis of virulence in Mycobacterium tuberculosis. BMC Genomics 18: 302.
English A C, Salerno W J, Reid J G. 2014. PBHoney: identifying genomic variants via long-read discordance and interrupted mapping. BMC Bioinformatics 15: 180. http://bmcbioinformatics.biomedcentral.com/articles/10.1186/1471-2105-15-180 (Accessed Jun. 3, 2018).
Fang Z, Doig C, Kenna D T, Smittipat N, Palittapongarnpim P, Watt B, Forbes K J. 1999. IS6110-mediated deletions of wild-type chromosomes of Mycobacterium tuberculosis. J Bacteriol 181: 1014-20.
Fang Z, Forbes K J. 1997. A Mycobacterium tuberculosis IS6110 preferential locus (ipl) for insertion into the genome. J Clin Microbiol 35: 479-481.
Finan J E, Archer G L, Pucci M J, Climo M W. 2001. Role of penicillin-binding protein 4 in expression of vancomycin resistance among clinical isolates of oxacillin-resistant Staphylococcus aureus. Antimicrob Agents Chemother.
Fleischmann R D, Alland D, Eisen J A, Carpenter L, White O, Peterson J, DeBoy R,
Dodson R, Gwinn M, Haft D, et al. 2002. Whole-genome comparison of Mycobacterium tuberculosis clinical and laboratory strains. J Bacteriol 184: 5479-90. http://www.ncbi.nlm.nih.gov/pubmed/12218036 (Accessed Jan. 27, 2019).
Francino M P. 2005. An adaptive radiation model for the origin of new gene functions. Nat Genet 37: 573-577.
Fu L, Niu B, Zhu Z, Wu S, Li W. 2012. CD-HIT: Accelerated for clustering the next-generation sequencing data. Bioinformatics 28: 3150-3152.
Gagneux S, Small P M. 2007. Global phylogeography of Mycobacterium tuberculosis and implications for tuberculosis product development. Lancet Infect Dis 7: 328-337.
Gautam S S, et al. 2017. Differential carriage of virulence-associated loci in the New Zealand Rangipo outbreak strain of Mycobacterium tuberculosis. Infect Dis (Auckl) 49: 680-688. https://www.tandfonline.com/doi/full/10.1080/23744235.2017.1330553.
Gellert et al, 1962. Helix formation by guanylic acid. Proc Natl Acad Sci USA 48: 2013-8.
Gene Ontology Consortium. 2017. Expansion of the Gene Ontology knowledgebase and resources. Nucleic Acids Res 45: D331-D338.
Gevers D, et al, 2004. Gene duplication and biased functional retention of paralogs in bacterial genomes. Trends Microbiol 12: 148-154.
Gey van Pittius N C, et al, 2006. Evolution and expansion of the Mycobacterium tuberculosis PE and PPE multigene families and their association with the duplication of the ESAT-6 (esx) gene cluster regions. http://www.ncbi.nlm.nih.gov/pubmed/17105670%5Cnhttp://www.pubmedcentra 1.nih.gov/articlerender.fcgi?artid=PMC1660551.
Gibbs M J, Armstrong J S, Gibbs A J. 2000. Sister-Scanning: a Monte Carlo procedure for assessing signals in recombinant sequences. Bioinformatics 16: 573-582.
Gordon S V., Brosch R, Billault A, Gamier T, Eiglmeier K, Cole S T. 1999.
Identification of variable regions in the genomes of tubercle bacilli using bacterial artificial chromosome arrays. Mol Microbiol 32: 643-655. http://doi.wiley.com/10.1046/j.1365-2958.1999.01383.x (Accessed Oct. 14, 2019).
Grote S. 2018. GOfuncR: Gene ontology enrichment using FDNC.
Hershberg R, et al. 2008. High Functional Diversity in Mycobacterium tuberculosis Driven by Genetic Drift and Human Demography ed. M. J. Blaser. PLoS Biol 6: e311. https://dx.plos.org/10.1371/journal.pbio.0060311 (Accessed Jan. 27, 2019).
Hillery N, et al. 2014. The Global Consortium for Drug-resistant Tuberculosis Diagnostics (GCDD): design of a multi-site, head-to-head study of three rapid tests to detect extensively drug-resistant tuberculosis. Trials 15: 434. http://trialsjournal.biomedcentral.com/articles/10.1186/1745-6215-15-434.
Ho T B L, Robertson B D, Taylor G M, Shaw R J, Young D B. 2000. Comparison of Mycobacterium tuberculosis Genomes Reveals Frequent Deletions in a 20 kb Variable Region in Clinical Isolates. Yeast 1: 272-282.
Huerta-Cepas J, et al, 2017. Fast genome-wide functional annotation through orthology assignment by eggNOG-mapper. Mol Biol Evol.
Huerta-Cepas J, et al. 2019. eggNOG 5.0: a hierarchical, functionally and phylogenetically annotated orthology resource based on 5090 organisms and 2502 viruses. Nucleic Acids Res 47: D309-D314.
Huerta-Cepas J, Szklarczyk D, Heller D, Hernandez-Plaza A, Forslund S K, Cook H, Mende D R, Letunic I, Rattei T, Jensen L J, et al. 2018. eggNOG 5.0: a hierarchical, functionally and phylogenetically annotated orthology resource based on 5090 organisms and 2502 viruses. Nucleic Acids Res 1-6.
Hunt M, Silva N De, Otto T D, Parkhill J, Keane J A, Harris S R. 2015. Circlator: automated circularization of genome assemblies using long sequencing reads. Genome Biol 16: 294. http://genomebiology.com/2015/16/1/294 (Accessed Jun. 3, 2018).
Hyatt D, Chen G-L, Locascio P F, Land M L, Larimer F W, Hauser L J. 2010. Prodigal: prokaryotic gene recognition and translation initiation site identification. BMC Bioinformatics 11: 119. http://www.ncbi.nlm.nih.gov/pubmed/20211023 (Accessed Oct. 27, 2017).
Jespersen M C, Peters B, Nielsen M, Marcatili P. 2017. BepiPred-2.0: Improving sequence-based B-cell epitope prediction using conformational epitopes. Nucleic Acids Res 45: W24-W29.
Jurtz V, Paul S, Andreatta M, Marcatili P, Peters B, Nielsen M. 2017. NetIVII-1Cpan-4.0: Improved Peptide—MHC Class I Interaction Predictions Integrating Eluted Ligand and Peptide Binding Affinity Data. J Immunol199: 3360-3368.
Kapopoulou A, Lew J M, Cole S T. 2011. The MycoBrowser portal: A comprehensive and manually annotated resource for mycobacterial genomes. Tuberculosis 91: 8-13. http://www.ncbi.nlm.nih.gov/pubmed/20980200 (Accessed Feb. 2, 2018).
Karboul A, Van Pittius N C G, Namouchi A, Vincent V, Sola C, Rastogi N, Suffys P, Fabre M, Cataldi A, Huard R C, et al. 2006. Insights into the evolutionary history of tubercle bacilli as disclosed by genetic rearrangements within a PE_PGRS duplicated gene pair. BMC Evol Biol 6: 1-18.
Kato-Maeda M, Ho C, Passarelli B, Banaei N, Grinsdale J, Flores L, Anderson J, Murray M, Rose G, Kawamura L M, et al. 2013. Use of whole genome sequencing to determine the microevolution of Mycobacterium tuberculosis during an outbreak. ed. A. Noymer. PLoS One 8: e58235.
Klingström T, Bongcam-Rudloff E, Pettersson O V. 2018. A comprehensive model of DNA fragmentation for the preservation of High Molecular Weight DNA. bioRxiv.
Koren S, Phillippy A M. 2015. One chromosome, one contig: Complete microbial genomes from long-read sequencing and assembly. Curr Opin Microbiol 23: 110-120. http://dx.doi.org/10.1016/j.mib.2014.11.014.
Koren S, Walenz B P, Berlin K, Miller J R, Bergman N H, Phillippy A M. 2017. Canu: scalable and accurate long-read assembly via adaptive k-mer weighting and repeat separation. Genome Res 27: 722-736. http://www.ncbi.nlm.nih.gov/pubmed/28298431 (Accessed Jun. 3, 2018).
Kuryavyi V, Cahoon L A, Seifert H S, Patel D J. 2012. RecA-binding pilE G4 sequence essential for pilin antigenic variation forms monomeric and 5′ end-stacked dimeric parallel G-quadruplexes. Structure 20: 2090-2102.
Lam H M, Ratmann O, Boni M F. 2018. Improved Algorithmic Complexity for the 3 SEQ Recombination Detection Algorithm. Mol Biol Evol 35: 247-251.
Land M L, Hyatt D, Jun S R, Kora G H, Hauser L J, Lukjancenko O, Ussery D W. 2014. Quality scores for 32,000 genomes. Stand Genomic Sci 9: 1-10.
Laslett D, Canback B. 2004. ARAGORN, a program to detect tRNA genes and tmRNA genes in nucleotide sequences. Nucleic Acids Res 32: 11-6. http://www.ncbi.nlm.nih.gov/pubmed/14704338 (Accessed Jun. 2, 2018).
Lassalle F, P??rian S, Bataillon T, Nesme X, Duret L, Daubin V. 2015. GC-Content evolution in bacterial genomes: the biased gene conversion hypothesis expands. PLoS Genet 11: e1004941.
Lawrence J G. 2005. Common themes in the genome strategies of pathogens. Curr Opin Genet Dev 15: 584-588.
Leinonen R, Sugawara H, Shumway M. 2011. The sequence read archive. Nucleic Acids Res 39: 2010-2012.
Letunic I, Bork P. 2016. Interactive tree of life (iTOL) v3: an online tool for the display and annotation of phylogenetic and other trees. Nucleic Acids Res 44: W242-W245.
Levillain F, et al. 2017. Horizontal acquisition of a hypoxia-responsive molybdenum cofactor biosynthesis pathway contributed to Mycobacterium tuberculosis pathoadaptation. PLoS Pathog 13: 1-24.
Lew JJMJJM, et al. 2011a. TubercuList—10 years after. Tuberculosis 91: 1-7.
Lew JMJJM, et al. 2011b. TubercuList—10 years after. Tuberculosis 91: 1-7.
Li W, Godzik A. 2006. Cd-hit: A fast program for clustering and comparing large sets of protein or nucleotide sequences. Bioinformatics 22: 1658-1659.
Liu X, Gutacker M M, Musser J M, Fu Y X. 2006. Evidence for recombination in Mycobacterium tuberculosis. J Bacteriol 188: 8169-8177.
Lynch M, Conery J S. 2000. The evolutionary fate and consequences of duplicate genes. Science (80-) 290: 1151-1155.
Lynch M, Force A G. 2000. The origin of interspecific genomic incompatibility via gene duplication. Am Nat 156: 590-605.
MacRaild C A, Seow J, Das S C, Norton R S. 2018. Disordered epitopes as peptide vaccines. Pept Sci 110: e24067.
Maisonneuve E, et al. 2011. Bacterial persistence by RNA endonucleases. Proc Natl Acad Sci 108: 13206-13211.
Manavalan B, et al. 2018. iBCE-EL: A New Ensemble Learning Framework for Improved Linear B-Cell Epitope Prediction. Front Immunol 9: 1695.
Marçais G, Delcher A L, Phillippy A M, Coston R, Salzberg S L, Zimin A. 2018. MUMmer4: A fast and versatile genome alignment system. PLoS Comput Biol 14: 1-14.
Martin D, Rybicki E. 2000. RDP: detection of recombination amongst aligned sequences. Bioinformatics 16: 562-563.
Martin D P, et al. 2017. Detecting and Analyzing Genetic Recombination Using RDP4. Methods Mol Biol 1525: 433-460.
Martin D P, Posada D, Crandall K A, Williamson C. 2005. A Modified Bootscan Algorithm for Automated Identification of Recombinant Sequences and Recombination Breakpoints. AIDS Res Hum Retroviruses 21: 98-102.
McLaren W, et al. 2016. The Ensembl Variant Effect Predictor. Genome Biol 17: 122. http://genomebiology.biomedcentral.com/articles/10.1186/s13059-016-0974-4.
Miller M A, Pfeiffer W, Schwartz T. 2010. Creating the CIPRES Science Gateway for inference of large phylogenetic trees. 2010 Gatew Comput Environ Work G C E 2010.
Modlin S J, et al. 2018. Resolving the hypotheticome: annotating M. tuberculosis gene function through bibliomic reconciliation and structural modeling. bioRxiv 358986. https://www.biorxiv.org/content/early/2018/07/03/358986 (Accessed Dec. 1, 2018).
Molle V, et al. 2008. EmbR2, a structural homologue of EmbR, inhibits the Mycobacterium tuberculosis kinase/substrate pair PknH/EmbR. Biochem J410: 309-317.
O'Toole R F, Gautam S S. 2017. Limitations of the Mycobacterium tuberculosis reference genome H37Rv in the detection of virulence-related loci. Genomics 18-21.
Ohno S. 1970. Evolution by Gene Duplication. Springer Berlin Heidelberg, Berlin, Heidelberg, Heidelberg.
Oliphant T E. 2007. Python for Scientific Computing. Comput Sci Eng 9: 10-20.
Otto T D, Dillon G P, Degrave W S, Berriman M. 2011. RATT: Rapid Annotation Transfer Tool. Nucleic Acids Res 39: e57. http://www.ncbi.nlm.nih.gov/pubmed/21306991 (Accessed Oct. 27, 2017).
PacificBiosciences. 2014. Template Preparation and Sequencing Guide.
Padidam M, Sawyer S, Fauquet C M. 1999. Possible emergence of new geminiviruses by frequent recombination. Virology 265: 218-225.
Page A J, et al. 2015. Roary: Rapid large-scale prokaryote pan genome analysis. Bioinformatics 1-3.
Pajón R, Yero D, Lage A, Llanes A, Borroto C J. 2006. Computational identification of beta-barrel outer-membrane proteins in Mycobacterium tuberculosis predicted proteomes as putative vaccine candidates. Tuberculosis 86: 290-302.
Palmer G H, Bankhead T, Seifert H S. 2016. Antigenic Variation in Bacterial Pathogens. Virulence Mech Bact Pathog Fifth Ed 445-480.
Pays E, et al, 1983. Gene conversion as a mechanism for antigenic variation in Trypanosomes. Cell 34: 371-381.
Pearson W R. 2000. Flexible sequence similarity searching with the FASTA3 program package. Methods Mol Biol.
Pérez-Lago L, et al. 2014. Whole genome sequencing analysis of intrapatient microevolution in Mycobacterium tuberculosis: potential impact on the inference of tuberculosis transmission. J Infect Dis 209: 98-108.
Phelan J, et al. 2018. Methylation in Mycobacterium tuberculosis is lineage specific with associated mutations present globally. Sci Rep 8: 1-7. http://dx.doi.org/10.1038/s41598-017-18188-y.
Phelan J E, et al. 2016. Recombination in pe/ppe genes contributes to genetic variation in Mycobacterium tuberculosis lineages. BMC Genomics 17: 151. http://www.pubmedcentral.nih.gov/articlerender.fcgi?artid=4770551&tool=pmce ntrez&rendertype=abstract.
Posada D, Crandall K A. 2001. Evaluation of methods for detecting recombination from DNA sequences: Computer simulations. Proc Natl Acad Sci 98: 13757-13762.
Prister L L, Xu J, Seifert H S. 2019. A Double-Strand Break Does Not Promote Neisseria gonorrhoeae Pilin Antigenic Variation ed. M. J. Federle. J Bacteriol 201: 1-10.
Riley R, Pellegrini M, Eisenberg D. 2008. Identifying Cognate Binding Pairs among a Large Set of Paralogs: The Case of PE/PPE Proteins of Mycobacterium tuberculosis ed. T. Lengauer. PLoS Comput Biol 4: e1000174.
Rodwell T C, Valafar F, Douglas J, Qian L, Garfein R S, Chawla A, Tones J, Zadorozhny V, Soo Kim M, Hoshide M, et al. 2013. Predicting Extensively Drug-resistant Tuberculosis (XDR-TB) Phenotypes with Genetic Mutations. J Clin Microbiol 52: 781-9.
Roetzer A, et al. 2013. Whole Genome Sequencing versus Traditional Genotyping for Investigation of a Mycobacterium tuberculosis Outbreak: A Longitudinal Molecular Epidemiological Study. PLoS Med.
Ross M G, et al, 2013. Characterizing and measuring bias in sequence data. Genome Biol 14: R51. http://genomebiology.biomedcentral.com/articles/10.1186/gb-2013-14-5-r51 (Accessed May 21, 2019).
Rotman E, Steven Seifert H. 2015. Neisseria gonorrhoeae muts affects pilin antigenic variation through mismatch correction and not by pilE guanine quartet binding. J Bacteriol 197: 1828-1838.
Rustad T R, et al. 2014. Mapping and manipulating the Mycobacterium tuberculosis transcriptome using a transcription factor overexpression-derived regulatory network. Genome Biol 15: 502. http://genomebiology.com/2014/15/11/502.
Salzberg S L, et al. 2012. GAGE: A critical evaluation of genome assemblies and assembly algorithms methods sequence data. Genome Res 557-567.
Sampson S L. 2011. Mycobacterial P E/PPE proteins at the host-pathogen interface. Clin Dev Immunol 2011.
Sarojini S, et al. 2005. Identification of moaA3 gene in patient isolates Mycobacterium tuberculosis in Kerala, which is absent in M. tuberculosis H37Rv and H37Ra. BMC Infect Dis 5: 1-7.
Sayes F, et al. 2012. Strong immunogenicity and cross-reactivity of Mycobacterium tuberculosis ESX-5 type VII secretion-encoded PE-PPE proteins predicts vaccine potential. Cell Host Microbe 11: 352-363.
Seemann T. 2014. Prokka: rapid prokaryotic genome annotation. Bioinformatics 30: 2068-2069. http://www.ncbi.nlm.nih.gov/pubmed/24642063 (Accessed Oct. 27, 2017).
Sekar B, et al. 2009. Low frequency of moaA3 gene among the clinical isolates of Mycobacterium tuberculosis from Tamil Nadu and Pondicherry—South eastern coastal states of India. BMC Infect Dis 9: 1-6.
Shabbeer A, et al. 2012. TB-Lineage: An online tool for classification and analysis of strains of Mycobacterium tuberculosis complex. Infect Genet Evol 12: 789-797. http://dx.doi.org/10.1016/j.meegid.2012.02.010.
shitikov E A, et al. 2014. Unusual large-scale chromosomal rearrangements in Mycobacterium tuberculosis Beijing BO/W148 cluster isolates. PLoS One 9: 1-9.
Sievers F, Higgins D G. 2014. Clustal Omega. In Current Protocols in Bioinformatics, pp. 3.13.1-3.13.16, John Wiley & Sons, Inc., Hoboken, N.J., USA.
Singh P, Cole S T. 2011. Mycobacterium leprae: Genes, pseudogenes and genetic diversity. Future Microbiol.
Sinha S, et al. 2005. Immunogenic membrane-associated proteins of Mycobacterium tuberculosis revealed by proteomics. Microbiology 151: 2411-2419.
Smith J M. 1992. Analyzing the mosaic structure of genes. J Mot Evol 34: 126-9.
Soo V W C, Cheng H Y, Kwan B W, Wood T K. 2014. De novo Synthesis of a Bacterial Toxin/Antitoxin System. Sci Rep.
Stamatakis A. 2014. RAxML version 8: a tool for phylogenetic analysis and post-analysis of large phylogenies. Bioinformatics 30: 1312-3.
Stavrum R, Valvatne H, Bø T H, Jonassen I, Hinds J, Butcher P D, Grewal HMS. 2008. Genomic diversity among Beijing and non-Beijing Mycobacterium tuberculosis isolates from Myanmar. PLoS One 3: 1-11.
Stefanova M E, Tomberg J, Davies C, Nicholas R A, Gutheil W G. 2004. Overexpression and enzymatic characterization of Neisseria gonorrhoeae penicillin-binding protein. Eur J Biochem.
Stucki D, et al. 2012. Two new rapid SNP-typing methods for classifying Mycobacterium tuberculosis complex into the main phylogenetic lineages. PLoS One 7.
Swanson J, et al. 1986. Gene conversion involving the pilin structural gene correlates with pilus+ [symbol] pilus—changes in Neisseria gonorrhoeae. Cell 47: 267-276.
Tandon H, et al. 2019. Bioinformatic and mutational studies of related toxin—antitoxin pairs in Mycobacterium tuberculosis predict and identify key functional residues. J Biol Chem 294: 9048-9063.
Van Soolingen D, et al. 1995. Predominance of a single genotype of Mycobacterium tuberculosis in countries of East Asia. J Clin Microbiol.
Vernikos G, et al. 2015. Ten years of pan-genome analyses. Curr Opin Microbiol 23: 148-54.
Vinuesa C G, Chang P P. 2013. Innate B cell helpers reveal novel types of antibody responses. Nat Immunol 14: 119-126.
Virtanen P, Gommers R, Oliphant T E, Haberland M, Reddy T, Cournapeau D,
Burovski E, Peterson P, Weckesser W, Bright J, et al. 2019. SciPy 1.0—Fundamental Algorithms for Scientific Computing in Python.
Vita R, et al. 2019. The Immune Epitope Database (IEDB): 2018 update. Nucleic Acids Res 47: D339-D343.
Walia R, Chaconas G. 2013. Suggested Role for G4 DNA in Recombinational Switching at the Antigenic Variation Locus of the Lyme Disease Spirochete ed. B. Stevenson. PLoS One 8: e57792.
Williams J J, Hergenrother P J. 2012. Artificial activation of toxin—antitoxin systems as an antibacterial strategy. Trends Microbiol 20: 291-298.
Winther K, et al. 2016. VapCs of Mycobacterium tuberculosis cleave RNAs essential for translation. Nucleic Acids Res 44: 9860-9871.
World Health Organization. 2018. Global Tuberculosis Report 2018. Geneva, Switzerland.
Yang J D, et al. 2018a. Mycobacterium tuberculosis-specific CD4+ and CD8+T cells differ in their capacity to recognize infected macrophages. PLoS Pathog 14: 1-30.
Yang T, et al. 2018b. Pan-Genomic Study of Mycobacterium tuberculosis Reflecting the Primary/Secondary Genes, Generality/Individuality, and the Interconversion Through Copy Number Variations. Front Microbiol 9: 1886. https://www.frontiersin.org/article/10.3389/fmicb.2018.01886/full (Accessed Aug. 18, 2018).
Yimer S A, et al. 2016. Deciphering the recent phylogenetic expansion of the originally deeply rooted Mycobacterium tuberculosis lineage 7. BMC Evol Biol 16: 1-10. http://dx.doi.org/10.1186/s12862-016-0715-z.
Zawadzka-Skomial J, et al. 2006. Characterization of the bifunctional glycosyltransferase/acyltransferase penicillin-binding protein 4 of Listeria monocytogenes. J Bacteriol.
Zhang J. 2003. Evolution by gene duplication: an update. Trends Ecol Evol 18: 292-298.
Zhang J R, et al. 1997. Antigenic variation in Lyme disease borreliae by promiscuous recombination of VMP-like sequence cassettes. Cell.
Zheng H, et al. 2008. Genetic Basis of Virulence Attenuation Revealed by Comparative Genomic Analysis of Mycobacterium tuberculosis Strain H37Ra versus H37Rv. PLoS One 3: e2375.
Zhu L, et al. 2015. Precision methylome characterization of Mycobacterium tuberculosis complex (MTBC) using PacBio single-molecule real-time (SMRT) technology. Nucleic Acids Res 1-14.

A number of embodiments of the invention have been described. Nevertheless, it can be understood that various modifications may be made without departing from the spirit and scope of the invention. Accordingly, other embodiments are within the scope of the following claims.

1. A method for treating, preventing or ameliorating infection by a member of the Mycobacterium tuberculosis complex (MTBC), comprising administering to an individual in need thereof at least one inhibitory molecule or inhibitory composition of:
a. the expression or activity of one or more of the 38 Pro-Glu (PE)/Pro-Pro-Glu (PPE) TB or MTBC genes as identified in Table A, or a transcript or a polypeptide encoded by a gene as identified in Table A,
b. the expression or activity of one or more of the 366 identical MTBC genes as identified in Table C, or a transcript or a polypeptide encoded by a gene as identified in Table C; or
c. the antigenic variation of one or more of 52 immune-evading MTBC PE/PPE genes as identified in Table B, or a transcript or a polypeptide encoded by a gene as identified in Table B.

2. The method of claim 1, wherein the inhibitory molecule or composition:
(a) is formulated as a complementary or a sole therapeutic for treating, preventing or ameliorating an MTBC infection;
(b) acts as a NAC (Non-Antibiotic Chemotherapeutic) molecule that inhibits the MTBC PE/PPE genes listed in 1(c) from changing and evading the immune system,
wherein optionally the inhibitory molecule acts as a NAC (Non-Antibiotic Chemotherapeutic) molecule that inhibits the targets listed in claim 1(c) from changing and evading the immune system;
(c) is or comprises an inhibitory small molecule, an inhibitory nucleic acid (optionally the inhibitory nucleic acid comprises a miRNA or an antisense molecule), an inhibitory polypeptide or peptide,
and optionally the inhibitory polypeptide comprises or is an antibody or antigen binding protein capable of specifically binding to a polypeptide encoded by any of the genes listed in 1(a), (b), or (c);
and optionally the inhibitory polypeptide is contained in or is expressed by a phage, and optionally the inhibitory peptide or polypeptide is expressed on the surface of the phage;
and optionally the inhibitory nucleic acid is or comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme.

3-5. (canceled)

6. The method of claim 1, wherein the inhibitory molecule or composition is formulated as a pharmaceutical composition or formulation, or is formulated for administration in vivo; or is formulated for enteral or parenteral administration, or for oral, intramuscular (IM), intravenous (IV) or intrathecal (IT) administration, wherein optionally the pharmaceutical compound or formulation is administered orally, parenterally, by inhalation spray, nasally, topically, intrathecally, intrathecally, intracerebrally, epidurally, intracranially or rectally,
and optionally the inhibitory molecule or composition, or the formulation or pharmaceutical composition, is contained in or on, or expressed on, or is carried in: a nanoparticle, a particle, a micelle or a liposome or lipoplex, a polymersome, a polyplex, a phage or a dendrimer
and optionally the inhibitory molecule or composition, or the formulation or pharmaceutical composition, is formulated as, or contained in or expressed on: a tablet, a pill, a capsule, a gel, a geltab, a liquid, a powder, an emulsion, a lotion, an aerosol, a spray, a lozenge, an aqueous or a sterile or an injectable solution, or an implant,
and optionally the inhibitory nucleic acid is contained in a nucleic acid construct or a chimeric or a recombinant nucleic acid, or an expression cassette, vector, plasmid, phagemid or artificial chromosome, optionally stably integrated into a TB cell's chromosome, or optionally stably episomally expressed in a TB cell.

7-9. (canceled)

10. A kit for or treating, preventing or ameliorating an MTBC infection, comprising an inhibitory molecule or composition used in claim 1, and optionally further comprising instructions for practicing a method of any of the preceding claims.

11-12. (canceled)

13. A method for selecting environmentally-derived or a chimeric genetically engineered phage for formulating a phage or a cocktail of phages that can act as a therapeutic for treating, preventing or ameliorating an MTBC infection,
wherein the environmentally-derived phage or chimeric genetically engineered phage recognizes at least one peptide or a polypeptide encoded by:
(a) one or more of the 38 Pro-Glu (PE)/Pro-Pro-Glu (PPE) TB or M. tuberculosis genes as identified in TABLE A, or a transcript or a polypeptide encoded by a gene as identified in TABLE A,
(b) one or more of the 366 identical MTBC genes as identified in Table C, or a transcript or a polypeptide encoded by a gene as identified in TABLE C; or
(c) one or more of 52 special immune-evading MTBC PE/PPE genes as identified in TABLE B, or a transcript or a polypeptide encoded by a gene as identified in TABLE B,
wherein optionally the at least one peptide or a polypeptide recognized by the environmentally-derived phage or chimeric genetically engineered phage is expressed on the cell surface of an MTBC, or the at least one peptide or a polypeptide recognized by the environmentally-derived or chimeric genetically engineered phage is not expressed on the cell surface of an MTBC bacterium,
and optionally the at least one peptide or a polypeptide is responsible for synthesis and/or localization of surface molecules and targeted by the phage, optionally a mycobacteriophage,
wherein the method comprises:
(i) selecting a set of environmentally-derived or chimeric genetically engineered phages that can attack a desired gene target(s), and/or using the gene targets as a guide to engineer new phages that can target the desired genes,
(ii) combinatorial selecting subsets of the selected phages for therapy,
(iii)
delivering the phage subsets to an MTBC-infected tissue for elimination or amelioration of TB infection, or
delivering the subsets to healthy tissues for prevention of an MTBC infection, and
(iv) selecting the effective or most effective subsets for therapeutic application.

14. The method of claim 13, wherein the environmentally-derived phage or chimeric genetically engineered phage is a lytic phage or a non-lytic phage, optionally a mycobacteriophage, and optionally the phage is formulated as a therapeutic or a pharmaceutical composition, and optionally the therapeutic or a pharmaceutical composition comprises a plurality of phages, or comprises a plurality of environmentally-derived phage or phages, and/or or comprises a plurality of chimeric genetically engineered phage or phages, that are synergistically effective for treating, prevention or ameliorating TB or MTBC infection,
and optionally the phage encodes or comprises a protein toxic to or inhibitory to:
(a) one or more of the 38 Pro-Glu (PE)/Pro-Pro-Glu (PPE) TB genes as identified in TABLE A, or a transcript or a polypeptide encoded by a gene as identified in TABLE A,
(b) one or more of the 366 identical TB genes as identified in Table C, or a transcript or a polypeptide encoded by a gene as identified in TABLE C; or
(c) one or more of 52 special immune-evading TB PE/PPE genes as identified in TABLE B, or a transcript or a polypeptide encoded by a gene as identified in TABLE B,
and optionally the phage is a chimeric phage or chimeric genetically engineered phage, and optionally the chimeric phage or chimeric genetically engineered phage is engineered through phage refactoring,
wherein optionally the chimeric or genetically engineered phage is a lytic chimeric phage comprising a DNA conferring lytic properties and/or a DNA conferring phage-target recognition,
and optionally the DNA conferring lytic capabilities is derived from a lytic mycobacteriophage and the DNA responsible for phage-target recognition is derived from a lysogenic phage\,
and optionally the phage is formulated into a combination therapy of a phage therapeutic and an existing chemotherapeutic.
wherein optionally the mechanism of action of the existing chemotherapeutic and the mechanism of action of the phage act antagonistically with respect to one another.

15-18. (canceled)

19. A method for analyzing sequencing data that can be assembled, de novo, into single contig genomes from DNA isolated from samples of a subspecies, species, collection of species, or genus of interest to identify and prioritize genomic elements as targets for various research and industrial applications where genomic elements' suitability as targets for the application of interest are defined by their essentiality for survival of the organisms,
wherein the method comprises providing or having provided (optionally from publicly available databases) DNA sequencing data, such as that obtained from long-read single molecule sequencing of DNA isolated from a collection of samples of interest isolated for a subspecies, species, collection of species, or genus of interest, and for each sample among the set, the method further comprises:
(a) assembling the DNA sequence or sequences that comprise the genome from the sequencing data without reference to a previously known genome structure,
(b) refining the assembled genome by correcting probable errors through mapping of sequencing reads onto the assembled DNA sequence and correcting the consensus where a majority of mapped reads contradict the consensus sequence; and iteratively repeating this process until convergence (no more consensus corrections) or oscillation between consensus states, or true heterogeneity is otherwise supported. In regions of heterogeneity, alternative consensus sequences are offered representing differences between subpopulations.
(c) inferring coordinates defining genomic elements within the assembled DNA sequence through a hierarchical annotation prioritizing transfer of coordinates from the annotated genome of a related, well-characterized strain, and secondarily integrating annotations assigned through ab initio and/or orthology-based approaches, and
(d) determining the core and accessory sets of genomic elements of the set of genomes under examination.

20. The method of claim 19, further comprising use of Artificial Intelligence (AI), Machine Learning (ML), or statistical pattern recognition to analyze the accessory genome of the genomes under examination and to determine a basis for the accessory genome of any species with an open genome to identify patterns that precede and constitute creation of a novel genomic element (optionally a gene),
and optionally the method comprises identifying mechanisms of new genomic element creation, and thereby identifying which genomic elements are actively creating new elements. These identified genomic elements will create new genomic elements in at least two senses; First, in the sense that their genetic material are used to create the new genomic elements or features through a wide range of processes (for example mutation, duplication, recombination, gene conversion, fusing, splitting, inversion, or some combination thereof); second, in the sense that the ultimate functional products these genomic elements encode (in the case of proteins, for example) or features they comprise (for example, in the case of G4-quadruplexes and other genomic elements with consequences for DNA structure, conformation, or configuration), catalyze, are required for, increase the frequency of events required for, or are otherwise involved in mechanisms of creating new genomic elements. Genomic elements may fulfill both these senses, such as IS6110 movement in M. tuberculosis, wherein the IS6110 element comprises both the DNA sequence being duplicated or transposed and encodes the enzyme catalyzing the requisite biochemical steps for its transposition
and optionally the method comprises predicting upcoming genomic elements and their categorization according to their potential effect on clinical outcome, and/or their clinical utility, wherein potential effects are predicted through one or more of:
a. biological function, as inferred by orthology, homology, or experimentation
b. membership of the predicted element to a mutually exclusive set that includes a member or members with known clinical utility or effect on clinical outcome. This would be taken to imply that the predicted element possesses the same attributes.
c. use metabolic or regulatory modeling to predict the clinical consequence.
and optionally the method comprises use as a prognostic tool and development of preventative therapeutics or measures,
and optionally the genomic elements are analyzed across samples to discern sets of genomic elements defined according to their pattern of variability among the samples comprising: invariably present genomic elements with little to no sequence variability, or invariably present elements with extensive variability in sequence across the entire element or a segment of the element; and optionally elements that are functionally identical, but different in sequence, and collectively invariably present (one is always present), but invariably, or nearly invariably, mutually exclusive within any single genome,
and optionally the method comprises use of a series of sequencing data processing steps that take sequencing data from a collection of samples for a species of interest and returns or identifies a prioritized list of genomic elements as targets for an application of interest, and tailored according to the species and application of interest, by the class of genomic element and type of variability suitable or desired for the application, for example, expression and invariability for antigenic constituents in vaccine formulations; or conservation and essentiality for drug targets, and optionally the prioritized list of genomic elements comprises priority targets for:
(i) intervening in the viability or behavior in a prokaryotic species of interest; or
(ii) rendering a prokaryotic species of interest non-infective either through:
(1) killing the organisms of the species of interest;
(2) rendering the organisms of the species of interest hypersusceptible to common mechanisms of host immunity; or
(3) inoculating the host to the species of interest through exposure to peptides encoded by the identified targets and the molecules they synthesize.
and optionally the prokaryotic species of interest is a bacterial pathogen, and optionally the bacterial pathogen is of the genus Mycobacterium, and optionally a species with the genus Mycobacterium is a member species of the Mycobacterium tuberculosis complex,
and optionally the host of the bacterial pathogen is a human being
and optionally the bacterial pathogen is formulated in or into an inoculum, and optionally the inoculum is or comprises an acellular vaccine, and optionally the inoculum is or comprises a live attenuated vaccine,
and optionally the identified prioritized list of genomic elements comprises a minimal set for engineering viable strains for an industrial application,
and optionally the industrial application is to efficiently or specifically yield a chemical species, and optionally the chemical species comprises or is an antimicrobial compound,
and optionally the industrial application is to efficiently or specifically degrade a chemical species, and optionally the chemical species is or comprises a pollutant, and optionally the pollutant is or comprises a petroleum-derived hydrocarbon.

21-30. (canceled)

31. A vaccine or pharmaceutical formulation for providing immunity against a Mycobacterium tuberculosis complex (MTBC) infection, comprising an immunologically protective dose of an inoculum and a vehicle,
wherein optionally the vehicle is for subdural, intravenous, intranasal, aerosol, or intramuscular delivery (IM), administration to an immunocompetent individual,
wherein the inoculum comprises at least one of:
(a) an acellular vaccine comprising a peptide or a polypeptide encoded by at least one of:
(i) the 38 PE/PPE MTBC genes as identified in TABLE A; or
(ii) the 366 identical MTBC genes as identified in TABLE C; or
(b) a live attenuated vaccine comprising attenuated Mycobacterium tuberculosis (MTB) engineered through functional deletion in the M. tuberculosis (MTB) one or more of:
(i) the 38 PE/PPE MTBC genes as identified in TABLE A; or
(ii) the conserved region of the 52 special immune-evading MTBC PE/PPE genes as identified in TABLE B.

32-37. (canceled)

38. A method for prognosing emergence of new phenotype or phenotype-conferring sequence variations in pathogens for Clinical Decision Support (CDS) comprising the steps of:
a. If a knowledgebase does not yet exist:
(i) Sequencing of unamplified DNA isolated from pathogenic prokaryotic organisms of a species of interest directly from samples of tissue or biological fluid, collected serially from the infected host over time,
(ii) Assembling the sequencing data from each sample, de novo, into one or more complete or partial consensus genomes,
(iii) Calling minority variants within each serially collected sample with respect to the consensus genome(s) assembled from the sample, wherein variants include single or multiple-base polymorphisms, insertions, deletions, inversions, relocations, or translocations, and wherein the position(s) with minority variants are considered as “heterogeneous” when the existence of the minority variant(s) is supported by a number and/or proportion of reads greater than the discordance expected in a homogenous sample,
(iv) Calling variant DNA bases with respect to the consensus genome(s) of prior samples from the patient, wherein variants include single or multiple-base polymorphisms, insertions, deletions, inversions, relocations, or translocations,
(v) Constructing of a catalog of correlations between heterogeneous and consensus variants' with the emergence of new phenotype-conferring sequence variations or new phenotype in subsequent samples,
b. Once a knowledgebase exists:
(i) Sequencing of unamplified DNA isolated from pathogenic prokaryotic organisms of a species of interest directly from a sample or samples of tissue or biological fluid,
(ii) Assembling the sequencing data from each sample, de novo, into one or more complete or partial consensus genomes,
(iii) Calling minority variants within the sample or samples with respect to the consensus genome(s) assembled from the sample, wherein variants include single or multiple-base polymorphisms, insertions, deletions, inversions, relocations, or translocations, and wherein the position(s) with minority variants are considered as “heterogeneous” when the existence of the minority variant(s) is supported by a number and/or proportion of reads greater than the discordance expected in a homogenous sample,
(iv) Calling variant DNA bases with respect to the consensus genome(s) of prior samples from the patient, or from related samples, wherein variants include single or multiple-base polymorphisms, insertions, deletions, inversions, relocations, or translocations,
(vi) Prospectively computing probabilities of emergence of new phenotype-conferring sequence variations or new phenotypes in subsequent samples according to the aforementioned catalog,
(vii) Classifying infections as likely to change phenotype according to the calculated probabilities of future emergent phenotypes and/or new phenotype-conferring sequence variations to classify, over multiple time frames.

39-51. (canceled)
